(19) United States
(12) Patent Application Publication
(10) Pub. No.: US 2025/0011778 A1
(43) Pub. Date: Jan. 9, 2025

(54) APP iRNA Compositions and Methods of Use Thereof for Treating or Preventing Diseases Characterized by Enlarged Endosomes

(71) Applicant: ALNYLAM PHARMACEUTICALS, INC., CAMBRIDGE, MA (US)

(72) Inventors: KIRK BROWN, SWAMPSCOTT, MA (US); LAN THI HOANG DANG, ARLINGTON, MA (US)

(73) Assignee: ALNYLAM PHARMACEUTICALS, INC., CAMBRIDGE, MA (US)

(21) Appl. No.: 18/690,200

(22) PCT Filed: Sep. 9, 2022
(86) PCT No.: PCT/US2022/076159

§ 371 Date: Mar. 7, 2024

Related U.S. Application Data
(60) Provisional application No. 63/345,731, filed on May 25, 2022.
(60) Provisional application No. 63/288,452, filed on Dec. 10, 2021.
(60) Provisional application No. 63/242,798, filed on Sep. 10, 2021.

(51) Int. Cl. C12N 15/113; A61P 25/28; G01N 33/68

(52) U.S. Cl. CPC C12N 15/113; A61P 25/28; G01N 33/6896; C12N 2310/14; G01N 2333/4709; G01N 2800/2821

(57) ABSTRACT

The disclosure relates to use of double stranded ribonucleic acid (dsRNAi) agents and compositions targeting an amyloid precursor protein (APP) gene, including methods of inhibiting expression of an APP gene and methods of treating subjects having a disease or disorder characterized by enlarged endosomes, e.g., Alzheimer's disease (AD) and Down syndrome (DS), particularly occurrences of such neurodegenerative diseases associated with one or more mutations in presenilin 1 (PSEN1), using such dsRNAi agents and compositions.

### CROSS-REFERENCE TO RELATED APPLICATIONS

The present application is related to and claims priority under 35 U.S.C. § 119(e) to U.S. provisional patent application No. 63/242,798, entitled “APP iRNA Compositions and Methods of Use Thereof for Treating or Preventing Diseases Characterized by Enlarged Endosomes,” filed Sep. 10, 2021; to U.S. provisional patent application No. 63/288,452, entitled “APP iRNA Compositions and Methods of Use Thereof for Treating or Preventing Diseases Characterized by Enlarged Endosomes,” filed Dec. 10, 2021; and to U.S. provisional patent application No. 63/345,731, entitled “APP iRNA Compositions and Methods of Use Thereof for Treating or Preventing Diseases Characterized by Enlarged Endosomes,” filed May 25, 2022. The entire content of the aforementioned patent applications are incorporated herein by this reference.

### FIELD OF THE INVENTION

The instant disclosure relates generally to methods involving amyloid precursor protein (APP)-targeting RNAi agents.

### SEQUENCE LISTING

The instant application contains a Sequence Listing which has been filed electronically in eXtensible Markup Language (XML) format and is hereby incorporated by reference in its entirety. Said XML copy, created on Sep. 9, 2022, is named BN00005_0363_ALN_442WO_SL.xml and is 3,663 KB in size.

### BACKGROUND OF THE INVENTION

The amyloid precursor protein (APP) gene encodes an integral membrane protein expressed in neurons and glia. While the primary function of APP is unknown, secretase-cleaved forms of APP—particularly the Aβ cleavage forms of APP, e.g., Aβ(1-42) (aka Aβ42) and Aβ(1-40) (aka Aβ40) commonly found as the predominant protein in amyloid beta plaques—have long been described as associated with the development and progression of Alzheimer's disease (AD) in affected individuals. Altered APP function has also been described as associated with Down syndrome (DS), among other conditions.

Current treatment options for APP-associated diseases and disorders are both limited and largely ineffective. Accordingly, there is a need for therapies for subjects suffering from APP-associated diseases and disorders, including a particular need for therapies for subjects suffering from AD and DS disorders characterized by enlarged neuronal cell early endosomes.

### BRIEF SUMMARY OF THE INVENTION

The present disclosure provides for use of RNAi agent compositions which effect the RNA-induced silencing complex (RISC)-mediated cleavage of RNA transcripts of an amyloid precursor protein (APP) gene, for treatment or prevention of diseases or disorders that are characterized by enlarged endosomes in neuronal cells. The APP gene may be within a cell, e.g., a cell within a subject, such as a human. The present disclosure more specifically provides methods of using the RNAi agent compositions of the disclosure for inhibiting the expression of an APP gene or for treating a subject who would benefit from inhibiting or reducing the expression of an APP gene, e.g., a subject suffering or prone to suffering from an APP-associated neurodegenerative disease or disorder characterized by enlarged neuronal cell endosomes, e.g., Alzheimer's disease (AD) and Down syndrome (DS).

Accordingly, in one aspect, the instant disclosure provides a method for reducing endosome size in a mammalian cell having enlarged endosomes, the method involving contacting the mammalian cell with an amyloid precursor protein (APP)-targeting double stranded ribonucleic acid inhibitory (dsRNAi) agent in an amount sufficient to reduce endosome size in the mammalian cell, thereby reducing endosome size in the mammalian cell.

In certain embodiments, the mammalian cell has a mutation that results in enlarged endosomes. In related embodiments, the mutation that results in enlarged endosomes is a presenilin 1 (PSEN1) mutation or an APP mutation, or a combination thereof. Optionally, the PSEN1 mutation encodes for an amino acid substitution in presenilin 1 polypeptide that is A136G, A231T, A246E, A260V, A275V, A285V, A396T, A409T, A426P, A431E, A434C, A79V, C263R, C410Y, C92S, D333G, ΔD40, ΔE9, ΔI167, ΔI83/M84, ΔL166, ΔS169, ΔT440, E120D, E120K, E123K, E184D, E184G, E273A, E280A, E280G, E318G, F105I, F176L, F237I, F386S, FI77L, G183V, G206A, G206S, G209R, G209V, G217R, G266S, G378E, G378V, G384A, G394V, H131R, H163R, H163Y, H214D, I143T, I143V, I168T, I202F, I213L, I229F, I238M, I437V, I439V, InsR352, K155_insFI, K239N, L113Q, L134R, L150P, L153V, L166P, L171P, L173W, L174M, L219F, L226F, L235P, L235R, L235V, L248R, L250S, L262F, L271V, L282R, L282V, L286V, L381V, L392V, L418F, L420R, L424V, L435F, L85P, M139V, M146L, M146V, M233L, M233T, N135D, N405S, P117A, P264L, P267S, P284S, P436S, Q222R, Q223R, R108Q, R269G, R278K, R352C, R358Q, R35Q, R377W, S169P, S170F, S178P, S212Y, S230I, S365A, S390I, T116N, T147I, T245P, T274R, T291P, T354I, T99A, V261F, V272A, V391F, V412I, V82L, V89L, V94M, V96F, V97L, W165G, Y115H, Y154N, Y256S, or a combination thereof, with residue numbering as in SEQ ID NO: 3 (exemplary Hs PSEN1 polypeptide sequence). Optionally, the PSEN1 mutation encodes for an amino acid substitution in presenilin 1 polypeptide that is M146V, L166P, M233L or A246E, or a combination thereof.

In certain embodiments, the APP mutation encodes for an amino acid substitution in amyloid precursor protein (APP) from among the following: KM670/671NL (Swedish), A673V, D678H (Taiwanese), D678N (Tottori), E682K (Leuven), K687N, F690_V695del, A692G (Flemish), E693del, E693G, E693K, E693Q (Dutch), D694N (Iowa), T714A (Iranian), T714I (Austrian), V715A (German), V715M (French), I716F (Iberian), I716M, I716T, I716V (Florida), V717F (Indiana), V717G, V717I (London), V717L, T719N, T719P, M722K, L723P (Australian), and K724N (Belgian), or a combination thereof, with residue numbering as in SEQ ID NO: 12 (exemplary Hs APP polypeptide sequence). Optionally, the APP mutation encodes for an amino acid substitution in amyloid precursor protein that is KM670/671NL (Swedish), A692G or V717G, or a combination thereof.

In one embodiment, the mammalian cell is homozygous for the mutation that results in enlarged endosomes.

In another embodiment, the mammalian cell is a neuronal cell. Optionally, the mammalian cell is a human neuronal cell. Optionally, the mammalian cell is a human induced pluripotent stem cell (iPSC)-derived neuron.

In certain embodiments, the amount of dsRNAi agent sufficient to reduce endosome size in the mammalian cell is less than 10 nM in the environment of the cell. Optionally, the amount of dsRNAi agent sufficient to reduce endosome size in the mammalian cell is less than 1 nM in the environment of the cell. Optionally, the amount of dsRNAi agent sufficient to reduce endosome size in the mammalian cell is less than 0.1 nM in the environment of the cell.

In some embodiments, average endosome size in the mammalian cell contacted with the dsRNAi agent is reduced by at least 30%, as compared to a mammalian cell in the absence of the dsRNAi agent. Optionally, average endosome size in the mammalian cell contacted with the dsRNAi agent is reduced by at least 50%, as compared to a mammalian cell in the absence of the dsRNAi agent.

In embodiments, the amount of dsRNAi agent is sufficient to reduce the average endosome size in a mammalian cell by at least 30%, as compared to a mammalian cell in the absence of the dsRNAi agent. Optionally, the amount of dsRNAi agent sufficient to reduce the average endosome size in a mammalian cell by at least 50%, as compared to a mammalian cell in the absence of the dsRNAi agent. Optionally, endosome size is assayed via immunofluorescent imaging of Rab5.

In one embodiment, endosome size is determined by detecting the size of Rab5-containing intracellular compartments in the mammalian cell. Optionally, the size of Rab5-containing intracellular compartments is determined via immunofluorescent imaging of Rab5.

In another embodiment, the level of one or more APP C-terminal fragment (CTF), α-CTF and/or β-CTF, is reduced in the contacted mammalian cell, as compared to an appropriate control mammalian cell.

In certain embodiments, the amount of dsRNAi agent is sufficient to reduce β-CTF levels in a mammalian cell by at least 30%, as compared to a mammalian cell in the absence of the dsRNAi agent. Optionally, the amount of dsRNAi agent is sufficient to reduce β-CTF levels in a mammalian cell by at least 50%, as compared to a mammalian cell in the absence of the dsRNAi agent.

In some embodiments, the dsRNAi agent is selected from Tables 2-21.

In one embodiment, the mammalian cell is within a subject. In a related embodiment, the subject is a human. In an alternative embodiment, the subject is a rhesus monkey, a cynomolgous monkey, a mouse, or a rat.

In certain embodiments, the human subject suffers from an APP-associated disorder characterized by enlarged neuronal cell endosomes. Optionally, the human subject suffers from Alzheimer's disease (AD) or Down syndrome (DS). In a related embodiment, the APP-associated disorder characterized by enlarged neuronal cell endosomes is AD. Optionally, the APP-associated disorder characterized by enlarged neuronal cell endosomes is early onset familial AD (EOFAD).

In an embodiment, APP expression is reduced by at least about 30% in the cell administered the APP-targeting dsRNAi agent. Optionally, APP expression is reduced by at least about 50% in the cell administered the APP-targeting dsRNAi agent. Optionally, APP expression is reduced by at least about 80% in the cell administered the APP-targeting dsRNAi agent.

Another aspect of the instant disclosure provides a method for identifying a subject as having or at risk of developing a disease or disorder characterized by enlarged endosomes in neuronal cells and selecting a treatment for the subject, the method involving: a) obtaining a nucleic acid sample from the subject; b) identifying the subject as having a mutation in presenilin 1 (PSEN1) or amyloid precursor protein (APP) associated with enlargement of endosomes in neuronal cells having the PSEN1 or APP mutation; and c) selecting an amyloid precursor protein (APP)-targeting double stranded ribonucleic acid inhibitory (dsRNAi) agent for administration to the subject in an amount sufficient to reduce APP levels in neuronal cells of the subject, thereby identifying the subject as having or at risk of developing a disease or disorder characterized by enlarged endosomes in neuronal cells and selecting a treatment for the subject.

In one embodiment, the disease or disorder characterized by enlarged endosomes in neuronal cells is Alzheimer's disease (AD), Down syndrome (DS) or frontotemporal dementia (FTD). Optionally, the AD is early onset familial AD (EOFAD).

In certain embodiments, the mutation in PSEN1 or APP associated with enlargement of endosomes in neuronal cells having the PSEN1 or APP mutation is a PSEN1 mutation. Optionally, the PSEN1 mutation encodes for one or more amino acid substitution in presenilin 1 polypeptide selected from M146V, L166P and A246E, including combinations thereof.

In some embodiments, the mutation in PSEN1 or APP associated with enlargement of endosomes in neuronal cells having the PSEN1 or APP mutation is an APP mutation. Optionally, the APP mutation encodes for one or more amino acid substitution in amyloid precursor protein selected from KM670/671NL (Swedish), A673V, D678H (Taiwanese), D678N (Tottori), E682K (Leuven), K687N, F690_V695del, A692G (Flemish), E693del, E693G, E693K, E693Q (Dutch), D694N (Iowa), T714A (Iranian), T714I (Austrian), V715A (German), V715M (French), I716F (Iberian), I716M, I716T, I716V (Florida), V717F (Indiana), V717G, V717I (London), V717L, T719N, T719P, M722K, L723P (Australian), and K724N (Belgian), including combinations thereof. Optionally, the APP mutation encodes for one or more amino acid substitution in amyloid precursor protein selected from of KM670/671NL (Swedish), A692G and V717G, including combinations thereof.

In one embodiment, the subject is homozygous for the mutation in PSEN1 or APP.

In a further embodiment, the method also involves administering the selected APP-targeting dsRNAi agent to the subject.

In a related embodiment, endosome size in neuronal cells of the subject administered the selected APP-targeting dsRNAi agent is reduced, as compared to an appropriate control and/or an untreated subject. Optionally, average endosome size in neuronal cells of the subject administered the selected APP-targeting dsRNAi agent is reduced by at least 30%, as compared to an appropriate control and/or an untreated subject. Optionally, average endosome size in neuronal cells of the subject administered the selected APP-targeting dsRNAi agent is reduced by at least 50%, as compared to an appropriate control and/or an untreated subject.

In some embodiments, endosome size is determined by detecting the size of Rab5-containing intracellular compartments in the neuronal cells of the subject. Optionally, the size of Rab5-containing intracellular compartments is determined via immunofluorescent imaging of Rab5.

In an embodiment, synaptic transmission of neuronal cells of the subject administered the selected APP-targeting dsRNAi agent is improved, as compared to an appropriate control and/or an untreated subject.

In another embodiment, a symptom of AD or DS, such as short-term memory or cognition (or both) is improved in the subject administered the selected APP-targeting dsRNAi agent, as compared to an appropriate control and/or an untreated subject.

In some embodiments, the dose of the selected APP-targeting dsRNAi agent sufficient to reduce APP levels in neuronal cells of the subject is a dose of about 0.01 mg/kg to about 50 mg/kg. Optionally, the dose of the selected APP-targeting dsRNAi agent sufficient to reduce APP levels in neuronal cells of the subject is a dose of about 2-10 mg/kg.

In one embodiment, the level of one or more of the APP C-terminal fragment (CTF) polypeptides α-CTF and β-CTF is reduced in the subject administered the selected APP-targeting dsRNAi agent, as compared to an appropriate control and/or an untreated subject.

In another embodiment, the method further involves administering an additional therapeutic agent to the subject.

In certain embodiments, the double stranded RNAi agent is administered to the subject intrathecally.

In some embodiments, APP expression is reduced by at least about 30% in the subject administered the APP-targeting dsRNAi agent. Optionally, APP expression is reduced by at least about 50% in the subject administered the APP-targeting dsRNAi agent. Optionally, APP expression is reduced by at least about 80% in the subject administered the APP-targeting dsRNAi agent.

Another aspect of the instant disclosure provides a method for identifying a subject as having a disease or disorder characterized by enlarged endosomes in neuronal cells and selecting a treatment for the subject, the method involving: a) obtaining a neuronal cell sample or fluid sample from a neuronal cell environment of the subject; b) identifying the subject as having elevated β-CTF levels in the neuronal cell or neuronal cell-proximate fluid sample as an indicator for enlarged endosomes in neuronal cells of the subject; and c) selecting an amyloid precursor protein (APP)-targeting double stranded ribonucleic acid inhibitory (dsRNAi) agent for administration to the subject in an amount sufficient to reduce β-CTF levels in neuronal cells of the subject, thereby identifying a subject as having a disease or disorder characterized by enlarged endosomes in neuronal cells and selecting a treatment for the subject.

In certain embodiments, the neuronal cell sample or fluid sample from the neuronal cell environment is obtained from the central nervous system or peripheral nervous system of the subject.

In some embodiments, the method further involves administering the selected APP-targeting dsRNAi agent to the subject.

In one embodiment, average endosome size in neuronal cells of the subject administered the selected APP-targeting dsRNAi agent is reduced by at least 30%, as compared to an appropriate control and/or an untreated subject. Optionally, average endosome size in neuronal cells of the subject administered the selected APP-targeting dsRNAi agent is reduced by at least 50%, as compared to an appropriate control and/or an untreated subject.

In certain embodiments, synaptic transmission of neuronal cells of the subject administered the selected APP-targeting dsRNAi agent is improved, as compared to an appropriate control and/or an untreated subject.

In one embodiment, a symptom of AD or DS, such as short-term memory and/or cognition is improved in the subject administered the selected APP-targeting dsRNAi agent, as compared to an appropriate control and/or an untreated subject.

In embodiments, the dose of the selected APP-targeting dsRNAi agent sufficient to reduce β-CTF levels in neuronal cells of the subject is a dose of about 0.01 mg/kg to about 50 mg/kg.

Optionally, the dose is a dose of about 2-10 mg/kg.

In some embodiments, the level of one or more of the APP C-terminal fragments (CTF) α-CTF and β-CTF is reduced in the subject administered the selected APP-targeting dsRNAi agent, as compared to an appropriate control and/or an untreated subject.

In an embodiment, the method further involves administering an additional therapeutic agent to the subject.

In one embodiment, the double stranded RNAi agent is administered to the subject intrathecally.

In certain embodiments, APP expression is reduced by at least about 30% in the subject administered the APP-targeting dsRNAi agent. Optionally, APP expression is reduced by at least about 50% in the subject administered the APP-targeting dsRNAi agent. Optionally, APP expression is reduced by at least about 80% in the subject administered the APP-targeting dsRNAi agent.

In another embodiment, the level of β-CTF is reduced in the subject administered the selected APP-targeting dsRNAi agent, as compared to an appropriate control and/or an untreated subject.

In a further aspect, the instant disclosure provides a method for reducing inflammation in a subject having or at risk of developing Alzheimer's Disease (AD), the method involving administering to the subject an amyloid precursor protein (APP)-targeting double stranded ribonucleic acid inhibitory (dsRNAi) agent in an amount sufficient to reduce inflammation in the subject. For example, reducing inflammation includes, but is not limited to reducing expression of Iba1 mRNA. Reducing expression of Iba1 mRNA may be within the CNS of a patient in need of such treatment.

### Definitions

That the present disclosure may be more readily understood, certain terms are first defined. In addition, it should be noted that whenever a value or range of values of a parameter are recited, it is intended that values and ranges intermediate to the recited values are also intended to be part of this disclosure.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise.

The term “about” is used herein to mean within the typical ranges of tolerances in the art. For example, “about” can be understood as about 2 standard deviations from the mean. In certain embodiments, about means ±10%. In certain embodiments, about means ±5%. When about is present before a series of numbers or a range, it is understood that “about” can modify each of the numbers in the series or range.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleotides of a 21 nucleotide nucleic acid molecule” means that 18, 19, 20, or 21 nucleotides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, a duplex with an overhang of “no more than 2 nucleotides” has a 2, 1, or 0 nucleotide overhang. When “no more than” is present before a series of numbers or a range, it is understood that “no more than” can modify each of the numbers in the series or range. As used herein, ranges include both the upper and lower limit.

As used herein, methods of detection can include determination that the amount of analyte present is below the level of detection of the method.

In the event of a conflict between an indicated target site and the nucleotide sequence for a sense or antisense strand, the indicated sequence takes precedence.

In the event of a conflict between a chemical structure and a chemical name, the chemical structure takes precedence.

The term “PSEN1” refers to presenilin 1 (PSEN1), also known as PS1, S182, FAD, Alzheimer Disease 3 (ΔD3), Presenilin-1, PS-1, Protein S182, EC 3.4.23-, EC 3.4.23⁵⁰, ACNINV3 and PSNL1, among other names, having an amino acid sequence from any vertebrate or mammalian source, including, but not limited to, human, bovine, chicken, rodent, mouse, rat, porcine, ovine, primate, monkey, and guinea pig, unless specified otherwise. The term also refers to fragments and variants of native PSEN1 that maintain at least one in vivo or in vitro activity of a native PSEN1. The nucleotide and amino acid sequence of a human PSEN1 can be found at, for example, NM_000021.4 (SEQ ID NOs: 1 and 2 (reverse complement)) and NP_000012.1, SEQ ID NO: 3). The nucleotide and amino acid sequence of a human APP may also be found at, for example, NM_007318.3 (SEQ ID NOs: 4 and 5 (reverse complement)) and NP_015557.2 (SEQ ID NO: 6).

The nucleotide and amino acid sequence of a Cynomolgus monkey PSEN1 can be found at, for example, GenBank Accession No. GI: 148717220 (AB083326.2, SEQ ID NOs: 2940 and 2941 (reverse complement), and BAC20605.1, SEQ ID NO: 2942). The nucleotide and amino acid sequence of a mouse PSEN1 can be found at, for example, GenBank Accession No. GI: 8131957 (AF149111.1, SEQ ID NOs: 2943 and 2944 (reverse complement), and AAF73153.1, SEQ ID NO: 2945). The nucleotide and amino acid sequence of a rat APP can be found at, for example, GenBank Accession No. GI: 1777325 (D82363.1, SEQ ID NOs: 2946 and 2947 (reverse complement), and BAA11564.1, SEQ ID NO: 2948). Additional examples of PSEN1 sequences are readily available using publicly available databases, e.g., GenBank, UniProt, and OMIM.

The term “PSEN1” as used herein also refers to a particular polypeptide expressed in a cell by naturally occurring DNA sequence variations of the PSEN1 gene, such as a single nucleotide polymorphism in the PSEN1 gene. Numerous SNPs within the PSEN1 gene have been identified and may be found at, for example, NCBI dbSNP (see, e.g., www.ncbi.nlm.nih.gov/snp?LinkName=gene_snp&from_uid=5663, the entire contents of which is incorporated herein by reference as of the date of filing this application). Non-limiting examples of SNPs within the PSEN1 gene may be found at NCBI dbSNP, particularly noting PSEN1 variants that produce the following presenilin-1 polypeptide mutations: A136G, A231T, A246E, A260V, A275V, A285V, A396T, A409T, A426P, A431E, A434C, A79V, C263R, C410Y, C92S, D333G, ΔD40, ΔE9, ΔI167, ΔI83/M84, ΔL166, ΔS169, ΔT440, E120D, E120K, E123K, E184D, E184G, E273A, E280A, E280G, E318G, F105I, F176L, F237I, F386S, FI77L, G183V, G206A, G206S, G209R, G209V, G217R, G266S, G378E, G378V, G384A, G394V, H131R, H163R, H163Y, H214D, I143T, I143V, I168T, I202F, I213L, I229F, I238M, I437V, I439V, InsR352, K155_insFI, K239N, L113Q, L134R, L150P, L153V, L166P, L171P, L173W, L174M, L219F, L226F, L235P, L235R, L235V, L248R, L250S, L262F, L271V, L282R, L282V, L286V, L381V, L392V, L418F, L420R, L424V, L435F, L85P, M139V, M146L, M146V, M233L, M233T, N135D, N405S, P117A, P264L, P267S, P284S, P436S, Q222R, Q223R, R108Q, R269G, R278K, R352C, R358Q, R35Q, R377W, S169P, S170F, S178P, S212Y, S230I, S365A, S390I, T116N, T147I, T245P, T274R, T291P, T354I, T99A, V261F, V272A, V391F, V412I, V82L, V89L, V94M, V96F, V97L, W165G, Y115H, Y154N, Y256S, as well as combinations thereof, with residue numbering as in SEQ ID NO: 3.

The term “APP” refers to amyloid precursor protein (APP), also known as amyloid beta precursor protein, Alzheimer disease amyloid protein and cerebral vascular amyloid peptide, among other names, having an amino acid sequence from any vertebrate or mammalian source, including, but not limited to, human, bovine, chicken, rodent, mouse, rat, porcine, ovine, primate, monkey, and guinea pig, unless specified otherwise. The term also refers to fragments and variants of native APP that maintain at least one in vivo or in vitro activity of a native APP (including, e.g., the beta-amyloid peptide(1-40), beta-amyloid peptide(1-38) and beta-amyloid peptide(1-42) forms of Aβ peptide, among others), including variants of APP fragments that maintain one or more activities of an APP fragment that are neurotoxic in character (e.g., variant forms of Aβ42 peptide that maintain neurotoxic character are expressly contemplated). The term encompasses full-length unprocessed precursor forms of APP as well as mature forms resulting from post-translational cleavage of the signal peptide. The term also encompasses peptides that derive from APP via further cleavage, including, e.g., Aβ peptides. The nucleotide and amino acid sequence of a human APP can be found at, for example, GenBank Accession No. GI: 228008405 (NM 201414, SEQ ID NOs: 7 and 8 (reverse complement), and NP_958817, SEQ ID NO: 9). The nucleotide and amino acid sequence of a human APP may also be found at, for example, GenBank Accession No. GI: 228008403 (NM_000484.4, SEQ ID NOs: 10 and 11 (reverse complement), and NP_000475, SEQ ID NO: 12); GenBank Accession No. GI: 228008404 (NM 201413.3, SEQ ID NOs: 13 and 14 (reverse complement), and NP_958816, SEQ ID NO: 15); GenBank Accession No. GI: 324021746 (NM_001136016.3, SEQ ID NOs: 16 and 17 (reverse complement), and NP_001129488, SEQ ID NO: 18); GenBank Accession No. GI: 228008402 (NM_001136129.3, SEQ ID NOs: 19 and 20 (reverse complement), and NP_001129601, SEQ ID NO: 21); GenBank Accession No. GI: 228008401 (NM_001136130.3, SEQ ID NOs: 22 and 23 (reverse complement), and NP_001129602, SEQ ID NO: 24); GenBank Accession No. GI: 324021747 (NM_001136131.3, SEQ ID NOs: 25 and 26 (reverse complement), and NP_001129603, SEQ ID NO: 27); GenBank Accession No. GI: 324021737 (NM_001204301.2, SEQ ID NOs: 28 and 29 (reverse complement), and NP_001191230, SEQ ID NO: 30); GenBank Accession No. GI: 324021735 (NM_001204302.2, SEQ ID NOs: 31 and 32 (reverse complement), and NP_001191231, SEQ ID NO: 33); GenBank Accession No. GI: 324021739 (NM_001204303.2, SEQ ID NOs: 34 and 35 (reverse complement), and NP_001191232, SEQ ID NO: 36); and GenBank Accession No. GI: 1370481385 (XM_024452075.1, SEQ ID NOs: 37 and 38 (reverse complement), and XP_024307843, SEQ ID NO: 39).

The nucleotide and amino acid sequence of a Cynomolgus monkey APP can be found at, for example, GenBank Accession No. GI: 982237868 (XM_005548883.2, SEQ ID NOs: 40 and 41 (reverse complement), and XP_005548940, SEQ ID NO: 42). The nucleotide and amino acid sequence of a mouse APP can be found at, for example, GenBank Accession No. GI: 311893400 (NM_001198823, SEQ ID NOs: 43 and 44 (reverse complement), and NP_001185752, SEQ ID NO: 45). The nucleotide and amino acid sequence of a rat APP can be found at, for example, GenBank Accession No. GI: 402692725 (NM_019288.2, SEQ ID NOs: 46 and 47 (reverse complement), and NP_062161, SEQ ID NO: 48). Additional examples of APP sequences are readily available using publicly available databases, e.g., GenBank, UniProt, and OMIM.

The term “APP” as used herein also refers to a particular polypeptide expressed in a cell by naturally occurring DNA sequence variations of the APP gene, such as a single nucleotide polymorphism in the APP gene. Numerous SNPs within the APP gene have been identified and may be found at, for example, NCBI dbSNP (see, e.g., www.ncbi.nlm.nih.gov/snp?LinkName=gene_snp&from_uid=351, the entire contents of which is incorporated herein by reference as of the date of filing this application). Non-limiting examples of SNPs within the APP gene may be found at, NCBI dbSNP Accession Nos. rs193922916, rs145564988, rs193922916, rs214484, rs281865161, rs364048, rs466433, rs466448, rs532876832, rs63749810, rs63749964, rs63750064, rs63750066, rs63750151, rs63750264, rs63750363, rs63750399, rs63750445, rs63750579, rs63750643, rs63750671, rs63750734, rs63750847, rs63750851, rs63750868, rs63750921, rs63750973, rs63751039, rs63751122 and rs63751263. Certain exemplary rare APP variants that have been previously described to play a role in development of EOFAD were identified in Hooli et al. (Neurology 78: 1250-57). In addition, various “non-classical” APP variants that harbor an intraexonic junction within sequenced cDNA have recently been identified as associated with the occurrence of somatic gene recombination in the brains of AD patients (PCT/US2018/030520, which is incorporated herein by reference in its entirety). Examples of such “non-classical” APP variants include cAPP-R3/16 (SEQ ID NO: 49), cAPP-R3/16-2 (SEQ ID NO: 50), cAPP-R2/18 (SEQ ID NO: 51), cAPP-R6/18 (SEQ ID NO: 52), cAPP-R3/14 (SEQ ID NO: 53), cAPP-R3/17 (SEQ ID NO: 54), cAPP-R1/11 (SEQ ID NO: 55), cAPP-R1/13 (SEQ ID NO: 56), cAPP-R1/11-2 (SEQ ID NO: 57), cAPP-R1/14 (SEQ ID NO: 58), cAPP-R2/17 (SEQ ID NO: 59), cAPP-R2/16 (SEQ ID NO: 60), cAPP-R6/17 (SEQ ID NO: 61), cAPP-R2/14 (SEQ ID NO: 62), cAPP-R14/17-d8 (SEQ ID NO: 63) and cAPP-D2/18-3 (SEQ ID NO: 64). It is expressly contemplated that RNAi agents of the instant disclosure can be used to target “non-classical” APP variants and/or that RNAi agents optionally specific for such “non-classical” APP variants can be designed and used, optionally in combination with other RNAi agents of the instant disclosure, including those that target native forms of APP. Such “non-classical” APP variants were described as notably absent from an assayed HIV patient population, with prevalence of AD in the HIV patient population significantly diminished as compared to expected levels, which indicated that reverse transcriptase inhibitors and/or other anti-retroviral therapies commonly used to treat HIV patients likely also exerted a therapeutic/preventative role against AD. It is therefore expressly contemplated that the RNAi agents of the instant disclosure can optionally be employed in combination with reverse transcriptase inhibitors and/or other anti-retroviral therapies, for therapeutic and/or preventative purposes.

The entire contents of each of the foregoing GenBank Accession numbers and the Gene database numbers are incorporated herein by reference as of the date of filing this application.

As used herein, “target sequence” refers to a contiguous portion of the nucleotide sequence of an mRNA molecule formed during the transcription of an APP gene, including mRNA that is a product of RNA processing of a primary transcription product. In one embodiment, the target portion of the sequence will be at least long enough to serve as a substrate for RNAi-directed cleavage at or near that portion of the nucleotide sequence of an mRNA molecule formed during the transcription of an APP gene. In one embodiment, the target sequence is within the protein coding region of the APP gene. In another embodiment, the target sequence is within the 3′ UTR of the APP gene.

The target sequence may be from about 9-36 nucleotides in length, e.g., about 15-30 nucleotides in length. For example, the target sequence can be from about 15-30 nucleotides, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length. In some embodiments, the target sequence is about 19 to about 30 nucleotides in length. In other embodiments, the target sequence is about 19 to about 25 nucleotides in length. In still other embodiments, the target sequence is about 19 to about 23 nucleotides in length. In some embodiments, the target sequence is about 21 to about 23 nucleotides in length. Ranges and lengths intermediate to the above recited ranges and lengths are also contemplated to be part of the disclosure.

As used herein, the term “strand comprising a sequence” refers to an oligonucleotide comprising a chain of nucleotides that is described by the sequence referred to using the standard nucleotide nomenclature.

“G,” “C,” “A,” “T”, and “U” each generally stand for a nucleotide that contains guanine, cytosine, adenine, thymidine, and uracil as a base, respectively in the context of a modified or unmodified nucleotide. However, it will be understood that the term “ribonucleotide” or “nucleotide” can also refer to a modified nucleotide, as further detailed below, or a surrogate replacement moiety (see, e.g., Table 1). The skilled person is well aware that guanine, cytosine, adenine, thymidine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the disclosure by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the disclosure.

The terms “iRNA”, “RNAi agent,” “iRNA agent,” “RNA interference agent” as used interchangeably herein, refer to an agent that contains RNA as that term is defined herein, and which mediates the targeted cleavage of an RNA transcript via an RNA-induced silencing complex (RISC) pathway. RNA interference (RNAi) is a process that directs the sequence-specific degradation of mRNA. RNAi modulates, e.g., inhibits, the expression of APP in a cell, e.g., a cell within a subject, such as a mammalian subject.

In one embodiment, an RNAi agent of the disclosure includes a single stranded RNAi that interacts with a target RNA sequence, e.g., an APP target mRNA sequence, to direct the cleavage of the target RNA. Without wishing to be bound by theory it is believed that long double stranded RNA introduced into cells is broken down into double-stranded short interfering RNAs (siRNAs) comprising a sense strand and an antisense strand by a Type III endonuclease known as Dicer (Sharp et al. (2001) Genes Dev. 15: 485). Dicer, a ribonuclease-III-like enzyme, processes these dsRNA into 19-23 base pair short interfering RNAs with characteristic two base 3′ overhangs (Bernstein, et al., (2001) Nature 409: 363). These siRNAs are then incorporated into an RNA-induced silencing complex (RISC) where one or more helicases unwind the siRNA duplex, enabling the complementary antisense strand to guide target recognition (Nykanen, et al., (2001) Cell 107: 309). Upon binding to the appropriate target mRNA, one or more endonucleases within the RISC cleave the target to induce silencing (Elbashir, et al., (2001) Genes Dev. 15: 188). Thus, in one aspect the disclosure relates to a single stranded RNA (ssRNA) (the antisense strand of a siRNA duplex) generated within a cell and which promotes the formation of a RISC complex to effect silencing of the target gene, i.e., an APP gene. Accordingly, the term “siRNA” is also used herein to refer to an RNAi as described above.

In another embodiment, the RNAi agent may be a single-stranded RNA that is introduced into a cell or organism to inhibit a target mRNA. Single-stranded RNAi agents bind to the RISC endonuclease, Argonaute 2, which then cleaves the target mRNA. The single-stranded siRNAs are generally 15-30 nucleotides and are chemically modified. The design and testing of single-stranded RNAs are described in U.S. Pat. No. 8,101,348 and in Lima et al., (2012) Cell 150: 883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150: 883-894.

In another embodiment, a “RNAi agent” for use in the compositions and methods of the disclosure is a double stranded RNA and is referred to herein as a “double stranded RNAi agent,” “double stranded RNA (dsRNA) molecule,” “dsRNA agent,” or “dsRNA”. The term “dsRNA” refers to a complex of ribonucleic acid molecules, having a duplex structure comprising two anti-parallel and substantially complementary nucleic acid strands, referred to as having “sense” and “antisense” orientations with respect to a target RNA, i.e., an APP gene. In some embodiments of the disclosure, a double stranded RNA (dsRNA) triggers the degradation of a target RNA, e.g., an mRNA, through a post-transcriptional gene-silencing mechanism referred to herein as RNA interference or RNAi.

In general, a dsRNA molecule can include ribonucleotides, but as described in detail herein, each or both strands can also include one or more non-ribonucleotides, e.g., a deoxyribonucleotide, a modified nucleotide. In addition, as used in this specification, an “RNAi agent” may include ribonucleotides with chemical modifications; an RNAi agent may include substantial modifications at multiple nucleotides.

As used herein, the term “modified nucleotide” refers to a nucleotide having, independently, a modified sugar moiety, a modified internucleotide linkage, or a modified nucleobase. Thus, the term modified nucleotide encompasses substitutions, additions or removal of, e.g., a functional group or atom, to internucleoside linkages, sugar moieties, or nucleobases. The modifications suitable for use in the agents of the disclosure include all types of modifications disclosed herein or known in the art. Any such modifications, as used in a siRNA type molecule, are encompassed by “RNAi agent” for the purposes of this specification and claims.

In certain embodiments of the instant disclosure, inclusion of a deoxy-nucleotide—which is acknowledged as a naturally occurring form of nucleotide—if present within an RNAi agent can be considered to constitute a modified nucleotide.

The duplex region may be of any length that permits specific degradation of a desired target RNA through a RISC pathway, and may range from about 9 to 36 base pairs in length, e.g., about 15-30 base pairs in length, for example, about 9, 10, 11, 12, 13, 14, 15, 16, 17, 18, 19, 20, 21, 22, 23, 24, 25, 26, 27, 28, 29, 30, 31, 32, 33, 34, 35, or 36 base pairs in length, such as about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. Ranges and lengths intermediate to the above recited ranges and lengths are also contemplated to be part of the invention.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” A hairpin loop can comprise at least one unpaired nucleotide. In some embodiments, the hairpin loop can comprise at at least 4, at least 5, at least 6, at least 7, at least 8, at least 9, at least 10, at least 20, at least 23 or more unpaired nucleotides or nucleotides not directed to the target site of the dsRNA. In some embodiments, the hairpin loop can be 10 or fewer nucleotides. In some embodiments, the hairpin loop can be 8 or fewer unpaired nucleotides. In some embodiments, the hairpin loop can be 4-10 unpaired nucleotides. In some embodiments, the hairpin loop can be 4-8 nucleotides.

In certain embodiments, the two strands of double-stranded oligomeric compound can be linked together. The two strands can be linked to each other at both ends, or at one end only. By linking at one end is meant that 5′-end of first strand is linked to the 3′-end of the second strand or 3′-end of first strand is linked to 5′-end of the second strand. When the two strands are linked to each other at both ends, 5′-end of first strand is linked to 3′-end of second strand and 3′-end of first strand is linked to 5′-end of second strand. The two strands can be linked together by an oligonucleotide linker including, but not limited to, (N)n; wherein N is independently a modified or unmodified nucleotide and n is 3-23. In some embodiments, n is 3-10, e.g., 3, 4, 5, 6, 7, 8, 9, or 10. In some embodiments, the oligonucleotide linker is selected from the group consisting of GNRA, (G)4, (U)4, and (dT)4, wherein N is a modified or unmodified nucleotide and R is a modified or unmodified purine nucleotide. Some of the nucleotides in the linker can be involved in base-pair interactions with other nucleotides in the linker. The two strands can also be linked together by a non-nucleosidic linker, e.g. a linker described herein. It will be appreciated by one of skill in the art that any oligonucleotide chemical modifications or variations describe herein can be used in the oligonucleotide linker.

Hairpin and dumbbell type oligomeric compounds will have a duplex region equal to or at least 14, 15, 15, 16, 17, 18, 19, 29, 21, 22, 23, 24, or 25 nucleotide pairs. The duplex region can be equal to or less than 200, 100, or 50, in length. In some embodiments, ranges for the duplex region are 15-30, 17 to 23, 19 to 23, and 19 to 21 nucleotides pairs in length.

The hairpin oligomeric compounds can have a single strand overhang or terminal unpaired region, in some embodiments at the 3′, and in some embodiments on the antisense side of the hairpin. In some embodiments, the overhangs are 1-4, more generally 2-3 nucleotides in length. The hairpin oligomeric compounds that can induce RNA interference are also referred to as “shRNA” herein.

Where the two substantially complementary strands of a dsRNA are comprised by separate RNA molecules, those molecules need not, but can be covalently connected. Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, an RNAi may comprise one or more nucleotide overhangs.

In one embodiment, an RNAi agent of the disclosure is a dsRNA, each strand of which is 24-30 nucleotides in length, that interacts with a target RNA sequence, e.g., an APP target mRNA sequence, to direct the cleavage of the target RNA. Without wishing to be bound by theory, long double stranded RNA introduced into cells is broken down into siRNA by a Type III endonuclease known as Dicer (Sharp et al. (2001) Genes Dev. 15: 485). Dicer, a ribonuclease-III-like enzyme, processes the dsRNA into 19-23 base pair short interfering RNAs with characteristic two base 3′ overhangs (Bernstein, et al., (2001) Nature 409: 363). The siRNAs are then incorporated into an RNA-induced silencing complex (RISC) where one or more helicases unwind the siRNA duplex, enabling the complementary antisense strand to guide target recognition (Nykanen, et al., (2001) Cell 107: 309). Upon binding to the appropriate target mRNA, one or more endonucleases within the RISC cleave the target to induce silencing (Elbashir, et al., (2001) Genes Dev. 15: 188).

In one embodiment, an RNAi agent of the disclosure is a dsRNA agent, each strand of which comprises 19-23 nucleotides that interacts with an APP RNA sequence to direct the cleavage of the target RNA. Without wishing to be bound by theory, long double stranded RNA introduced into cells is broken down into siRNA by a Type III endonuclease known as Dicer (Sharp et al. (2001) Genes Dev. 15: 485). Dicer, a ribonuclease-III-like enzyme, processes the dsRNA into 19-23 base pair short interfering RNAs with characteristic two base 3′ overhangs (Bernstein, et al., (2001) Nature 409: 363). The siRNAs are then incorporated into an RNA-induced silencing complex (RISC) where one or more helicases unwind the siRNA duplex, enabling the complementary antisense strand to guide target recognition (Nykanen, et al., (2001) Cell 107: 309). Upon binding to the appropriate target mRNA, one or more endonucleases within the RISC cleave the target to induce silencing (Elbashir, et al., (2001) Genes Dev. 15: 188). In one embodiment, an RNAi agent of the disclosure is a dsRNA of 24-30 nucleotides that interacts with an APP RNA sequence to direct the cleavage of the target RNA.

As used herein, the term “nucleotide overhang” refers to at least one unpaired nucleotide that protrudes from the duplex structure of an RNAi agent, e.g., a dsRNA. For example, when a 3′-end of one strand of a dsRNA extends beyond the 5′-end of the other strand, or vice versa, there is a nucleotide overhang. A dsRNA can comprise an overhang of at least one nucleotide; alternatively, the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′-end, 3′-end or both ends of either an antisense or sense strand of a dsRNA.

In one embodiment of the dsRNA, at least one strand comprises a 3′ overhang of at least 1 nucleotide. In another embodiment, at least one strand comprises a 3′ overhang of at least 2 nucleotides, e.g., 2, 3, 4, 5, 6, 7, 9, 10, 11, 12, 13, 14, or 15 nucleotides. In other embodiments, at least one strand of the RNAi agent comprises a 5′ overhang of at least 1 nucleotide. In certain embodiments, at least one strand comprises a 5′ overhang of at least 2 nucleotides, e.g., 2, 3, 4, 5, 6, 7, 9, 10, 11, 12, 13, 14, or 15 nucleotides. In still other embodiments, both the 3′ and the 5′ end of one strand of the RNAi agent comprise an overhang of at least 1 nucleotide.

In one embodiment, the antisense strand of a dsRNA has a 1-10 nucleotide, e.g., 0-3, 1-3, 2-4, 2-5, 4-10, 5-10, e.g., a 1, 2, 3, 4, 5, 6, 7, 8, 9, or 10 nucleotide, overhang at the 3′-end, the 5′-end, at both ends, or at neither end. In one embodiment, the sense strand of a dsRNA has a 1-10 nucleotide, e.g., a 1, 2, 3, 4, 5, 6, 7, 8, 9, or 10 nucleotide, overhang at the 3′-end, the 5′-end, at both ends, or at neither end. In another embodiment, one or more of the nucleotides in the overhang is replaced with a nucleoside thiophosphate.

In certain embodiments, the overhang on the sense strand or the antisense strand, or both, can include extended lengths longer than 10 nucleotides, e.g., 1-30 nucleotides, 2-30 nucleotides, 10-30 nucleotides, or 10-15 nucleotides in length. In certain embodiments, an extended overhang is on the sense strand of the duplex. In certain embodiments, an extended overhang is present on the 3′end of the sense strand of the duplex. In certain embodiments, an extended overhang is present on the 5′end of the sense strand of the duplex. In certain embodiments, an extended overhang is on the antisense strand of the duplex. In certain embodiments, an extended overhang is present on the 3′end of the antisense strand of the duplex. In certain embodiments, an extended overhang is present on the 5′end of the antisense strand of the duplex. In certain embodiments, one or more of the nucleotides in the overhang is replaced with a nucleoside thiophosphate. In certain embodiments, the overhang includes a self-complementary portion such that the overhang is capable of forming a hairpin structure that is stable under physiological conditions.

The terms “blunt” or “blunt ended” as used herein in reference to a dsRNA mean that there are no unpaired nucleotides or nucleotide analogs at a given terminal end of a dsRNA, i.e., no nucleotide overhang. One or both ends of a dsRNA can be blunt. Where both ends of a dsRNA are blunt, the dsRNA is said to be blunt ended. To be clear, a “blunt ended” dsRNA is a dsRNA that is blunt at both ends, i.e., no nucleotide overhang at either end of the molecule. Most often such a molecule will be double stranded over its entire length.

The term “antisense strand” or “guide strand” refers to the strand of an RNAi agent, e.g., a dsRNA, which includes a region that is substantially complementary to a target sequence, e.g., an APP mRNA.

As used herein, the term “region of complementarity” refers to the region on the antisense strand that is substantially complementary to a sequence, for example a target sequence, e.g., an APP nucleotide sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- or 3′-terminus of the RNAi agent.

In some embodiments, a double stranded RNA agent of the disclosure includes a nucleotide mismatch in the antisense strand.

In some embodiments, the antisense strand of the double stranded RNA agent of the disclosure includes no more than 4 mismatches with the target mRNA, e.g., the antisense strand includes 4, 3, 2, 1, or 0 mismatches with the target mRNA. In some embodiments, the antisense strand double stranded RNA agent of the disclosure includes no more than 4 mismatches with the sense strand, e.g., the antisense strand includes 4, 3, 2, 1, or 0 mismatches with the sense strand. In some embodiments, a double stranded RNA agent of the disclosure includes a nucleotide mismatch in the sense strand. In some embodiments, the sense strand of the double stranded RNA agent of the invention includes no more than 4 mismatches with the antisense strand, e.g., the sense strand includes 4, 3, 2, 1, or 0 mismatches with the antisense strand. In some embodiments, the nucleotide mismatch is, for example, within 5, 4, 3 nucleotides from the 3′-end of the iRNA. In another embodiment, the nucleotide mismatch is, for example, in the 3′-terminal nucleotide of the iRNA agent. In some embodiments, the mismatch(s) is not in the seed region.

Thus, an RNAi agent as described herein can contain one or more mismatches to the target sequence. In one embodiment, an RNAi agent as described herein contains no more than 3 mismatches (i.e., 3, 2, 1, or 0 mismatches). In one embodiment, an RNAi agent as described herein contains no more than 2 mismatches. In one embodiment, an RNAi agent as described herein contains no more than 1 mismatch. In one embodiment, an RNAi agent as described herein contains 0 mismatches. In certain embodiments, if the antisense strand of the RNAi agent contains mismatches to the target sequence, the mismatch can optionally be restricted to be within the last 5 nucleotides from either the 5′- or 3′-end of the region of complementarity. For example, in such embodiments, for a 23 nucleotide RNAi agent, the strand which is complementary to a region of an APP gene, generally does not contain any mismatch within the central 13 nucleotides. The methods described herein or methods known in the art can be used to determine whether an RNAi agent containing a mismatch to a target sequence is effective in inhibiting the expression of an APP gene. Consideration of the efficacy of RNAi agents with mismatches in inhibiting expression of an APP gene is important, especially if the particular region of complementarity in an APP gene is known to have polymorphic sequence variation within the population.

The term “sense strand” or “passenger strand” as used herein, refers to the strand of an RNAi agent that includes a region that is substantially complementary to a region of the antisense strand as that term is defined herein.

As used herein, “substantially all of the nucleotides are modified” are largely but not wholly modified and can include not more than 5, 4, 3, 2, or 1 unmodified nucleotides.

As used herein, the term “cleavage region” refers to a region that is located immediately adjacent to the cleavage site. The cleavage site is the site on the target at which cleavage occurs. In some embodiments, the cleavage region comprises three bases on either end of, and immediately adjacent to, the cleavage site. In some embodiments, the cleavage region comprises two bases on either end of, and immediately adjacent to, the cleavage site. In some embodiments, the cleavage site specifically occurs at the site bound by nucleotides 10 and 11 of the antisense strand, and the cleavage region comprises nucleotides 11, 12 and 13.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences within an RNAi agent, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs, while retaining the ability to hybridize under the conditions most relevant to their ultimate application, e.g., inhibition of gene expression via a RISC pathway. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs or base pairs formed from non-natural and modified nucleotides, in so far as the above requirements with respect to their ability to hybridize are fulfilled. Such non-Watson-Crick base pairs include, but are not limited to, G:U Wobble or Hoogstein base pairing.

The terms “complementary,” “fully complementary” and “substantially complementary” herein can be used with respect to the base matching between the sense strand and the antisense strand of a dsRNA, or between the antisense strand of an RNAi agent and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding APP). For example, a polynucleotide is complementary to at least a part of an APP mRNA if the sequence is substantially complementary to a non-interrupted portion of an mRNA encoding APP.

Accordingly, in some embodiments, the antisense strand polynucleotides disclosed herein are fully complementary to the target APP sequence.

In certain embodiments, the antisense strand polynucleotides disclosed herein are substantially complementary to the target APP sequence and comprise a contiguous nucleotide sequence which is at least about 80% complementary over its entire length to the equivalent region of the nucleotide sequence of SEQ ID NOs: 4, 7, 10, 13, 16, 19, 22, 25, 28, 31, 34, 37, 40 or 43 for APP, or a fragment of SEQ ID NOs: 4, 7, 10, 13, 16, 19, 22, 25, 28, 31, 34, 37, 40 or 43, such as about 85%, about 86%, about 87%, about 88%, about 89%, about 90%, about 91%, about 92%, about 93%, about 94%, about 95%, about 96%, about 97%, about 98%, or about 99% complementary.

In other embodiments, the antisense polynucleotides disclosed herein are substantially complementary to the target APP sequence and comprise a contiguous nucleotide sequence which is at least about 80% complementary over its entire length to any one of the sense strand nucleotide sequences in any one of Tables 2-21, or a fragment of any one of the sense strand nucleotide sequences in any one of Tables 2-21, such as about 85%, about 86%, about 87%, about 88%, about 89%, about 90%, about 91%, about 92%, about 93%, about 94%, about 95%, about 96%, about 97%, about 98%, or about 99% complementary.

In one embodiment, an RNAi agent of the disclosure includes a sense strand that is substantially complementary to an antisense polynucleotide which, in turn, is the same as a target APP sequence, and wherein the sense strand polynucleotide comprises a contiguous nucleotide sequence which is at least about 80% complementary over its entire length to the equivalent region of the nucleotide sequence of SEQ ID NOs: 5, 8, 11, 14, 17, 20, 23, 26, 29, 32, 35, 38, 41 or 44, or a fragment of any one of SEQ ID NOs: 5, 8, 11, 14, 17, 20, 23, 26, 29, 32, 35, 38, 41 or 44, such as about 85%, about 86%, about 87%, about 88%, about 89%, about 90%, about 91%, about 92%, about 93%, about 94%, about 95%, about 96%, about 97%, about 98%, or about 99% complementary.

In some embodiments, an iRNA of the disclosure includes a sense strand that is substantially complementary to an antisense polynucleotide which, in turn, is complementary to a target APP sequence, and wherein the sense strand polynucleotide comprises a contiguous nucleotide sequence which is at least about 80% complementary over its entire length to any one of the antisense strand nucleotide sequences in any one of any one of Tables 2-21, or a fragment of any one of the antisense strand nucleotide sequences in any one of Tables 2-21, such as about 85%, about 90%, about 91%, about 92%, about 93%, about 94%, about 95%, about 96%, about 97%, about 98%, about 99%, or 100% complementary

In some embodiments, the double-stranded region of a double-stranded iRNA agent is equal to or at least, 10, 11, 12, 13, 14, 15, 16, 17, 18, 19, 20, 21, 22, 23, 23, 24, 25, 26, 27, 28, 29, 30 or more nucleotide pairs in length.

In some embodiments, the antisense strand of a double-stranded iRNA agent is equal to or at least 14, 15, 16, 17, 18, 19, 20, 21, 22, 23, 23, 24, 25, 26, 27, 28, 29, or 30 nucleotides in length.

In some embodiments, the sense strand of a double-stranded iRNA agent is equal to or at least 10, 11, 12, 13, 14, 15, 16, 17, 18, 19, 20, 21, 22, 23, 23, 24, 25, 26, 27, 28, 29, or 30 nucleotides in length.

In one embodiment, the sense and antisense strands of the double-stranded iRNA agent are each 15 to 30 nucleotides in length.

In one embodiment, the sense and antisense strands of the double-stranded iRNA agent are each 19 to 25 nucleotides in length.

In one embodiment, the sense and antisense strands of the double-stranded iRNA agent are each 21 to 23 nucleotides in length.

In one embodiment, the sense strand of the iRNA agent is 21-nucleotides in length, and the antisense strand is 23-nucleotides in length, wherein the strands form a double-stranded region of 21 consecutive base pairs having a 2-nucleotide long single stranded overhangs at the 3′-end.

In some embodiments, the majority of nucleotides of each strand are ribonucleotides, but as described in detail herein, each or both strands can also include one or more non-ribonucleotides, e.g., a deoxyribonucleotide or a modified nucleotide. In addition, an “iRNA” may include ribonucleotides with chemical modifications. Such modifications may include all types of modifications disclosed herein or known in the art. Any such modifications, as used in an iRNA molecule, are encompassed by “iRNA” for the purposes of this specification and claims.

In one aspect of the disclosure, an agent for use in the methods and compositions of the disclosure is a single-stranded antisense nucleic acid molecule that inhibits a target mRNA via an antisense inhibition mechanism. The single-stranded antisense RNA molecule is complementary to a sequence within the target mRNA. The single-stranded antisense oligonucleotides can inhibit translation in a stoichiometric manner by base pairing to the mRNA and physically obstructing the translation machinery, see Dias, N. et al., (2002) Mol Cancer Ther 1: 347-355. The single-stranded antisense RNA molecule may be about 15 to about 30 nucleotides in length and have a sequence that is complementary to a target sequence. For example, the single-stranded antisense RNA molecule may comprise a sequence that is at least about 15, 16, 17, 18, 19, 20, or more contiguous nucleotides from any one of the antisense sequences described herein.

In one embodiment, at least partial suppression of the expression of an APP gene, is assessed by a reduction of the amount of APP mRNA which can be isolated from or detected in a first cell or group of cells in which an APP gene is transcribed and which has or have been treated such that the expression of an APP gene is inhibited, as compared to a second cell or group of cells substantially identical to the first cell or group of cells but which has or have not been so treated (control cells). The degree of inhibition may be expressed in terms of:

$$\frac{( mRNA in control cells ) - ( mRNA in treated cells )}{( mRNA in control cells )} \cdot 100 \%$$

The phrase “contacting a cell with an RNAi agent,” such as a dsRNA, as used herein, includes contacting a cell by any possible means. Contacting a cell with an RNAi agent includes contacting a cell in vitro with the RNAi agent or contacting a cell in vivo with the RNAi agent. The contacting may be done directly or indirectly. Thus, for example, the RNAi agent may be put into physical contact with the cell by the individual performing the method, or alternatively, the RNAi agent may be put into a situation that will permit or cause it to subsequently come into contact with the cell.

Contacting a cell in vitro may be done, for example, by incubating the cell with the RNAi agent. Contacting a cell in vivo may be done, for example, by injecting the RNAi agent into or near the tissue where the cell is located, or by injecting the RNAi agent into another area, e.g., the central nervous system (CNS), optionally via intrathecal, intravitreal or other injection, or to the bloodstream or the subcutaneous space, such that the agent will subsequently reach the tissue where the cell to be contacted is located. For example, the RNAi agent may contain or be coupled to a ligand, e.g., a lipophilic moiety or moieties as described below and further detailed, e.g., in PCT/US2019/031170, which is incorporated herein by reference, that directs or otherwise stabilizes the RNAi agent at a site of interest, e.g., the CNS. In some embodiments, the RNAi agent may contain or be coupled to a ligand, e.g., one or more GalNAc derivatives as described below, that directs or otherwise stabilizes the RNAi agent at a site of interest, e.g., the liver. In other embodiments, the RNAi agent may contain or be coupled to a lipophilic moiety or moieties and one or more GalNAc derivatives. Combinations of in vitro and in vivo methods of contacting are also possible. For example, a cell may also be contacted in vitro with an RNAi agent and subsequently transplanted into a subject.

In one embodiment, contacting a cell with an RNAi agent includes “introducing” or “delivering the RNAi agent into the cell” by facilitating or effecting uptake or absorption into the cell. Absorption or uptake of an RNAi agent can occur through unaided diffusive or active cellular processes, or by auxiliary agents or devices. Introducing an RNAi agent into a cell may be in vitro or in vivo. For example, for in vivo introduction, an RNAi agent can be injected into a tissue site or administered systemically. In vitro introduction into a cell includes methods known in the art such as electroporation and lipofection. Further approaches are described herein below or are known in the art.

The term “lipophile” or “lipophilic moiety” broadly refers to any compound or chemical moiety having an affinity for lipids. One way to characterize the lipophilicity of the lipophilic moiety is by the octanol-water partition coefficient, log K_{ow}, where K_{ow} is the ratio of a chemical's concentration in the octanol-phase to its concentration in the aqueous phase of a two-phase system at equilibrium. The octanol-water partition coefficient is a laboratory-measured property of a substance. However, it may also be predicted by using coefficients attributed to the structural components of a chemical which are calculated using first-principle or empirical methods (see, for example, Tetko et al., J. Chem. Inf Comput. Sci. 41: 1407-21 (2001), which is incorporated herein by reference in its entirety). It provides a thermodynamic measure of the tendency of the substance to prefer a non-aqueous or oily milieu rather than water (i.e. its hydrophilic/lipophilic balance). In principle, a chemical substance is lipophilic in character when its log K_{ow} exceeds 0. Typically, the lipophilic moiety possesses a log K_{ow} exceeding 1, exceeding 1.5, exceeding 2, exceeding 3, exceeding 4, exceeding 5, or exceeding 10. For instance, the log K_{ow} of 6-amino hexanol, for instance, is predicted to be approximately 0.7. Using the same method, the log K_{ow} of cholesteryl N-(hexan-6-ol) carbamate is predicted to be 10.7.

The lipophilicity of a molecule can change with respect to the functional group it carries. For instance, adding a hydroxyl group or amine group to the end of a lipophilic moiety can increase or decrease the partition coefficient (e.g., log K_{ow}) value of the lipophilic moiety.

Alternatively, the hydrophobicity of the double-stranded RNAi agent, conjugated to one or more lipophilic moieties, can be measured by its protein binding characteristics. For instance, in certain embodiments, the unbound fraction in the plasma protein binding assay of the double-stranded RNAi agent could be determined to positively correlate to the relative hydrophobicity of the double-stranded RNAi agent, which could then positively correlate to the silencing activity of the double-stranded RNAi agent.

In one embodiment, the plasma protein binding assay determined is an electrophoretic mobility shift assay (EMSA) using human serum albumin protein. An exemplary protocol of this binding assay is illustrated in detail in, e.g., PCT/US2019/031170. The hydrophobicity of the double-stranded RNAi agent, measured by fraction of unbound siRNA in the binding assay, exceeds 0.15, exceeds 0.2, exceeds 0.25, exceeds 0.3, exceeds 0.35, exceeds 0.4, exceeds 0.45, or exceeds 0.5 for an enhanced in vivo delivery of siRNA.

Accordingly, conjugating the lipophilic moieties to the internal position(s) of the double-stranded RNAi agent provides optimal hydrophobicity for the enhanced in vivo delivery of siRNA.

The term “lipid nanoparticle” or “LNP” refers to a vesicle comprising a lipid layer encapsulating a pharmaceutically active molecule, such as a nucleic acid molecule, e.g., an RNAi agent or a plasmid from which an RNAi agent is transcribed. LNPs are described in, for example, U.S. Pat. Nos. 6,858,225, 6,815,432, 8,158,601, and 8,058,069, the entire contents of which are hereby incorporated herein by reference.

As used herein, a “subject” is an animal, such as a mammal, including a primate (such as a human, a non-human primate, e.g., a monkey, and a chimpanzee), or a non-primate (such as a rat, or a mouse). In a preferred embodiment, the subject is a human, such as a human being treated or assessed for a disease, disorder, or condition that would benefit from reduction in APP expression; a human at risk for a disease, disorder, or condition that would benefit from reduction in APP expression; a human having a disease, disorder, or condition that would benefit from reduction in APP expression; or human being treated for a disease, disorder, or condition that would benefit from reduction in APP expression as described herein.

As used herein, the terms “treating” or “treatment” refer to a beneficial or desired result including, but not limited to, alleviation or amelioration of one or more signs or symptoms associated with APP gene expression or APP protein production further characterized by enlarged neuronal cell endosomes, e.g., APP-associated neurodegenerative disease characterized by enlarged neuronal cell endosomes, Alzheimer's disease (AD) or Down syndrome (DS), specifically including, e.g., decreased expression or activity of APP, e.g., in regions of increased or stable behavior/cognition during and/or following treatment; decreased or inhibited neuroinflammation, gliosis, amyloidosis and/or tauopathy during and/or following treatment; lessening of the rate of decline, stabilization and/or improvement of speech and movement, etc. during and/or following treatment, in subjects having such neurodegenerative diseases. “Treatment” can also mean prolonging survival as compared to expected survival in the absence of treatment.

The term “lower” in the context of the level of APP in a subject or a disease marker or symptom refers to a statistically significant decrease in such level. The decrease can be, for example, at least 10%, 15%, 20%, 25%, 30%, %, 40%, 45%, 50%, 55%, 60%, 65%, 70%, 75%, 80%, 85%, 90%, 95%, or more. In certain embodiments, a decrease is at least 20%. In certain embodiments, the decrease is at least 50% in a disease marker, e.g., protein or gene expression level. “Lower” in the context of the level of APP in a subject is optionally down to a level accepted as within the range of normal for an individual without such disorder. In certain embodiments, “lower” is the decrease in the difference between the level of a marker or symptom for a subject suffering from a disease and a level accepted within the range of normal for an individual, e.g., the level of decrease in the observed gap between behavior/cognition, speech, etc., in an individual having AD or DS and an individual not having AD or DS or having symptoms that are within the range of normal.

As used herein, “prevention” or “preventing,” when used in reference to a disease or disorder characterized by enlarged neuronal cell endosomes, that would benefit from a reduction in expression of an APP gene or production of APP protein, e.g., in a subject susceptible to an APP-associated disorder characterized by enlarged neuronal cell endosomes due to, e.g., genetic factors or age, wherein the subject does not yet meet the diagnostic criteria for the APP-associated disorder characterized by enlarged neuronal cell endosomes. As used herein, prevention can be understood as administration of an agent to a subject who does not yet meet the diagnostic criteria for the APP-associated disorder characterized by enlarged neuronal cell endosomes to delay or reduce the likelihood that the subject will develop the APP-associated disorder characterized by enlarged neuronal cell endosomes. As the agent is a pharmaceutical agent, it is understood that administration typically would be under the direction of a health care professional capable of identifying a subject who does not yet meet the diagnostic criteria for an APP-associated disorder characterized by enlarged neuronal cell endosomes as being susceptible to developing an APP-associated disorder characterized by enlarged neuronal cell endosomes. Diagnostic criteria for AD and DS, and risk factors for these disorders are provided herein, and include identification of a genetic predisposition to AD, among others. The likelihood of developing, e.g., AD or DS, is reduced, for example, when an individual having one or more risk factors for AD or DS either fails to develop AD or DS or develops AD or DS with less severity relative to a population having the same risk factors and not receiving treatment as described herein. The failure to develop an APP-associated disorder characterized by enlarged neuronal cell endosomes, e.g., AD or DS, or a delay in the time to develop AD or DS by months or years is considered effective prevention. Prevention may require administration of more than one dose of the iRNA agent. Provided with appropriate methods to identify subjects at risk to develop any of the APP-associated diseases characterized by enlarged neuronal cell endosomes above, the iRNA agents provided herein can be used as pharmaceutical agents for or in methods of prevention of APP-associated diseases characterized by enlarged neuronal cell endosomes. Risk factors for various APP-associated diseases characterized by enlarged neuronal cell endosomes are discussed herein.

As used herein, the term “APP-associated disease characterized by enlarged endosomes” or “APP-associated disorder characterized by enlarged endosomes” is understood as Alzheimer's disease (AD) or Down syndrome (DS), in certain embodiments also including Parkinson's Disease (PD), amyotrophic lateral sclerosis (ALS), frontotemporal dementia (FTD), Niemann-Pick Disease type C (NPC), macular degeneration, and Huntington's Disease (HD), or in certain embodiments, only AD. The majority of people with Alzheimer's disease are 65 and older, and the greatest risk factor for developing AD is increasing age. AD is a progressive disease, where dementia symptoms gradually get worse over a number of years. In the early stages, there may be a mild amount of memory loss, but in late stage AD, a person loses the ability to converse, and has confusion over the identity of people, places and things. Early signs of AD are memory loss that disrupts daily life, difficulty completing familiar tasks, inability to plan or solve problems, trouble understanding visual images and spatial relationships, confusion about the time or place, problems with words in speaking or writing, decreased or poor judgment, misplacing things, withdrawing from social activities, and changes in mood and personality.

In one embodiment, an APP-associated disease characterized by enlarged neuronal cell endosomes is “Down syndrome” (“DS”). Down syndrome (DS) is a genetic disorder caused when abnormal cell division results in an extra full or partial copy of chromosome 21. This extra genetic material causes the developmental changes and physical features of Down syndrome, which include (with varying severity) lifelong intellectual disability (cognitive impairment) and developmental delays, among other medical abnormalities, such as heart and gastrointestinal (GI) disorders.

The term “dementia”, as used herein, refers to the term commonly known by a person skilled in the art. According to the World Health Organization (WHO), dementia is a syndrome—usually of a chronic or progressive nature—in which there is deterioration in cognitive function beyond what might be expected from normal ageing. It affects memory, thinking, orientation, comprehension, calculation, learning capacity, language, and judgement. Consciousness is not affected. The impairment in cognitive function is commonly accompanied, and occasionally preceded, by deterioration in emotional control, social behavior, or motivation. Dementia results from a variety of diseases and injuries that primarily or secondarily affect the brain, such as Alzheimer's disease or stroke. Alzheimer disease is the most common form and may contribute to 60-70% of cases. Other major forms include vascular dementia, dementia with Lewy bodies (abnormal aggregates of protein that develop inside nerve cells), and a group of diseases that contribute to frontotemporal dementia (degeneration of the frontal lobe of the brain).

“Therapeutically effective amount,” as used herein, is intended to include the amount of an RNAi agent that, when administered to a subject having an APP-associated disease characterized by enlarged neuronal cell endosomes, is sufficient to effect treatment of the disease (e.g., by diminishing, ameliorating, or maintaining the existing disease or one or more symptoms of disease). The “therapeutically effective amount” may vary depending on the RNAi agent, how the agent is administered, the disease and its severity and the history, age, weight, family history, genetic makeup, the types of preceding or concomitant treatments, if any, and other individual characteristics of the subject to be treated.

“Prophylactically effective amount,” as used herein, is intended to include the amount of an RNAi agent that, when administered to a subject having an APP-associated disorder characterized by enlarged neuronal cell endosomes, is sufficient to prevent or ameliorate the disease or one or more symptoms of the disease. Ameliorating the disease includes slowing the course of the disease or reducing the severity of later-developing disease. The “prophylactically effective amount” may vary depending on the RNAi agent, how the agent is administered, the degree of risk of disease, and the history, age, weight, family history, genetic makeup, the types of preceding or concomitant treatments, if any, and other individual characteristics of the patient to be treated.

A “therapeutically-effective amount” or “prophylactically effective amount” also includes an amount of an RNAi agent that produces some desired local or systemic effect at a reasonable benefit/risk ratio applicable to any treatment. An RNAi agent employed in the methods of the present disclosure may be administered in a sufficient amount to produce a reasonable benefit/risk ratio applicable to such treatment.

The phrase “pharmaceutically acceptable” is employed herein to refer to those compounds, materials, compositions, or dosage forms which are, within the scope of sound medical judgment, suitable for use in contact with the tissues of human subjects and animal subjects without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates or polyanhydrides; (22) bulking agents, such as polypeptides and amino acids (23) serum component, such as serum albumin, HDL and LDL; and (22) other non-toxic compatible substances employed in pharmaceutical formulations.

The term “sample,” as used herein, includes a collection of similar fluids, cells, or tissues isolated from a subject, as well as fluids, cells, or tissues present within a subject. Examples of biological fluids include blood, serum and serosal fluids, plasma, cerebrospinal fluid, ocular fluids, lymph, urine, saliva, and the like. Tissue samples may include samples from tissues, organs or localized regions. For example, samples may be derived from particular organs, parts of organs, or fluids or cells within those organs. In certain embodiments, samples may be derived from the brain (e.g., whole brain or certain segments of brain, e.g., frontal lobe, entorhinal cortex of medial temporal lobe, hippocampus, cerebral cortex, basal ganglia, substantia nigra, or certain types of cells in the brain, such as, e.g., neurons and glial cells (astrocytes, oligodendrocytes, microglial cells)). In other embodiments, a “sample derived from a subject” refers to liver tissue (or subcomponents thereof) derived from the subject. In some embodiments, a “sample derived from a subject” refers to blood drawn from the subject or plasma or serum derived therefrom. In further embodiments, a “sample derived from a subject” refers to brain tissue (or subcomponents thereof) or retinal tissue (or subcomponents thereof) derived from the subject.

It will be understood that, although the sequences in Tables 2, 3, 5, 7, 9, 11, 13-17, 19 and 20 are described or shown as modified or conjugated sequences, the RNA of the RNAi agent of the disclosure e.g., a dsRNA of the disclosure, may comprise any one of the sequences set forth in any one of Tables 2-21 that is un-modified, un-conjugated, or modified or conjugated differently than described therein. That is, for example, the modified sequences provided in Table 2 do not require the L96 ligand, or any ligand. Similarly, the exemplary modified sequences provided in Tables 3 and 5 do not require the exemplary C16 lipophilic ligand shown, or a lipophilic ligand in the position shown. A lipophilic ligand can be included in any of the positions provided in the instant application.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIG. 1 shows a study design for evaluation of APP-targeting RNAi agents in culture cells.

FIGS. 2A and 2B show results of examining spheroids in culture for morphology following treatment with APP-targeting RNAi agent. FIG. 2A shows pre-treatment morphology. FIG. 2B shows unchanged morphology at a 14 day time point after administration of APP-targeting siRNAs/compounds.

FIG. 3 shows a waveform analysis performed upon spheroid data, in which parameters that described the number, size and shape of spontaneous calcium oscillations in each spheroid were extracted. In total, eight parameters were evaluated: peak count, peak height, peak height standard deviation (SD), peak width, peak spacing, peak spacing standard deviation (SD), peak rise time and peak decay time. All values were normalized to vehicle control and were plotted as a percentage.

FIG. 4 shows a series of graphs that demonstrate that minimal modulation of peak count and peak height was observed across treatment groups and concentrations, regardless of whether an APP-targeting RNAi agent (ΔD454844.47) or a small molecule β-Site Amyloid Precursor Protein Cleaving Enzyme (BACE) inhibitor (LY2886721) was administered.

FIGS. 5A and 5B show bar graphs that demonstrate observed sAPPα and sAPPβ levels in cell media. FIG. 5A shows that in spheroid cell media, the APP-targeting siRNA exhibited dose-dependent inhibition of sAPPα levels, whereas the BACE inhibitor LY2886721 exhibited no impact on sAPPα levels in cell media. FIG. 5B shows that when sAPPβ levels were assessed in spheroid cell media, both the APP-targeting siRNA and the BACE inhibitor LY2886721 exhibited dose-dependent inhibition of sAPPβ levels.

FIGS. 6A and 6B show bar graphs that demonstrate observed sAPPα and sAPPβ levels in spheroid cell lysates. FIG. 6A shows that in spheroid cell lysates, the APP-targeting siRNA again exhibited dose-dependent inhibition of sAPPα levels, whereas the BACE inhibitor LY2886721 exhibited no impact on sAPPα levels. FIG. 6A shows that when sAPPβ levels were assessed in spheroid cell lysates, both the APP-targeting siRNA and the BACE inhibitor LY2886721 exhibited dose-dependent inhibition of sAPPβ levels.

FIGS. 7A and 7B show the bar graphs of FIGS. 5B and 6B above, in a manner that provides for easier comparison of results.

FIG. 8 shows a flow-chart depiction of the process of generating iPSC-derived neurons from a PSEN1 A246E patient, which were then administered either a control (AD-1397409, anti-luciferase, see Table 22 below for AD-1397409 sequences) siRNA or the APP-targeting siRNA AD-454844.

FIGS. 9A and 9B show that APP knockdown via administration of an APP-targeting siRNA robustly reduced enlarged endosome size otherwise observed in untreated PSEN1-mutant cells. Significantly elevated Rab5+ endosome sizes were previously observed in cells homozygous for PSEN1 A246E. FIG. 9A shows immunofluorescent Rab5 imaging, which revealed that treatment of cells homozygous for PSEN1 A246E with the APP-targeting siRNA AD-454844 significantly reduced endosome size, as compared to control (CTL) luciferase-targeting siRNA AD-1397409. Specifically, PSEN1^{A246E} patient induced pluripotent stem cell (iPSC)-derived cortical neuron model cells were treated with an APP-targeted siRNA or the luciferase-targeting control siRNA AD-1397409. FIG. 9B shows a series of bar graphs that quantitate: in the left panel, the extent of APP mRNA knockdown observed in AD-454844-treated and control (AD-1397409)-treated mutant cells; in the middle panel, the immunofluorescent Rab5+(early endosome) imaging data obtained, which revealed a statistically significant reduction in maximum early endosome size in APP-targeting siRNA AD-454844-treated cells; and in the right panel, the immunofluorescent Rab7+(Rab7 localizes to both early and late endosomes/multivesicular bodies (LEs/MVBs) and was labeled with Alexa 528) imaging data obtained, which revealed a statistically significant yet more modest reduction in maximum Rab7-associated endosome size in APP-targeting siRNA AD-454844-treated cells, consistent with the APP-targeting siRNA exerting a preferential impact upon early endosome size (as compared to LEs/MVBs).

FIG. 10 shows that siRNA-mediated APP knockdown was significantly more effective than administration of the APP-targeting small molecule BACE inhibitor LY2886721 in reducing both PCTF levels and early endosome (Rab5+ endosome) size in PSEN1^{A246E} patient iPSC-derived cortical neuron model cells. In all such experiments, dose-response of the APP-targeting siRNA AD-454844 was evaluated at 23 days post-transfection, at later stages (DIV30) of differentiation, while in parallel, the APP-targeting small molecule BACE inhibitor LY2886721 was administered to cells for four days, with evaluation for dose-response performed at DIV30. In the left-hand panel, dose-dependent inhibition of sAPPβ levels was observed for both the APP-targeting siRNA AD-454844 and for the APP-targeting small molecule BACE inhibitor LY2886721, though the APP-targeting siRNA AD-454844 exhibited enhanced potency of sAPPβ knockdown across all concentrations assessed. In the second panel from left, dose-dependent inhibition of sAPPα levels was only observed for the APP-targeting siRNA AD-454844, whereas the APP-targeting small molecule BACE inhibitor LY2886721 exhibited no sAPPα knockdown at any dose tested. In the third panel from left, dose-dependent reduction of APP β-C-terminal fragment (β-CTF) levels was observed for both the APP-targeting siRNA AD-454844 and for the APP-targeting small molecule BACE inhibitor LY2886721, yet significantly more potent reduction of β-CTF was observed for the APP-targeting siRNA AD-454844, as compared to LY2886721. The right-hand panel shows that early endosome (RAB5+ endosome) size was significantly more reduced in PSEN1^{A246E} patient iPSC-derived cortical neuron model cells treated with AD-454844 at 10 nM, as compared to the more modest levels of early endosome size reduction observed for such cells treated with LY2886721 at 10 nM. APP-targeting siRNAs are therefore capable of exerting a preferential effect upon APP forms found in early endosomes and upon early endosome size, that is distinct from that observed for the APP-targeting small molecule BACE inhibitor LY2886721, and that also is distinct from antibody-based therapies (as antibody-based therapeutics do not target intracellular β-CTF).

FIGS. 11A to 11I demonstrate the efficacy of siRNA-mediated APP silencing in the CVN mouse model; throughout FIGS. 11A to 11I, “siRNA XVIII” represents AD-454972. FIG. 1A shows that the human AD-454972 APP-targeting siRNA reduced APP mRNA and sAPPα protein (aCSF, n=6 per group; AD-454972, n=3 per group). FIG. 11B shows that a single 120 μg ICV bolus dose showed an approximate 75% reduction of APP mRNA at 30 days and >50% reduction at 60 days post-dose. Day 30 and Day 180, n=4 per group; Day 60, n=1 per group; Day 90, n=11 per group). FIG. 11C depicts an overview of the experimental design and disease progression in the CVN mice. Animals were dosed pre-symptomatically and assessed by immunohistochemistry (IHC) for changes in deposition of AB40 (FIGS. 11E and 11F below) and inflammation (IBA1) (FIGS. 11E and 11G below) within the cortex and hippocampus at 3 months or 6 months post-dose. FIG. 11D shows that after 3 months, a reduction of approximately 25% and approximately 50% of APP mRNA was observed in the cortex and hippocampus, respectively, which corresponded to an approximate 50% reduction in sAPPα protein. (aCSF, n=3 per group; AD-454972 APP-targeting siRNA, n=4 per group). FIG. 11E shows changes in deposition of AB40 and inflammation (IBA1) within the cortex and hippocampus at 9 months post-dose of either the aCSF control or the AD-454972 APP-targeting siRNA. FIG. 1F shows tissue AB40 deposits assessed by IHC (aCSF, n=2 per group at 6 months; n=4 for the remaining groups). FIG. 11G shows tissue IBA1 levels assessed by IHC and qPCR (Iba1; aCSF, n=2 per group at 6 months; n=4 for the remaining groups). Simple linear regression was used to compare the slopes. * P<0.05 and P=0.0237 in the AD-454972 APP-targeting siRNA group. FIG. 11H shows glutamate and N-acetylaspartate levels as measured by rH-MRS at 12 months of age (6 months post-dose) showed normalization of glutamate levels in the siRNA-treated group. (WT aCSF, n=9 per group; n=8 per group for the remaining groups. All error bars represent standard error. * P<0.05. Unpaired t-test assuming equal variance was used. CR, creatine. FIG. 1I shows that AD-454972 APP-targeting siRNA-treated animals showed normalization of total distance traveled and rearing frequency. WT aCSF, n=9 per group; n=8 per group for the remaining groups. All error bars represent standard error. * P<0.05; ** P<0.005. Unpaired t-test assuming equal variance was used unless indicated otherwise. NS, not significant.

The present invention is further illustrated by the following detailed description.

### DETAILED DESCRIPTION OF THE INVENTION

The present disclosure provides for use of RNAi compositions, which effect the RNA-induced silencing complex (RISC)-mediated cleavage of RNA transcripts of an APP gene, for preclinical, therapeutic or prophylactic purpose to effect robust endosomal size reductions in cells or subjects contacted with such agents, particularly in cells or subjects harboring mutations in presenilin 1 (PSEN1). The APP gene may be within a cell, e.g., a cell within a subject, such as a human. The present disclosure in specific aspects provides methods of using the RNAi compositions of the disclosure for inhibiting the expression of an APP gene or for treating a subject having a disorder that would benefit from inhibiting or reducing the expression of an APP gene, e.g., an APP-associated disease characterized by enlarged neuronal cell endosomes, e.g., Alzheimer's disease (AD) or Down syndrome (DS).

The RNAi agents of the disclosure include an RNA strand (the antisense strand) having a region which is about 30 nucleotides or less in length, e.g., 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length, which region is substantially complementary to at least part of an mRNA transcript of an APP gene. In certain embodiments, the RNAi agents of the disclosure include an RNA strand (the antisense strand) having a region which is about 21-23 nucleotides in length, which region is substantially complementary to at least part of an mRNA transcript of an APP gene.

In certain embodiments, the RNAi agents of the disclosure include an RNA strand (the antisense strand) which can include longer lengths, for example up to 66 nucleotides, e.g., 36-66, 26-36, 25-36, 31-60, 22-43, 27-53 nucleotides in length with a region of at least 19 contiguous nucleotides that is substantially complementary to at least a part of an mRNA transcript of an APP gene. These RNAi agents with the longer length antisense strands optionally include a second RNA strand (the sense strand) of 20-60 nucleotides in length wherein the sense and antisense strands form a duplex of 18-30 contiguous nucleotides.

The use of these RNAi agents enables the targeted degradation of mRNAs of an APP gene in mammals. Thus, methods and compositions including these RNAi agents are useful for treating a subject who would benefit by a reduction in the levels or activity of an APP protein, such as a subject having an APP-associated neurodegenerative disease characterized by enlarged neuronal cell endosomes, e.g. Alzheimer's disease (AD) or Down syndrome (DS), particularly those associated with mutations in presenilin 1 (PSEN1) in an affected subject.

It was recently identified that overactivation of Rab5 could induce endosomal dysfunction and produce prodromal and neurodegenerative features of AD (Pensalfini et al. Cell Reports 33, 108420, Nov. 24, 2020), with endosomal size abnormalities also associated with PD, ALS, FTD and HD (Pensalfini et al.), among other conditions. Investigation of endosome size in neuronal cells performed herein has now identified that APP-targeting siRNAs can robustly decrease endosome size in neuronal cells otherwise characterized by enlarged endosomes (caused, e.g., by mutations in PSEN1), with such effect likely exerting a therapeutic benefit to subjects having or at risk of developing mutations that enlarge neuronal cell endosome size. Therapeutic and preventive administration of APP-targeting RNAi agents to subjects having or at risk of developing conditions that produce enlarged neuronal cell endosomes, is therefore contemplated, including, e.g., selection of a subject or group of subjects for administration of APP-targeting RNAi agents as disclosed herein, based upon detection of, e.g., a PSEN1 mutation that characteristically produces enlarged neuronal cell endosomes, or direct detection of enlarged neuronal cell endosomes in a subject.

Early endosomal abnormalities have been detected in a range of diseases, including, e.g., not only AD and DS (Cataldo et al. J Neurosci Off J Soc Neurosc 23:6788-6792; Cataldo et al. Neurobiol Aging 25:1263-1272; Nixon, R. A. Neurobiol Aging 26:373-382), but also PD (Xu et al., Traffic 19(4): 253-262), Niemann-Pick Disease type C (NPC) (Jin et al. Am J Pathol 164:975-985) and Stargardt macular degeneration (Lakkaraju et al. Proc Natl Acad Sci USA 104:11026-11031; Toops et al. Exp Eye Res 124:74-85; Tan et al. Proc Natl Acad Sci USA 113:8789-8794), as discussed, e.g., in Kaur and Lakkaraju. Adv Exp Med Biol. 2018; 1074: 335-343. It is expressly contemplated herein that RNAi-mediated knockdown of APP as disclosed herein could exert therapeutic or even preventive benefit to subjects having or at risk of developing any of these diseases or disorders.

The following detailed description discloses how to make and use compositions containing RNAi agents to inhibit the expression of an APP gene, as well as compositions and methods for treating subjects having diseases and disorders that would benefit from inhibition or reduction of the expression of the genes.

### I. RNAi Agents of the Disclosure

Described herein are RNAi agents which inhibit the expression of an APP gene. In one embodiment, the RNAi agent includes double stranded ribonucleic acid (dsRNA) molecules for inhibiting the expression of an APP gene in a cell, such as a cell within a subject, e.g., a mammal, such as a human having an APP-associated neurodegenerative disease characterized by enlarged neuronal cell endosomes, e.g. Alzheimer's disease (AD) or Down syndrome (DS), particularly examples of such conditions that are associated with mutations in presenilin 1 (PSEN1) in an affected subject. The dsRNA includes an antisense strand having a region of complementarity which is complementary to at least a part of an mRNA formed in the expression of an APP gene. In embodiments, the region of complementarity is about 15-30 nucleotides or less in length. Upon contact with a cell expressing the APP gene, the RNAi agent inhibits the expression of the APP gene (e.g., a human gene, a primate gene, a non-primate gene) by at least 50% as assayed by, for example, a PCR or branched DNA (bDNA)-based method, or by a protein-based method, such as by immunofluorescence analysis, using, for example, western blotting or flowcytometric techniques.

A dsRNA includes two RNA strands that are complementary and hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of an APP gene. The other strand (the sense strand) includes a region that is complementary to the antisense strand, such that the two strands hybridize and form a duplex structure when combined under suitable conditions. As described elsewhere herein and as known in the art, the complementary sequences of a dsRNA can also be contained as self-complementary regions of a single nucleic acid molecule, as opposed to being on separate oligonucleotides.

Generally, the duplex structure is 15 to 30 base pairs in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length. In certain preferred embodiments, the duplex structure is 18 to 25 base pairs in length, e.g., 18-25, 18-24, 18-23, 18-22, 18-21, 18-20, 19-25, 19-24, 19-23, 19-22, 19-21, 19-20, 20-25, 20-24, 20-23, 20-22, 20-21, 21-25, 21-24, 21-23, 21-22, 22-25, 22-24, 22-23, 23-25, 23-24 or 24-25 base pairs in length, for example, 19-21 basepairs in length. Ranges and lengths intermediate to the above recited ranges and lengths are also contemplated to be part of the disclosure.

Similarly, the region of complementarity to the target sequence is 15 to 30 nucleotides in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length, for example 19-23 nucleotides in length or 21-23 nucleotides in length. Ranges and lengths intermediate to the above recited ranges and lengths are also contemplated to be part of the disclosure.

In some embodiments, the dsRNA is 15 to 23 nucleotides in length, or 24 to 30 nucleotides in length (optionally, 25 to 30 nucleotides in length). In general, the dsRNA can be long enough to serve as a substrate for the Dicer enzyme. For example, it is well known in the art that dsRNAs longer than about 21-23 nucleotides can serve as substrates for Dicer. As the ordinarily skilled person will also recognize, the region of an RNA targeted for cleavage will most often be part of a larger RNA molecule, often an mRNA molecule. Where relevant, a “part” of an mRNA target is a contiguous sequence of an mRNA target of sufficient length to allow it to be a substrate for RNAi-directed cleavage (i.e., cleavage through a RISC pathway).

One of skill in the art will also recognize that the duplex region is a primary functional portion of a dsRNA, e.g., a duplex region of about 15 to 36 base pairs, e.g., 15-36, 15-35, 15-34, 15-33, 15-32, 15-31, 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs, for example, 19-21 base pairs. Thus, in one embodiment, to the extent that it becomes processed to a functional duplex, of e.g., 15-30 base pairs, that targets a desired RNA for cleavage, an RNA molecule or complex of RNA molecules having a duplex region greater than 30 base pairs is a dsRNA. Thus, an ordinarily skilled artisan will recognize that in one embodiment, a miRNA is a dsRNA. In another embodiment, a dsRNA is not a naturally occurring miRNA. In another embodiment, an RNAi agent useful to target APP expression is not generated in the target cell by cleavage of a larger dsRNA.

A dsRNA as described herein can further include one or more single-stranded nucleotide overhangs e.g., 1, 2, 3, or 4 nucleotides. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′-end, 3′-end or both ends of either an antisense or sense strand of a dsRNA. In certain embodiments, longer, extended overhangs are possible.

A dsRNA can be synthesized by standard methods known in the art as further discussed below, e.g., by use of an automated DNA synthesizer, such as are commercially available from, for example, Biosearch, Applied Biosystems, Inc.

iRNA compounds of the disclosure may be prepared using a two-step procedure. First, the individual strands of the double stranded RNA molecule are prepared separately. Then, the component strands are annealed. The individual strands of the siRNA compound can be prepared using solution-phase or solid-phase organic synthesis or both. Organic synthesis offers the advantage that the oligonucleotide strands comprising unnatural or modified nucleotides can be easily prepared. Single-stranded oligonucleotides of the disclosure can be prepared using solution-phase or solid-phase organic synthesis or both.

An siRNA can be produced, e.g., in bulk, by a variety of methods. Exemplary methods include: organic synthesis and RNA cleavage, e.g., in vitro cleavage.

An siRNA can be made by separately synthesizing a single stranded RNA molecule, or each respective strand of a double-stranded RNA molecule, after which the component strands can then be annealed.

A large bioreactor, e.g., the OligoPilot II from Pharmacia Biotec AB (Uppsala Sweden), can be used to produce a large amount of a particular RNA strand for a given siRNA. The OligoPilotII reactor can efficiently couple a nucleotide using only a 1.5 molar excess of a phosphoramidite nucleotide. To make an RNA strand, ribonucleotides amidites are used. Standard cycles of monomer addition can be used to synthesize the 21 to 23 nucleotide strand for the siRNA. Typically, the two complementary strands are produced separately and then annealed, e.g., after release from the solid support and deprotection.

Organic synthesis can be used to produce a discrete siRNA species. The complementary of the species to an APP gene can be precisely specified. For example, the species may be complementary to a region that includes a polymorphism, e.g., a single nucleotide polymorphism.

Further the location of the polymorphism can be precisely defined. In some embodiments, the polymorphism is located in an internal region, e.g., at least 4, 5, 7, or 9 nucleotides from one or both of the termini.

In one embodiment, RNA generated is carefully purified to remove ends. iRNA is cleaved in vitro into siRNAs, for example, using a Dicer or comparable RNAse III-based activity. For example, the dsiRNA can be incubated in an in vitro extract from Drosophila or using purified components, e.g., a purified RNAse or RISC (RNA-induced silencing complex). See, e.g., Ketting et al. Genes Dev 2001 Oct. 15; 15(20): 2654-9 and Hammond Science 2001 Aug. 10; 293(5532): 1146-50.

dsiRNA cleavage generally produces a plurality of siRNA species, each being a particular 21 to 23 nt fragment of a source dsiRNA molecule. For example, siRNAs that include sequences complementary to overlapping regions and adjacent regions of a source dsiRNA molecule may be present.

Regardless of the method of synthesis, the siRNA preparation can be prepared in a solution (e.g., an aqueous or organic solution) that is appropriate for formulation. For example, the siRNA preparation can be precipitated and redissolved in pure double-distilled water, and lyophilized. The dried siRNA can then be resuspended in a solution appropriate for the intended formulation process.

In one aspect, a dsRNA of the disclosure includes at least two nucleotide sequences, a sense sequence and an antisense sequence. The sense strand sequence for APP may be selected from the group of sequences provided in any one of Tables 2-21, and the corresponding nucleotide sequence of the antisense strand of the sense strand may be selected from the group of sequences of any one of Tables 2-21. In this aspect, one of the two sequences is complementary to the other of the two sequences, with one of the sequences being substantially complementary to a sequence of an mRNA generated in the expression of an APP gene. As such, in this aspect, a dsRNA will include two oligonucleotides, where one oligonucleotide is described as the sense strand (passenger strand) in any one of Tables 2-21, and the second oligonucleotide is described as the corresponding antisense strand (guide strand) of the sense strand in any one of Tables 2-21 for APP.

In one embodiment, the substantially complementary sequences of the dsRNA are contained on separate oligonucleotides. In another embodiment, the substantially complementary sequences of the dsRNA are contained on a single oligonucleotide.

It will be understood that, although the sequences provided herein are described as modified or conjugated sequences, the RNA of the RNAi agent of the disclosure e.g., a dsRNA of the disclosure, may comprise any one of the sequences set forth in any one of Tables 2-21 that is un-modified, un-conjugated, or modified or conjugated differently than described therein. One or more lipophilic ligands or one or more GalNAc ligands can be included in any of the positions of the RNAi agents provided in the instant application.

The skilled person is well aware that dsRNAs having a duplex structure of about 20 to 23 base pairs, e.g., 21, base pairs have been hailed as particularly effective in inducing RNA interference (Elbashir et al., (2001) EMBO J., 20: 6877-6888). However, others have found that shorter or longer RNA duplex structures can also be effective (Chu and Rana (2007) RNA 14: 1714-1719; Kim et al. (2005) Nat Biotech 23: 222-226). In the embodiments described above, by virtue of the nature of the oligonucleotide sequences provided herein, dsRNAs described herein can include at least one strand of a length of minimally 21 nucleotides. It can be reasonably expected that shorter duplexes minus only a few nucleotides on one or both ends can be similarly effective as compared to the dsRNAs described above. Hence, dsRNAs having a sequence of at least 15, 16, 17, 18, 19, 20, or more contiguous nucleotides derived from one of the sequences provided herein, and differing in their ability to inhibit the expression of an APP gene by not more than 10, 15, 20, 25, or 30% inhibition from a dsRNA comprising the full sequence using the in vitro assay with Be(2)-C cells and a 10 nM concentration of the RNA agent and the PCR assay as provided in the examples herein, are contemplated to be within the scope of the present disclosure.

One benchmark assay for inhibition of APP involves contacting human Be(2)-C cells with a dsRNA agent as disclosed herein, where sufficient or effective APP inhibition is identified if at least 5% reduction, at least 10% reduction, at least 15% reduction, at least 20% reduction, at least 25% reduction, at least 30% reduction, at least 35% reduction, at least 40% reduction, at least 45% reduction, at least 50% reduction, at least 55% reduction, at least 60% reduction, at least 65% reduction, at least 70% reduction, at least 75% reduction, at least 80% reduction, at least 85% reduction, at least 90% reduction, at least 95% reduction, at least 97% reduction, at least 98% reduction, at least 99% reduction, or more of APP transcript or protein is observed in contacted cells, as compared to an appropriate control (e.g., cells not contacted with APP-targeting dsRNA). Optionally, a dsRNA agent of the disclosure is administered at 10 nM concentration, and the PCR assay is performed as provided in the examples herein (e.g., Example 2 below).

In addition, the RNAs described herein identify a site(s) in an APP transcript that is susceptible to RISC-mediated cleavage. As such, the present disclosure further features RNAi agents that target within this site(s). As used herein, an RNAi agent is said to target within a particular site of an RNA transcript if the RNAi agent promotes cleavage of the transcript anywhere within that particular site. Such an RNAi agent will generally include at least about 15 contiguous nucleotides, optionally at least 19 nucleotides, from one of the sequences provided herein coupled to additional nucleotide sequences taken from the region contiguous to the selected sequence in an APP gene.

An RNAi agent as described herein can contain one or more mismatches to the target sequence. In one embodiment, an RNAi agent as described herein contains no more than 3 mismatches (i.e., 3, 2, 1, or 0 mismatches). In one embodiment, an RNAi agent as described herein contains no more than 2 mismatches. In one embodiment, an RNAi agent as described herein contains no more than 1 mismatch. In one embodiment, an RNAi agent as described herein contains 0 mismatches. In certain embodiments, if the antisense strand of the RNAi agent contains mismatches to the target sequence, the mismatch can optionally be restricted to be within the last 5 nucleotides from either the 5′- or 3′-end of the region of complementarity. For example, in such embodiments, for a 23 nucleotide RNAi agent, the strand which is complementary to a region of an APP gene generally does not contain any mismatch within the central 13 nucleotides. The methods described herein or methods known in the art can be used to determine whether an RNAi agent containing a mismatch to a target sequence is effective in inhibiting the expression of an APP gene. Consideration of the efficacy of RNAi agents with mismatches in inhibiting expression of an APP gene is important, especially if the particular region of complementarity in an APP gene is known to have polymorphic sequence variation within the population.

### II. Modified RNAi Agents of the Disclosure

In one embodiment, the RNA of the RNAi agent of the disclosure e.g., a dsRNA, is un-modified, and does not comprise, e.g., chemical modifications or conjugations known in the art and described herein. In preferred embodiments, the RNA of an RNAi agent of the disclosure, e.g., a dsRNA, is chemically modified to enhance stability or other beneficial characteristics. In certain embodiments of the disclosure, substantially all of the nucleotides of an RNAi agent of the disclosure are modified. In other embodiments of the disclosure, all of the nucleotides of an RNAi agent of the disclosure are modified. RNAi agents of the disclosure in which “substantially all of the nucleotides are modified” are largely but not wholly modified and can include not more than 5, 4, 3, 2, or 1 unmodified nucleotides. In still other embodiments of the disclosure, RNAi agents of the disclosure can include not more than 5, 4, 3, 2 or 1 modified nucleotides.

The nucleic acids featured in the disclosure can be synthesized or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; or backbone modifications, including modification or replacement of the phosphodiester linkages. Specific examples of RNAi agents useful in the embodiments described herein include, but are not limited to, RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments, a modified RNAi agent will have a phosphorus atom in its internucleoside backbone.

Modified RNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′. Various salts, e.g., sodium salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, the entire contents of each of which are hereby incorporated herein by reference.

In other embodiments, suitable RNA mimetics are contemplated for use in RNAi agents, in which both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in the RNAi agents of the disclosure are described in, for example, in Nielsen et al., Science, 1991, 254, 1497-1500.

Some embodiments featured in the disclosure include RNAs with phosphorothioate backbones and oligonucleosides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —N(CH₃)—CH₂—CH₂— [wherein the native phosphodiester backbone is represented as —O—P—O—CH₂—] of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506.

Modified RNAs can also contain one or more substituted sugar moieties. The RNAi agents, e.g., dsRNAs, featured herein can include one of the following at the 2′-position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂)·ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an RNAi agent, or a group for improving the pharmacodynamic properties of an RNAi agent, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78: 486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂. Further exemplary modifications include: 5′-Me-2′-F nucleotides, 5′-Me-2′-OMe nucleotides, 5′-Me-2′-deoxynucleotides, (both R and S isomers in these three families); 2′-alkoxyalkyl; and 2′-NMA (N-methylacetamide).

Other modifications include 2′-methoxy (2′-OCH₃), 2′-aminopropoxy (2′-OCH₂CH₂CH₂NH₂), 2′-O-hexadecyl, and 2′-fluoro (2′-F). Similar modifications can also be made at other positions on the RNA of an RNAi agent, particularly the 3′ position of the sugar on the 3′ terminal nucleotide or in 2′-5′ linked dsRNAs and the 5′ position of 5′ terminal nucleotide. RNAi agents can also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application. The entire contents of each of the foregoing are hereby incorporated herein by reference.

An RNAi agent of the disclosure can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., (1991) Angewandte Chemie, International Edition, 30: 613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the disclosure. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

An RNAi agent of the disclosure can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1): 439-447; Mook, O R. et al., (2007) Mol Canc Ther 6(3): 833-843; Grunweller, A. et al., (2003) Nucleic Acids Research 31(12): 3185-3193).

An RNAi agent of the disclosure can also be modified to include one or more bicyclic sugar moities. A “bicyclic sugar” is a furanosyl ring modified by the bridging of two atoms. A “bicyclic nucleoside” (“BNA”) is a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring. Thus, in some embodiments an agent of the disclosure may include one or more locked nucleic acids (LNA). A locked nucleic acid is a nucleotide having a modified ribose moiety in which the ribose moiety comprises an extra bridge connecting the 2′ and 4′ carbons. In other words, an LNA is a nucleotide comprising a bicyclic sugar moiety comprising a 4′-CH₂—O-2′ bridge. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1): 439-447; Mook, O R. et al., (2007) Mol Canc Ther 6(3): 833-843; Grunweller, A. et al., (2003) Nucleic Acids Research 31(12): 3185-3193). Examples of bicyclic nucleosides for use in the polynucleotides of the disclosure include without limitation nucleosides comprising a bridge between the 4′ and the 2′ ribosyl ring atoms. In certain embodiments, the antisense polynucleotide agents of the disclosure include one or more bicyclic nucleosides comprising a 4′ to 2′ bridge. Examples of such 4′ to 2′ bridged bicyclic nucleosides, include but are not limited to 4′-(CH2)-O-2′ (LNA); 4′-(CH2)S-2′; 4′-(CH2)2-O-2′ (ENA); 4′-CH(CH3)-O-2′ (also referred to as “constrained ethyl” or “cEt”) and 4′-CH(CH2OCH3)-O-2′ (and analogs thereof, see, e.g., U.S. Pat. No. 7,399,845); 4′-C(CH3)(CH3)-O-2′ (and analogs thereof; see e.g., U.S. Pat. No. 8,278,283); 4′-CH2 N(OCH3)-2′ (and analogs thereof; see e.g., U.S. Pat. No. 8,278,425); 4′-CH2-O—N(CH3)-2′ (see, e.g., U.S. Patent Publication No. 2004/0171570); 4′-CH2-N(R)—O-2′, wherein R is H, C1-C12 alkyl, or a protecting group (see, e.g., U.S. Pat. No. 7,427,672); 4′-CH2-C(H)(CH3)-2′ (see, e.g., Chattopadhyaya et al., J. Org. Chem., 2009, 74, 118-134); and 4′-CH2-C(═CH2)-2′ (and analogs thereof, see, e.g., U.S. Pat. No. 8,278,426). The entire contents of each of the foregoing are hereby incorporated herein by reference.

Additional representative US patents and US Patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleosides can be prepared having one or more stereochemical sugar configurations including for example α-L-ribofuranose and 3-D-ribofuranose (see WO 99/14226).

An RNAi agent of the disclosure can also be modified to include one or more constrained ethyl nucleotides. As used herein, a “constrained ethyl nucleotide” or “cEt” is a locked nucleic acid comprising a bicyclic sugar moiety comprising a 4′-CH(CH₃)—O-2′ bridge. In one embodiment, a constrained ethyl nucleotide is in the S conformation referred to herein as “S-cEt.”

An RNAi agent of the disclosure may also include one or more “conformationally restricted nucleotides” (“CRN”). CRN are nucleotide analogs with a linker connecting the C2′ and C4′ carbons of ribose or the C3′ and C5′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, US 2013/0190383; and WO 2013/036868, the entire contents of each of which are hereby incorporated herein by reference.

In some embodiments, an RNAi agent of the disclosure comprises one or more monomers that are UNA (unlocked nucleic acid) nucleotides. UNA is unlocked acyclic nucleic acid, wherein any of the bonds of the sugar has been removed, forming an unlocked “sugar” residue. In one example, UNA also encompasses monomer with bonds between C1′-C4′ have been removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and U.S. Patent Publication Nos. 2013/0096289; 2013/0011922; and 2011/0313020, the entire contents of each of which are hereby incorporated herein by reference.

Potentially stabilizing modifications to the ends of RNA molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in WO 2011/005861.

Other modifications of an RNAi agent of the disclosure include a 5′ phosphate or 5′ phosphate mimic, e.g., a 5′-terminal phosphate or phosphate mimic on the antisense strand of an RNAi agent. Suitable phosphate mimics are disclosed in, for example US 2012/0157511, the entire contents of which are incorporated herein by reference.

A. Modified RNAi agents Comprising Motifs of the Disclosure

In certain aspects of the disclosure, the double-stranded RNAi agents of the disclosure include agents with chemical modifications as disclosed, for example, in WO 2013/075035, the entire contents of which are incorporated herein by reference. As shown herein and in WO 2013/075035, a superior result may be obtained by introducing one or more motifs of three identical modifications on three consecutive nucleotides into a sense strand or antisense strand of an RNAi agent, particularly at or near the cleavage site. In some embodiments, the sense strand and antisense strand of the RNAi agent may otherwise be completely modified. The introduction of these motifs interrupts the modification pattern, if present, of the sense or antisense strand. The RNAi agent may be optionally conjugated with a lipophilic ligand, e.g., a C16 ligand, for instance on the sense strand. The RNAi agent may be optionally modified with a (S)-glycol nucleic acid (GNA) modification, for instance on one or more residues of the antisense strand. The resulting RNAi agents present superior gene silencing activity.

Accordingly, the disclosure provides double stranded RNAi agents capable of inhibiting the expression of a target gene (i.e., an APP gene) in vivo. The RNAi agent comprises a sense strand and an antisense strand. Each strand of the RNAi agent may be 15-30 nucleotides in length. For example, each strand may be 16-30 nucleotides in length, 17-30 nucleotides in length, 25-30 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length. In certain embodiments, each strand is 19-23 nucleotides in length.

The sense strand and antisense strand typically form a duplex double stranded RNA (“dsRNA”), also referred to herein as an “RNAi agent.” The duplex region of an RNAi agent may be 15-30 nucleotide pairs in length. For example, the duplex region can be 16-30 nucleotide pairs in length, 17-30 nucleotide pairs in length, 27-30 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotides in length. In preferred embodiments, the duplex region is 19-21 nucleotide pairs in length.

In one embodiment, the RNAi agent may contain one or more overhang regions or capping groups at the 3′-end, 5′-end, or both ends of one or both strands. The overhang can be 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. In preferred embodiments, the nucleotide overhang region is 2 nucleotides in length. The overhangs can be the result of one strand being longer than the other, or the result of two strands of the same length being staggered. The overhang can form a mismatch with the target mRNA or it can be complementary to the gene sequences being targeted or can be another sequence. The first and second strands can also be joined, e.g., by additional bases to form a hairpin, or by other non-base linkers.

In one embodiment, the nucleotides in the overhang region of the RNAi agent can each independently be a modified or unmodified nucleotide including, but no limited to 2′-sugar modified, such as, 2′-F, 2′-O-methyl, thymidine (T), and any combinations thereof.

For example, TT can be an overhang sequence for either end on either strand. The overhang can form a mismatch with the target mRNA or it can be complementary to the gene sequences being targeted or can be another sequence.

The 5′- or 3′-overhangs at the sense strand, antisense strand or both strands of the RNAi agent may be phosphorylated. In some embodiments, the overhang region(s) contains two nucleotides having a phosphorothioate between the two nucleotides, where the two nucleotides can be the same or different. In one embodiment, the overhang is present at the 3′-end of the sense strand, antisense strand, or both strands. In one embodiment, this 3′-overhang is present in the antisense strand. In one embodiment, this 3′-overhang is present in the sense strand.

The RNAi agent may contain only a single overhang, which can strengthen the interference activity of the RNAi, without affecting its overall stability. For example, the single-stranded overhang may be located at the 3′-terminal end of the sense strand or, alternatively, at the 3′-terminal end of the antisense strand. The RNAi may also have a blunt end, located at the 5′-end of the antisense strand (or the 3′-end of the sense strand) or vice versa. Generally, the antisense strand of the RNAi has a nucleotide overhang at the 3′-end, and the 5′-end is blunt. While not wishing to be bound by theory, the asymmetric blunt end at the 5′-end of the antisense strand and 3′-end overhang of the antisense strand favor the guide strand loading into RISC process.

In one embodiment, the RNAi agent is a double ended bluntmer of 19 nucleotides in length, wherein the sense strand contains at least one motif of three 2′-F modifications on three consecutive nucleotides at positions 7, 8, 9 from the 5′end. The antisense strand contains at least one motif of three 2′-O-methyl modifications on three consecutive nucleotides at positions 11, 12, 13 from the 5′end.

In another embodiment, the RNAi agent is a double ended bluntmer of 20 nucleotides in length, wherein the sense strand contains at least one motif of three 2′-F modifications on three consecutive nucleotides at positions 8, 9, 10 from the 5′end. The antisense strand contains at least one motif of three 2′-O-methyl modifications on three consecutive nucleotides at positions 11, 12, 13 from the 5′end.

In yet another embodiment, the RNAi agent is a double ended bluntmer of 21 nucleotides in length, wherein the sense strand contains at least one motif of three 2′-F modifications on three consecutive nucleotides at positions 9, 10, 11 from the 5′end. The antisense strand contains at least one motif of three 2′-O-methyl modifications on three consecutive nucleotides at positions 11, 12, 13 from the 5′end.

In one embodiment, the RNAi agent comprises a 21 nucleotide sense strand and a 23 nucleotide antisense strand, wherein the sense strand contains at least one motif of three 2′-F modifications on three consecutive nucleotides at positions 9, 10, 11 from the 5′end; the antisense strand contains at least one motif of three 2′-O-methyl modifications on three consecutive nucleotides at positions 11, 12, 13 from the 5′end, wherein one end of the RNAi agent is blunt, while the other end comprises a 2 nucleotide overhang. Optionally, the 2 nucleotide overhang is at the 3′-end of the antisense strand. When the 2 nucleotide overhang is at the 3′-end of the antisense strand, there may be two phosphorothioate internucleotide linkages between the terminal three nucleotides, wherein two of the three nucleotides are the overhang nucleotides, and the third nucleotide is a paired nucleotide next to the overhang nucleotide. In one embodiment, the RNAi agent additionally has two phosphorothioate internucleotide linkages between the terminal three nucleotides at both the 5′-end of the sense strand and at the 5′-end of the antisense strand. In one embodiment, every nucleotide in the sense strand and the antisense strand of the RNAi agent, including the nucleotides that are part of the motifs are modified nucleotides. In one embodiment each residue is independently modified with a 2′-O-methyl or 3′-fluoro, e.g., in an alternating motif. Optionally, the RNAi agent further comprises a ligand (e.g., a lipophilic ligand, optionally a C16 ligand).

In one embodiment, the RNAi agent comprises a sense and an antisense strand, wherein the sense strand is 25-30 nucleotide residues in length, wherein starting from the 5′ terminal nucleotide (position 1) positions 1 to 23 of the first strand comprise at least 8 ribonucleotides; the antisense strand is 36-66 nucleotide residues in length and, starting from the 3′ terminal nucleotide, comprises at least 8 ribonucleotides in the positions paired with positions 1-23 of sense strand to form a duplex; wherein at least the 3′ terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when the double stranded nucleic acid is introduced into a mammalian cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In one embodiment, the RNAi agent comprises sense and antisense strands, wherein the RNAi agent comprises a first strand having a length which is at least 25 and at most 29 nucleotides and a second strand having a length which is at most 30 nucleotides with at least one motif of three 2′-O-methyl modifications on three consecutive nucleotides at position 11, 12, 13 from the 5′ end; wherein the 3′ end of the first strand and the 5′ end of the second strand form a blunt end and the second strand is 1-4 nucleotides longer at its 3′ end than the first strand, wherein the duplex region which is at least 25 nucleotides in length, and the second strand is sufficiently complementary to a target mRNA along at least 19 nucleotide of the second strand length to reduce target gene expression when the RNAi agent is introduced into a mammalian cell, and wherein dicer cleavage of the RNAi agent preferentially results in an siRNA comprising the 3′ end of the second strand, thereby reducing expression of the target gene in the mammal. Optionally, the RNAi agent further comprises a ligand.

In one embodiment, the sense strand of the RNAi agent contains at least one motif of three identical modifications on three consecutive nucleotides, where one of the motifs occurs at the cleavage site in the sense strand.

In one embodiment, the antisense strand of the RNAi agent can also contain at least one motif of three identical modifications on three consecutive nucleotides, where one of the motifs occurs at or near the cleavage site in the antisense strand.

For an RNAi agent having a duplex region of 17-23 nucleotide in length, the cleavage site of the antisense strand is typically around the 10, 11 and 12 positions from the 5′-end. Thus the motifs of three identical modifications may occur at the 9, 10, 11 positions; 10, 11, 12 positions; 11, 12, 13 positions; 12, 13, 14 positions; or 13, 14, 15 positions of the antisense strand, the count starting from the 1^{st} nucleotide from the 5′-end of the antisense strand, or, the count starting from the 1^{st} paired nucleotide within the duplex region from the 5′-end of the antisense strand. The cleavage site in the antisense strand may also change according to the length of the duplex region of the RNAi from the 5′-end.

The sense strand of the RNAi agent may contain at least one motif of three identical modifications on three consecutive nucleotides at the cleavage site of the strand; and the antisense strand may have at least one motif of three identical modifications on three consecutive nucleotides at or near the cleavage site of the strand. When the sense strand and the antisense strand form a dsRNA duplex, the sense strand and the antisense strand can be so aligned that one motif of the three nucleotides on the sense strand and one motif of the three nucleotides on the antisense strand have at least one nucleotide overlap, i.e., at least one of the three nucleotides of the motif in the sense strand forms a base pair with at least one of the three nucleotides of the motif in the antisense strand. Alternatively, at least two nucleotides may overlap, or all three nucleotides may overlap.

In one embodiment, the sense strand of the RNAi agent may contain more than one motif of three identical modifications on three consecutive nucleotides. The first motif may occur at or near the cleavage site of the strand and the other motifs may be a wing modification. The term “wing modification” herein refers to a motif occurring at another portion of the strand that is separated from the motif at or near the cleavage site of the same strand. The wing modification is either adjacent to the first motif or is separated by at least one or more nucleotides. When the motifs are immediately adjacent to each other, the chemistry of the motifs are distinct from each other; and when the motifs are separated by one or more nucleotide, the chemistries can be the same or different. Two or more wing modifications may be present. For instance, when two wing modifications are present, each wing modification may occur at one end relative to the first motif which is at or near cleavage site or on either side of the lead motif.

Like the sense strand, the antisense strand of the RNAi agent may contain more than one motif of three identical modifications on three consecutive nucleotides, with at least one of the motifs occurring at or near the cleavage site of the strand. This antisense strand may also contain one or more wing modifications in an alignment similar to the wing modifications that may be present on the sense strand.

In one embodiment, the wing modification on the sense strand or antisense strand of the RNAi agent typically does not include the first one or two terminal nucleotides at the 3′-end, 5′-end or both ends of the strand.

In another embodiment, the wing modification on the sense strand or antisense strand of the RNAi agent typically does not include the first one or two paired nucleotides within the duplex region at the 3′-end, 5′-end or both ends of the strand.

When the sense strand and the antisense strand of the RNAi agent each contain at least one wing modification, the wing modifications may fall on the same end of the duplex region, and have an overlap of one, two or three nucleotides.

When the sense strand and the antisense strand of the RNAi agent each contain at least two wing modifications, the sense strand and the antisense strand can be so aligned that two modifications each from one strand fall on one end of the duplex region, having an overlap of one, two or three nucleotides; two modifications each from one strand fall on the other end of the duplex region, having an overlap of one, two or three nucleotides; two modifications one strand fall on each side of the lead motif, having an overlap of one, two, or three nucleotides in the duplex region.

In one embodiment, the RNAi agent comprises mismatch(es) with the target, within the duplex, or combinations thereof. The mismatch may occur in the overhang region or the duplex region. The base pair may be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C (I=inosine) is preferred over G:C. Mismatches, e.g., non-canonical or other than canonical pairings (as described elsewhere herein) are preferred over canonical (A:T, A:U, G:C) pairings; and pairings which include a universal base are preferred over canonical pairings.

In one embodiment, the RNAi agent comprises at least one of the first 1, 2, 3, 4, or 5 base pairs within the duplex regions from the 5′-end of the antisense strand independently selected from the group of: A:U, G:U, I:C, and mismatched pairs, e.g., non-canonical or other than canonical pairings or pairings which include a universal base, to promote the dissociation of the antisense strand at the 5′-end of the duplex.

In one embodiment, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

In another embodiment, the nucleotide at the 3′-end of the sense strand is deoxy-thymine (dT). In another embodiment, the nucleotide at the 3′-end of the antisense strand is deoxy-thymine (dT). In one embodiment, there is a short sequence of deoxy-thymine nucleotides, for example, two dT nucleotides on the 3′-end of the sense or antisense strand.

In one embodiment, the sense strand sequence may be represented by formula (I):

(I)
5′ nₚ-Nₐ-(X X X )ᵢ-N_{b}-Y Y Y-N_{b}-(Z Z Z )ⱼ-Nₐ-n_{q} 3′

wherein:
i and j are each independently 0 or 1;
p and q are each independently 0-6;
each Nₐ independently represents an oligonucleotide sequence comprising 0-25 modified nucleotides, each sequence comprising at least two differently modified nucleotides;
each N_{b} independently represents an oligonucleotide sequence comprising 0-10 modified nucleotides;
each nₚ and n_{q} independently represent an overhang nucleotide;
wherein N_{b} and Y do not have the same modification; and
XXX, YYY and ZZZ each independently represent one motif of three identical modifications on three consecutive nucleotides. Optionally YYY is all 2′-F modified nucleotides.

In one embodiment, the Nₐ or N_{b} comprise modifications of alternating pattern.

In one embodiment, the YYY motif occurs at or near the cleavage site of the sense strand. For example, when the RNAi agent has a duplex region of 17-23 nucleotides in length, the YYY motif can occur at or the vicinity of the cleavage site (e.g.: can occur at positions 6, 7, 8, 7, 8, 9, 8, 9, 10, 9, 10, 11, 10, 11, 12 or 11, 12, 13) of—the sense strand, the count starting from the 1^{st} nucleotide, from the 5′-end; or optionally, the count starting at the 1^{st} paired nucleotide within the duplex region, from the 5′-end.

In one embodiment, i is 1 and j is 0, or i is 0 and j is 1, or both i and j are 1. The sense strand can therefore be represented by the following formulas:

(Ib)
5′ nₚ-Nₐ-YYY-N_{b}-ZZZ-Nₐ-n_{q} 3′;
(Ic)
5′ nₚ-Nₐ-XXX-N_{b}-YYY-Nₐ-n_{q} 3′;
or
(Id)
5′ nₚ-Nₐ-XXX-N_{b}-YYY-N_{b}-ZZZ-Nₐ-n_{q} 3′.

When the sense strand is represented by formula (Ib), N_{b} represents an oligonucleotide sequence comprising 0-10, 0-7, 0-5, 0-4, 0-2 or 0 modified nucleotides.

Each Nₐ independently can represent an oligonucleotide sequence comprising 2-20, 2-15, or 2-10 modified nucleotides.

When the sense strand is represented as formula (Ic), N_{b} represents an oligonucleotide sequence comprising 0-10, 0-7, 0-10, 0-7, 0-5, 0-4, 0-2 or 0 modified nucleotides. Each Nₐ can independently represent an oligonucleotide sequence comprising 2-20, 2-15, or 2-10 modified nucleotides.

When the sense strand is represented as formula (Id), each N_{b} independently represents an oligonucleotide sequence comprising 0-10, 0-7, 0-5, 0-4, 0-2 or 0 modified nucleotides. Optionally, N_{b} is 0, 1, 2, 3, 4, 5 or 6. Each Nₐ can independently represent an oligonucleotide sequence comprising 2-20, 2-15, or 2-10 modified nucleotides.

Each of X, Y and Z may be the same or different from each other.

In other embodiments, i is 0 and j is 0, and the sense strand may be represented by the formula:

(Ia)
5′ nₚ-Nₐ-YYY-Nₐ-n_{q} 3′.

When the sense strand is represented by formula (Ia), each Nₐ independently can represent an oligonucleotide sequence comprising 2-20, 2-15, or 2-10 modified nucleotides.

In one embodiment, the antisense strand sequence of the RNAi may be represented by formula (II):

(II)
5′ n_{q′}-Nₐ′-(Z′Z′Z′)ₖ-N_{b}′-Y′Y′Y′-N_{b}′-
(X′X′X′)ₗ-N′ₐ-nₚ′ 3′

wherein:
k and l are each independently 0 or 1;
p′ and q′ are each independently 0-6;
each Nₐ′ independently represents an oligonucleotide sequence comprising 0-25 modified nucleotides, each sequence comprising at least two differently modified nucleotides;
each N_{b}′ independently represents an oligonucleotide sequence comprising 0-10 modified nucleotides;
each nₚ′ and n_{q}′ independently represent an overhang nucleotide;
wherein N_{b}′ and Y′ do not have the same modification;
and
X′X′X′, Y′Y′Y′ and Z′Z′Z′ each independently represent one motif of three identical modifications on three consecutive nucleotides.

In one embodiment, the Nₐ′ or N_{b}′ comprise modifications of alternating pattern.

The Y′Y′Y′ motif occurs at or near the cleavage site of the antisense strand. For example, when the RNAi agent has a duplex region of 17-23 nucleotide in length, the Y′Y′Y′ motif can occur at positions 9, 10, 11; 10, 11, 12; 11, 12, 13; 12, 13, 14; or 13, 14, 15 of the antisense strand, with the count starting from the 1^{st} nucleotide, from the 5′-end; or optionally, the count starting at the 1^{st} paired nucleotide within the duplex region, from the 5′-end. Optionally, the Y′Y′Y′ motif occurs at positions 11, 12, 13.

In one embodiment, Y′Y′Y′ motif is all 2′-OMe modified nucleotides.

In one embodiment, k is 1 and 1 is 0, or k is 0 and 1 is 1, or both k and 1 are 1.

The antisense strand can therefore be represented by the following formulas:

(IIb)
5′ n_{q′}-Nₐ′-Z′Z′Z′-N_{b}′-Y′Y′Y′-Nₐ′-nₚ′ 3′;
(IIc)
5′ n_{q′}-Nₐ′-Y′Y′Y′-N_{b}′-X′X′X′-n_{p′} 3′;
or
(IId)
5′ n_{q′}-Nₐ′-Z′Z′Z′-N_{b}′-Y′Y′Y′-N_{b}′-X′X′X′-Nₐ′-n_{p′} 3′.

When the antisense strand is represented by formula (IIb), N_{b} represents an oligonucleotide sequence comprising 0-10, 0-7, 0-10, 0-7, 0-5, 0-4, 0-2 or 0 modified nucleotides. Each Nₐ′ independently represents an oligonucleotide sequence comprising 2-20, 2-15, or 2-10 modified nucleotides.

When the antisense strand is represented as formula (IIc), N_{b}′ represents an oligonucleotide sequence comprising 0-10, 0-7, 0-10, 0-7, 0-5, 0-4, 0-2 or 0 modified nucleotides. Each Nₐ′ independently represents an oligonucleotide sequence comprising 2-20, 2-15, or 2-10 modified nucleotides.

When the antisense strand is represented as formula (IId), each N_{b}′ independently represents an oligonucleotide sequence comprising 0-10, 0-7, 0-10, 0-7, 0-5, 0-4, 0-2 or 0 modified nucleotides. Each Nₐ′ independently represents an oligonucleotide sequence comprising 2-20, 2-15, or 2-10 modified nucleotides. Optionally, N_{b} is 0, 1, 2, 3, 4, 5 or 6.

In other embodiments, k is 0 and 1 is 0 and the antisense strand may be represented by the formula:

(Ia)
5′ n_{p′}-Nₐ′-Y′Y′Y′-N_{a′}-n_{q′} 3′.

When the antisense strand is represented as formula (IIa), each Nₐ′ independently represents an oligonucleotide sequence comprising 2-20, 2-15, or 2-10 modified nucleotides.

Each of X′, Y′ and Z′ may be the same or different from each other.

Each nucleotide of the sense strand and antisense strand may be independently modified with LNA, HNA, CeNA, 2′-methoxyethyl, 2′-O-methyl, 2′-O-allyl, 2′-C-allyl, 2′-hydroxyl, or 2′-fluoro. For example, each nucleotide of the sense strand and antisense strand is independently modified with 2′-O-methyl or 2′-fluoro. Each X, Y, Z, X′, Y′ and Z′, in particular, may represent a 2′-O-methyl modification or a 2′-fluoro modification.

In one embodiment, the sense strand of the RNAi agent may contain YYY motif occurring at 9, 10 and 11 positions of the strand when the duplex region is 21 nt, the count starting from the 1^{st} nucleotide from the 5′-end, or optionally, the count starting at the 1^{st} paired nucleotide within the duplex region, from the 5′-end; and Y represents 2′-F modification. The sense strand may additionally contain XXX motif or ZZZ motifs as wing modifications at the opposite end of the duplex region; and XXX and ZZZ each independently represents a 2′-OMe modification or 2′-F modification.

In one embodiment the antisense strand may contain Y′Y′Y′ motif occurring at positions 11, 12, 13 of the strand, the count starting from the 1^{st} nucleotide from the 5′-end, or optionally, the count starting at the 1^{st} paired nucleotide within the duplex region, from the 5′-end; and Y′ represents 2′-O-methyl modification. The antisense strand may additionally contain X′X′X′ motif or Z′Z′Z′ motifs as wing modifications at the opposite end of the duplex region; and X′X′X′ and Z′Z′Z′ each independently represents a 2′-OMe modification or 2′-F modification.

The sense strand represented by any one of the above formulas (Ia), (Ib), (Ic), and (Id) forms a duplex with an antisense strand being represented by any one of formulas (IIa), (IIb), (IIc), and (IId), respectively.

Accordingly, the RNAi agents for use in the methods of the disclosure may comprise a sense strand and an antisense strand, each strand having 14 to 30 nucleotides, the RNAi duplex represented by formula (III):

(III)
sense:
5′ nₚ-Nₐ-(X X X)ᵢ-N_{b}-Y Y Y-N_{b}-(ZZ Z)ⱼ-Nₐ-n_{q} 3′
antisense:
3′ nₚ′-Nₐ′-(X′X′X′)ₖ-N_{b}′-Y′Y′Y′-N_{b}′-
(Z′Z′Z′)ₗ-Nₐ′-n_{q}′ 5′

wherein:
i, j, k, and 1 are each independently 0 or 1;
p, p′, q, and q′ are each independently 0-6;
each Nₐ and Nₐ′ independently represents an oligonucleotide sequence comprising 0-25 modified nucleotides, each sequence comprising at least two differently modified nucleotides;
each N_{b} and N_{b}′ independently represents an oligonucleotide sequence comprising 0-10 modified nucleotides;
wherein
each nₚ′, nₚ, n_{q}′, and n_{q}, each of which may or may not be present, independently represents an overhang nucleotide; and
XXX, YYY, ZZZ, X′X′X′, Y′Y′Y′, and Z′Z′Z′ each independently represent one motif of three identical modifications on three consecutive nucleotides.

In one embodiment, i is 0 and j is 0; or i is 1 and j is 0; or i is 0 and j is 1; or both i and j are 0; or both i and j are 1. In another embodiment, k is 0 and 1 is 0; or k is 1 and 1 is 0; k is 0 and 1 is 1; or both k and 1 are 0; or both k and 1 are 1.

Exemplary combinations of the sense strand and antisense strand forming an RNAi duplex include the formulas below:

(IIIa)
5′ nₚ-Nₐ-Y Y Y-Nₐ-n_{q} 3′
3′ nₚ′-Nₐ′-Y′Y′Y′-Nₐ′ n_{q}′ 5′
(IIIb)
5′ nₚ-Nₐ-Y Y Y-N_{b}-ZZZ-Nₐ-n_{q} 3′
3′ nₚ′-Nₐ′-Y′Y′Y′-N_{b}′-Z′Z′Z′-Nₐ′ n_{q}′ 5′
(IIIc)
5′ nₚ-Nₐ-X X X-N_{b}-Y Y Y-Nₐ-n_{q} 3′
3′ nₚ′-Nₐ′-X′X′X′-N_{b}-Y′Y′Y′-Nₐ′-n_{q}′ 5′
(IIId)
5′ nₚ-Nₐ-X X X-N_{b}-Y Y Y-N_{b}-ZZZ-Nₐ-n_{q} 3′
3′ nₚ′-Nₐ′-X′X′X′-N_{b}′-Y′Y′Y′-N_{b}′-Z′Z′Z′-Nₐ-n_{q}′ 5′

When the RNAi agent is represented by formula (IIIa), each Nₐ independently represents an oligonucleotide sequence comprising 2-20, 2-15, or 2-10 modified nucleotides.

When the RNAi agent is represented by formula (IIIb), each N_{b} independently represents an oligonucleotide sequence comprising 1-10, 1-7, 1-5 or 1-4 modified nucleotides. Each Nₐ independently represents an oligonucleotide sequence comprising 2-20, 2-15, or 2-10 modified nucleotides.

When the RNAi agent is represented as formula (IIIc), each N_{b}, N_{b}′ independently represents an oligonucleotide sequence comprising 0-10, 0-7, 0-10, 0-7, 0-5, 0-4, 0-2 or 0 modified nucleotides. Each Nₐ independently represents an oligonucleotide sequence comprising 2-20, 2-15, or 2-10 modified nucleotides.

When the RNAi agent is represented as formula (IIId), each N_{b}, N_{b}′ independently represents an oligonucleotide sequence comprising 0-10, 0-7, 0-10, 0-7, 0-5, 0-4, 0-2 or 0 modified nucleotides. Each Nₐ, Nₐ′ independently represents an oligonucleotide sequence comprising 2-20, 2-15, or 2-10 modified nucleotides. Each of Nₐ, Nₐ′, N_{b} and N_{b}′ independently comprises modifications of alternating pattern.

In one embodiment, when the RNAi agent is represented by formula (IIId), the Nₐ modifications are 2′-O-methyl or 2′-fluoro modifications. In another embodiment, when the RNAi agent is represented by formula (IIId), the Nₐ modifications are 2′-O-methyl or 2′-fluoro modifications and nₚ′>0 and at least one nₚ′ is linked to a neighboring nucleotide a via phosphorothioate linkage. In yet another embodiment, when the RNAi agent is represented by formula (IIId), the Nₐ modifications are 2′-O-methyl or 2′-fluoro modifications, nₚ′>0 and at least one nₚ′ is linked to a neighboring nucleotide via phosphorothioate linkage, and the sense strand is conjugated to one or more C16 (or related) moieties attached through a bivalent or trivalent branched linker (described below). In another embodiment, when the RNAi agent is represented by formula (IIId), the Nₐ modifications are 2′-O-methyl or 2′-fluoro modifications, nₚ′>0 and at least one nₚ′ is linked to a neighboring nucleotide via phosphorothioate linkage, the sense strand comprises at least one phosphorothioate linkage, and the sense strand is conjugated to one or more lipophilic, e.g., C16 (or related) moieties, optionally attached through a bivalent or trivalent branched linker.

In one embodiment, when the RNAi agent is represented by formula (IIIa), the Nₐ modifications are 2′-O-methyl or 2′-fluoro modifications, nₚ′>0 and at least one nₚ′ is linked to a neighboring nucleotide via phosphorothioate linkage, the sense strand comprises at least one phosphorothioate linkage, and the sense strand is conjugated to one or more lipophilic, e.g., C16 (or related) moieties attached through a bivalent or trivalent branched linker.

In one embodiment, the RNAi agent is a multimer containing at least two duplexes represented by formula (III), (IIIa), (IIIb), (IIIc), and (IIId), wherein the duplexes are connected by a linker. The linker can be cleavable or non-cleavable. Optionally, the multimer further comprises a ligand. Each of the duplexes can target the same gene or two different genes; or each of the duplexes can target same gene at two different target sites.

In one embodiment, the RNAi agent is a multimer containing three, four, five, six or more duplexes represented by formula (III), (IIIa), (IIIb), (IIIc), and (IIId), wherein the duplexes are connected by a linker. The linker can be cleavable or non-cleavable. Optionally, the multimer further comprises a ligand. Each of the duplexes can target the same gene or two different genes; or each of the duplexes can target same gene at two different target sites.

In one embodiment, two RNAi agents represented by formula (III), (IIIa), (IIIb), (IIIc), and (IIId) are linked to each other at the 5′ end, and one or both of the 3′ ends and are optionally conjugated to a ligand. Each of the agents can target the same gene or two different genes; or each of the agents can target same gene at two different target sites.

Various publications describe multimeric RNAi agents that can be used in the methods of the disclosure. Such publications include WO2007/091269, WO2010/141511, WO2007/117686, WO2009/014887, and WO2011/031520; and U.S. Pat. No. 7,858,769, the entire contents of each of which are hereby incorporated herein by reference.

In certain embodiments, the compositions and methods of the disclosure include a vinyl phosphonate (VP) modification of an RNAi agent as described herein. In exemplary embodiments, a vinyl phosphonate of the disclosure has the following structure:

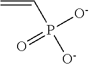

A vinyl phosphonate of the instant disclosure may be attached to either the antisense or the sense strand of a dsRNA of the disclosure. In certain preferred embodiments, a vinyl phosphonate of the instant disclosure is attached to the antisense strand of a dsRNA, optionally at the 5′ end of the antisense strand of the dsRNA.

Vinyl phosphate modifications are also contemplated for the compositions and methods of the instant disclosure. An exemplary vinyl phosphate structure is:

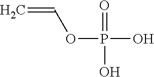

### B. Thermally Destabilizing Modifications

In certain embodiments, a dsRNA molecule can be optimized for RNA interference by incorporating thermally destabilizing modifications in the seed region of the antisense strand (i.e., at positions 2-9 of the 5′-end of the antisense strand) to reduce or inhibit off-target gene silencing. It has been discovered that dsRNAs with an antisense strand comprising at least one thermally destabilizing modification of the duplex within the first 9 nucleotide positions, counting from the 5′ end, of the antisense strand have reduced off-target gene silencing activity. Accordingly, in some embodiments, the antisense strand comprises at least one (e.g., one, two, three, four, five or more) thermally destabilizing modification of the duplex within the first 9 nucleotide positions of the 5′ region of the antisense strand. In some embodiments, one or more thermally destabilizing modification(s) of the duplex is/are located in positions 2-9, or optionally positions 4-8, from the 5′-end of the antisense strand. In some further embodiments, the thermally destabilizing modification(s) of the duplex is/are located at position 6, 7 or 8 from the 5′-end of the antisense strand. In still some further embodiments, the thermally destabilizing modification of the duplex is located at position 7 from the 5′-end of the antisense strand. The term “thermally destabilizing modification(s)” includes modification(s) that would result with a dsRNA with a lower overall melting temperature (Tm) (optionally a Tm with one, two, three or four degrees lower than the Tm of the dsRNA without having such modification(s). In some embodiments, the thermally destabilizing modification of the duplex is located at position 2, 3, 4, 5 or 9 from the 5′-end of the antisense strand.

The thermally destabilizing modifications can include, but are not limited to, abasic modification; mismatch with the opposing nucleotide in the opposing strand; and sugar modification such as 2′-deoxy modification or acyclic nucleotide, e.g., unlocked nucleic acids (UNA) or glycol nucleic acid (GNA).

Exemplified abasic modifications include, but are not limited to the following:

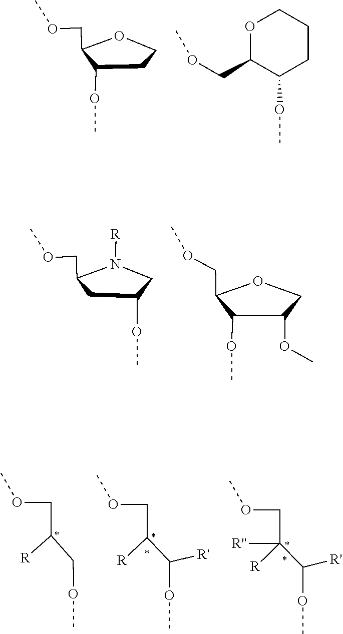

### Wherein R=H, Me, Et or OMe; R′=H, Me, Et or OMe; R″=H, Me, Et or OMe

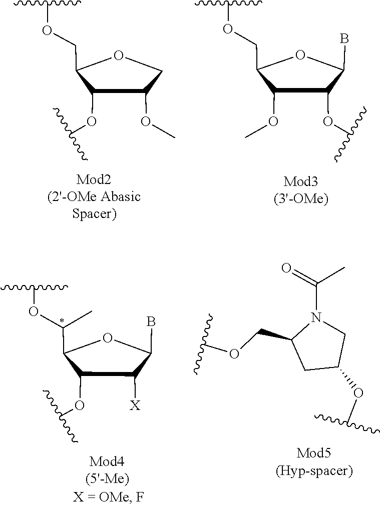

wherein B is a modified or unmodified nucleobase.

Exemplified sugar modifications include, but are not limited to the following.

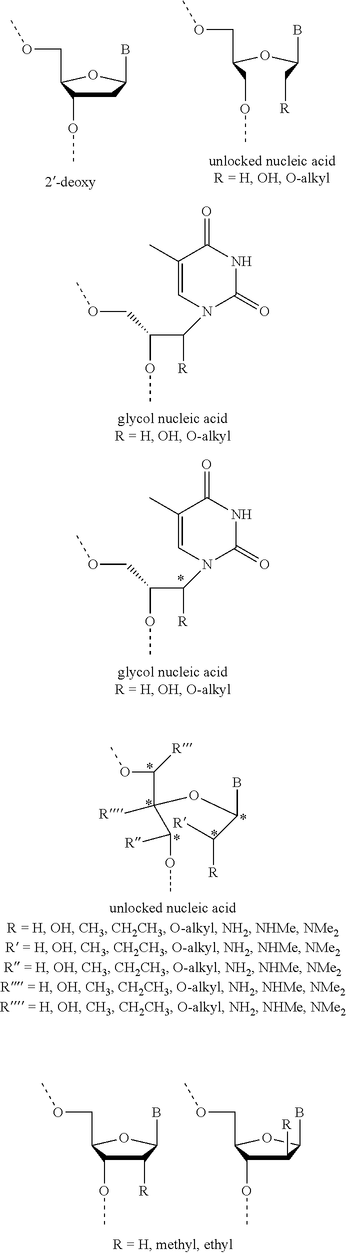

wherein B is a modified or unmodified nucleobase.

In some embodiments the thermally destabilizing modification of the duplex is selected from the group consisting of:

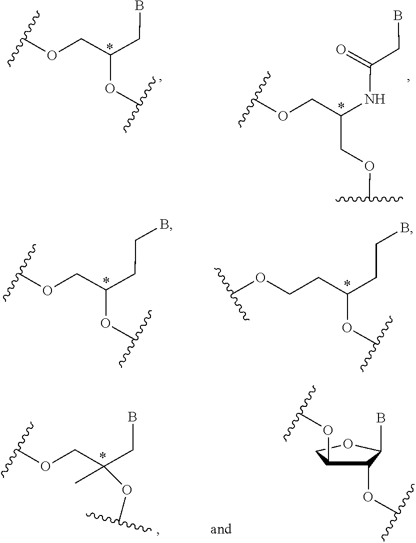

wherein B is a modified or unmodified nucleobase and the asterisk on each structure represents either R, S or racemic.

The term “acyclic nucleotide” refers to any nucleotide having an acyclic ribose sugar, for example, where any of bonds between the ribose carbons (e.g., C1′-C2′, C2′-C3′, C3′-C4′, C4′-O4′, or C1′-O4′) is absent or at least one of ribose carbons or oxygen (e.g., C1′, C2′, C3′, C4′ or O4′) are independently or in combination absent from the nucleotide. In some embodiments, acyclic nucleotide is

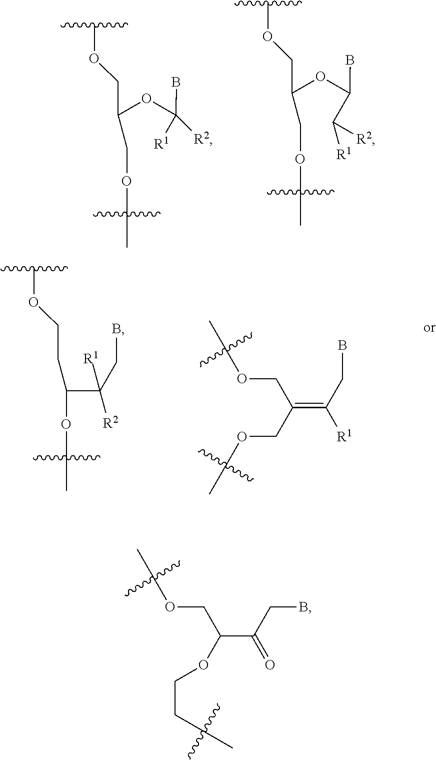

wherein B is a modified or unmodified nucleobase, R¹ and R² independently are H, halogen, OR₃, or alkyl; and R₃ is H, alkyl, cycloalkyl, aryl, aralkyl, heteroaryl or sugar). The term “UNA” refers to unlocked acyclic nucleic acid, wherein any of the bonds of the sugar has been removed, forming an unlocked “sugar” residue. In one example, UNA also encompasses monomers with bonds between C1′-C4′ being removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

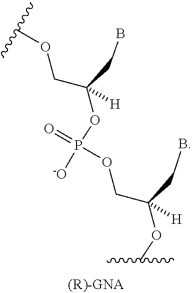

The thermally destabilizing modification of the duplex can be mismatches (i.e., noncomplementary base pairs) between the thermally destabilizing nucleotide and the opposing nucleotide in the opposite strand within the dsRNA duplex. Exemplary mismatch base pairs include G:G, G:A, G:U, G:T, A:A, A:C, C:C, C:U, C:T, U:U, T:T, U:T, or a combination thereof. Other mismatch base pairings known in the art are also amenable to the present disclosure. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the dsRNA molecule contains at least one nucleobase in the mismatch pairing that is a 2′-deoxy nucleobase; e.g., the 2′-deoxy nucleobase is in the sense strand.

In some embodiments, the thermally destabilizing modification of the duplex in the seed region of the antisense strand includes nucleotides with impaired W—C H-bonding to complementary base on the target mRNA, such as:

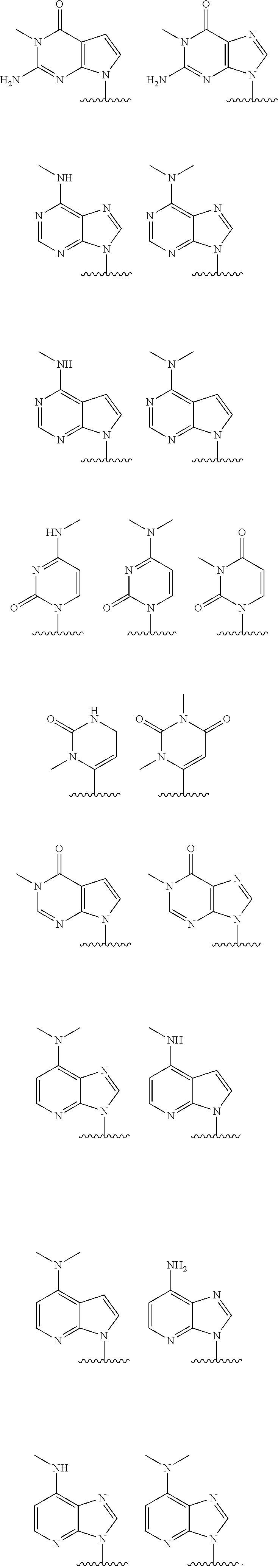

More examples of abasic nucleotide, acyclic nucleotide modifications (including UNA and GNA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

In some embodiments, the thermally destabilizing modification of the duplex includes nucleotides with non-canonical bases such as, but not limited to, nucleobase modifications with impaired or completely abolished capability to form hydrogen bonds with bases in the opposite strand. These nucleobase modifications have been evaluated for destabilization of the central region of the dsRNA duplex as described in WO 2010/0011895, which is herein incorporated by reference in its entirety. Exemplary nucleobase modifications are:

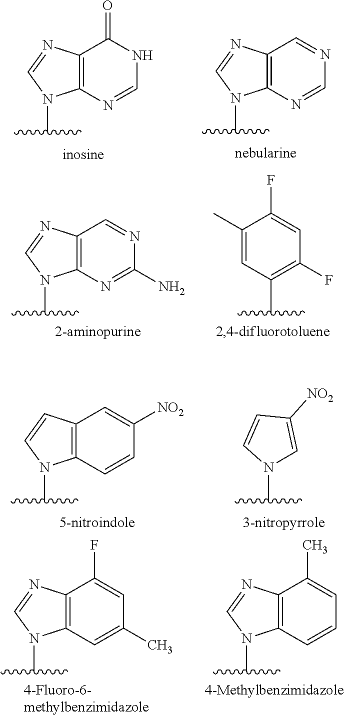

In some embodiments, the thermally destabilizing modification of the duplex in the seed region of the antisense strand includes one or more α-nucleotide complementary to the base on the target mRNA, such as:

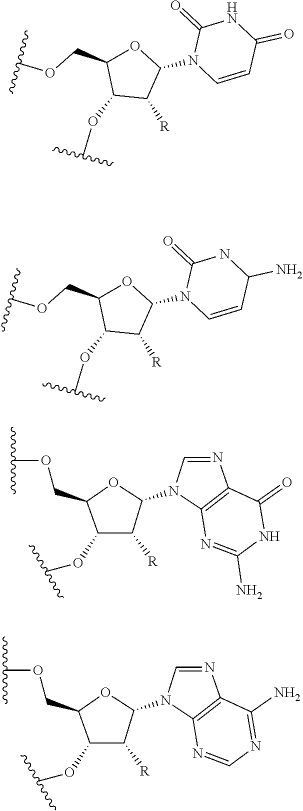

wherein R is H, OH, OCH₃, F, NH₂, NHMe, NMe₂ or O-alkyl.

Exemplary phosphate modifications known to decrease the thermal stability of dsRNA duplexes compared to natural phosphodiester linkages are:

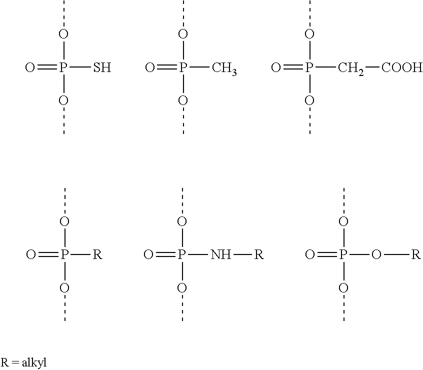

The alkyl for the R group can be a C₁-C₆alkyl. Specific alkyls for the R group include, but are not limited to methyl, ethyl, propyl, isopropyl, butyl, pentyl and hexyl.

As the skilled artisan will recognize, in view of the functional role of nucleobases is defining specificity of an RNAi agent of the disclosure, while nucleobase modifications can be performed in the various manners as described herein, e.g., to introduce destabilizing modifications into an RNAi agent of the disclosure, e.g., for purpose of enhancing on-target effect relative to off-target effect, the range of modifications available and, in general, present upon RNAi agents of the disclosure tends to be much greater for non-nucleobase modifications, e.g., modifications to sugar groups or phosphate backbones of polyribonucleotides. Such modifications are described in greater detail in other sections of the instant disclosure and are expressly contemplated for RNAi agents of the disclosure, either possessing native nucleobases or modified nucleobases as described above or elsewhere herein.

In addition to the antisense strand comprising a thermally destabilizing modification, the dsRNA can also comprise one or more stabilizing modifications. For example, the dsRNA can comprise at least two (e.g., two, three, four, five, six, seven, eight, nine, ten or more) stabilizing modifications. Without limitations, the stabilizing modifications all can be present in one strand. In some embodiments, both the sense and the antisense strands comprise at least two stabilizing modifications. The stabilizing modification can occur on any nucleotide of the sense strand or antisense strand. For instance, the stabilizing modification can occur on every nucleotide on the sense strand or antisense strand; each stabilizing modification can occur in an alternating pattern on the sense strand or antisense strand; or the sense strand or antisense strand comprises both stabilizing modification in an alternating pattern. The alternating pattern of the stabilizing modifications on the sense strand may be the same or different from the antisense strand, and the alternating pattern of the stabilizing modifications on the sense strand can have a shift relative to the alternating pattern of the stabilizing modifications on the antisense strand.

In some embodiments, the antisense strand comprises at least two (e.g., two, three, four, five, six, seven, eight, nine, ten or more) stabilizing modifications. Without limitations, a stabilizing modification in the antisense strand can be present at any positions. In some embodiments, the antisense comprises stabilizing modifications at positions 2, 6, 8, 9, 14, and 16 from the 5′-end. In some other embodiments, the antisense comprises stabilizing modifications at positions 2, 6, 14, and 16 from the 5′-end. In still some other embodiments, the antisense comprises stabilizing modifications at positions 2, 14, and 16 from the 5′-end.

In some embodiments, the antisense strand comprises at least one stabilizing modification adjacent to the destabilizing modification. For example, the stabilizing modification can be the nucleotide at the 5′-end or the 3′-end of the destabilizing modification, i.e., at position −1 or +1 from the position of the destabilizing modification. In some embodiments, the antisense strand comprises a stabilizing modification at each of the 5′-end and the 3′-end of the destabilizing modification, i.e., positions −1 and +1 from the position of the destabilizing modification.

In some embodiments, the antisense strand comprises at least two stabilizing modifications at the 3′-end of the destabilizing modification, i.e., at positions +1 and +2 from the position of the destabilizing modification.

In some embodiments, the sense strand comprises at least two (e.g., two, three, four, five, six, seven, eight, nine, ten or more) stabilizing modifications. Without limitations, a stabilizing modification in the sense strand can be present at any positions. In some embodiments, the sense strand comprises stabilizing modifications at positions 7, 10, and 11 from the 5′-end. In some other embodiments, the sense strand comprises stabilizing modifications at positions 7, 9, 10, and 11 from the 5′-end. In some embodiments, the sense strand comprises stabilizing modifications at positions opposite or complimentary to positions 11, 12, and 15 of the antisense strand, counting from the 5′-end of the antisense strand. In some other embodiments, the sense strand comprises stabilizing modifications at positions opposite or complimentary to positions 11, 12, 13, and 15 of the antisense strand, counting from the 5′-end of the antisense strand. In some embodiments, the sense strand comprises a block of two, three, or four stabilizing modifications.

In some embodiments, the sense strand does not comprise a stabilizing modification in position opposite or complimentary to the thermally destabilizing modification of the duplex in the antisense strand.

Exemplary thermally stabilizing modifications include, but are not limited to, 2′-fluoro modifications. Other thermally stabilizing modifications include, but are not limited to, LNA.

In some embodiments, the dsRNA of the disclosure comprises at least four (e.g., four, five, six, seven, eight, nine, ten, or more) 2′-fluoro nucleotides. Without limitations, the 2′-fluoro nucleotides all can be present in one strand. In some embodiments, both the sense and the antisense strands comprise at least two 2′-fluoro nucleotides. The 2′-fluoro modification can occur on any nucleotide of the sense strand or antisense strand. For instance, the 2′-fluoro modification can occur on every nucleotide on the sense strand or antisense strand; each 2′-fluoro modification can occur in an alternating pattern on the sense strand or antisense strand; or the sense strand or antisense strand comprises both 2′-fluoro modifications in an alternating pattern. The alternating pattern of the 2′-fluoro modifications on the sense strand may be the same or different from the antisense strand, and the alternating pattern of the 2′-fluoro modifications on the sense strand can have a shift relative to the alternating pattern of the 2′-fluoro modifications on the antisense strand.

In some embodiments, the antisense strand comprises at least two (e.g., two, three, four, five, six, seven, eight, nine, ten, or more) 2′-fluoro nucleotides. Without limitations, a 2′-fluoro modification in the antisense strand can be present at any positions. In some embodiments, the antisense comprises 2′-fluoro nucleotides at positions 2, 6, 8, 9, 14, and 16 from the 5′-end. In some other embodiments, the antisense comprises 2′-fluoro nucleotides at positions 2, 6, 14, and 16 from the 5′-end. In still some other embodiments, the antisense comprises 2′-fluoro nucleotides at positions 2, 14, and 16 from the 5′-end.

In some embodiments, the antisense strand comprises at least one 2′-fluoro nucleotide adjacent to the destabilizing modification. For example, the 2′-fluoro nucleotide can be the nucleotide at the 5′-end or the 3′-end of the destabilizing modification, i.e., at position −1 or +1 from the position of the destabilizing modification. In some embodiments, the antisense strand comprises a 2′-fluoro nucleotide at each of the 5′-end and the 3′-end of the destabilizing modification, i.e., positions −1 and +1 from the position of the destabilizing modification.

In some embodiments, the antisense strand comprises at least two 2′-fluoro nucleotides at the 3′-end of the destabilizing modification, i.e., at positions +1 and +2 from the position of the destabilizing modification.

In some embodiments, the sense strand comprises at least two (e.g., two, three, four, five, six, seven, eight, nine, ten or more) 2′-fluoro nucleotides. Without limitations, a 2′-fluoro modification in the sense strand can be present at any positions. In some embodiments, the antisense comprises 2′-fluoro nucleotides at positions 7, 10, and 11 from the 5′-end. In some other embodiments, the sense strand comprises 2′-fluoro nucleotides at positions 7, 9, 10, and 11 from the 5′-end. In some embodiments, the sense strand comprises 2′-fluoro nucleotides at positions opposite or complimentary to positions 11, 12, and 15 of the antisense strand, counting from the 5′-end of the antisense strand. In some other embodiments, the sense strand comprises 2′-fluoro nucleotides at positions opposite or complimentary to positions 11, 12, 13, and 15 of the antisense strand, counting from the 5′-end of the antisense strand. In some embodiments, the sense strand comprises a block of two, three or four 2′-fluoro nucleotides.

In some embodiments, the sense strand does not comprise a 2′-fluoro nucleotide in position opposite or complimentary to the thermally destabilizing modification of the duplex in the antisense strand.

In some embodiments, the dsRNA molecule of the disclosure comprises a 21 nucleotides (nt) sense strand and a 23 nucleotides (nt) antisense, wherein the antisense strand contains at least one thermally destabilizing nucleotide, where the at least one thermally destabilizing nucleotide occurs in the seed region of the antisense strand (i.e., at position 2-9 of the 5′-end of the antisense strand), wherein one end of the dsRNA is blunt, while the other end is comprises a 2 nt overhang, and wherein the dsRNA optionally further has at least one (e.g., one, two, three, four, five, six or all seven) of the following characteristics: (i) the antisense comprises 2, 3, 4, 5 or 6 2′-fluoro modifications; (ii) the antisense comprises 1, 2, 3, 4 or 5 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3, 4 or 5 phosphorothioate internucleotide linkages; (vi) the dsRNA comprises at least four 2′-fluoro modifications; and (vii) the dsRNA comprises a blunt end at 5′-end of the antisense strand. Optionally, the 2 nt overhang is at the 3′-end of the antisense.

In some embodiments, the dsRNA molecule of the disclosure comprising a sense and antisense strands, wherein: the sense strand is 25-30 nucleotide residues in length, wherein starting from the 5′ terminal nucleotide (position 1), positions 1 to 23 of said sense strand comprise at least 8 ribonucleotides; antisense strand is 36-66 nucleotide residues in length and, starting from the 3′ terminal nucleotide, at least 8 ribonucleotides in the positions paired with positions 1-23 of sense strand to form a duplex; wherein at least the 3′ terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when said double stranded nucleic acid is introduced into a mammalian cell; and wherein the antisense strand contains at least one thermally destabilizing nucleotide, where at least one thermally destabilizing nucleotide is in the seed region of the antisense strand (i.e. at position 2-9 of the 5′-end of the antisense strand). For example, the thermally destabilizing nucleotide occurs between positions opposite or complimentary to positions 14-17 of the 5′-end of the sense strand, and wherein the dsRNA optionally further has at least one (e.g., one, two, three, four, five, six or all seven) of the following characteristics: (i) the antisense comprises 2, 3, 4, 5, or 6 2′-fluoro modifications; (ii) the antisense comprises 1, 2, 3, 4, or 5 phosphorothioate internucleotide linkages; (iii) the sense strand is conjugated with a ligand; (iv) the sense strand comprises 2, 3, 4, or 5 2′-fluoro modifications; (v) the sense strand comprises 1, 2, 3, 4, or 5 phosphorothioate internucleotide linkages; and (vi) the dsRNA comprises at least four 2′-fluoro modifications; and (vii) the dsRNA comprises a duplex region of 12-30 nucleotide pairs in length.

In some embodiments, the dsRNA molecule of the disclosure comprises a sense and antisense strands, wherein said dsRNA molecule comprises a sense strand having a length which is at least 25 and at most 29 nucleotides and an antisense strand having a length which is at most 30 nucleotides with the sense strand comprises a modified nucleotide that is susceptible to enzymatic degradation at position 11 from the 5′end, wherein the 3′ end of said sense strand and the 5′ end of said antisense strand form a blunt end and said antisense strand is 1-4 nucleotides longer at its 3′ end than the sense strand, wherein the duplex region which is at least 25 nucleotides in length, and said antisense strand is sufficiently complementary to a target mRNA along at least 19 nt of said antisense strand length to reduce target gene expression when said dsRNA molecule is introduced into a mammalian cell, and wherein dicer cleavage of said dsRNA preferentially results in an siRNA comprising said 3′ end of said antisense strand, thereby reducing expression of the target gene in the mammal, wherein the antisense strand contains at least one thermally destabilizing nucleotide, where the at least one thermally destabilizing nucleotide is in the seed region of the antisense strand (i.e. at position 2-9 of the 5′-end of the antisense strand), and wherein the dsRNA optionally further has at least one (e.g., one, two, three, four, five, six or all seven) of the following characteristics: (i) the antisense comprises 2, 3, 4, 5, or 6 2′-fluoro modifications; (ii) the antisense comprises 1, 2, 3, 4, or 5 phosphorothioate internucleotide linkages; (iii) the sense strand is conjugated with a ligand; (iv) the sense strand comprises 2, 3, 4, or 5 2′-fluoro modifications; (v) the sense strand comprises 1, 2, 3, 4, or 5 phosphorothioate internucleotide linkages; and (vi) the dsRNA comprises at least four 2′-fluoro modifications; and (vii) the dsRNA has a duplex region of 12-29 nucleotide pairs in length.

In some embodiments, every nucleotide in the sense strand and antisense strand of the dsRNA molecule may be modified. Each nucleotide may be modified with the same or different modification which can include one or more alteration of one or both of the non-linking phosphate oxygens or of one or more of the linking phosphate oxygens; alteration of a constituent of the ribose sugar, e.g., of the 2′ hydroxyl on the ribose sugar; wholesale replacement of the phosphate moiety with “dephospho” linkers; modification or replacement of a naturally occurring base; and replacement or modification of the ribose-phosphate backbone.

As nucleic acids are polymers of subunits, many of the modifications occur at a position which is repeated within a nucleic acid, e.g., a modification of a base, or a phosphate moiety, or a non-linking O of a phosphate moiety. In some cases, the modification will occur at all of the subject positions in the nucleic acid but in many cases it will not. By way of example, a modification may only occur at a 3′ or 5′ terminal position, may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of an RNA or may only occur in a single strand region of an RNA. E.g., a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′ end or ends can be phosphorylated.

It may be possible, e.g., to enhance stability, to include particular bases in overhangs, or to include modified nucleotides or nucleotide surrogates, in single strand overhangs, e.g., in a 5′ or 3′ overhang, or in both. E.g., it can be desirable to include purine nucleotides in overhangs. In some embodiments all or some of the bases in a 3′ or 5′ overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous with the target sequence.

In some embodiments, each residue of the sense strand and antisense strand is independently modified with LNA, HNA, CeNA, 2′-methoxyethyl, 2′-O-methyl, 2′-O-allyl, 2′-C-allyl, 2′-deoxy, or 2′-fluoro. The strands can contain more than one modification. In some embodiments, each residue of the sense strand and antisense strand is independently modified with 2′-O-methyl or 2′-fluoro. It is to be understood that these modifications are in addition to the at least one thermally destabilizing modification of the duplex present in the antisense strand.

At least two different modifications are typically present on the sense strand and antisense strand. Those two modifications may be the 2′-deoxy, 2′-O-methyl or 2′-fluoro modifications, acyclic nucleotides or others. In some embodiments, the sense strand and antisense strand each comprises two differently modified nucleotides selected from 2′-O-methyl or 2′-deoxy. In some embodiments, each residue of the sense strand and antisense strand is independently modified with 2′-O-methyl nucleotide, 2′-deoxy nucleotide, 2′-deoxy-2′-fluoro nucleotide, 2′-O—N-methylacetamido (2′-O—NMA) nucleotide, a 2′-O-dimethylaminoethoxyethyl (2′-O-DMAEOE) nucleotide, 2′-O-aminopropyl (2′-O-AP) nucleotide, or 2′-ara-F nucleotide. Again, it is to be understood that these modifications are in addition to the at least one thermally destabilizing modification of the duplex present in the antisense strand.

In some embodiments, the dsRNA molecule of the disclosure comprises modifications of an alternating pattern, particular in the B1, B2, B3, B1′, B2′, B3′, B4′ regions. The term “alternating motif” or “alternative pattern” as used herein refers to a motif having one or more modifications, each modification occurring on alternating nucleotides of one strand. The alternating nucleotide may refer to one per every other nucleotide or one per every three nucleotides, or a similar pattern. For example, if A, B and C each represent one type of modification to the nucleotide, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ,” etc.

The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . ”, “ACACAC . . . ” “BDBDBD . . . ” or “CDCDCD . . . ,” etc.

In some embodiments, the dsRNA molecule of the disclosure comprises the modification pattern for the alternating motif on the sense strand relative to the modification pattern for the alternating motif on the antisense strand is shifted. The shift may be such that the modified group of nucleotides of the sense strand corresponds to a differently modified group of nucleotides of the antisense strand and vice versa. For example, the sense strand when paired with the antisense strand in the dsRNA duplex, the alternating motif in the sense strand may start with “ABABAB” from 5′-3′ of the strand and the alternating motif in the antisense strand may start with “BABABA” from 3′-5′ of the strand within the duplex region. As another example, the alternating motif in the sense strand may start with “AABBAABB” from 5′-3′ of the strand and the alternating motif in the antisense strand may start with “BBAABBAA” from 3′-5′ of the strand within the duplex region, so that there is a complete or partial shift of the modification patterns between the sense strand and the antisense strand.

The dsRNA molecule of the disclosure may further comprise at least one phosphorothioate or methylphosphonate internucleotide linkage. The phosphorothioate or methylphosphonate internucleotide linkage modification may occur on any nucleotide of the sense strand or antisense strand or both in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand comprises both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In some embodiments, the dsRNA molecule comprises the phosphorothioate or methylphosphonate internucleotide linkage modification in the overhang region. For example, the overhang region comprises two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. Optionally, these terminal three nucleotides may be at the 3′-end of the antisense strand.

In some embodiments, the sense strand of the dsRNA molecule comprises 1-10 blocks of two to ten phosphorothioate or methylphosphonate internucleotide linkages separated by 1, 2, 3, 4, 5, 6, 7, 8, 9, 10, 11, 12, 13, 14, 15, or 16 phosphate internucleotide linkages, wherein one of the phosphorothioate or methylphosphonate internucleotide linkages is placed at any position in the oligonucleotide sequence and the said sense strand is paired with an antisense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of two phosphorothioate or methylphosphonate internucleotide linkages separated by 1, 2, 3, 4, 5, 6, 7, 8, 9, 10, 11, 12, 13, 14, 15, 16, 17, or 18 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of three phosphorothioate or methylphosphonate internucleotide linkages separated by 1, 2, 3, 4, 5, 6, 7, 8, 9, 10, 11, 12, 13, 14, 15, or 16 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of four phosphorothioate or methylphosphonate internucleotide linkages separated by 1, 2, 3, 4, 5, 6, 7, 8, 9, 10, 11, 12, 13, or 14 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of five phosphorothioate or methylphosphonate internucleotide linkages separated by 1, 2, 3, 4, 5, 6, 7, 8, 9, 10, 11, or 12 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of six phosphorothioate or methylphosphonate internucleotide linkages separated by 1, 2, 3, 4, 5, 6, 7, 8, 9, or 10 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of seven phosphorothioate or methylphosphonate internucleotide linkages separated by 1, 2, 3, 4, 5, 6, 7, or 8 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of eight phosphorothioate or methylphosphonate internucleotide linkages separated by 1, 2, 3, 4, 5, or 6 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of nine phosphorothioate or methylphosphonate internucleotide linkages separated by 1, 2, 3, or 4 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the dsRNA molecule of the disclosure further comprises one or more phosphorothioate or methylphosphonate internucleotide linkage modification within 1-10 of the termini position(s) of the sense or antisense strand. For example, at least 2, 3, 4, 5, 6, 7, 8, 9, or 10 nucleotides may be linked through phosphorothioate or methylphosphonate internucleotide linkage at one end or both ends of the sense or antisense strand.

In some embodiments, the dsRNA molecule of the disclosure further comprises one or more phosphorothioate or methylphosphonate internucleotide linkage modification within 1-10 of the internal region of the duplex of each of the sense or antisense strand. For example, at least 2, 3, 4, 5, 6, 7, 8, 9, or 10 nucleotides may be linked through phosphorothioate methylphosphonate internucleotide linkage at position 8-16 of the duplex region counting from the 5′-end of the sense strand; the dsRNA molecule can optionally further comprise one or more phosphorothioate or methylphosphonate internucleotide linkage modification within 1-10 of the termini position(s).

In some embodiments, the dsRNA molecule of the disclosure further comprises one to five phosphorothioate or methylphosphonate internucleotide linkage modification(s) within position 1-5 and one to five phosphorothioate or methylphosphonate internucleotide linkage modification(s) within position 18-23 of the sense strand (counting from the 5′-end), and one to five phosphorothioate or methylphosphonate internucleotide linkage modification at positions 1 and 2 and one to five within positions 18-23 of the antisense strand (counting from the 5′-end).

In some embodiments, the dsRNA molecule of the disclosure further comprises one phosphorothioate internucleotide linkage modification within position 1-5 and one phosphorothioate or methylphosphonate internucleotide linkage modification within position 18-23 of the sense strand (counting from the 5′-end), and one phosphorothioate internucleotide linkage modification at positions 1 and 2 and two phosphorothioate or methylphosphonate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and one phosphorothioate internucleotide linkage modification within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and two phosphorothioate internucleotide linkage modifications within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and two phosphorothioate internucleotide linkage modifications within position 18-23 of the sense strand (counting from the 5′-end), and one phosphorothioate internucleotide linkage modification at positions 1 and 2 and one phosphorothioate internucleotide linkage modification within positions 18-23 of the antisense strand (counting from the 5′-end).

In some embodiments, the dsRNA molecule of the disclosure further comprises one phosphorothioate internucleotide linkage modification within position 1-5 and one phosphorothioate internucleotide linkage modification within position 18-23 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In some embodiments, the dsRNA molecule of the disclosure further comprises one phosphorothioate internucleotide linkage modification within position 1-5 and one within position 18-23 of the sense strand (counting from the 5′-end), and two phosphorothioate internucleotide linkage modification at positions 1 and 2 and one phosphorothioate internucleotide linkage modification within positions 18-23 of the antisense strand (counting from the 5′-end).

In some embodiments, the dsRNA molecule of the disclosure further comprises one phosphorothioate internucleotide linkage modification within position 1-5 (counting from the 5′-end) of the sense strand, and two phosphorothioate internucleotide linkage modifications at positions 1 and 2 and one phosphorothioate internucleotide linkage modification within positions 18-23 of the antisense strand (counting from the 5′-end).

In some embodiments, the dsRNA molecule of the disclosure further comprises two phosphorothioate internucleotide linkage modifications within position 1-5 (counting from the 5′-end) of the sense strand, and one phosphorothioate internucleotide linkage modification at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and one within position 18-23 of the sense strand (counting from the 5′-end), and two phosphorothioate internucleotide linkage modifications at positions 1 and 2 and one phosphorothioate internucleotide linkage modification within positions 18-23 of the antisense strand (counting from the 5′-end).

In some embodiments, the dsRNA molecule of the disclosure further comprises two phosphorothioate internucleotide linkage modifications within position 1-5 and one phosphorothioate internucleotide linkage modification within position 18-23 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and one phosphorothioate internucleotide linkage modification within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications at position 1 and 2, and two phosphorothioate internucleotide linkage modifications at position 20 and 21 of the sense strand (counting from the 5′-end), and one phosphorothioate internucleotide linkage modification at positions 1 and one at position 21 of the antisense strand (counting from the 5′-end).

In some embodiments, the dsRNA molecule of the disclosure further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 20 and 21 the antisense strand (counting from the 5′-end).

In some embodiments, the dsRNA molecule of the disclosure further comprises two phosphorothioate internucleotide linkage modifications at position 1 and 2, and two phosphorothioate internucleotide linkage modifications at position 21 and 22 of the sense strand (counting from the 5′-end), and one phosphorothioate internucleotide linkage modification at positions 1 and one phosphorothioate internucleotide linkage modification at position 21 of the antisense strand (counting from the 5′-end).

In some embodiments, the dsRNA molecule of the disclosure further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 21 and 22 the antisense strand (counting from the 5′-end).

In some embodiments, the dsRNA molecule of the disclosure further comprises two phosphorothioate internucleotide linkage modifications at position 1 and 2, and two phosphorothioate internucleotide linkage modifications at position 22 and 23 of the sense strand (counting from the 5′-end), and one phosphorothioate internucleotide linkage modification at positions 1 and one phosphorothioate internucleotide linkage modification at position 21 of the antisense strand (counting from the 5′-end).

In some embodiments, the dsRNA molecule of the disclosure further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 23 and 23 the antisense strand (counting from the 5′-end).

In some embodiments, compound of the disclosure comprises a pattern of backbone chiral centers. In some embodiments, a common pattern of backbone chiral centers comprises at least 5 internucleotidic linkages in the Sp configuration. In some embodiments, a common pattern of backbone chiral centers comprises at least 6 internucleotidic linkages in the Sp configuration. In some embodiments, a common pattern of backbone chiral centers comprises at least 7 internucleotidic linkages in the Sp configuration. In some embodiments, a common pattern of backbone chiral centers comprises at least 8 internucleotidic linkages in the Sp configuration. In some embodiments, a common pattern of backbone chiral centers comprises at least 9 internucleotidic linkages in the Sp configuration. In some embodiments, a common pattern of backbone chiral centers comprises at least 10 internucleotidic linkages in the Sp configuration. In some embodiments, a common pattern of backbone chiral centers comprises at least 11 internucleotidic linkages in the Sp configuration. In some embodiments, a common pattern of backbone chiral centers comprises at least 12 internucleotidic linkages in the Sp configuration. In some embodiments, a common pattern of backbone chiral centers comprises at least 13 internucleotidic linkages in the Sp configuration. In some embodiments, a common pattern of backbone chiral centers comprises at least 14 internucleotidic linkages in the Sp configuration. In some embodiments, a common pattern of backbone chiral centers comprises at least 15 internucleotidic linkages in the Sp configuration. In some embodiments, a common pattern of backbone chiral centers comprises at least 16 internucleotidic linkages in the Sp configuration. In some embodiments, a common pattern of backbone chiral centers comprises at least 17 internucleotidic linkages in the Sp configuration. In some embodiments, a common pattern of backbone chiral centers comprises at least 18 internucleotidic linkages in the Sp configuration. In some embodiments, a common pattern of backbone chiral centers comprises at least 19 internucleotidic linkages in the Sp configuration. In some embodiments, a common pattern of backbone chiral centers comprises no more than 8 internucleotidic linkages in the Rp configuration. In some embodiments, a common pattern of backbone chiral centers comprises no more than 7 internucleotidic linkages in the Rp configuration. In some embodiments, a common pattern of backbone chiral centers comprises no more than 6 internucleotidic linkages in the Rp configuration. In some embodiments, a common pattern of backbone chiral centers comprises no more than 5 internucleotidic linkages in the Rp configuration. In some embodiments, a common pattern of backbone chiral centers comprises no more than 4 internucleotidic linkages in the Rp configuration. In some embodiments, a common pattern of backbone chiral centers comprises no more than 3 internucleotidic linkages in the Rp configuration. In some embodiments, a common pattern of backbone chiral centers comprises no more than 2 internucleotidic linkages in the Rp configuration. In some embodiments, a common pattern of backbone chiral centers comprises no more than 1 internucleotidic linkages in the Rp configuration. In some embodiments, a common pattern of backbone chiral centers comprises no more than 8 internucleotidic linkages which are not chiral (as a non-limiting example, a phosphodiester). In some embodiments, a common pattern of backbone chiral centers comprises no more than 7 internucleotidic linkages which are not chiral. In some embodiments, a common pattern of backbone chiral centers comprises no more than 6 internucleotidic linkages which are not chiral. In some embodiments, a common pattern of backbone chiral centers comprises no more than 5 internucleotidic linkages which are not chiral. In some embodiments, a common pattern of backbone chiral centers comprises no more than 4 internucleotidic linkages which are not chiral. In some embodiments, a common pattern of backbone chiral centers comprises no more than 3 internucleotidic linkages which are not chiral. In some embodiments, a common pattern of backbone chiral centers comprises no more than 2 internucleotidic linkages which are not chiral. In some embodiments, a common pattern of backbone chiral centers comprises no more than 1 internucleotidic linkages which are not chiral. In some embodiments, a common pattern of backbone chiral centers comprises at least 10 internucleotidic linkages in the Sp configuration, and no more than 8 internucleotidic linkages which are not chiral. In some embodiments, a common pattern of backbone chiral centers comprises at least 11 internucleotidic linkages in the Sp configuration, and no more than 7 internucleotidic linkages which are not chiral. In some embodiments, a common pattern of backbone chiral centers comprises at least 12 internucleotidic linkages in the Sp configuration, and no more than 6 internucleotidic linkages which are not chiral. In some embodiments, a common pattern of backbone chiral centers comprises at least 13 internucleotidic linkages in the Sp configuration, and no more than 6 internucleotidic linkages which are not chiral. In some embodiments, a common pattern of backbone chiral centers comprises at least 14 internucleotidic linkages in the Sp configuration, and no more than 5 internucleotidic linkages which are not chiral. In some embodiments, a common pattern of backbone chiral centers comprises at least 15 internucleotidic linkages in the Sp configuration, and no more than 4 internucleotidic linkages which are not chiral. In some embodiments, the internucleotidic linkages in the Sp configuration are optionally contiguous or not contiguous. In some embodiments, the internucleotidic linkages in the Rp configuration are optionally contiguous or not contiguous. In some embodiments, the internucleotidic linkages which are not chiral are optionally contiguous or not contiguous.

In some embodiments, compound of the disclosure comprises a block is a stereochemistry block. In some embodiments, a block is an Rp block in that each internucleotidic linkage of the block is Rp. In some embodiments, a 5′-block is an Rp block. In some embodiments, a 3′-block is an Rp block. In some embodiments, a block is an Sp block in that each internucleotidic linkage of the block is Sp. In some embodiments, a 5′-block is an Sp block. In some embodiments, a 3′-block is an Sp block. In some embodiments, provided oligonucleotides comprise both Rp and Sp blocks. In some embodiments, provided oligonucleotides comprise one or more Rp but no Sp blocks. In some embodiments, provided oligonucleotides comprise one or more Sp but no Rp blocks. In some embodiments, provided oligonucleotides comprise one or more PO blocks wherein each internucleotidic linkage in a natural phosphate linkage.

In some embodiments, compound of the disclosure comprises a 5′-block is an Sp block wherein each sugar moiety comprises a 2′-F modification. In some embodiments, a 5′-block is an Sp block wherein each of internucleotidic linkage is a modified internucleotidic linkage and each sugar moiety comprises a 2′-F modification. In some embodiments, a 5′-block is an Sp block wherein each of internucleotidic linkage is a phosphorothioate linkage and each sugar moiety comprises a 2′-F modification. In some embodiments, a 5′-block comprises 4 or more nucleoside units. In some embodiments, a 5′-block comprises 5 or more nucleoside units. In some embodiments, a 5′-block comprises 6 or more nucleoside units. In some embodiments, a 5′-block comprises 7 or more nucleoside units. In some embodiments, a 3′-block is an Sp block wherein each sugar moiety comprises a 2′-F modification. In some embodiments, a 3′-block is an Sp block wherein each of internucleotidic linkage is a modified internucleotidic linkage and each sugar moiety comprises a 2′-F modification. In some embodiments, a 3′-block is an Sp block wherein each of internucleotidic linkage is a phosphorothioate linkage and each sugar moiety comprises a 2′-F modification. In some embodiments, a 3′-block comprises 4 or more nucleoside units. In some embodiments, a 3′-block comprises 5 or more nucleoside units. In some embodiments, a 3′-block comprises 6 or more nucleoside units. In some embodiments, a 3′-block comprises 7 or more nucleoside units.

In some embodiments, compound of the disclosure comprises a type of nucleoside in a region or an oligonucleotide is followed by a specific type of internucleotidic linkage, e.g., natural phosphate linkage, modified internucleotidic linkage, Rp chiral internucleotidic linkage, Sp chiral internucleotidic linkage, etc. In some embodiments, A is followed by Sp. In some embodiments, A is followed by Rp. In some embodiments, A is followed by natural phosphate linkage (PO). In some embodiments, U is followed by Sp. In some embodiments, U is followed by Rp. In some embodiments, U is followed by natural phosphate linkage (PO). In some embodiments, C is followed by Sp. In some embodiments, C is followed by Rp. In some embodiments, C is followed by natural phosphate linkage (PO). In some embodiments, G is followed by Sp. In some embodiments, G is followed by Rp. In some embodiments, G is followed by natural phosphate linkage (PO). In some embodiments, C and U are followed by Sp. In some embodiments, C and U are followed by Rp. In some embodiments, C and U are followed by natural phosphate linkage (PO). In some embodiments, A and G are followed by Sp. In some embodiments, A and G are followed by Rp.

In some embodiments, the antisense strand comprises phosphorothioate internucleotide linkages between nucleotide positions 21 and 22, and between nucleotide positions 22 and 23, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, four, five, six, seven or all eight) of the following characteristics: (i) the antisense comprises 2, 3, 4, 5 or 6 2′-fluoro modifications; (ii) the antisense comprises 3, 4 or 5 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3, 4 or 5 phosphorothioate internucleotide linkages; (vi) the dsRNA comprises at least four 2′-fluoro modifications; (vii) the dsRNA comprises a duplex region of 12-40 nucleotide pairs in length; and (viii) the dsRNA has a blunt end at 5′-end of the antisense strand.

In some embodiments, the antisense strand comprises phosphorothioate internucleotide linkages between nucleotide positions 1 and 2, between nucleotide positions 2 and 3, between nucleotide positions 21 and 22, and between nucleotide positions 22 and 23, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, four, five, six, seven or all eight) of the following characteristics: (i) the antisense comprises 2, 3, 4, 5 or 6 2′-fluoro modifications; (ii) the sense strand is conjugated with a ligand; (iii) the sense strand comprises 2, 3, 4 or 5 2′-fluoro modifications; (iv) the sense strand comprises 1, 2, 3, 4 or 5 phosphorothioate internucleotide linkages; (v) the dsRNA comprises at least four 2′-fluoro modifications; (vi) the dsRNA comprises a duplex region of 12-40 nucleotide pairs in length; (vii) the dsRNA comprises a duplex region of 12-40 nucleotide pairs in length; and (viii) the dsRNA has a blunt end at 5′-end of the antisense strand.

In some embodiments, the sense strand comprises phosphorothioate internucleotide linkages between nucleotide positions 1 and 2, and between nucleotide positions 2 and 3, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, four, five, six, seven or all eight) of the following characteristics: (i) the antisense comprises 2, 3, 4, 5 or 6 2′-fluoro modifications; (ii) the antisense comprises 1, 2, 3, 4 or 5 phosphorothioate internucleotide linkages; (iii) the sense strand is conjugated with a ligand; (iv) the sense strand comprises 2, 3, 4 or 5 2′-fluoro modifications; (v) the sense strand comprises 3, 4 or 5 phosphorothioate internucleotide linkages; (vi) the dsRNA comprises at least four 2′-fluoro modifications; (vii) the dsRNA comprises a duplex region of 12-40 nucleotide pairs in length; and (viii) the dsRNA has a blunt end at 5′-end of the antisense strand.

In some embodiments, the sense strand comprises phosphorothioate internucleotide linkages between nucleotide positions 1 and 2, and between nucleotide positions 2 and 3, the antisense strand comprises phosphorothioate internucleotide linkages between nucleotide positions 1 and 2, between nucleotide positions 2 and 3, between nucleotide positions 21 and 22, and between nucleotide positions 22 and 23, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, four, five, six or all seven) of the following characteristics: (i) the antisense comprises 2, 3, 4, 5 or 6 2′-fluoro modifications; (ii) the sense strand is conjugated with a ligand; (iii) the sense strand comprises 2, 3, 4 or 5 2′-fluoro modifications; (iv) the sense strand comprises 3, 4 or 5 phosphorothioate internucleotide linkages; (v) the dsRNA comprises at least four 2′-fluoro modifications; (vi) the dsRNA comprises a duplex region of 12-40 nucleotide pairs in length; and (vii) the dsRNA has a blunt end at 5′-end of the antisense strand.

In some embodiments, the dsRNA molecule of the disclosure comprises mismatch(es) with the target, within the duplex, or combinations thereof. The mismatch can occur in the overhang region or the duplex region. The base pair can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In some embodiments, the dsRNA molecule of the disclosure comprises at least one of the first 1, 2, 3, 4, or 5 base pairs within the duplex regions from the 5′-end of the antisense strand can be chosen independently from the group of: A:U, G:U, I:C, and mismatched pairs, e.g., non-canonical or other than canonical pairings or pairings which include a universal base, to promote the dissociation of the antisense strand at the 5′-end of the duplex.

In some embodiments, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

It was found that introducing 4′-modified or 5′-modified nucleotide to the 3′-end of a phosphodiester (PO), phosphorothioate (PS), or phosphorodithioate (PS2) linkage of a dinucleotide at any position of single stranded or double stranded oligonucleotide can exert steric effect to the internucleotide linkage and, hence, protecting or stabilizing it against nucleases.

In some embodiments, 5′-modified nucleoside is introduced at the 3′-end of a dinucleotide at any position of single stranded or double stranded siRNA. For instance, a 5′-alkylated nucleoside may be introduced at the 3′-end of a dinucleotide at any position of single stranded or double stranded siRNA. The alkyl group at the 5′ position of the ribose sugar can be racemic or chirally pure R or S isomer. An exemplary 5′-alkylated nucleoside is 5′-methyl nucleoside. The 5′-methyl can be either racemic or chirally pure R or S isomer.

In some embodiments, 4′-modified nucleoside is introduced at the 3′-end of a dinucleotide at any position of single stranded or double stranded siRNA. For instance, a 4′-alkylated nucleoside may be introduced at the 3′-end of a dinucleotide at any position of single stranded or double stranded siRNA. The alkyl group at the 4′ position of the ribose sugar can be racemic or chirally pure R or S isomer. An exemplary 4′-alkylated nucleoside is 4′-methyl nucleoside. The 4′-methyl can be either racemic or chirally pure R or S isomer. Alternatively, a 4′-O-alkylated nucleoside may be introduced at the 3′-end of a dinucleotide at any position of single stranded or double stranded siRNA. The 4′-O-alkyl of the ribose sugar can be racemic or chirally pure R or S isomer. An exemplary 4′-O-alkylated nucleoside is 4′-O-methyl nucleoside. The 4′-O-methyl can be either racemic or chirally pure R or S isomer.

In some embodiments, 5′-alkylated nucleoside is introduced at any position on the sense strand or antisense strand of a dsRNA, and such modification maintains or improves potency of the dsRNA. The 5′-alkyl can be either racemic or chirally pure R or S isomer. An exemplary 5′-alkylated nucleoside is 5′-methyl nucleoside. The 5′-methyl can be either racemic or chirally pure R or S isomer.

In some embodiments, 4′-alkylated nucleoside is introduced at any position on the sense strand or antisense strand of a dsRNA, and such modification maintains or improves potency of the dsRNA. The 4′-alkyl can be either racemic or chirally pure R or S isomer. An exemplary 4′-alkylated nucleoside is 4′-methyl nucleoside. The 4′-methyl can be either racemic or chirally pure R or S isomer.

In some embodiments, 4′-O-alkylated nucleoside is introduced at any position on the sense strand or antisense strand of a dsRNA, and such modification maintains or improves potency of the dsRNA. The 5′-alkyl can be either racemic or chirally pure R or S isomer. An exemplary 4′-O-alkylated nucleoside is 4′-O-methyl nucleoside. The 4′-O-methyl can be either racemic or chirally pure R or S isomer.

In some embodiments, the dsRNA molecule of the disclosure can comprise 2′-5′ linkages (with 2′-H, 2′-OH and 2′-OMe and with P═O or P═S). For example, the 2′-5′ linkages modifications can be used to promote nuclease resistance or to inhibit binding of the sense to the antisense strand, or can be used at the 5′ end of the sense strand to avoid sense strand activation by RISC.

In another embodiment, the dsRNA molecule of the disclosure can comprise L sugars (e.g., L ribose, L-arabinose with 2′-H, 2′-OH and 2′-OMe). For example, these L sugars modifications can be used to promote nuclease resistance or to inhibit binding of the sense to the antisense strand, or can be used at the 5′ end of the sense strand to avoid sense strand activation by RISC.

Various publications describe multimeric siRNA which can all be used with the dsRNA of the disclosure. Such publications include WO2007/091269, U.S. Pat. No. 7,858,769, WO2010/141511, WO2007/117686, WO2009/014887, and WO2011/031520 which are hereby incorporated by their entirely.

As described in more detail below, the RNAi agent that contains conjugations of one or more carbohydrate moieties to an RNAi agent can optimize one or more properties of the RNAi agent. In many cases, the carbohydrate moiety will be attached to a modified subunit of the RNAi agent. For example, the ribose sugar of one or more ribonucleotide subunits of a dsRNA agent can be replaced with another moiety, e.g., a non-carbohydrate (optionally cyclic) carrier to which is attached a carbohydrate ligand. A ribonucleotide subunit in which the ribose sugar of the subunit has been so replaced is referred to herein as a ribose replacement modification subunit. A cyclic carrier may be a carbocyclic ring system, i.e., all ring atoms are carbon atoms, or a heterocyclic ring system, i.e., one or more ring atoms may be a heteroatom, e.g., nitrogen, oxygen, sulfur. The cyclic carrier may be a monocyclic ring system, or may contain two or more rings, e.g. fused rings. The cyclic carrier may be a fully saturated ring system, or it may contain one or more double bonds.

The ligand may be attached to the polynucleotide via a carrier. The carriers include (i) at least one “backbone attachment point,” optionally two “backbone attachment points” and (ii) at least one “tethering attachment point.” A “backbone attachment point” as used herein refers to a functional group, e.g. a hydroxyl group, or generally, a bond available for, and that is suitable for incorporation of the carrier into the backbone, e.g., the phosphate, or modified phosphate, e.g., sulfur containing, backbone, of a ribonucleic acid. A “tethering attachment point” (TAP) in some embodiments refers to a constituent ring atom of the cyclic carrier, e.g., a carbon atom or a heteroatom (distinct from an atom which provides a backbone attachment point), that connects a selected moiety. The moiety can be, e.g., a carbohydrate, e.g. monosaccharide, disaccharide, trisaccharide, tetrasaccharide, oligosaccharide and polysaccharide. Optionally, the selected moiety is connected by an intervening tether to the cyclic carrier. Thus, the cyclic carrier will often include a functional group, e.g., an amino group, or generally, provide a bond, that is suitable for incorporation or tethering of another chemical entity, e.g., a ligand to the constituent ring.

The RNAi agents may be conjugated to a ligand via a carrier, wherein the carrier can be a cyclic group or an acyclic group. Optionally, the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and decalin. Optionally, the acyclic group is selected from serinol backbone and diethanolamine backbone.

In certain specific embodiments, the RNAi agent for use in the methods of the disclosure is an agent selected from the group of agents listed in any one of Tables 2-21. These agents may further comprise a ligand, such as one or more lipophilic moieties, one or more GalNAc derivatives, or both of one of more lipophilic moieties and one or more GalNAc derivatives.

III. iRNAs Conjugated to Ligands

Another modification of the RNA of an iRNA of the disclosure involves chemically linking to the iRNA one or more ligands, moieties or conjugates that enhance the activity, cellular distribution or cellular uptake of the iRNA, e.g., into a cell. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4: 1053-1060), a thioether, e.g., beryl-S-tritylthiol (Manoharan et al., Ann. N.Y. Acad. Sci., 1992, 660: 306-309; Manoharan et al., Biorg. Med. Chem. Let., 1993, 3: 2765-2770), a thiocholesterol (Oberhauser et al., Nucl. Acids Res., 1992, 20: 533-538), an aliphatic chain, e.g., dodecandiol or undecyl residues (Saison-Behmoaras et al., EMBO J, 1991, 10: 1111-1118; Kabanov et al., FEBS Lett., 1990, 259: 327-330; Svinarchuk et al., Biochimie, 1993, 75: 49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36: 3651-3654; Shea et al., Nucl. Acids Res., 1990, 18: 3777-3783), a polyamine or a polyethylene glycol chain (Manoharan et al., Nucleosides & Nucleotides, 1995, 14: 969-973), or adamantane acetic acid (Manoharan et al., Tetrahedron Lett., 1995, 36: 3651-3654), a palmityl moiety (Mishra et al., Biochim. Biophys. Acta, 1995, 1264: 229-237), or an octadecylamine or hexylamino-carbonyloxycholesterol moiety (Crooke et al., J. Pharmacol. Exp. Ther., 1996, 277: 923-937).

In certain embodiments, a ligand alters the distribution, targeting or lifetime of an iRNA agent into which it is incorporated. In some embodiments, a ligand provides an enhanced affinity for a selected target, e.g., molecule, cell or cell type, compartment, e.g., a cellular or organ compartment, tissue, organ or region of the body, as, e.g., compared to a species absent such a ligand. Typical ligands will not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an a helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic. In certain embodiments, the ligand is a multivalent galactose, e.g., an N-acetyl-galactosamine.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the iRNA agent into the cell, for example, by disrupting the cell's cytoskeleton, e.g., by disrupting the cell's microtubules, microfilaments, or intermediate filaments. The drug can be, for example, taxon, vincristine, vinblastine, cytochalasin, nocodazole, japlakinolide, latrunculin A, phalloidin, swinholide A, indanocine, or myoservin.

In some embodiments, a ligand attached to an iRNA as described herein acts as a pharmacokinetic modulator (PK modulator). PK modulators include lipophiles, bile acids, steroids, phospholipid analogues, peptides, protein binding agents, PEG, vitamins etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that comprise a number of phosphorothioate linkages are also known to bind to serum protein, thus short oligonucleotides, e.g., oligonucleotides of about 5 bases, 10 bases, 15 bases or 20 bases, comprising multiple of phosphorothioate linkages in the backbone are also amenable to the present disclosure as ligands (e.g. as PK modulating ligands). In addition, aptamers that bind serum components (e.g. serum proteins) are also suitable for use as PK modulating ligands in the embodiments described herein.

Ligand-conjugated iRNAs of the disclosure may be synthesized by the use of an oligonucleotide that bears a pendant reactive functionality, such as that derived from the attachment of a linking molecule onto the oligonucleotide (described below). This reactive oligonucleotide may be reacted directly with commercially-available ligands, ligands that are synthesized bearing any of a variety of protecting groups, or ligands that have a linking moiety attached thereto.

The oligonucleotides used in the conjugates of the present disclosure may be conveniently and routinely made through the well-known technique of solid-phase synthesis. Equipment for such synthesis is sold by several vendors including, for example, Applied Biosystems® (Foster City, Calif.). Any other means for such synthesis known in the art may additionally or alternatively be employed. It is also known to use similar techniques to prepare other oligonucleotides, such as the phosphorothioates and alkylated derivatives.

In the ligand-conjugated oligonucleotides and ligand-molecule bearing sequence-specific linked nucleosides of the present disclosure, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. In some embodiments, the oligonucleotides or linked nucleosides of the present disclosure are synthesized by an automated synthesizer using phosphoramidites derived from ligand-nucleoside conjugates in addition to the standard phosphoramidites and non-standard phosphoramidites that are commercially available and routinely used in oligonucleotide synthesis.

### A. Lipid Conjugates

In certain embodiments, the ligand or conjugate is a lipid or lipid-based molecule. Such a lipid or lipid-based molecule can typically bind a serum protein, such as human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, naproxen or aspirin can be used. A lipid or lipid-based ligand can (a) increase resistance to degradation of the conjugate, (b) increase targeting or transport into a target cell or cell membrane, or (c) can be used to adjust binding to a serum protein, e.g., HSA.

A lipid-based ligand can be used to modulate, e.g., control (e.g., inhibit) the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In certain embodiments, the lipid-based ligand binds HSA. For example, the ligand can bind HSA with a sufficient affinity such that distribution of the conjugate to a non-kidney tissue is enhanced. However, the affinity is typically not so strong that the HSA-ligand binding cannot be reversed.

In certain embodiments, the lipid-based ligand binds HSA weakly or not at all, such that distribution of the conjugate to the kidney is enhanced. Other moieties that target to kidney cells can also be used in place of or in addition to the lipid-based ligand.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by cancer cells. Also included are HSA and low density lipoprotein (LDL).

### B. Cell Permeation Agents

In another aspect, the ligand is a cell-permeation agent, such as a helical cell-permeation agent. In certain embodiments, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is typically an α-helical agent and can have a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to iRNA agents can affect pharmacokinetic distribution of the iRNA, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp, or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO: 16). An RFGF analogue (e.g., amino acid sequence AALLPVLLAAP (SEQ ID NO: 17)) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ (SEQ ID NO: 18)) and the Drosophila Antennapedia protein (RQIKIWFQNRRMKWKK (SEQ ID NO: 19)) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354: 82-84, 1991). Typically, the peptide or peptidomimetic tethered to a dsRNA agent via an incorporated monomer unit is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

An RGD peptide for use in the compositions and methods of the disclosure may be linear or cyclic, and may be modified, e.g., glycosylated or methylated, to facilitate targeting to a specific tissue(s). RGD-containing peptides and peptidiomimemtics may include D-amino acids, as well as synthetic RGD mimics. In addition to RGD, one can use other moieties that target the integrin ligand. Preferred conjugates of this ligand target PECAM-1 or VEGF.

An RGD peptide moiety can be used to target a particular cell type, e.g., a tumor cell, such as an endothelial tumor cell or a breast cancer tumor cell (Zitzmann et al., Cancer Res., 62: 5139-43, 2002). An RGD peptide can facilitate targeting of a dsRNA agent to tumors of a variety of other tissues, including the lung, kidney, spleen, or liver (Aoki et al., Cancer Gene Therapy 8: 783-787, 2001). Typically, the RGD peptide will facilitate targeting of an iRNA agent to the kidney. The RGD peptide can be linear or cyclic, and can be modified, e.g., glycosylated or methylated to facilitate targeting to specific tissues. For example, a glycosylated RGD peptide can deliver an iRNA agent to a tumor cell expressing αvß₃ (Haubner et al., Jour. Nucl. Med., 42: 326-336, 2001).

A “cell permeation peptide” is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31: 2717-2724, 2003).

### C. Carbohydrate Conjugates

In some embodiments of the compositions and methods of the disclosure, an iRNA further comprises a carbohydrate. The carbohydrate conjugated iRNA are advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri- and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di- and tri-saccharides include sugars having two or three monosaccharide units (e.g., C5, C6, C7, or C8).

In certain embodiments, a carbohydrate conjugate comprises a monosaccharide.

In certain embodiments, the monosaccharide is an N-acetylgalactosamine (GalNAc). GalNAc conjugates, which comprise one or more N-acetylgalactosamine (GalNAc) derivatives, are described, for example, in U.S. Pat. No. 8,106,022, the entire content of which is hereby incorporated herein by reference. In some embodiments, the GalNAc conjugate serves as a ligand that targets the iRNA to particular cells. In some embodiments, the GalNAc conjugate targets the iRNA to liver cells, e.g., by serving as a ligand for the asialoglycoprotein receptor of liver cells (e.g., hepatocytes).

In some embodiments, the carbohydrate conjugate comprises one or more GalNAc derivatives. The GalNAc derivatives may be attached via a linker, e.g., a bivalent or trivalent branched linker. In some embodiments the GalNAc conjugate is conjugated to the 3′ end of the sense strand. In some embodiments, the GalNAc conjugate is conjugated to the iRNA agent (e.g., to the 3′ end of the sense strand) via a linker, e.g., a linker as described herein. In some embodiments the GalNAc conjugate is conjugated to the 5′ end of the sense strand. In some embodiments, the GalNAc conjugate is conjugated to the iRNA agent (e.g., to the 5′ end of the sense strand) via a linker, e.g., a linker as described herein.

In certain embodiments of the disclosure, the GalNAc or GalNAc derivative is attached to an iRNA agent of the disclosure via a monovalent linker. In some embodiments, the GalNAc or GalNAc derivative is attached to an iRNA agent of the disclosure via a bivalent linker. In yet other embodiments of the disclosure, the GalNAc or GalNAc derivative is attached to an iRNA agent of the disclosure via a trivalent linker. In other embodiments of the disclosure, the GalNAc or GalNAc derivative is attached to an iRNA agent of the disclosure via a tetravalent linker.

In certain embodiments, the double stranded RNAi agents of the disclosure comprise one GalNAc or GalNAc derivative attached to the iRNA agent. In certain embodiments, the double stranded RNAi agents of the disclosure comprise a plurality (e.g., 2, 3, 4, 5, or 6) GalNAc or GalNAc derivatives, each independently attached to a plurality of nucleotides of the double stranded RNAi agent through a plurality of monovalent linkers.

In some embodiments, for example, when the two strands of an iRNA agent of the disclosure are part of one larger molecule connected by an uninterrupted chain of nucleotides between the 3′-end of one strand and the 5′-end of the respective other strand forming a hairpin loop comprising, a plurality of unpaired nucleotides, each unpaired nucleotide within the hairpin loop may independently comprise a GalNAc or GalNAc derivative attached via a monovalent linker. The hairpin loop may also be formed by an extended overhang in one strand of the duplex.

In some embodiments, for example, when the two strands of an iRNA agent of the disclosure are part of one larger molecule connected by an uninterrupted chain of nucleotides between the 3′-end of one strand and the 5′-end of the respective other strand forming a hairpin loop comprising, a plurality of unpaired nucleotides, each unpaired nucleotide within the hairpin loop may independently comprise a GalNAc or GalNAc derivative attached via a monovalent linker. The hairpin loop may also be formed by an extended overhang in one strand of the duplex.

In some embodiments, the GalNAc conjugate is

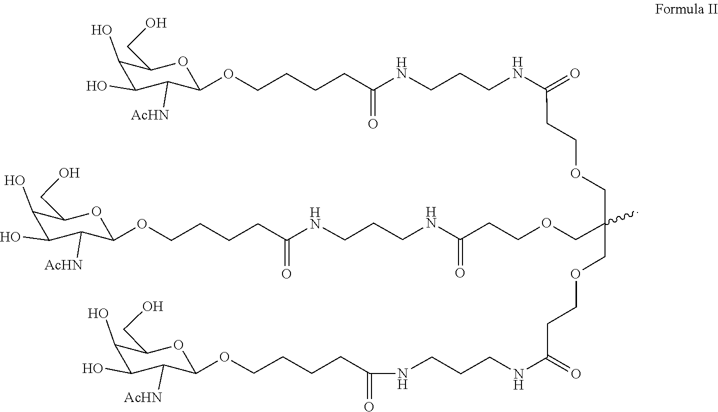 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In some embodiments, the RNAi agent is attached to the carbohydrate conjugate via a linker as shown in the following schematic, wherein X is O or S

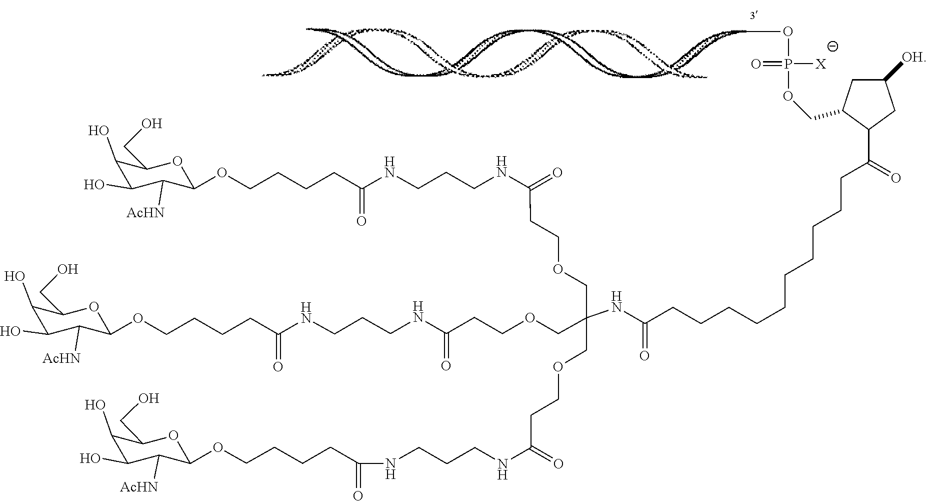

In some embodiments, the RNAi agent is conjugated to L96 as defined in Table 1 and shown below.

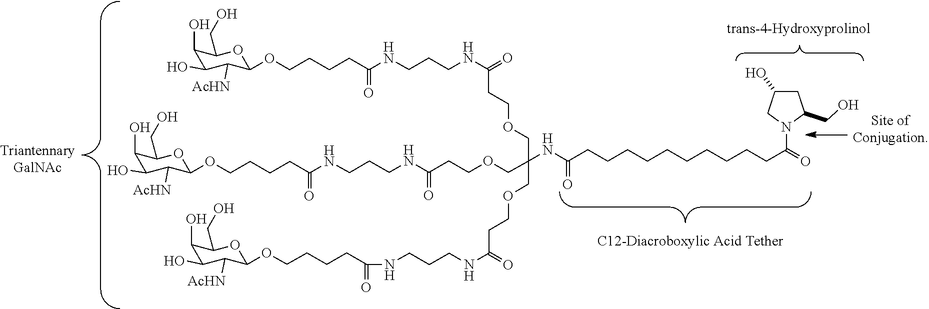

In certain embodiments, a carbohydrate conjugate for use in the compositions and methods of the disclosure is selected from the group consisting of:

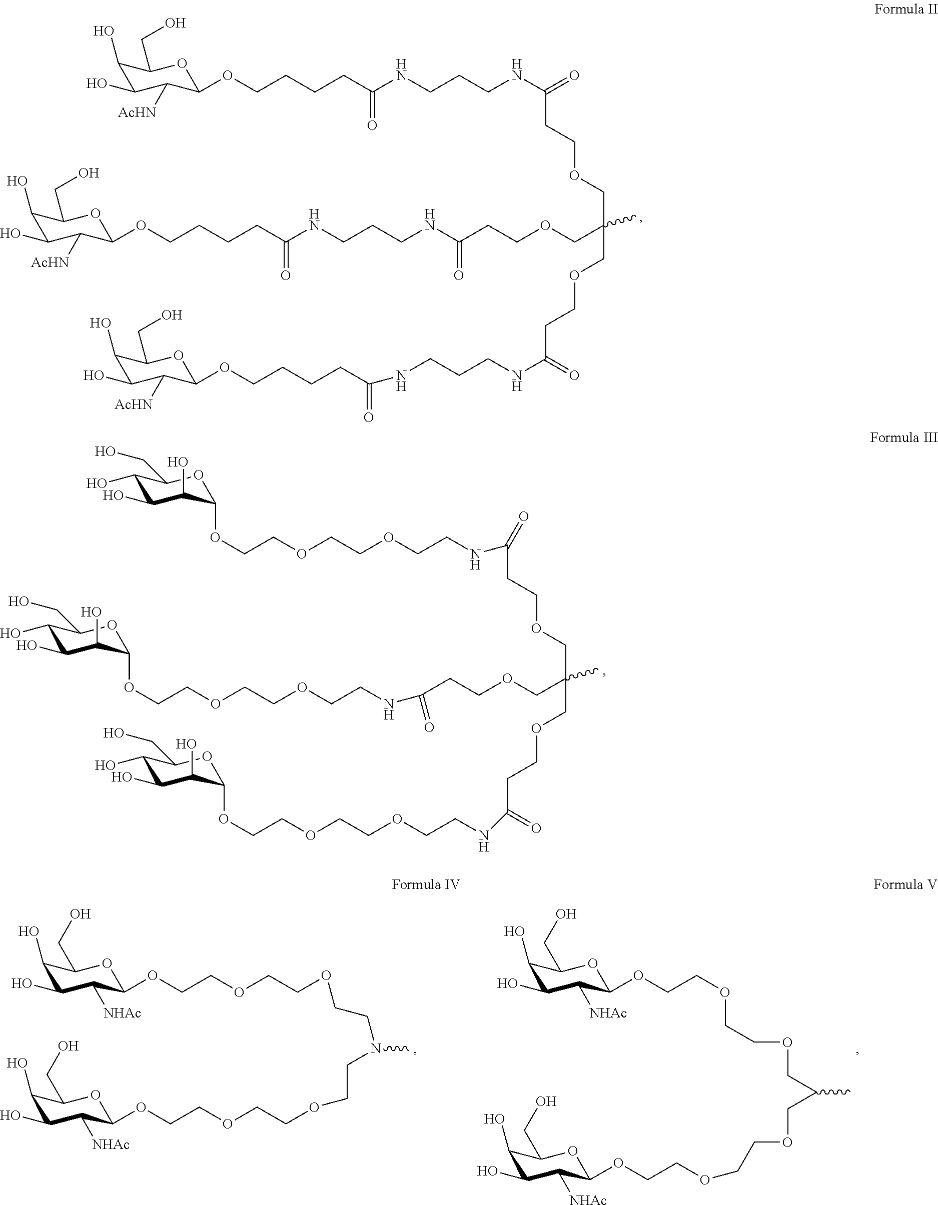

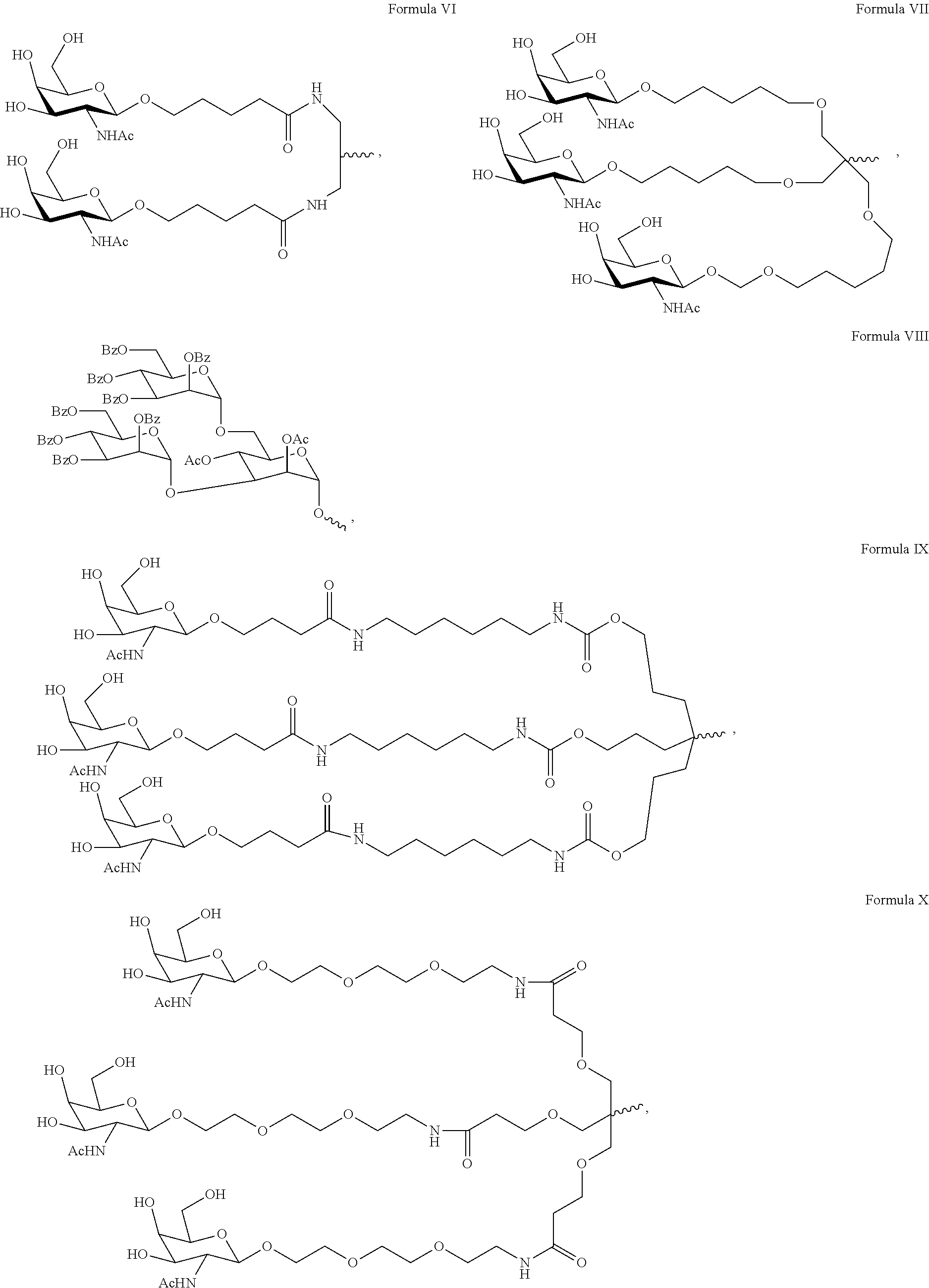

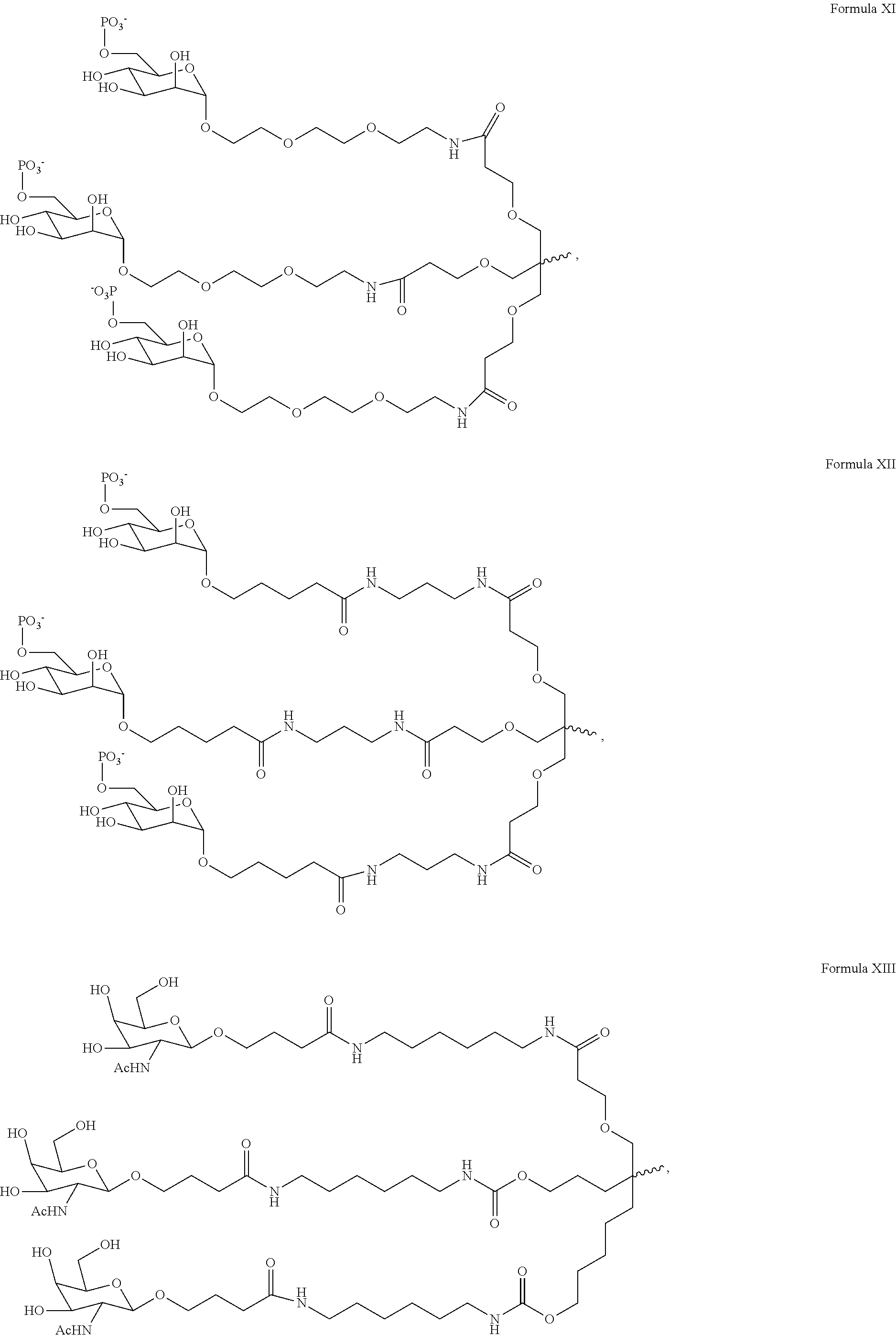

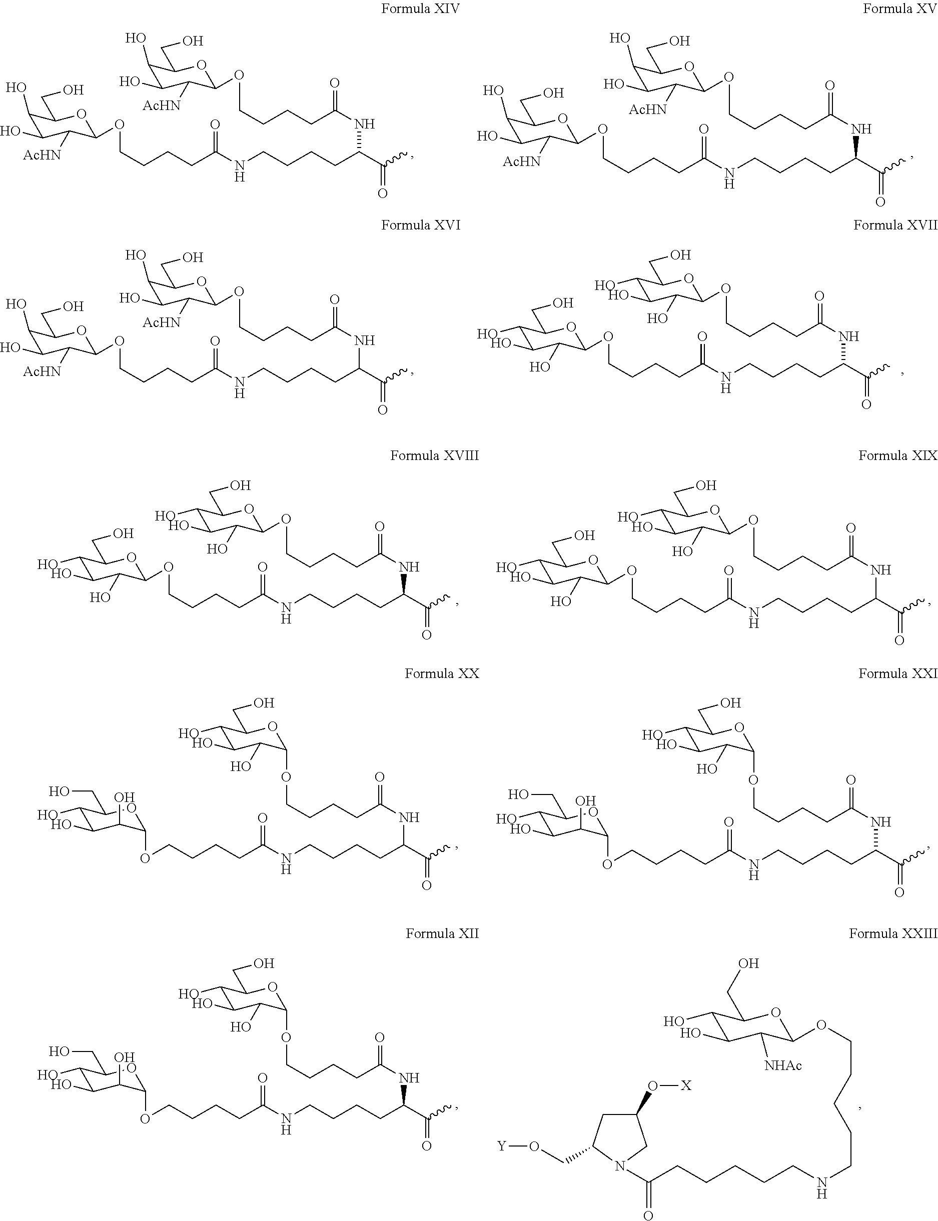

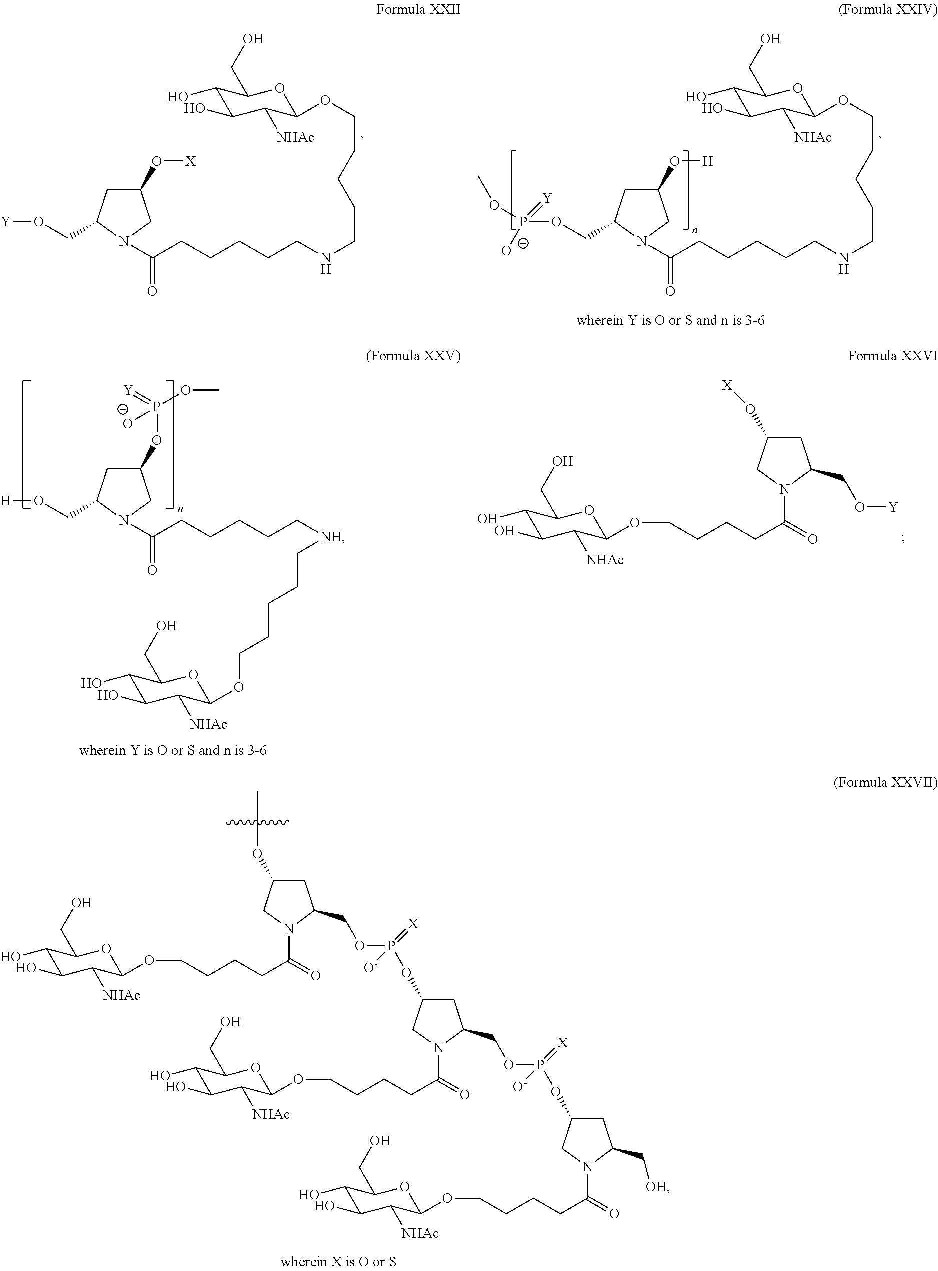

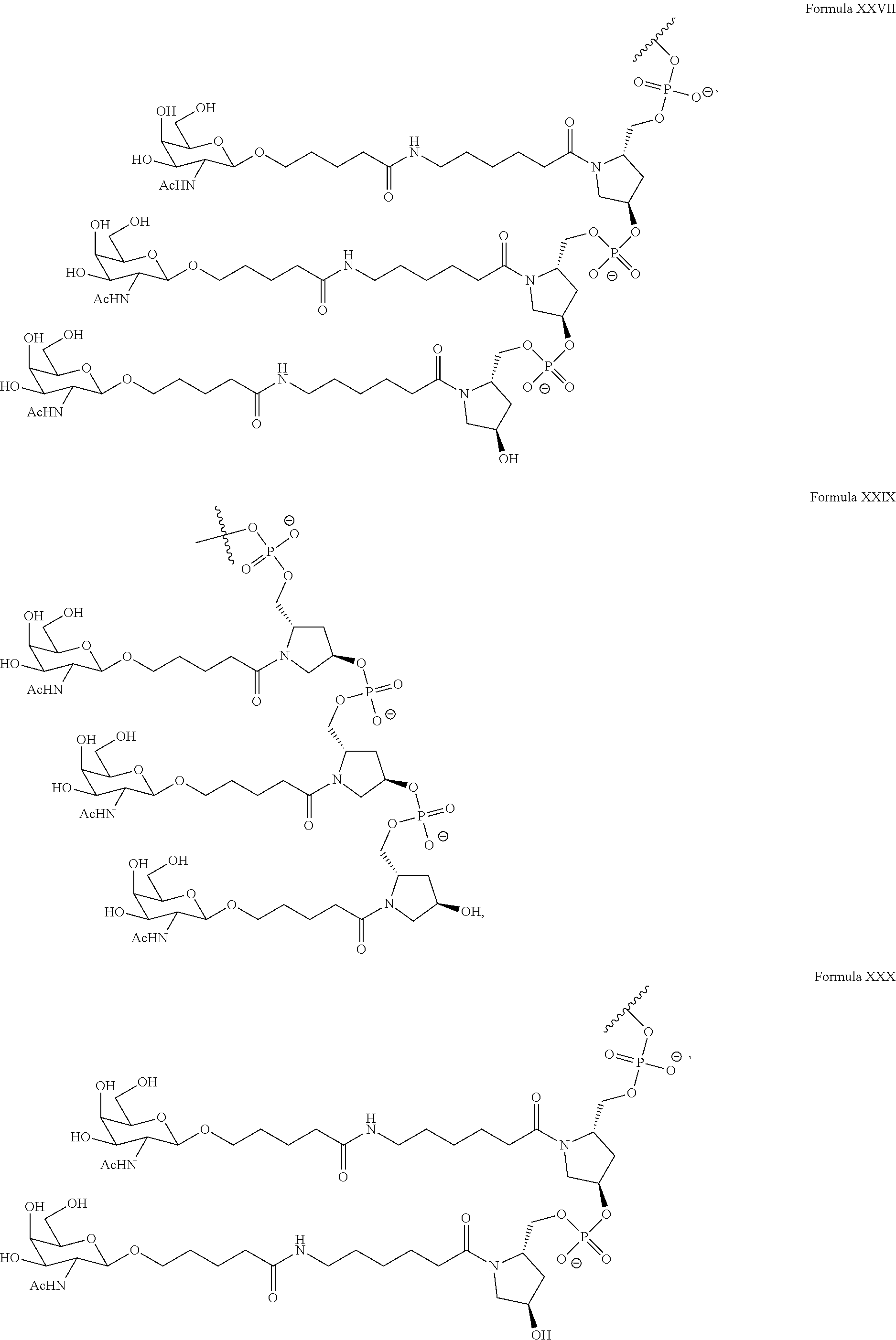

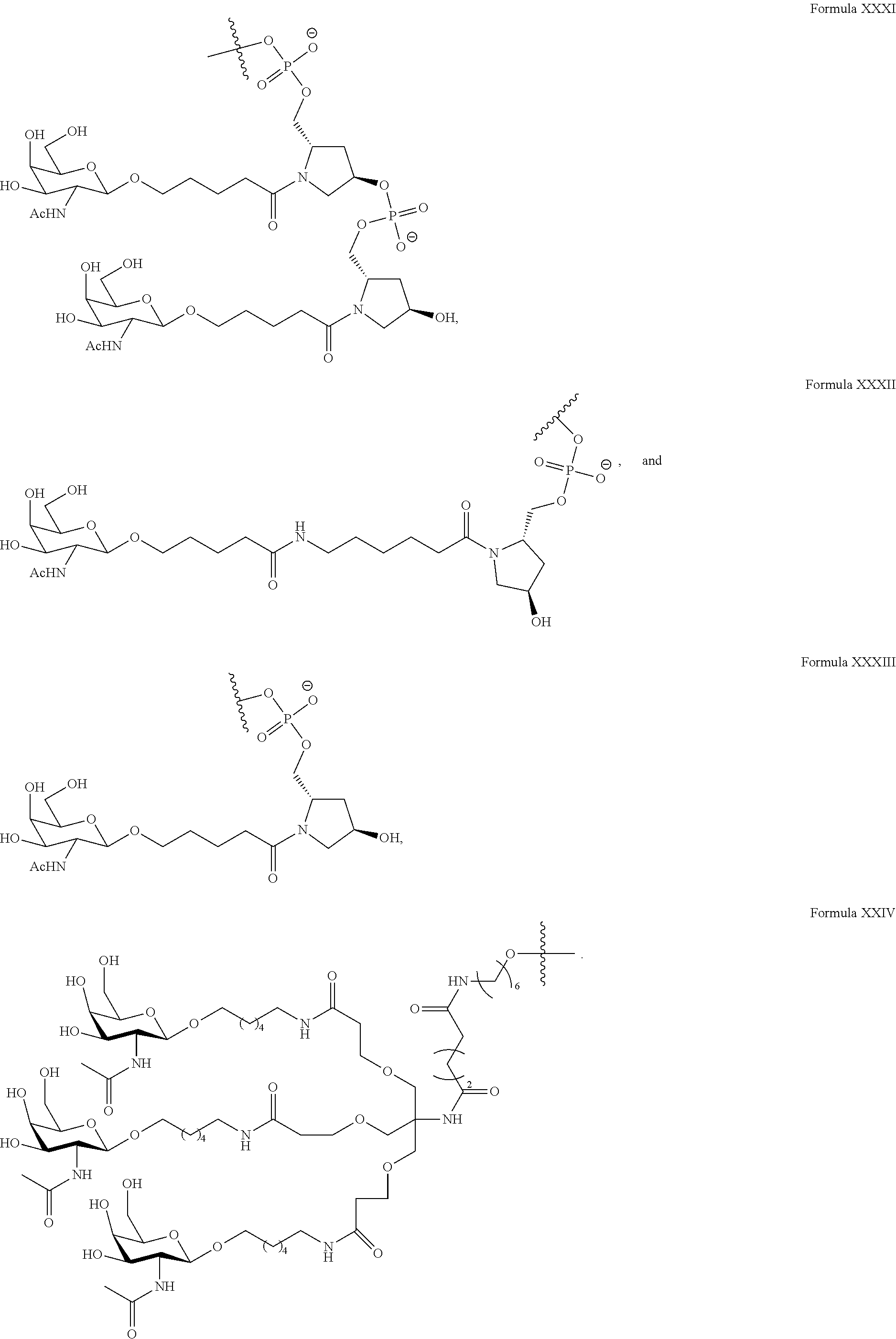

In certain embodiments, a carbohydrate conjugate for use in the compositions and methods of the disclosure is a monosaccharide. In certain embodiments, the monosaccharide is an N-acetylgalactosamine, such as

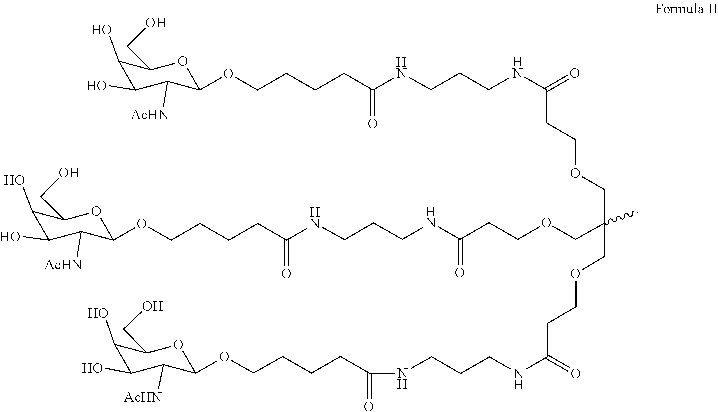 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

Another representative carbohydrate conjugate for use in the embodiments described herein includes, but is not limited to,

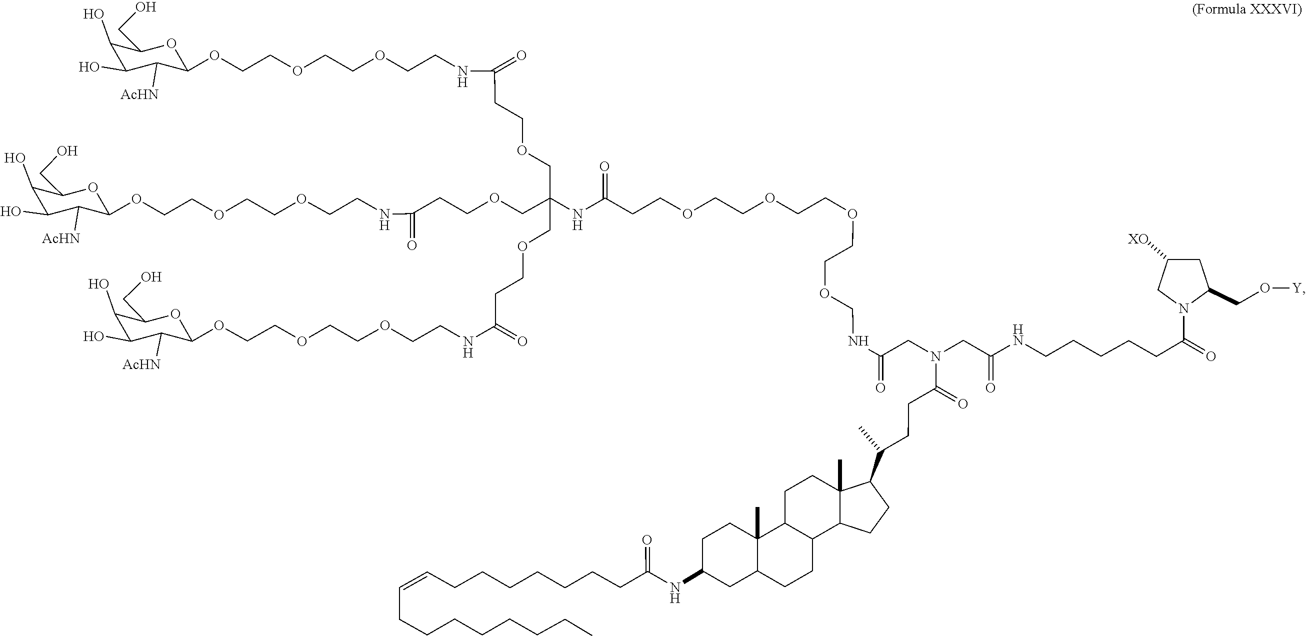

when one of X or Y is an oligonucleotide, the other is a hydrogen.

In some embodiments, a suitable ligand is a ligand disclosed in WO 2019/055633, the entire contents of which are incorporated herein by reference. In one embodiment the ligand comprises the structure below.

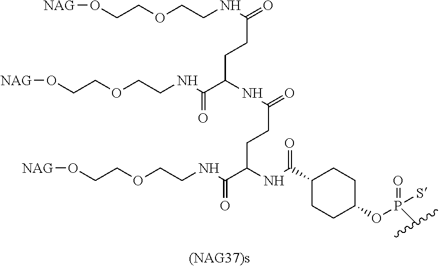

In certain embodiments, the RNAi agents of the disclosure may include GalNAc ligands, even if such GalNAc ligands are currently projected to be of limited value for the preferred intrathecal/CNS delivery route(s) of the instant disclosure.

In certain embodiments of the disclosure, the GalNAc or GalNAc derivative is attached to an iRNA agent of the disclosure via a monovalent linker. In some embodiments, the GalNAc or GalNAc derivative is attached to an iRNA agent of the disclosure via a bivalent linker. In yet other embodiments of the disclosure, the GalNAc or GalNAc derivative is attached to an iRNA agent of the disclosure via a trivalent linker. In other embodiments of the disclosure, the GalNAc or GalNAc derivative is attached to an iRNA agent of the disclosure via a tetravalent linker.

In certain embodiments, the double stranded RNAi agents of the disclosure comprise one GalNAc or GalNAc derivative attached to the iRNA agent, e.g., the 5′end of the sense strand of a dsRNA agent, or the 5′ end of one or both sense strands of a dual targeting RNAi agent as described herein. In certain embodiments, the double stranded RNAi agents of the disclosure comprise a plurality (e.g., 2, 3, 4, 5, or 6) GalNAc or GalNAc derivatives, each independently attached to a plurality of nucleotides of the double stranded RNAi agent through a plurality of monovalent linkers.

In some embodiments, for example, when the two strands of an iRNA agent of the disclosure are part of one larger molecule connected by an uninterrupted chain of nucleotides between the 3′-end of one strand and the 5′-end of the respective other strand forming a hairpin loop comprising, a plurality of unpaired nucleotides, each unpaired nucleotide within the hairpin loop may independently comprise a GalNAc or GalNAc derivative attached via a monovalent linker.

In some embodiments, the carbohydrate conjugate further comprises one or more additional ligands as described above, such as, but not limited to, a PK modulator or a cell permeation peptide.

Additional carbohydrate conjugates and linkers suitable for use in the present disclosure include those described in WO 2014/179620 and WO 2014/179627, the entire contents of each of which are incorporated herein by reference.

### D. Linkers

In some embodiments, the conjugate or ligand described herein can be attached to an iRNA oligonucleotide with various linkers that can be cleavable or non-cleavable.

The term “linker” or “linking group” means an organic moiety that connects two parts of a compound, e.g., covalently attaches two parts of a compound. Linkers typically comprise a direct bond or an atom such as oxygen or sulfur, a unit such as NR8, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R8), C(O), substituted or unsubstituted aryl, substituted or unsubstituted heteroaryl, substituted or unsubstituted heterocyclic; where R8 is hydrogen, acyl, aliphatic or substituted aliphatic. In certain embodiments, the linker is of a length of about 1-24 atoms, 2-24, 3-24, 4-24, 5-24, 6-24, 6-18, 7-18, 8-18 atoms, 7-17, 8-17, 6-16, 7-16, or 8-16 atoms.

A cleavable linking group is one which is sufficiently stable outside the cell, but which upon entry into a target cell is cleaved to release the two parts the linker is holding together. In a preferred embodiment, the cleavable linking group is cleaved at least about 10 times, 20, times, 30 times, 40 times, 50 times, 60 times, 70 times, 80 times, 90 times or more, or at least about 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In preferred embodiments, useful candidate compounds are cleaved at least about 2, 4, 10, 20, 30, 40, 50, 60, 70, 80, 90, or about 100 times faster in the cell (or under in vitro conditions selected to mimic intracellular conditions) as compared to blood or serum (or under in vitro conditions selected to mimic extracellular conditions).

i. Redox Cleavable Linking Groups

In certain embodiments, a cleavable linking group is a redox cleavable linking group that is cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In one, candidate compounds are cleaved by at most about 10% in the blood. In other embodiments, useful candidate compounds are degraded at least about 2, 4, 10, 20, 30, 40, 50, 60, 70, 80, 90, or about 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

ii. Phosphate-Based Cleavable Linking Groups

In certain embodiments, a cleavable linker comprises a phosphate-based cleavable linking group. A phosphate-based cleavable linking group is cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O, —S—P(S)(H)—O—, —S—P(O)(H)—S—, —O—P(S)(H)—S—. A preferred embodiment is —O—P(O)(OH)—O—. These candidates can be evaluated using methods analogous to those described above.

iii. Acid Cleavable Linking Groups

In certain embodiments, a cleavable linker comprises an acid cleavable linking group. An acid cleavable linking group is a linking group that is cleaved under acidic conditions. In preferred embodiments acid cleavable linking groups are cleaved in an acidic environment with a pH of about 6.5 or lower (e.g., about 6.0, 5.75, 5.5, 5.25, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl.

These candidates can be evaluated using methods analogous to those described above.

iv. Ester-Based Cleavable Linking Groups

In certain embodiments, a cleavable linker comprises an ester-based cleavable linking group. An ester-based cleavable linking group is cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

v. Peptide-Based Cleavable Linking Groups

In yet another embodiment, a cleavable linker comprises a peptide-based cleavable linking group. A peptide-based cleavable linking group is cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide-based cleavage group is generally limited to the peptide bond (i.e., the amide bond) formed between amino acids yielding peptides and proteins and does not include the entire amide functional group. Peptide-based cleavable linking groups have the general formula —NHCHR_{A}C(O)NHCHR_{B}C(O)—, where R_{A} and R_{B} are the R groups of the two adjacent amino acids. These candidates can be evaluated using methods analogous to those described above.

In some embodiments, an iRNA of the disclosure is conjugated to a carbohydrate through a linker. Non-limiting examples of iRNA carbohydrate conjugates with linkers of the compositions and methods of the disclosure include, but are not limited to,

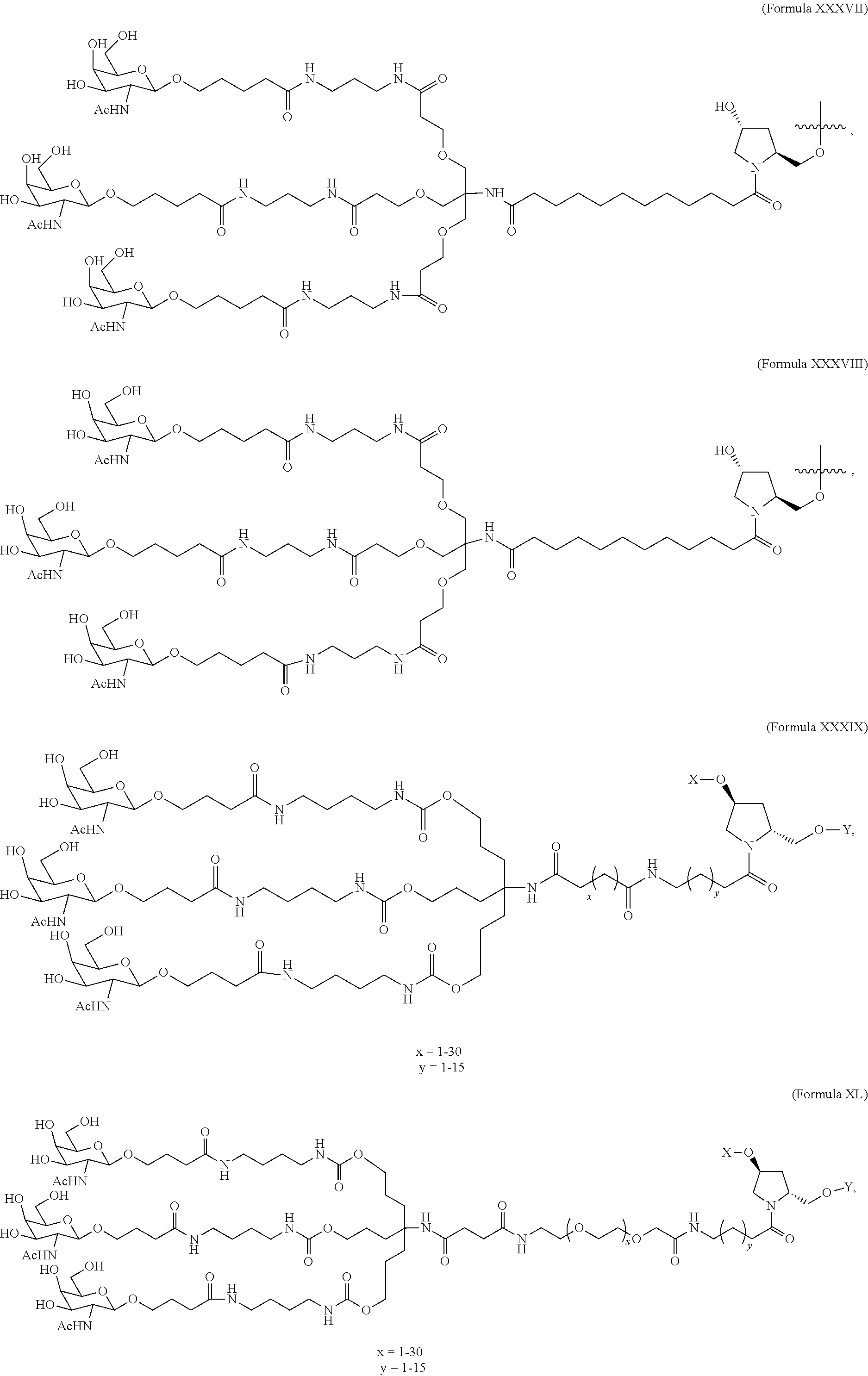

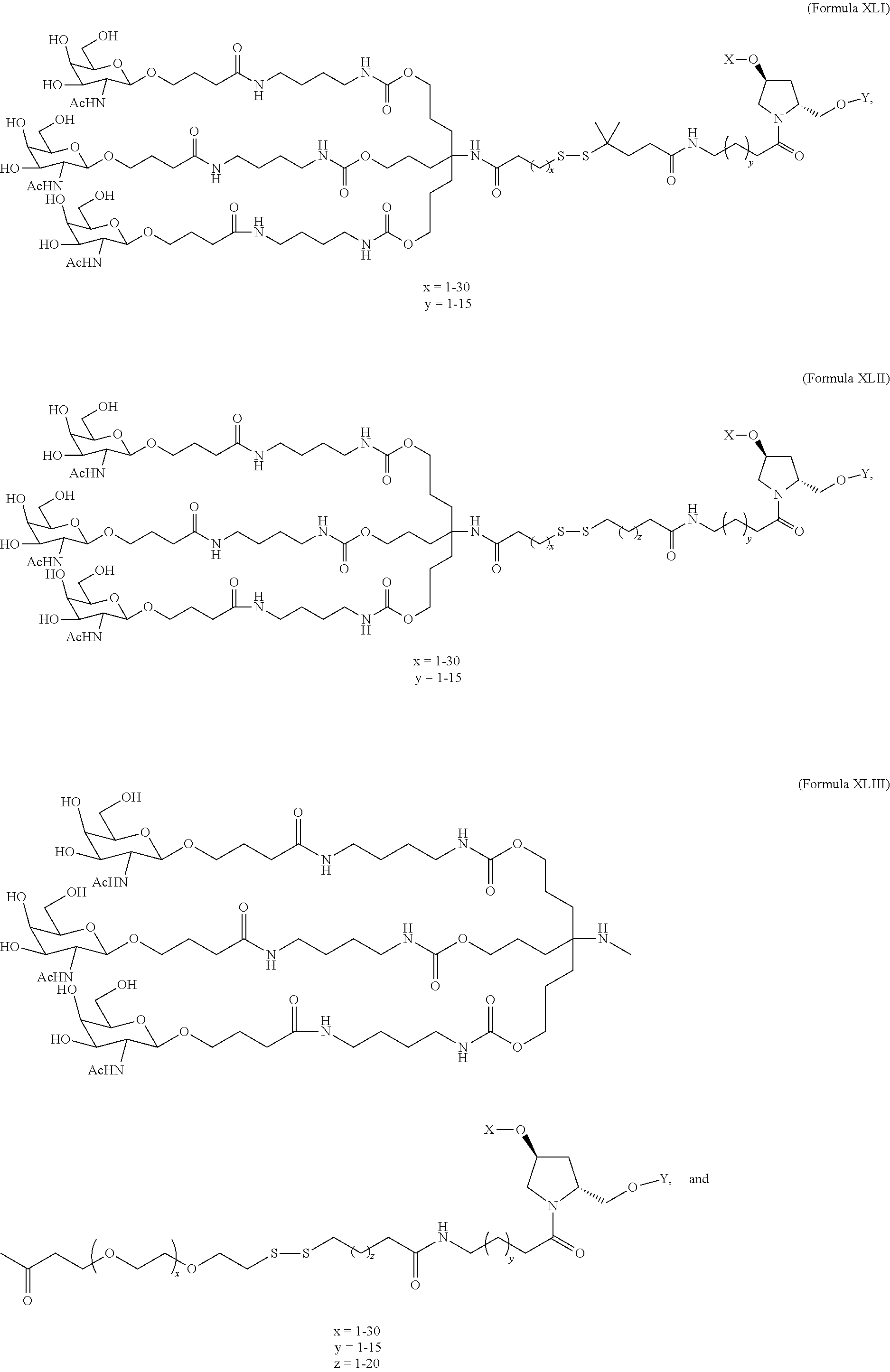

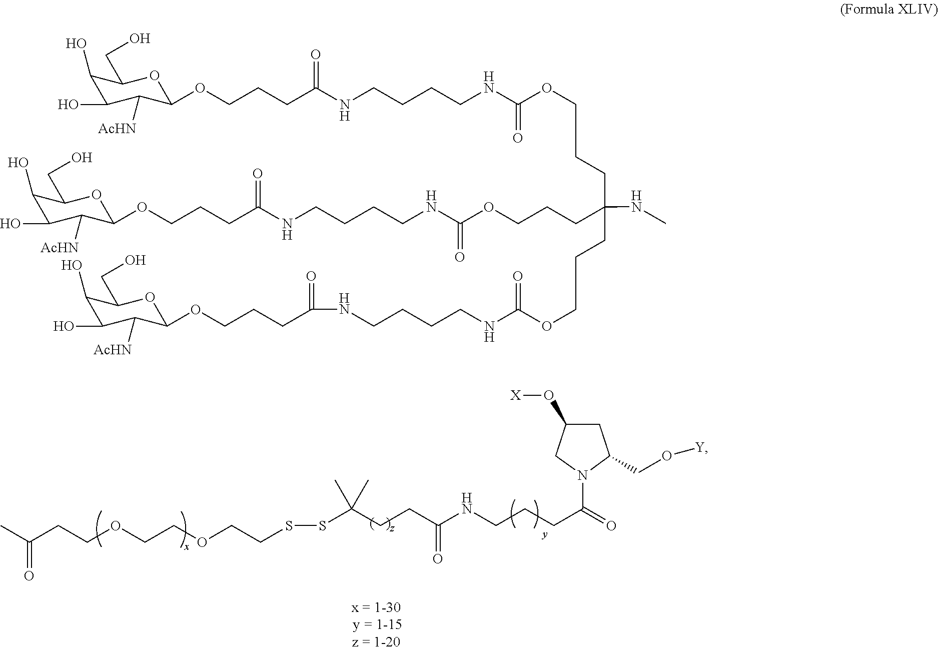

when one of X or Y is an oligonucleotide, the other is a hydrogen.

In certain embodiments of the compositions and methods of the disclosure, a ligand is one or more “GalNAc” (N-acetylgalactosamine) derivatives attached through a bivalent or trivalent branched linker.

In certain embodiments, a dsRNA of the disclosure is conjugated to a bivalent or trivalent branched linker selected from the group of structures shown in any of formula (XLV)-(XLVI):

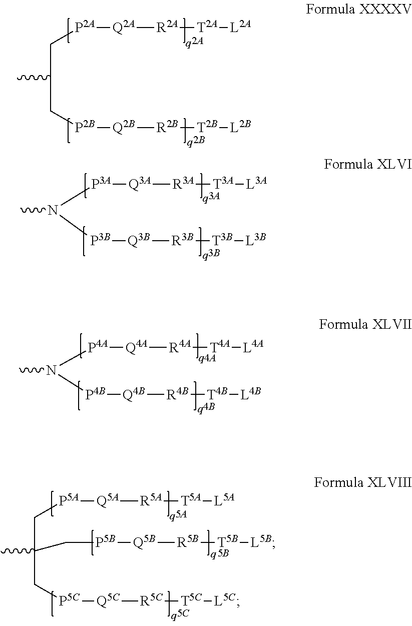

wherein:
q2A, q2B, q3A, q3B, q4A, q4B, q5A, q5B and q5C represent independently for each occurrence 0-20 and wherein the repeating unit can be the same or different;
P^{2A}, P^{2B}, P^{3A}, P^{3B}, P^{4A}, P^{4B}, P^{5A}, P^{5B}, P^{5C}, T^{2A}, T^{2B}, T^{3A}, T^{3B}, T^{4A}, T^{4B}, T^{4A}, T^{5B}, T^{5C} are each independently for each occurrence absent, CO, NH, O, S, OC(O), NHC(O), CH₂, CH₂NH or CH₂O;
Q^{2A}, Q^{2B}, Q^{3A}, Q^{3B}, Q^{4A}, Q^{4B}, Q^{5A}, Q^{5B}, Q^{5C} are independently for each occurrence absent, alkylene, substituted alkylene wherein one or more methylenes can be interrupted or terminated by one or more of O, S, S(O), SO₂, N(RN), C(R′)═C(R″), C≡C or C(O);
R^{2A}, R^{2B}, R^{3A}, R^{3B}, R^{4A}, R^{4B}, R^{5A}, R^{5B}, R^{5C} are each independently for each occurrence absent, NH, O, S, CH₂, C(O)O, C(O)NH, NHCH(R^{a})C(O), —C(O)—CH(R^{a})—NH—, CO, CH═N—O,

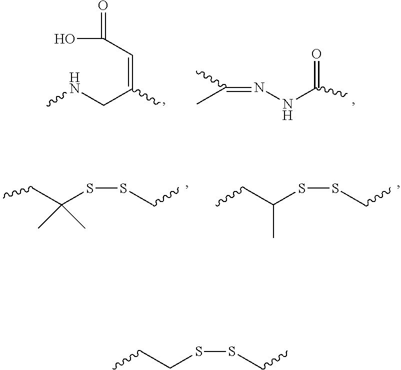

or heterocyclyl;
L^{2A}, L^{2B}, L^{3A}, L^{3B}, L^{4A}, L^{4B}, L^{5A}, L^{5B} and L^{5C} represent the ligand; i.e. each independently for each occurrence a monosaccharide (such as GalNAc), disaccharide, trisaccharide, tetrasaccharide, oligosaccharide, or polysaccharide; and R^{a} is H or amino acid side chain. Trivalent conjugating GalNAc derivatives are particularly useful for use with RNAi agents for inhibiting the expression of a target gene, such as those of formula (XLIX):

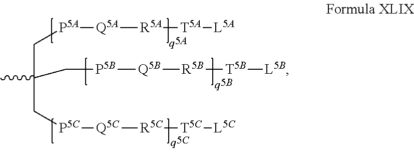

wherein L^{5A}, L^{5B}, and L^{5C} represent a monosaccharide, such as GalNAc derivative.

Examples of suitable bivalent and trivalent branched linker groups conjugating GalNAc derivatives include, but are not limited to, the structures recited above as formulas II, VII, XI, X, and XIII.

Representative U.S. patents that teach the preparation of RNA conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; 5,688,941; 6,294,664; 6,320,017; 6,576,752; 6,783,931; 6,900,297; 7,037,646; and 8,106,022, the entire contents of each of which are hereby incorporated herein by reference.

It is not necessary for all positions in a given compound to be uniformly modified, and in fact more than one of the aforementioned modifications can be incorporated in a single compound or even at a single nucleoside within an iRNA. The present disclosure also includes iRNA compounds that are chimeric compounds.

“Chimeric” iRNA compounds or “chimeras,” in the context of this disclosure, are iRNA compounds, optionally dsRNA agents, that contain two or more chemically distinct regions, each made up of at least one monomer unit, i.e., a nucleotide in the case of a dsRNA compound. These iRNAs typically contain at least one region wherein the RNA is modified so as to confer upon the iRNA increased resistance to nuclease degradation, increased cellular uptake, or increased binding affinity for the target nucleic acid. An additional region of the iRNA can serve as a substrate for enzymes capable of cleaving RNA: DNA or RNA: RNA hybrids. By way of example, RNase H is a cellular endonuclease which cleaves the RNA strand of an RNA: DNA duplex. Activation of RNase H, therefore, results in cleavage of the RNA target, thereby greatly enhancing the efficiency of iRNA inhibition of gene expression. Consequently, comparable results can often be obtained with shorter iRNAs when chimeric dsRNAs are used, compared to phosphorothioate deoxy dsRNAs hybridizing to the same target region. Cleavage of the RNA target can be routinely detected by gel electrophoresis and, if necessary, associated nucleic acid hybridization techniques known in the art.

In certain instances, the RNA of an iRNA can be modified by a non-ligand group. A number of non-ligand molecules have been conjugated to iRNAs in order to enhance the activity, cellular distribution or cellular uptake of the iRNA, and procedures for performing such conjugations are available in the scientific literature. Such non-ligand moieties have included lipid moieties, such as cholesterol (Kubo, T. et al., Biochem. Biophys. Res. Comm., 2007, 365(1): 54-61; Letsinger et al., Proc. Natl. Acad. Sci. USA, 1989, 86: 6553), cholic acid (Manoharan et al., Bioorg. Med. Chem. Lett., 1994, 4: 1053), a thioether, e.g., hexyl-S-tritylthiol (Manoharan et al., Ann. N.Y. Acad. Sci., 1992, 660: 306; Manoharan et al., Bioorg. Med. Chem. Let., 1993, 3: 2765), a thiocholesterol (Oberhauser et al., Nucl. Acids Res., 1992, 20: 533), an aliphatic chain, e.g., dodecandiol or undecyl residues (Saison-Behmoaras et al., EMBO J., 1991, 10: 111; Kabanov et al., FEBS Lett., 1990, 259: 327; Svinarchuk et al., Biochimie, 1993, 75: 49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36: 3651; Shea et al., Nucl. Acids Res., 1990, 18: 3777), a polyamine or a polyethylene glycol chain (Manoharan et al., Nucleosides & Nucleotides, 1995, 14: 969), or adamantane acetic acid (Manoharan et al., Tetrahedron Lett., 1995, 36: 3651), a palmityl moiety (Mishra et al., Biochim. Biophys. Acta, 1995, 1264: 229), or an octadecylamine or hexylamino-carbonyl-oxycholesterol moiety (Crooke et al., J. Pharmacol. Exp. Ther., 1996, 277: 923). Representative United States patents that teach the preparation of such RNA conjugates have been listed above. Typical conjugation protocols involve the synthesis of RNAs bearing an aminolinker at one or more positions of the sequence. The amino group is then reacted with the molecule being conjugated using appropriate coupling or activating reagents. The conjugation reaction can be performed either with the RNA still bound to the solid support or following cleavage of the RNA, in solution phase. Purification of the RNA conjugate by HPLC typically affords the pure conjugate.

### II. Role of APP in APP-Associated Neuropathologies

A role for APP in neuropathologies such as AD, PD and MS has recently been identified. Exemplary reports that have documented a role for APP in AD include the following:
(1) Comi et al. J Alzheimers Dis. 19: 1143-8: OPN is a molecule involved in macrophage recruitment and activation and implicated in neurodegeneration. To elucidate the role of OPN in AD, OPN levels were evaluated in serum and cerebrospinal fluid (CSF) of 67 AD patients, 46 frontotemporal dementia (FTD) patients, and 69 controls. OPN levels were identified as: significantly increased in the CSF of AD patients; ii) correlated with Mini-Mental State Exam (MMSE) score; and iii) were higher in the early disease phases (2 years). These findings support a role of OPN in AD pathogenesis.
(2) Sylvia Kang, Mayo Clinic grant in Neuroscience, “Role of APP on microglia and Alzheimer's disease”: The OPN-encoding APP gene is upregulated in microglia during aging, amyloidosis and tauopathy, with further increases in aged females over aged males. All of these contexts represent AD risk factors or AD associated pathologies, indicating that APP may play a significant role in disease. To date, little is known regarding how OPN functions in microglia and AD pathology. Using primary microglial cultures, it was demonstrated that OPN plays a pro-inflammatory role following stimulation with inflammatory mediators such as lipopolysaccharide (LPS) or aggregated tau. Additionally, APP^{-/-} mice (which do not express APP and therefore lack OPN) displayed reduced neuroinflammation following a systemic inflammatory challenge with LPS, again indicating a pro-inflammatory role for OPN. Accordingly, it appears that APP expression during amyloidosis and tauopathy significantly alters cognition, neuroinflammation, gliosis and other CNS sequelae observed in these settings.
(3) Frigerio et al. Cell Reports, 27: 1293-1306: Gene expression profiles of more than 10,000 individual microglial cells isolated from cortex and hippocampus of male and female App^{NL-G-F} mice over time demonstrated that progressive amyloid-β accumulation accelerates two main activated microglia states that are also present during normal aging. Activated response microglia (ARMs) are composed of specialized subgroups overexpressing MHC type II and putative tissue repair genes (Dkk2, Gpnmb, and APP) and are strongly enriched with Alzheimer's disease (AD) risk genes. Microglia from female mice progress faster in this activation trajectory. Similar activated states are also found in a second AD model and in human brain.
(4) Chai et al. Scientific Reports, 11, Article number: 4010 (2021): Cerebrovascular disease (CeVD) and neurodegenerative dementia such as Alzheimer's disease (AD) are frequently associated comorbidities in the elderly, sharing common risk factors and pathophysiological mechanisms including neuroinflammation. Osteopontin (OPN) is an inflammatory marker found upregulated in vascular diseases as well as in AD. However, its involvement in vascular dementia (VaD) and pre-dementia stages, namely cognitive impairment no dementia (CIND), both of which fall under the spectrum of vascular cognitive impairment (VCI), has yet to be examined. Its correlations with inflammatory cytokines in cognitive impairment also await investigation. 80 subjects with no cognitive impairment (NCI), 160 with CIND and 144 with dementia were included in a cross-sectional study on a Singapore-based memory clinic cohort. All subjects underwent comprehensive clinical, neuropsychological and brain neuroimaging assessments, together with clinical diagnoses based on established criteria. Blood samples were collected and OPN as well as inflammatory cytokines interleukin (IL)-6, IL-8 and tumor necrosis factor (TNF) were measured using immunoassays. Multivariate regression analyses showed significant associations between increased OPN and vascular cognitive impairment (VCI) groups, namely cognitive impairment no dementia (CIND) with CeVD, AD with CeVD and vascular dementia (VaD). Interestingly, higher OPN was also significantly associated with AD even in the absence of CeVD. It was further shown that increased OPN significantly associated with neuroimaging markers of CeVD and neurodegeneration, including cortical infarcts, lacunes, white matter hyperintensities and brain atrophy.

At least in view of the above, APP inhibition via administration of iRNA compositions of the instant disclosure to a subject having or at risk of developing an APP-associated neurodegenerative disease (e.g., AD, PD, MS) is projected to exert a therapeutic benefit in such a subject.

### IV. In Vivo Testing of APP Knockdown

Mouse models of APP-associated neurodegenerative disease have been generated that can be used to explore the role of APP in neurodegenerative diseases characterized by enlarged neuronal cell endosomes, such as AD and DS. Notably, an APP knock-out mouse model (APP^{-/-}) that does not produce osteopontin is known in the art and can be employed for transgenic expression of hsAPP, and such mice can also be crossed with any art-recognized mouse model of AD (e.g, CVN-AD mice, among others) and/or DS. Exemplary AD model mice include a number of transgenic mouse models obtained by transferring genes carrying mutations identified in familial AD, including APP, PS1, PS2 (Lee and Han, 2013) and tau (e.g., mmMAPT tau replaced with pathogenic variant hsMAPT tau by Michael Koob, International Conference on Alzheimer's and Parkinson's Diseases 2021 (Virtual): New Mouse Models Better Mimic Tauopathy, Alzheimer's), as well as APP/PS1 mice, which are double transgenic mice expressing a chimeric mouse/human amyloid precursor protein (Mo/HuAPP695swe) and a mutant human presenilin 1 (PS1-dE9), both directed to CNS neurons. Late-onset AD knock-in mouse models are also known in the art, including, e.g., “LOAD1” mice having human ApoE4 and the TREM2 R47H variant knocked in and “LOAD2” mice expressing knocked-in human ApoE4, TREM2 R47H, and humanized Aβ42 (“LOAD1” mice having human ApoE4 and the TREM2 R47H variant knocked in and “LOAD2” mice expressing knocked-in human ApoE4, TREM2 R47H, and humanized Aβ42 (Adrian Oblak, International Conference on Alzheimer's and Parkinson's Diseases 2021 (Virtual): New Mouse Models Better Mimic Tauopathy, Alzheimer's).

APPSwe/PSEN1(A246E) mice (Borchelt et al. Neuron. 19: 939-45) are also contemplated for use as a model of combined PSEN1 and APP mutation, as such mice likely also exhibit enlarged endosomes.

Murine models for DS in wide use include, without limitation, those profiled in Herault et al. Dis Model Mech. 10: 1165-1186, particularly including the TS65Dn mouse model (segmental trisomy of mouse chr. 16; Cataldo et al. J Neurosci Off J Soc Neurosc 23:6788-6792), which replicates neurological symptoms seen in Down syndrome patients (Galdzicki and Siarey Genes Brain Behav 2:167-178). Patients with DS tend to develop AD pathology by the age of 45. The early onset of Alzheimer's disease is thought to be a result of three copies of APP in DS patients.

### V. Delivery of an RNAi Agent of the Disclosure

The delivery of an RNAi agent of the disclosure to a cell e.g., a cell within a subject, such as a human subject (e.g., a subject in need thereof, such as a subject having an APP-associated disorder characterized by enlarged neuronal cell endosomes, e.g., Alzheimer's disease (AD) or Down syndrome (DS), can be achieved in a number of different ways. For example, delivery may be performed by contacting a cell with an RNAi agent of the disclosure either in vitro or in vivo. In vivo delivery may also be performed directly by administering a composition comprising an RNAi agent, e.g., a dsRNA, to a subject. Alternatively, in vivo delivery may be performed indirectly by administering one or more vectors that encode and direct the expression of the RNAi agent. These alternatives are discussed further below.

In general, any method of delivering a nucleic acid molecule (in vitro or in vivo) can be adapted for use with an RNAi agent of the disclosure (see e.g., Akhtar S. and Julian R L., (1992) Trends Cell. Biol. 2(5): 139-144 and WO94/02595, which are incorporated herein by reference in their entireties). For in vivo delivery, factors to consider for delivering an RNAi agent include, for example, biological stability of the delivered agent, prevention of non-specific effects, and accumulation of the delivered agent in the target tissue. The non-specific effects of an RNAi agent can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the RNAi agent to be administered. Several studies have shown successful knockdown of gene products when an RNAi agent is administered locally. For example, intraocular delivery of a VEGF dsRNA by intravitreal injection in cynomolgus monkeys (Tolentino, M J. et al., (2004) Retina 24: 132-138) and subretinal injections in mice (Reich, S J. et al. (2003) Mol. Vis. 9: 210-216) were both shown to prevent neovascularization in an experimental model of age-related macular degeneration. In addition, direct intratumoral injection of a dsRNA in mice reduces tumor volume (Pille, J. et al. (2005)Mol. Ther. 11: 267-274) and can prolong survival of tumor-bearing mice (Kim, W J. et al., (2006) Mol. Ther. 14: 343-350; Li, S. et al., (2007) Mol. Ther. 15: 515-523). RNA interference has also shown success with local delivery to the CNS by direct injection (Dorn, G. et al., (2004) Nucleic Acids 32: e49; Tan, P H. et al. (2005) Gene Ther. 12: 59-66; Makimura, H. et al. (2002) BMC Neurosci. 3: 18; Shishkina, G T., et al. (2004) Neuroscience 129: 521-528; Thakker, E R., et al. (2004) Proc. Natl. Acad. Sci. U.S.A. 101: 17270-17275; Akaneya, Y., et al. (2005) J. Neurophysiol. 93: 594-602) and to the lungs by intranasal administration (Howard, K A. et al., (2006) Mol. Ther. 14: 476-484; Zhang, X. et al., (2004) J. Biol. Chem. 279: 10677-10684; Bitko, V. et al., (2005) Nat. Med. 11: 50-55). For administering an RNAi agent systemically for the treatment of a disease, the RNA can be modified or alternatively delivered using a drug delivery system; both methods act to prevent the rapid degradation of the dsRNA by endo- and exo-nucleases in vivo. Modification of the RNA or the pharmaceutical carrier can also permit targeting of the RNAi agent to the target tissue and avoid undesirable off-target effects (e.g., without wishing to be bound by theory, use of GNAs as described herein has been identified to destabilize the seed region of a dsRNA, resulting in enhanced preference of such dsRNAs for on-target effectiveness, relative to off-target effects, as such off-target effects are significantly weakened by such seed region destabilization). RNAi agents can be modified by chemical conjugation to lipophilic groups such as cholesterol to enhance cellular uptake and prevent degradation. For example, an RNAi agent directed against ApoB conjugated to a lipophilic cholesterol moiety was injected systemically into mice and resulted in knockdown of apoB mRNA in both the liver and jejunum (Soutschek, J. et al., (2004) Nature 432: 173-178). Conjugation of an RNAi agent to an aptamer has been shown to inhibit tumor growth and mediate tumor regression in a mouse model of prostate cancer (McNamara, J O. et al., (2006) Nat. Biotechnol. 24: 1005-1015). In an alternative embodiment, the RNAi agent can be delivered using drug delivery systems such as a nanoparticle, a dendrimer, a polymer, liposomes, or a cationic delivery system. Positively charged cationic delivery systems facilitate binding of molecule RNAi agent (negatively charged) and also enhance interactions at the negatively charged cell membrane to permit efficient uptake of an RNAi agent by the cell. Cationic lipids, dendrimers, or polymers can either be bound to an RNAi agent, or induced to form a vesicle or micelle (see e.g., Kim S H. et al., (2008) Journal of Controlled Release 129(2): 107-116) that encases an RNAi agent. The formation of vesicles or micelles further prevents degradation of the RNAi agent when administered systemically. Methods for making and administering cationic-RNAi agent complexes are well within the abilities of one skilled in the art (see e.g., Sorensen, D R., et al. (2003) J. Mol. Biol 327: 761-766; Verma, U N. et al., (2003) Clin. Cancer Res. 9: 1291-1300; Arnold, A S et al. (2007) J. Hypertens. 25: 197-205, which are incorporated herein by reference in their entirety). Some non-limiting examples of drug delivery systems useful for systemic delivery of RNAi agents include DOTAP (Sorensen, D R., et al (2003), supra; Verma, U N. et al., (2003), supra), Oligofectamine, “solid nucleic acid lipid particles” (Zimmermann, T S. et al., (2006) Nature 441: 111-114), cardiolipin (Chien, P Y. et al., (2005) Cancer Gene Ther. 12: 321-328; Pal, A. et al., (2005) Int J. Oncol. 26: 1087-1091), polyethyleneimine (Bonnet M E. et al., (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3: 472-487), and polyamidoamines (Tomalia, D A. et al., (2007) Biochem. Soc. Trans. 35: 61-67; Yoo, H. et al., (1999) Pharm. Res. 16: 1799-1804). In some embodiments, an RNAi agent forms a complex with cyclodextrin for systemic administration. Methods for administration and pharmaceutical compositions of RNAi agents and cyclodextrins can be found in U.S. Pat. No. 7,427,605, which is herein incorporated by reference in its entirety.

Certain aspects of the instant disclosure relate to a method of reducing the expression of an APP target gene in a cell, comprising contacting said cell with the double-stranded RNAi agent of the disclosure. In one embodiment, the cell is a hepatic cell, optionally a hepatocyte. In one embodiment, the cell is an extrahepatic cell, optionally a CNS cell.

Another aspect of the disclosure relates to a method of reducing the expression of an APP target gene in a subject, comprising administering to the subject the double-stranded RNAi agent of the disclosure.

Another aspect of the disclosure relates to a method of treating a subject having an APP-associated disorder characterized by enlarged neuronal cell endosomes, comprising administering to the subject a therapeutically effective amount of the double-stranded RNAi agent of the disclosure, thereby treating the subject. Exemplary CNS disorders that can be treated by the method of the disclosure include Alzheimer's disease (AD) and Down syndrome (DS).

In one embodiment, the double-stranded RNAi agent is administered subcutaneously.

In one embodiment, the double-stranded RNAi agent is administered intrathecally. By intrathecal administration of the double-stranded RNAi agent, the method can reduce the expression of an APP target gene in a brain (e.g., frontal lobe) tissue, for instance, entorhinal cortex of medial temporal lobe, hippocampus, cerebral cortex, basal ganglia, substantia nigra, etc.

For ease of exposition the formulations, compositions and methods in this section are discussed largely with regard to modified siRNA compounds. It may be understood, however, that these formulations, compositions and methods can be practiced with other siRNA compounds, e.g., unmodified siRNA compounds, and such practice is within the disclosure. A composition that includes an RNAi agent can be delivered to a subject by a variety of routes. Exemplary routes include: intrathecal, intravenous, topical, rectal, anal, vaginal, nasal, pulmonary, and ocular.

The RNAi agents of the disclosure can be incorporated into pharmaceutical compositions suitable for administration. Such compositions typically include one or more species of RNAi agent and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present disclosure may be administered in a number of ways depending upon whether local or systemic treatment is desired and upon the area to be treated. Administration may be topical (including ophthalmic, vaginal, rectal, intranasal, transdermal), oral, or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering the RNAi agent in aerosol form. The vascular endothelial cells could be targeted by coating a balloon catheter with the RNAi agent and mechanically introducing the RNA.

Formulations for topical administration may include transdermal patches, ointments, lotions, creams, gels, drops, suppositories, sprays, liquids, and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water, syrups, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening or flavoring agents can be added.

Compositions for intrathecal or intraventricular administration may include sterile aqueous solutions which may also contain buffers, diluents, and other suitable additives.

Formulations for parenteral administration may include sterile aqueous solutions which may also contain buffers, diluents, and other suitable additives. Intraventricular injection may be facilitated by an intraventricular catheter, for example, attached to a reservoir. For intravenous use, the total concentration of solutes may be controlled to render the preparation isotonic.

In one embodiment, the administration of the siRNA compound, e.g., a double-stranded siRNA compound, or ssiRNA compound, composition is parenteral, e.g., intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral, or ocular. Administration can be provided by the subject or by another person, e.g., a health care provider. The medication can be provided in measured doses or in a dispenser which delivers a metered dose. Selected modes of delivery are discussed in more detail below.

### Intrathecal Administration

In one embodiment, the double-stranded RNAi agent is delivered by intrathecal injection (i.e., injection into the spinal fluid which bathes the brain and spinal cord tissue). Intrathecal injection of RNAi agents into the spinal fluid can be performed as a bolus injection or via minipumps which can be implanted beneath the skin, providing a regular and constant delivery of siRNA into the spinal fluid. The circulation of the spinal fluid from the choroid plexus, where it is produced, down around the spinal cord and dorsal root ganglia and subsequently up past the cerebellum and over the cortex to the arachnoid granulations, where the fluid can exit the CNS, that, depending upon size, stability, and solubility of the compounds injected, molecules delivered intrathecally could hit targets throughout the entire CNS.

In some embodiments, the intrathecal administration is via a pump. The pump may be a surgically implanted osmotic pump. In one embodiment, the osmotic pump is implanted into the subarachnoid space of the spinal canal to facilitate intrathecal administration.

In some embodiments, the intrathecal administration is via an intrathecal delivery system for a pharmaceutical including a reservoir containing a volume of the pharmaceutical agent, and a pump configured to deliver a portion of the pharmaceutical agent contained in the reservoir. More details about this intrathecal delivery system may be found in WO 2015/116658, which is incorporated by reference in its entirety.

The amount of intrathecally injected RNAi agents may vary from one target gene to another target gene and the appropriate amount that has to be applied may have to be determined individually for each target gene. Typically, this amount ranges from 10 μg to 2 mg, optionally 50 g to 1500 μg, more optionally 100 μg to 1000 μg.

### Vector-Encoded RNAi Agents of the Disclosure

RNAi agents targeting the APP gene can be expressed from transcription units inserted into DNA or RNA vectors (see, e.g., Couture, A, et al., TIG. (1996), 12: 5-10; WO 00/22113, WO 00/22114, and U.S. Pat. No. 6,054,299). Expression is optionally sustained (months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., (1995) Proc. Natl. Acad. Sci. USA 92: 1292).

The individual strand or strands of an RNAi agent can be transcribed from a promoter on an expression vector. Where two separate strands are to be expressed to generate, for example, a dsRNA, two separate expression vectors can be co-introduced (e.g., by transfection or infection) into a target cell. Alternatively, each individual strand of a dsRNA can be transcribed by promoters both of which are located on the same expression plasmid. In one embodiment, a dsRNA is expressed as inverted repeat polynucleotides joined by a linker polynucleotide sequence such that the dsRNA has a stem and loop structure.

RNAi agent expression vectors are generally DNA plasmids or viral vectors. Expression vectors compatible with eukaryotic cells, optionally those compatible with vertebrate cells, can be used to produce recombinant constructs for the expression of an RNAi agent as described herein. Delivery of RNAi agent expressing vectors can be systemic, such as by intravenous or intramuscular administration, by administration to target cells ex-planted from the patient followed by reintroduction into the patient, or by any other means that allows for introduction into a desired target cell.

Viral vector systems which can be utilized with the methods and compositions described herein include, but are not limited to, (a) adenovirus vectors; (b) retrovirus vectors, including but not limited to lentiviral vectors, moloney murine leukemia virus, etc.; (c) adeno-associated virus (AAV) vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g. EPV and EBV vectors. Constructs for the recombinant expression of an RNAi agent will generally require regulatory elements, e.g., promoters, enhancers, etc., to ensure the expression of the RNAi agent in target cells. Other aspects to consider for vectors and constructs are known in the art.

### VI. Pharmaceutical Compositions of the Invention

The present disclosure also includes pharmaceutical compositions and formulations which include the RNAi agents of the disclosure. In one embodiment, provided herein are pharmaceutical compositions containing an RNAi agent, as described herein, and a pharmaceutically acceptable carrier. The pharmaceutical compositions containing the RNAi agent are useful for treating a disease or disorder associated with the expression or activity of APP that is further characterized by enlarged neuronal cell endosomes, e.g., Alzheimer's disease (AD) or Down syndrome (DS).

Such pharmaceutical compositions are formulated based on the mode of delivery. One example is compositions that are formulated for systemic administration via parenteral delivery, e.g., by intravenous (IV), intramuscular (IM), or for subcutaneous (subQ) delivery. Another example is compositions that are formulated for direct delivery into the CNS, e.g., by intrathecal or intravitreal routes of injection, optionally by infusion into the brain (e.g., entorhinal cortex of medial temporal lobe, hippocampus, cerebral cortex, basal ganglia, substantia nigra, etc.), such as by continuous pump infusion.

In some embodiments, the pharmaceutical compositions of the disclosure are pyrogen free or non-pyrogenic.

The pharmaceutical compositions of the disclosure may be administered in dosages sufficient to inhibit expression of an APP gene. In general, a suitable dose of an RNAi agent of the disclosure will be in the range of about 0.001 to about 200.0 milligrams per kilogram body weight of the recipient per day, generally in the range of about 1 to 50 mg per kilogram body weight per day.

A repeat-dose regimen may include administration of a therapeutic amount of an RNAi agent on a regular basis, such as monthly to once every six months. In certain embodiments, the RNAi agent is administered about once per quarter (i.e., about once every three months) to about twice per year.

After an initial treatment regimen (e.g., loading dose), the treatments can be administered on a less frequent basis.

In other embodiments, a single dose of the pharmaceutical compositions can be long lasting, such that subsequent doses are administered at not more than 1, 2, 3, or 4 or more month intervals. In some embodiments of the disclosure, a single dose of the pharmaceutical compositions of the disclosure is administered once per month. In other embodiments of the disclosure, a single dose of the pharmaceutical compositions of the disclosure is administered once per quarter to twice per year.

The skilled artisan will appreciate that certain factors can influence the dosage and timing required to effectively treat a subject, including but not limited to the severity of the disease or disorder, previous treatments, the general health or age of the subject, and other diseases present. Moreover, treatment of a subject with a therapeutically effective amount of a composition can include a single treatment or a series of treatments.

Advances in mouse genetics have generated mouse models for the study of APP-associated diseases characterized by enlarged neuronal cell endosomes that would benefit from reduction in the expression of APP. Such models can be used for in vivo testing of RNAi agents, as well as for determining a therapeutically effective dose. Suitable mouse models are known in the art and include, for example, the mouse models described elsewhere herein.

The pharmaceutical compositions of the present disclosure can be administered in a number of ways depending upon whether local or systemic treatment is desired and upon the area to be treated. Administration can be topical (e.g., by a transdermal patch), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; subdermal, e.g., via an implanted device; or intracranial, e.g., by intraparenchymal, intrathecal or intraventricular, administration.

The RNAi agents can be delivered in a manner to target a particular tissue, such as the liver, the CNS (e.g., neuronal, glial or vascular tissue of the brain), or both the liver and CNS.

Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like can be necessary or desirable. Coated condoms, gloves and the like can also be useful. Suitable topical formulations include those in which the RNAi agents featured in the disclosure are in admixture with a topical delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). RNAi agents featured in the disclosure can be encapsulated within liposomes or can form complexes thereto, in particular to cationic liposomes. Alternatively, RNAi agents can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₂₀ alkyl ester (e.g., isopropylmyristate IPM), monoglyceride, diglyceride or pharmaceutically acceptable salt thereof. Topical formulations are described in detail in U.S. Pat. No. 6,747,014, which is incorporated herein by reference.

### A. RNAi Agent Formulations Comprising Membranous Molecular Assemblies

An RNAi agent for use in the compositions and methods of the disclosure can be formulated for delivery in a membranous molecular assembly, e.g., a liposome or a micelle. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the RNAi agent composition. The lipophilic material isolates the aqueous interior from an aqueous exterior, which typically does not include the RNAi agent composition, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the RNAi agent are delivered into the cell where the RNAi agent can specifically bind to a target RNA and can mediate RNAi. In some cases, the liposomes are also specifically targeted, e.g., to direct the RNAi agent to particular cell types.

A liposome containing an RNAi agent can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The RNAi agent preparation is then added to the micelles that include the lipid component. The cationic groups on the lipid interact with the RNAi agent and condense around the RNAi agent to form a liposome. After condensation, the detergent is removed, e.g., by dialysis, to yield a liposomal preparation of RNAi agent.

If necessary, a carrier compound that assists in condensation can be added during the condensation reaction, e.g., by controlled addition. For example, the carrier compound can be a polymer other than a nucleic acid (e.g., spermine or spermidine). pH can also be adjusted to favor condensation.

Methods for producing stable polynucleotide delivery vehicles, which incorporate a polynucleotide/cationic lipid complex as structural components of the delivery vehicle, are further described in, e.g., WO 96/37194, the entire contents of which are incorporated herein by reference. Liposome formation can also include one or more aspects of exemplary methods described in Felgner, P. L. et al., (1987) Proc. Natl. Acad. Sci. USA 8: 7413-7417; U.S. Pat. Nos. 4,897,355; 5,171,678; Bangham et al., (1965) M. Mol. Biol. 23: 238; Olson et al., (1979) Biochim. Biophys. Acta 557: 9; Szoka et al., (1978) Proc. Natl. Acad. Sci. 75: 4194; Mayhew et al., (1984) Biochim. Biophys. Acta 775: 169; Kim et al., (1983) Biochim. Biophys. Acta 728: 339; and Fukunaga et al., (1984) Endocrinol. 115: 757. Commonly used techniques for preparing lipid aggregates of appropriate size for use as delivery vehicles include sonication and freeze-thaw plus extrusion (see, e.g., Mayer et al., (1986) Biochim. Biophys. Acta 858: 161. Microfluidization can be used when consistently small (50 to 200 nm) and relatively uniform aggregates are desired (Mayhew et al., (1984) Biochim. Biophys. Acta 775: 169. These methods are readily adapted to packaging RNAi agent preparations into liposomes.

Liposomes fall into two broad classes. Cationic liposomes are positively charged liposomes which interact with the negatively charged nucleic acid molecules to form a stable complex. The positively charged nucleic acid/liposome complex binds to the negatively charged cell surface and is internalized in an endosome. Due to the acidic pH within the endosome, the liposomes are ruptured, releasing their contents into the cell cytoplasm (Wang et al. (1987) Biochem. Biophys. Res. Commun., 147: 980-985).

Liposomes, which are pH-sensitive or negatively charged, entrap nucleic acids rather than complex with them. Since both the nucleic acid and the lipid are similarly charged, repulsion rather than complex formation occurs. Nevertheless, some nucleic acid is entrapped within the aqueous interior of these liposomes. pH sensitive liposomes have been used to deliver nucleic acids encoding the thymidine kinase gene to cell monolayers in culture. Expression of the exogenous gene was detected in the target cells (Zhou et al. (1992) Journal of Controlled Release, 19: 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid or phosphatidylcholine or cholesterol.

Examples of other methods to introduce liposomes into cells in vitro and in vivo include U.S. Pat. Nos. 5,283,185; 5,171,678; WO 94/00569; WO 93/24640; WO 91/16024; Felgner, (1994) J. Biol. Chem. 269: 2550; Nabel, (1993) Proc. Natl. Acad. Sci. 90: 11307; Nabel, (1992) Human Gene Ther. 3: 649; Gershon, (1993) Biochem. 32: 7143; and Strauss, (1992) EMBO J. 11: 417.

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporine A into different layers of the skin (Hu et al., (1994) S.T.P.Pharma. Sci., 4(6): 466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside GM1, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., (1987) FEBS Letters, 223: 42; Wu et al., (1993) Cancer Research, 53: 3765).

Various liposomes comprising one or more glycolipids are known in the art. Papahadjopoulos et al. (Ann. N.Y. Acad. Sci., (1987), 507: 64) reported the ability of monosialoganglioside GM1, galactocerebroside sulfate and phosphatidylinositol to improve blood half-lives of liposomes. These findings were expounded upon by Gabizon et al. (Proc. Natl. Acad. Sci. U.S.A., (1988), 85: 6949). U.S. Pat. No. 4,837,028 and WO 88/04924, both to Allen et al., disclose liposomes comprising (1) sphingomyelin and (2) the ganglioside GM1 or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al).

In one embodiment, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver RNAi agents to macrophages.

Further advantages of liposomes include: liposomes obtained from natural phospholipids are biocompatible and biodegradable; liposomes can incorporate a wide range of water and lipid soluble drugs; liposomes can protect encapsulated RNAi agents in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of RNAi agent (see, e.g., Felgner, P. L. et al., (1987) Proc. Natl. Acad. Sci. USA 8: 7413-7417, and U.S. Pat. No. 4,897,355 for a description of DOTMA and its use with DNA).

A DOTMA analogue, 1,2-bis(oleoyloxy)-3-(trimethylammonia)propane (DOTAP) can be used in combination with a phospholipid to form DNA-complexing vesicles. Lipofectin™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Indiana) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (Transfectam™, Promega, Madison, Wisconsin) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., (1991) Biochim. Biophys. Res. Commun. 179: 280). Lipopolylysine, made by conjugating polylysine to DOPE, has been reported to be effective for transfection in the presence of serum (Zhou, X. et al., (1991) Biochim. Biophys. Acta 1065: 8). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, California) and Lipofectamine (DOSPA) (Life Technology, Inc., Gaithersburg, Maryland). Other cationic lipids suitable for the delivery of oligonucleotides are described in WO 98/39359 and WO 96/37194.

Liposomal formulations are particularly suited for topical administration, liposomes present several advantages over other formulations. Such advantages include reduced side effects related to high systemic absorption of the administered drug, increased accumulation of the administered drug at the desired target, and the ability to administer RNAi agent into the skin. In some implementations, liposomes are used for delivering RNAi agent to epidermal cells and also to enhance the penetration of RNAi agent into dermal tissues, e.g., into skin. For example, the liposomes can be applied topically. Topical delivery of drugs formulated as liposomes to the skin has been documented (see, e.g., Weiner et al., (1992) Journal of Drug Targeting, vol. 2,405-410 and du Plessis et al., (1992) Antiviral Research, 18: 259-265; Mannino, R. J. and Fould-Fogerite, S., (1998) Biotechniques 6: 682-690; Itani, T. et al., (1987) Gene 56: 267-276; Nicolau, C. et al. (1987) Meth. Enzymol. 149: 157-176; Straubinger, R. M. and Papahadjopoulos, D. (1983) Meth. Enzymol. 101: 512-527; Wang, C. Y. and Huang, L., (1987) Proc. Natl. Acad. Sci. USA 84: 7851-7855).

Non-ionic liposomal systems have also been examined to determine their utility in the delivery of drugs to the skin, in particular systems comprising non-ionic surfactant and cholesterol. Non-ionic liposomal formulations comprising Novasome I (glyceryl dilaurate/cholesterol/polyoxyethylene-10-stearyl ether) and Novasome II (glyceryl distearate/cholesterol/polyoxyethylene-10-stearyl ether) were used to deliver a drug into the dermis of mouse skin. Such formulations with RNAi agent are useful for treating a dermatological disorder.

Liposomes that include RNAi agents can be made highly deformable. Such deformability can enable the liposomes to penetrate through pore that are smaller than the average radius of the liposome. For example, transfersomes (highly deformable lipid aggregates which are attractive candidates for drug delivery vehicles) are a type of deformable liposomes. Transfersomes can be described as lipid droplets which are so highly deformable that they are easily able to penetrate through pores which are smaller than the droplet. Transfersomes are adaptable to the environment in which they are used, e.g., they are self-optimizing (adaptive to the shape of pores in the skin), self-repairing, frequently reach their targets without fragmenting, and often self-loading. Transferosomes can be made by adding surface edge activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin. Transfersomes that include RNAi agent can be delivered, for example, subcutaneously by infection in order to deliver RNAi agent to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading.

Other formulations amenable to the present disclosure are described in U.S. provisional application Ser. No. 61/018,616, filed Jan. 2, 2008; 61/018,611, filed Jan. 2, 2008; 61/039,748, filed Mar. 26, 2008; 61/047,087, filed Apr. 22, 2008 and 61/051,528, filed May 8, 2008. PCT application number PCT/US2007/080331, filed Oct. 3, 2007, also describes formulations that are amenable to the present disclosure.

Surfactants find wide application in formulations such as those described herein, particularly in emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, N.Y., 1988, p. 285).

The RNAi agent for use in the methods of the disclosure can also be provided as micellar formulations. “Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

A mixed micellar formulation suitable for delivery through transdermal membranes may be prepared by mixing an aqueous solution of the siRNA composition, an alkali metal C₈ to C22 alkyl sulphate, and a micelle forming compounds. Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof. The micelle forming compounds may be added at the same time or after addition of the alkali metal alkyl sulphate. Mixed micelles will form with substantially any kind of mixing of the ingredients but vigorous mixing in order to provide smaller size micelles.

In one method a first micellar composition is prepared which contains the siRNA composition and at least the alkali metal alkyl sulphate. The first micellar composition is then mixed with at least three micelle forming compounds to form a mixed micellar composition. In another method, the micellar composition is prepared by mixing the siRNA composition, the alkali metal alkyl sulphate and at least one of the micelle forming compounds, followed by addition of the remaining micelle forming compounds, with vigorous mixing.

Phenol or m-cresol may be added to the mixed micellar composition to stabilize the formulation and protect against bacterial growth. Alternatively, phenol or m-cresol may be added with the micelle forming ingredients. An isotonic agent such as glycerin may also be added after formation of the mixed micellar composition.

For delivery of the micellar formulation as a spray, the formulation can be put into an aerosol dispenser and the dispenser is charged with a propellant. The propellant, which is under pressure, is in liquid form in the dispenser. The ratios of the ingredients are adjusted so that the aqueous and propellant phases become one, i.e., there is one phase. If there are two phases, it is necessary to shake the dispenser prior to dispensing a portion of the contents, e.g., through a metered valve. The dispensed dose of pharmaceutical agent is propelled from the metered valve in a fine spray.

Propellants may include hydrogen-containing chlorofluorocarbons, hydrogen-containing fluorocarbons, dimethyl ether and diethyl ether. In certain embodiments, IFA 134a (1,1,1,2 tetrafluoroethane) may be used.

The specific concentrations of the essential ingredients can be determined by relatively straightforward experimentation. For absorption through the oral cavities, it is often desirable to increase, e.g., at least double or triple, the dosage for through injection or administration through the gastrointestinal tract.

### Lipid Particles

RNAi agents, e.g., dsRNAs of in the disclosure may be fully encapsulated in a lipid formulation, e.g., a LNP, or other nucleic acid-lipid particle.

As used herein, the term “LNP” refers to a stable nucleic acid-lipid particle. LNPs typically contain a cationic lipid, a non-cationic lipid, and a lipid that prevents aggregation of the particle (e.g., a PEG-lipid conjugate). LNPs are extremely useful for systemic applications, as they exhibit extended circulation lifetimes following intravenous (i.v.) injection and accumulate at distal sites (e.g., sites physically separated from the administration site). LNPs include “pSPLP,” which include an encapsulated condensing agent-nucleic acid complex as set forth in WO 00/03683. The particles of the present disclosure typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present disclosure are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; United States Patent publication No. 2010/0324120 and WO 96/40964.

In one embodiment, the lipid to drug ratio (mass/mass ratio) (e.g., lipid to dsRNA ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1. Ranges intermediate to the above recited ranges are also contemplated to be part of the disclosure.

Certain specific LNP formulations for delivery of RNAi agents have been described in the art, including, e.g., “LNP01” formulations as described in, e.g., WO 2008/042973, which is hereby incorporated by reference.

Additional exemplary lipid-dsRNA formulations are identified in the chart below.

| | | cationic lipid/non-cationic |
|---|---|---|
| | | lipid/cholesterol/PEG-lipid |
| | | conjugate |
| | Ionizable/Cationic Lipid | Lipid:siRNA ratio |
| SNALP-1 | 1,2-Dilinolenyloxy-N,N- | DLinDMA/DPPC/Cholesterol/PEG-cDMA |
| | dimethylaminopropane (DLinDMA) | (57.1/7.1/34.4/1.4) |
| | | lipid:siRNA~7:1 |
| 2-XTC | 2,2-Dilinoleyl-4-dimethylaminoethyl- | XTC/DPPC/Cholesterol/PEG-cDMA |
| | [1,3]-dioxolane (XTC) | 57.1/7.1/34.4/1.4 |
| | | lipid:siRNA~7:1 |
| LNP05 | 2,2-Dilinoleyl-4-dimethylaminoethyl- | XTC/DSPC/Cholesterol/PEG-DMG |
| | [1,3]-dioxolane (XTC) | 57.5/7.5/31.5/3.5 |
| | | lipid:siRNA~6:1 |
| LNP06 | 2,2-Dilinoleyl-4-dimethylaminoethyl- | XTC/DSPC/Cholesterol/PEG-DMG |
| | [1,3]-dioxolane (XTC) | 57.5/7.5/31.5/3.5 |
| | | lipid:siRNA~11:1 |
| LNP07 | 2,2-Dilinoleyl-4-dimethylaminoethyl- | XTC/DSPC/Cholesterol/PEG-DMG |
| | [1,3]-dioxolane (XTC) | 60/7.5/31/1.5, |
| | | lipid:siRNA~6:1 |
| LNP08 | 2,2-Dilinoleyl-4-dimethylaminoethyl- | XTC/DSPC/Cholesterol/PEG-DMG |
| | [1,3]-dioxolane (XTC) | 60/7.5/31/1.5, |
| | | lipid:siRNA~11:1 |
| LNP09 | 2,2-Dilinoleyl-4-dimethylaminoethyl- | XTC/DSPC/Cholesterol/PEG-DMG |
| | [1,3]-dioxolane (XTC) | 50/10/38.5/1.5 |
| | | Lipid:siRNA 10:1 |
| LNP10 | (3aR,5s,6aS)-N,N-dimethyl-2,2- | ALN100/DSPC/Cholesterol/PEG-DMG |
| | di((9Z,12Z)-octadeca-9,12- | 50/10/38.5/1.5 |
| | dienyl)tetrahydro-3aH- | Lipid:siRNA 10:1 |
| | cyclopenta[d][1,3]dioxol-5-amine | |
| | (ALN100) | |
| LNP11 | (6Z,9Z,28Z,31Z)-heptatriaconta- | MC-3/DSPC/Cholesterol/PEG-DMG |
| | 6,9,28,31-tetraen-19-yl 4- | 50/10/38.5/1.5 |
| | (dimethylamino)butanoate (MC3) | Lipid:siRNA 10:1 |
| LNP12 | 1,1′-(2-(4-(2-((2-(bis(2- | Tech G1/DSPC/Cholesterol/PEG-DMG |
| | hydroxydodecyl)amino)ethyl)(2- | 50/10/38.5/1.5 |
| | hydroxydodecyl)amino)ethyl)piperazin- | Lipid:siRNA 10:1 |
| | 1-yl)ethylazanediyl)didodecan-2-ol | |
| | (Tech G1) | |
| LNP13 | XTC | XTC/DSPC/Chol/PEG-DMG |
| | | 50/10/38.5/1.5 |
| | | Lipid:siRNA:33:1 |
| LNP14 | MC3 | MC3/DSPC/Chol/PEG-DMG |
| | | 40/15/40/5 |
| | | Lipid:siRNA:11:1 |
| LNP15 | MC3 | MC3/DSPC/Chol/PEG- |
| | | DSG/GalNAc-PEG-DSG |
| | | 50/10/35/4.5/0.5 |
| | | Lipid:siRNA:11:1 |
| LNP16 | MC3 | MC3/DSPC/Chol/PEG-DMG |
| | | 50/10/38.5/1.5 |
| | | Lipid:siRNA:7:1 |
| LNP17 | MC3 | MC3/DSPC/Chol/PEG-DSG |
| | | 50/10/38.5/1.5 |
| | | Lipid:siRNA:10:1 |
| LNP18 | MC3 | MC3/DSPC/Chol/PEG-DMG |
| | | 50/10/38.5/1.5 |
| | | Lipid:siRNA:12:1 |
| LNP19 | MC3 | MC3/DSPC/Chol/PEG-DMG |
| | | 50/10/35/5 |
| | | Lipid:siRNA:8:1 |
| LNP20 | MC3 | MC3/DSPC/Chol/PEG-DPG |
| | | 50/10/38.5/1.5 |
| | | Lipid:siRNA:10:1 |
| LNP21 | C12-200 | C12-200/DSPC/Chol/PEG-DSG |
| | | 50/10/38.5/1.5 |
| | | Lipid:siRNA:7:1 |
| LNP22 | XTC | XTC/DSPC/Chol/PEG-DSG |
| | | 50/10/38.5/1.5 |
| | | Lipid:siRNA:10:1 |

DSPC: distearoylphosphatidylcholine
DPPC: dipalmitoylphosphatidylcholine
PEG-DMG: PEG-didimyristoyl glycerol (C14-PEG, or PEG-C14) (PEG with avg mol wt of 2000)
PEG-DSG: PEG-distyryl glycerol (C18-PEG, or PEG-C18) (PEG with avg mol wt of 2000)
PEG-cDMA: PEG-carbamoyl-1,2-dimyristyloxypropylamine (PEG with avg mol wt of 2000)
SNALP (1,2-Dilinolenyloxy-N,N-dimethylaminopropane (DLinDMA)) comprising formulations are described in WO 2009/127060, which is hereby incorporated by reference.
XTC comprising formulations are described in WO 2010/088537, the entire contents of which are hereby incorporated herein by reference.
MC3 comprising formulations are described, e.g., in United States Patent Publication No. 2010/0324120, the entire contents of which are hereby incorporated by reference.
ALNY-100 comprising formulations are described in WO 2010/054406, the entire contents of which are hereby incorporated herein by reference.
C12-200 comprising formulations are described in WO 2010/129709, the entire contents of which are hereby incorporated herein by reference.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders can be desirable. In some embodiments, oral formulations are those in which dsRNAs featured in the disclosure are administered in conjunction with one or more penetration enhancer surfactants and chelators. Suitable surfactants include fatty acids or esters or salts thereof, bile acids or salts thereof. Suitable bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Suitable fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). In some embodiments, combinations of penetration enhancers are used, for example, fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether. DsRNAs featured in the disclosure can be delivered orally, in granular form including sprayed dried particles, or complexed to form micro or nanoparticles. DsRNA complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Suitable complexing agents include chitosan, N-trimethylchitosan, poly-L-lysine, polyhistidine, polyornithine, polyspermines, protamine, polyvinylpyridine, polythiodiethylaminomethylethylene P(TDAE), polyaminostyrene (e.g., p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for dsRNAs and their preparation are described in detail in U.S. Pat. No. 6,887,906, U.S. 2003/0027780, and U.S. Pat. No. 6,747,014, each of which is incorporated herein by reference.

Compositions and formulations for parenteral, intraparenchymal (into the brain), intrathecal, intraventricular or intrahepatic administration can include sterile aqueous solutions which can also contain buffers, diluents and other suitable additives such as, but not limited to, penetration enhancers, carrier compounds and other pharmaceutically acceptable carriers or excipients.

Pharmaceutical compositions of the present disclosure include, but are not limited to, solutions, emulsions, and liposome-containing formulations. These compositions can be generated from a variety of components that include, but are not limited to, preformed liquids, self-emulsifying solids and self-emulsifying semisolids. Particularly preferred are formulations that target the brain when treating APP-associated diseases or disorders characterized by enlarged neuronal cell endosomes.

The pharmaceutical formulations of the present disclosure, which can conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present disclosure can be formulated into any of many possible dosage forms such as, but not limited to, tablets, capsules, gel capsules, liquid syrups, soft gels, suppositories, and enemas. The compositions of the present disclosure can also be formulated as suspensions in aqueous, non-aqueous or mixed media. Aqueous suspensions can further contain substances which increase the viscosity of the suspension including, for example, sodium carboxymethylcellulose, sorbitol or dextran. The suspension can also contain stabilizers.

### Additional Formulations

i. Emulsions

The compositions of the present disclosure can be prepared and formulated as emulsions. Emulsions are typically heterogeneous systems of one liquid dispersed in another in the form of droplets usually exceeding 0.1p m in diameter (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions can be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions can contain additional components in addition to the dispersed phases, and the active drug which can be present as a solution in either aqueous phase, oily phase or itself as a separate phase. Pharmaceutical excipients such as emulsifiers, stabilizers, dyes, and anti-oxidants can also be present in emulsions as needed. Pharmaceutical emulsions can also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise, a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion can be a semisolid or a solid, as is the case of emulsion-style ointment bases and creams. Other means of stabilizing emulsions entail the use of emulsifiers that can be incorporated into either phase of the emulsion. Emulsifiers can broadly be classified into four categories: synthetic surfactants, naturally occurring emulsifiers, absorption bases, and finely dispersed solids (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants can be classified into different classes based on the nature of the hydrophilic group: nonionic, anionic, cationic and amphoteric (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that can readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used can be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of ease of formulation, as well as efficacy from an absorption and bioavailability standpoint (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

ii. Microemulsions

In one embodiment of the present disclosure, the compositions of RNAi agents and nucleic acids are formulated as microemulsions. A microemulsion can be defined as a system of water, oil and amphiphile which is a single optically isotropic and thermodynamically stable liquid solution (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Rosoff, in Pharmaceutical Dosage Forms, Lieberman, Rieger and Banker (Eds.), 1988, Marcel Dekker, Inc., New York, N.Y., volume 1, p. 245). Typically, microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used, and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (M0310), hexaglycerol monooleate (P0310), hexaglycerol pentaoleate (P0500), decaglycerol monocaprate (MCA750), decaglycerol monooleate (M0750), decaglycerol sequioleate (S0750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions can, however, be prepared without the use of cosurfactants and alcohol-free self-emulsifying microemulsion systems are known in the art. The aqueous phase can typically be, but is not limited to, water, an aqueous solution of the drug, glycerol, PEG300, PEG400, polyglycerols, propylene glycols, and derivatives of ethylene glycol. The oil phase can include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (see e.g., U.S. Pat. Nos. 6,191,105; 7,063,860; 7,070,802; 7,157,099; Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (see e.g., U.S. Pat. Nos. 6,191,105; 7,063,860; 7,070,802; 7,157,099; Constantinides et al., Pharmaceutical Research, 1994, 11, 1385; Ho et al., J. Pharm. Sci., 1996, 85, 138-143). Often microemulsions can form spontaneously when their components are brought together at ambient temperature. This can be particularly advantageous when formulating thermolabile drugs, peptides or RNAi agents. Microemulsions have also been effective in the transdermal delivery of active components in both cosmetic and pharmaceutical applications. It is expected that the microemulsion compositions and formulations of the present disclosure will facilitate the increased systemic absorption of RNAi agents and nucleic acids from the gastrointestinal tract, as well as improve the local cellular uptake of RNAi agents and nucleic acids.

Microemulsions of the present disclosure can also contain additional components and additives such as sorbitan monostearate (Grill 3), Labrasol, and penetration enhancers to improve the properties of the formulation and to enhance the absorption of the RNAi agents and nucleic acids of the present disclosure. Penetration enhancers used in the microemulsions of the present disclosure can be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

iii. Microparticles

An RNAi agent of the disclosure may be incorporated into a particle, e.g., a microparticle. Microparticles can be produced by spray-drying, but may also be produced by other methods including lyophilization, evaporation, fluid bed drying, vacuum drying, or a combination of these techniques.

iv. Penetration Enhancers

In one embodiment, the present disclosure employs various penetration enhancers to effect the efficient delivery of nucleic acids, particularly RNAi agents, to the skin of animals. Most drugs are present in solution in both ionized and nonionized forms. However, usually only lipid soluble or lipophilic drugs readily cross cell membranes. It has been discovered that even non-lipophilic drugs can cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers can be classified as belonging to one of five broad categories, i.e., surfactants, fatty acids, bile salts, chelating agents, and non-chelating non-surfactants (see e.g., Malmsten, M. Surfactants and polymers in drug delivery, Informa Health Care, New York, NY, 2002; Lee et al., Critical Reviews in Therapeutic Drug Carrier Systems, 1991, p. 92). Each of the above-mentioned classes of penetration enhancers are described below in greater detail.

Surfactants (or “surface-active agents”) are chemical entities which, when dissolved in an aqueous solution, reduce the surface tension of the solution or the interfacial tension between the aqueous solution and another liquid, with the result that absorption of RNAi agents through the mucosa is enhanced. In addition to bile salts and fatty acids, these penetration enhancers include, for example, sodium lauryl sulfate, polyoxyethylene-9-lauryl ether and polyoxyethylene-20-cetyl ether) (see e.g., Malmsten, M. Surfactants and polymers in drug delivery, Informa Health Care, New York, NY, 2002; Lee et al., Critical Reviews in Therapeutic Drug Carrier Systems, 1991, p. 92); and perfluorochemical emulsions, such as FC-43. Takahashi et al., J. Pharm. Pharmacol., 1988, 40, 252).

Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C₁₋₂₀ alkyl esters thereof (e.g., methyl, isopropyl and t-butyl), and mono- and di-glycerides thereof (i.e., oleate, laurate, caprate, myristate, palmitate, stearate, linoleate, etc.) (see e.g., Touitou, E., et al. Enhancement in Drug Delivery, CRC Press, Danvers, MA, 2006; Lee et al., Critical Reviews in Therapeutic Drug Carrier Systems, 1991, p. 92; Muranishi, Critical Reviews in Therapeutic Drug Carrier Systems, 1990, 7, 1-33; El Hariri et al., J. Pharm. Pharmacol., 1992, 44, 651-654).

The physiological role of bile includes the facilitation of dispersion and absorption of lipids and fat-soluble vitamins (see e.g., Malmsten, M. Surfactants and polymers in drug delivery, Informa Health Care, New York, NY, 2002; Brunton, Chapter 38 in: Goodman & Gilman's The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. Suitable bile salts include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (see e.g., Malmsten, M. Surfactants and polymers in drug delivery, Informa Health Care, New York, NY, 2002; Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating agents, as used in connection with the present disclosure, can be defined as compounds that remove metallic ions from solution by forming complexes therewith, with the result that absorption of RNAi agents through the mucosa is enhanced. With regards to their use as penetration enhancers in the present disclosure, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Suitable chelating agents include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(see e.g., Katdare, A. et al., Excipient development for pharmaceutical, biotechnology, and drug delivery, CRC Press, Danvers, MA, 2006; Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14, 43-51).

As used herein, non-chelating non-surfactant penetration enhancing compounds can be defined as compounds that demonstrate insignificant activity as chelating agents or as surfactants but that nonetheless enhance absorption of RNAi agents through the alimentary mucosa (see e.g., Muranishi, Critical Reviews in Therapeutic Drug Carrier Systems, 1990, 7, 1-33). This class of penetration enhancers includes, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621-626).

Agents that enhance uptake of RNAi agents at the cellular level can also be added to the pharmaceutical and other compositions of the present disclosure. For example, cationic lipids, such as lipofectin (Junichi et al, U.S. Pat. No. 5,705,188), cationic glycerol derivatives, and polycationic molecules, such as polylysine (WO 97/30731), are also known to enhance the cellular uptake of dsRNAs.

Other agents can be utilized to enhance the penetration of the administered nucleic acids, including glycols such as ethylene glycol and propylene glycol, pyrrols such as 2-pyrrol, azones, and terpenes such as limonene and menthone.

vi. Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient can be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc).

Pharmaceutically acceptable organic or inorganic excipients suitable for non-parenteral administration which do not deleteriously react with nucleic acids can also be used to formulate the compositions of the present disclosure. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids can include sterile and non-sterile aqueous solutions, non-aqueous solutions in common solvents such as alcohols, or solutions of the nucleic acids in liquid or solid oil bases. The solutions can also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

vii. Other Components

The compositions of the present disclosure can additionally contain other adjunct components conventionally found in pharmaceutical compositions, at their art-established usage levels. Thus, for example, the compositions can contain additional, compatible, pharmaceutically-active materials such as, for example, antipruritics, astringents, local anesthetics or anti-inflammatory agents, or can contain additional materials useful in physically formulating various dosage forms of the compositions of the present disclosure, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings or aromatic substances and the like which do not deleteriously interact with the nucleic acid(s) of the formulation.

Aqueous suspensions can contain substances which increase the viscosity of the suspension including, for example, sodium carboxymethylcellulose, sorbitol or dextran. The suspension can also contain stabilizers.

In some embodiments, pharmaceutical compositions featured in the disclosure include (a) one or more RNAi agents and (b) one or more agents which function by a non-RNAi mechanism and which are useful in treating an APP-associated neurodegenerative disorder.

Examples of such agents include, but are not limited to acetylcholinesterase inhibitors, NMDA receptor antagonists, gamma secretase inhibitors, antibodies directed against Abeta (e.g., aducanumab), agents directed against the tau protein anti-synuclein antibodies and fumarate compounds, among others disclosed herein or otherwise known in the art.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred.

The data obtained from cell culture assays and animal studies can be used in formulating a range of dosage for use in humans. The dosage of compositions featured herein in the disclosure lies generally within a range of circulating concentrations that include the ED₅₀ with little or no toxicity. The dosage can vary within this range depending upon the dosage form employed and the route of administration utilized. For any compound used in the methods featured in the disclosure, the therapeutically effective dose can be estimated initially from cell culture assays. A dose can be formulated in animal models to achieve a circulating plasma concentration range of the compound or, when appropriate, of the polypeptide product of a target sequence (e.g., achieving a decreased concentration of the polypeptide) that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

In addition to their administration, as discussed above, the RNAi agents featured in the disclosure can be administered in combination with other known agents effective in treatment of pathological processes mediated by nucleotide repeat expression. In any event, the administering physician can adjust the amount and timing of RNAi agent administration on the basis of results observed using standard measures of efficacy known in the art or described herein.

### VII. Kits

In certain aspects, the instant disclosure provides kits that include a suitable container containing a pharmaceutical formulation of a siRNA compound, e.g., a double-stranded siRNA compound, or siRNA compound, (e.g., a precursor, e.g., a larger siRNA compound which can be processed into a siRNA compound, or a DNA which encodes an siRNA compound, e.g., a double-stranded siRNA compound, or siRNA compound, or precursor thereof). In certain embodiments the individual components of the pharmaceutical formulation may be provided in one container. Alternatively, it may be desirable to provide the components of the pharmaceutical formulation separately in two or more containers, e.g., one container for a siRNA compound preparation, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

### VIII. Methods for Inhibiting APP Expression

The present disclosure also provides methods of inhibiting expression of an APP gene in a cell. The methods include contacting a cell with an RNAi agent, e.g., double stranded RNAi agent, in an amount effective to inhibit expression of APP in the cell, thereby inhibiting expression of APP in the cell. In certain embodiments of the disclosure, APP is inhibited preferentially in CNS (e.g., brain) cells. In other embodiments of the disclosure, APP is inhibited preferentially in the liver (e.g., hepatocytes). In certain embodiments of the disclosure, APP is inhibited in CNS (e.g., brain) cells and in liver (e.g., hepatocytes) cells.

Contacting of a cell with an RNAi agent, e.g., a double stranded RNAi agent, may be done in vitro or in vivo. Contacting a cell in vivo with the RNAi agent includes contacting a cell or group of cells within a subject, e.g., a human subject, with the RNAi agent. Combinations of in vitro and in vivo methods of contacting a cell are also possible.

Contacting a cell may be direct or indirect, as discussed above. Furthermore, contacting a cell may be accomplished via a targeting ligand, including any ligand described herein or known in the art. In some embodiments, the targeting ligand is a carbohydrate moiety, e.g., a GalNAc ligand, or any other ligand that directs the RNAi agent to a site of interest.

The term “inhibiting,” as used herein, is used interchangeably with “reducing,” “silencing,” “downregulating,” “suppressing” and other similar terms, and includes any level of inhibition. In certain embodiments, a level of inhibition, e.g., for an RNAi agent of the instant disclosure, can be assessed in cell culture conditions, e.g., wherein cells in cell culture are transfected via Lipofectamine™-mediated transfection at a concentration in the vicinity of a cell of 10 nM or less, 1 nM or less, etc. Knockdown of a given RNAi agent can be determined via comparison of pre-treated levels in cell culture versus post-treated levels in cell culture, optionally also comparing against cells treated in parallel with a scrambled or other form of control RNAi agent. Knockdown in cell culture of, e.g., optionally 50% or more, can thereby be identified as indicative of “inhibiting” or “reducing”, “downregulating” or “suppressing”, etc. having occurred. It is expressly contemplated that assessment of targeted mRNA or encoded protein levels (and therefore an extent of “inhibiting”, etc. caused by an RNAi agent of the disclosure) can also be assessed in in vivo systems for the RNAi agents of the instant disclosure, under properly controlled conditions as described in the art.

The phrase “inhibiting expression of an APP gene” or “inhibiting expression of APP,” as used herein, includes inhibition of expression of any APP gene (such as, e.g., a mouse APP gene, a rat APP gene, a monkey APP gene, or a human APP gene) as well as variants or mutants of an APP gene that encode an APP protein. Thus, the APP gene may be a wild-type APP gene, a mutant APP gene, or a transgenic APP gene in the context of a genetically manipulated cell, group of cells, or organism.

“Inhibiting expression of an APP gene” includes any level of inhibition of an APP gene, e.g., at least partial suppression of the expression of an APP gene, such as an inhibition by at least 20%. In certain embodiments, inhibition is by at least 30%, at least 40%, optionally at least 50%, at least about 60%, at least 70%, at least about 80%, at least 85%, at least 90%, at least 95%, at least 96%, at least 97%, at least 98%, or at least 99%; or to below the level of detection of the assay method.

The expression of an APP gene may be assessed based on the level of any variable associated with APP gene expression, e.g., APP mRNA level or APP (osteopontin) protein level, or, for example, the level of amyloid plaque deposition or dopamine signaling observed in the frontal lobe, entorhinal cortex of medial temporal lobe, hippocampus, cerebral cortex, basal ganglia, substantia nigra or other brain region of a treated subject, or by observation of symptoms (e.g., cognition difficulties, dementia, depression, spasticity, tremors, walking difficulties, etc.) of APP-associated disease as an indicator of APP gene activity.

Inhibition may be assessed by a decrease in an absolute or relative level of one or more of these variables compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control).

In some embodiments of the methods of the disclosure, expression of an APP gene is inhibited by at least 20%, 30%, 40%, optionally at least 50%, 60%, 70%, 80%, 85%, 90%, or 95%, or to below the level of detection of the assay. In certain embodiments, the methods include a clinically relevant inhibition of expression of APP, e.g. as demonstrated by a clinically relevant outcome after treatment of a subject with an agent to reduce the expression of APP.

Inhibition of the expression of an APP gene may be manifested by a reduction of the amount of mRNA expressed by a first cell or group of cells (such cells may be present, for example, in a sample derived from a subject) in which an APP gene is transcribed and which has or have been treated (e.g., by contacting the cell or cells with an RNAi agent of the disclosure, or by administering an RNAi agent of the disclosure to a subject in which the cells are or were present) such that the expression of an APP gene is inhibited, as compared to a second cell or group of cells substantially identical to the first cell or group of cells but which has not or have not been so treated (control cell(s) not treated with an RNAi agent or not treated with an RNAi agent targeted to the gene of interest). The degree of inhibition may be expressed in terms of:

$$\frac{( mRNA in control cells ) - ( mRNA in treated cells )}{( mRNA in control cells )} \cdot 100 \%$$

In other embodiments, inhibition of the expression of an APP gene may be assessed in terms of a reduction of a parameter that is functionally linked to an APP gene expression, e.g., APP protein expression. APP gene silencing may be determined in any cell expressing APP, either endogenous or heterologous from an expression construct, and by any assay known in the art.

Inhibition of the expression of an APP protein may be manifested by a reduction in the level of the APP protein that is expressed by a cell or group of cells (e.g., the level of protein expressed in a sample derived from a subject). As explained above, for the assessment of mRNA suppression, the inhibition of protein expression levels in a treated cell or group of cells may similarly be expressed as a percentage of the level of protein in a control cell or group of cells.

A control cell or group of cells that may be used to assess the inhibition of the expression of an APP gene includes a cell or group of cells that has not yet been contacted with an RNAi agent of the disclosure. For example, the control cell or group of cells may be derived from an individual subject (e.g., a human or animal subject) prior to treatment of the subject with an RNAi agent.

The level of APP mRNA that is expressed by a cell or group of cells may be determined using any method known in the art for assessing mRNA expression. In one embodiment, the level of expression of APP in a sample is determined by detecting a transcribed polynucleotide, or portion thereof, e.g., mRNA of the APP gene. RNA may be extracted from cells using RNA extraction techniques including, for example, using acid phenol/guanidine isothiocyanate extraction (RNAzol B; Biogenesis), RNeasy™ RNA preparation kits (Qiagen®) or PAXgene (PreAnalytix, Switzerland). Typical assay formats utilizing ribonucleic acid hybridization include nuclear run-on assays, RT-PCR, RNase protection assays, northern blotting, in situ hybridization, and microarray analysis. Circulating APP mRNA may be detected using methods the described in WO2012/177906, the entire contents of which are hereby incorporated herein by reference.

In some embodiments, the level of expression of APP is determined using a nucleic acid probe. The term “probe”, as used herein, refers to any molecule that is capable of selectively binding to a specific APP nucleic acid or protein, or fragment thereof. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses and probe arrays. One method for the determination of mRNA levels involves contacting the isolated mRNA with a nucleic acid molecule (probe) that can hybridize to APP mRNA. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix® gene chip array. A skilled artisan can readily adapt known mRNA detection methods for use in determining the level of APP mRNA.

An alternative method for determining the level of expression of APP in a sample involves the process of nucleic acid amplification or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88: 189-193), self-sustained sequence replication (Guatelli et al. (1990) Proc. Natl. Acad. Sci. USA 87: 1874-1878), transcriptional amplification system (Kwoh et al. (1989) Proc. Natl. Acad. Sci. USA 86: 1173-1177), Q-Beta Replicase (Lizardi et al. (1988) Bio/Technology 6: 1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the disclosure, the level of expression of APP is determined by quantitative fluorogenic RT-PCR (i.e., the TaqMan™ System), by a Dual-Glo® Luciferase assay, or by other art-recognized method for measurement of APP expression or mRNA level.

The expression level of APP mRNA may be monitored using a membrane blot (such as used in hybridization analysis such as northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The determination of APP expression level may also comprise using nucleic acid probes in solution.

In some embodiments, the level of mRNA expression is assessed using branched DNA (bDNA) assays or real time PCR (qPCR). The use of this PCR method is described and exemplified in the Examples presented herein. Such methods can also be used for the detection of APP nucleic acids.

The level of APP protein expression may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like. Such assays can also be used for the detection of proteins indicative of the presence or replication of APP proteins.

In some embodiments, the efficacy of the methods of the disclosure in the treatment of an APP-related disease is assessed by a decrease in APP mRNA level (e.g, by assessment of a CSF sample for APP level, by brain biopsy, or otherwise).

In some embodiments, the efficacy of the methods of the disclosure in the treatment of an APP-related disease is assessed by a decrease in APP mRNA level (e.g, by assessment of a liver sample for APP level, by biopsy, or otherwise).

In some embodiments of the methods of the disclosure, the RNAi agent is administered to a subject such that the RNAi agent is delivered to a specific site within the subject. The inhibition of expression of APP may be assessed using measurements of the level or change in the level of APP mRNA or APP protein in a sample derived from a specific site within the subject, e.g., CNS cells. In certain embodiments, the methods include a clinically relevant inhibition of expression of APP, e.g. as demonstrated by a clinically relevant outcome after treatment of a subject with an agent to reduce the expression of APP.

As used herein, the terms detecting or determining a level of an analyte are understood to mean performing the steps to determine if a material, e.g., protein, RNA, is present. As used herein, methods of detecting or determining include detection or determination of an analyte level that is below the level of detection for the method used.

### IX. Methods of Treating or Preventing APP-Associated Neurodegenerative Diseases

The present disclosure also provides methods of using an RNAi agent of the disclosure or a composition containing an RNAi agent of the disclosure to reduce or inhibit APP expression in a cell. The methods include contacting the cell with a dsRNA of the disclosure and maintaining the cell for a time sufficient to obtain degradation of the mRNA transcript of an APP gene, thereby inhibiting expression of the APP gene in the cell. Reduction in gene expression can be assessed by any methods known in the art. For example, a reduction in the expression of APP may be determined by determining the mRNA expression level of APP using methods routine to one of ordinary skill in the art, e.g., northern blotting, qRT-PCR; by determining the protein level of APP using methods routine to one of ordinary skill in the art, such as western blotting, immunological techniques.

In the methods of the disclosure the cell may be contacted in vitro or in vivo, i.e., the cell may be within a subject.

A cell suitable for treatment using the methods of the disclosure may be any cell that expresses an APP gene. A cell suitable for use in the methods of the disclosure may be a mammalian cell, e.g., a primate cell (such as a human cell or a non-human primate cell, e.g., a monkey cell or a chimpanzee cell), a non-primate cell (such as a rat cell, or a mouse cell). In one embodiment, the cell is a human cell, e.g., a human CNS cell. In one embodiment, the cell is a human cell, e.g., a human liver cell. In one embodiment, the cell is a human cell, e.g., a human CNS cell and a human liver cell.

APP expression is inhibited in the cell by at least about 30, 40, 50, 55, 60, 65, 70, 75, 80, 85, 90, 95, 96, 97, 98, 99, or about 100%, i.e., to below the level of detection. In preferred embodiments, APP expression is inhibited by at least 50%.

The in vivo methods of the disclosure may include administering to a subject a composition containing an RNAi agent, where the RNAi agent includes a nucleotide sequence that is complementary to at least a part of an RNA transcript of the APP gene of the mammal to be treated. When the organism to be treated is a mammal such as a human, the composition can be administered by any means known in the art including, but not limited to oral, intraperitoneal, or parenteral routes, including intracranial (e.g., intraventricular, intraparenchymal, and intrathecal), intravenous, intramuscular, intravitreal, subcutaneous, transdermal, airway (aerosol), nasal, rectal, and topical (including buccal and sublingual) administration. In certain embodiments, the compositions are administered by intravenous infusion or injection. In certain embodiments, the compositions are administered by subcutaneous injection. In certain embodiments, the compositions are administered by intrathecal injection.

In some embodiments, the administration is via a depot injection. A depot injection may release the RNAi agent in a consistent way over a prolonged time period. Thus, a depot injection may reduce the frequency of dosing needed to obtain a desired effect, e.g., a desired inhibition of APP, or a therapeutic or prophylactic effect. A depot injection may also provide more consistent serum concentrations. Depot injections may include subcutaneous injections or intramuscular injections. In preferred embodiments, the depot injection is a subcutaneous injection.

In some embodiments, the administration is via a pump. The pump may be an external pump or a surgically implanted pump. In certain embodiments, the pump is a subcutaneously implanted osmotic pump. In other embodiments, the pump is an infusion pump. An infusion pump may be used for intracranial, intravenous, subcutaneous, arterial, or epidural infusions. In preferred embodiments, the infusion pump is a subcutaneous infusion pump. In other embodiments, the pump is a surgically implanted pump that delivers the RNAi agent to the CNS.

The mode of administration may be chosen based upon whether local or systemic treatment is desired and based upon the area to be treated. The route and site of administration may be chosen to enhance targeting.

In one aspect, the present disclosure also provides methods for inhibiting the expression of an APP gene in a mammal. The methods include administering to the mammal a composition comprising a dsRNA that targets an APP gene in a cell of the mammal and maintaining the mammal for a time sufficient to obtain degradation of the mRNA transcript of the APP gene, thereby inhibiting expression of the APP gene in the cell. Reduction in gene expression can be assessed by any methods known it the art and by methods, e.g. qRT-PCR, described herein.

Reduction in protein production can be assessed by any methods known it the art and by methods, e.g. ELISA, described herein. In one embodiment, a CNS biopsy sample or a cerebrospinal fluid (CSF) sample serves as the tissue material for monitoring the reduction in APP gene or protein expression (or of a proxy therefore).

The present disclosure further provides methods of treatment of a subject in need thereof. The treatment methods of the disclosure include administering an RNAi agent of the disclosure to a subject, e.g., a subject that would benefit from inhibition of APP expression, in a therapeutically effective amount of an RNAi agent targeting an APP gene or a pharmaceutical composition comprising an RNAi agent targeting an APP gene.

In addition, the present disclosure provides methods of preventing, treating or inhibiting the progression of an APP-associated neurodegenerative disease or disorder characterized by enlarged neuronal cell endosomes, such as Alzheimer's disease (AD) or Down syndrome (DS), particularly examples of such diseases associated with mutations in presenilin 1 (PSEN1).

The methods include administering to the subject a therapeutically effective amount of any of the RNAi agent, e.g., dsRNA agents, or the pharmaceutical composition provided herein, thereby preventing, treating or inhibiting the progression of the APP-associated neurodegenerative disease or disorder characterized by enlarged neuronal cell endosomes in the subject.

An RNAi agent of the disclosure may be administered as a “free RNAi agent.” A free RNAi agent is administered in the absence of a pharmaceutical composition. The naked RNAi agent may be in a suitable buffer solution. The buffer solution may comprise acetate, citrate, prolamine, carbonate, or phosphate, or any combination thereof. In one embodiment, the buffer solution is phosphate buffered saline (PBS). The pH and osmolarity of the buffer solution containing the RNAi agent can be adjusted such that it is suitable for administering to a subject.

Alternatively, an RNAi agent of the disclosure may be administered as a pharmaceutical composition, such as a dsRNA liposomal formulation.

Subjects that would benefit from a reduction or inhibition of APP gene expression are those having an APP-associated neurodegenerative disease characterized by enlarged neuronal cell endosomes.

The disclosure further provides methods for the use of an RNAi agent or a pharmaceutical composition thereof, e.g., for treating a subject that would benefit from reduction or inhibition of APP expression, e.g., a subject having an APP-associated neurodegenerative disorder, in combination with other pharmaceuticals or other therapeutic methods, e.g., with known pharmaceuticals or known therapeutic methods, such as, for example, those which are currently employed for treating these disorders. For example, in certain embodiments, an RNAi agent targeting APP is administered in combination with, e.g., an agent useful in treating an APP-associated neurodegenerative disorder as described elsewhere herein or as otherwise known in the art. For example, additional agents and treatments suitable for treating a subject that would benefit from reduction in APP expression, e.g., a subject having an APP-associated neurodegenerative disorder, may include agents currently used to treat symptoms of APP. The RNAi agent and additional therapeutic agents may be administered at the same time or in the same combination, e.g., intrathecally, or the additional therapeutic agent can be administered as part of a separate composition or at separate times or by another method known in the art or described herein.

Exemplary additional therapeutics and treatments include, for example, sedatives, antidepressants, clonazepam, sodium valproate, opiates, antiepileptic drugs, cholinesterase inhibitors, memantine, benzodiazepines, levodopa, COMT inhibitors (e.g., tolcapone and entacapone), dopamine agonists (e.g., bromocriptine, pergolide, pramipexole, ropinirole, piribedil, cabergoline, apomorphine and lisuride), MAO-B inhibitors (e.g., safinamide, selegiline and rasagiline), amantadine, an anticholinergic, modafinil, pimavanserin, doxepin, rasagline, an antipsychotic, an atypical antipsychotic (e.g., amisulpride, olanzapine, risperidone, and clozapine), riluzole, edaravone, deep brain stimulation, non-invasive ventilation (NIV), invasive ventilation physical therapy, occupational therapy, speech therapy, dietary changes and swallowing technique a feeding tube, a PEG tube, probiotics, and psychological therapy.

In one embodiment, the method includes administering a composition featured herein such that expression of the target APP gene is decreased, for at least one month. In certain embodiments, expression is decreased for at least 2 months, 3 months, or 6 months.

Optionally, the RNAi agents useful for the methods and compositions featured herein specifically target RNAs (primary or processed) of the target APP gene. Compositions and methods for inhibiting the expression of these genes using RNAi agents can be prepared and performed as described herein.

Administration of the dsRNA according to the methods of the disclosure may result in a reduction of the severity, signs, symptoms, or markers of such diseases or disorders in a patient with an APP-associated neurodegenerative disorder. By “reduction” in this context is meant a statistically significant or clinically significant decrease in such level. The reduction can be, for example, at least 5%, 10%, 15%, 20%, 25%, 30%, 35%, 40%, 45%, 50%, 55%, 60%, 65%, 70%, 75%, 80%, 85%, 90%, 95%, or about 100%.

Efficacy of treatment or prevention of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. For example, efficacy of treatment of an APP-associated neurodegenerative disorder may be assessed, for example, by periodic monitoring of a subject's cognition, learning, or memory. Comparisons of the later readings with the initial readings provide a physician an indication of whether the treatment is effective. It is well within the ability of one skilled in the art to monitor efficacy of treatment or prevention by measuring any one of such parameters, or any combination of parameters. In connection with the administration of an RNAi agent targeting APP or pharmaceutical composition thereof, “effective against” an APP-associated neurodegenerative disorder indicates that administration in a clinically appropriate manner results in a beneficial effect for at least a statistically significant fraction of patients, such as an improvement of symptoms, a cure, a reduction in disease, extension of life, improvement in quality of life, or other effect generally recognized as positive by medical doctors familiar with treating APP-associated neurodegenerative disorders and the related causes.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and optionally at least 20%, 30%, 40%, 50% or more can be indicative of effective treatment. Efficacy for a given RNAi agent drug or formulation of that drug can also be judged using an experimental animal model for the given disease as known in the art. When using an experimental animal model, efficacy of treatment is evidenced when a statistically significant reduction in a marker or symptom is observed.

Alternatively, the efficacy can be measured by a reduction in the severity of disease as determined by one skilled in the art of diagnosis based on a clinically accepted disease severity grading scale. Any positive change resulting in e.g., lessening of severity of disease measured using the appropriate scale, represents adequate treatment using an RNAi agent or RNAi agent formulation as described herein.

Subjects can be administered a therapeutic amount of dsRNA, such as about 0.01 mg/kg to about 200 mg/kg.

The RNAi agent can be administered intrathecally, via intravitreal injection, or by intravenous infusion over a period of time, on a regular basis. In certain embodiments, after an initial treatment regimen, the treatments can be administered on a less frequent basis. Administration of the RNAi agent can reduce APP levels, e.g., in a cell, tissue, blood, CSF sample or other compartment of the patient by at least 20%, 30%, 40%, 50%, 55%, 60%, 65%, 70,% 75%, 80%, 85%, 90%, 95%, 96%, 97%, 98%, or at least about 99% or more. In a preferred embodiment, administration of the RNAi agent can reduce APP levels, e.g., in a cell, tissue, blood, CSF sample or other compartment of the patient by at least 50%.

Before administration of a full dose of the RNAi agent, patients can be administered a smaller dose, such as a 5% infusion reaction, and monitored for adverse effects, such as an allergic reaction. In another example, the patient can be monitored for unwanted immunostimulatory effects, such as increased cytokine (e.g., TNF-alpha or INF-alpha) levels.

Alternatively, the RNAi agent can be administered subcutaneously, i.e., by subcutaneous injection. One or more injections may be used to deliver the desired, e.g., monthly dose of RNAi agent to a subject. The injections may be repeated over a period of time. The administration may be repeated on a regular basis. In certain embodiments, after an initial treatment regimen, the treatments can be administered on a less frequent basis. A repeat-dose regimen may include administration of a therapeutic amount of RNAi agent on a regular basis, such as monthly or extending to once a quarter, twice per year, once per year. In certain embodiments, the RNAi agent is administered about once per month to about once per quarter (i.e., about once every three months).

Unless otherwise defined, all technical and scientific terms used herein have the same meaning as commonly understood by one of ordinary skill in the art to which this invention belongs. Although methods and materials similar or equivalent to those described herein can be used in the practice or testing of the RNAi agents and methods featured in the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

An informal Sequence Listing is also filed herewith and forms part of the specification as filed.

### EXAMPLES

### Example 1: Materials and Methods

### Bioinformatics

A set of siRNAs targeting the human amyloid precursor protein gene (APP; human NCBI refseqID NM_201414.3; NCBI GeneID: 351; SEQ ID NO: 7) as well as the toxicology-species APP orthologs from cynomolgus monkey were designed using custom R and Python scripts. All the siRNA were designed to have a perfect match to the human APP transcripts and a subset either perfect or near-perfect matches to the cynomolgus monkey ortholog. The human APP NM_201414 REFSEQ mRNA, version 3 (SEQ ID NO: 7), has a length of 3358 bases. The rationale and method for the set of siRNA designs follows. The predicted efficacy for every potential 23mer siRNA from position 10 through the end was determined with a random forest model derived from the direct measure of mRNA knockdown from several thousand distinct siRNA designs targeting a diverse set of vertebrate genes. For each strand of the siRNA, a custom Python script was used in a brute force search to measure the number and positions of mismatches between the siRNA and all potential alignments in the human transcriptome. Extra weight was given to mismatches in the seed region, defined here as positions 2-9 of the antisense oligonucleotide, as well the cleavage site of the siRNA, defined here as positions 10-11 of the antisense oligonucleotide. The relative weight of the mismatches was 2.8, 1.2, 1 for seed mismatches, cleavage site, and other positions up through antisense position 19. Mismatches in the first position were ignored. A specificity score was calculated for each strand by summing the value of each weighted mismatch. Preference was given to siRNAs whose antisense score in human and cynomolgus monkey was >=2 and predicted efficacy was >=50% knockdown.

### In Vitro Screening—Dual-Glo® Luciferase Assay

Cos-7 cells (ATCC, Manassas, VA) are grown to near confluence at 37° C. in an atmosphere of 5% CO₂ in DMEM (ATCC) supplemented with 10% FBS, before being released from the plate by trypsinization. Multi-dose experiments are performed at 10 nM and 0.1 nM. siRNA and psiCHECK2-APP (NM 201414) plasmid transfections are carried out with a plasmid containing the 3′ untranslated region (UTR). Transfection is carried out by adding 5 μL of siRNA duplexes and 5 μL (5 ng) of psiCHECK2 plasmid per well along with 4.9 μL of Opti-MEM plus 0.1 μL of Lipofectamine 2000 per well (Invitrogen, Carlsbad CA. cat #13778-150) and then incubating at room temperature for 15 minutes. The mixture is then added to the cells which are re-suspended in 35 μL of fresh complete media. The transfected cells are incubated at 37° C. in an atmosphere of 5% CO2.

Forty-eight hours after the siRNAs and psiCHECK2 plasmid are transfected, Firefly (transfection control) and Renilla (fused to APP target sequence) luciferase are measured. First, media is removed from cells. Then Firefly luciferase activity is measured by adding a mixture of 20 μL Dual-Glo® Luciferase Reagent and 20 μL DMEM to each well. The mixture is incubated at room temperature for 30 minutes before luminescence (500 nm) is measured on a Spectramax (Molecular Devices) to detect the Firefly luciferase signal. Renilla luciferase activity is measured by adding a mixture of 20 μL of room temperature of Dual-Glo® Stop & Glo® Buffer and 0.1 μL Dual-Glo® Stop & Glo® Substrate to each well and the plates are incubated for 10-15 minutes before luminescence is again measured to determine the Renilla luciferase signal. The Dual-Glo® Stop & Glo® mixture quenches the firefly luciferase signal and sustained luminescence for the Renilla luciferase reaction. siRNA activity is determined by normalizing the Renilla (APP) signal to the Firefly (control) signal within each well. The magnitude of siRNA activity is then assessed relative to cells that are transfected with the same vector but are not treated with siRNA or are treated with a non-targeting siRNA. All transfections are done with n=4.

### In Vitro Screening—Cell Culture and Transfections

Cells are transfected by adding 4.9 μL of Opti-MEM plus 0.1 μL of RNAiMAX per well (Invitrogen, Carlsbad CA. cat #13778-150) to 5 μL of siRNA duplexes per well, with 4 replicates of each siRNA duplex, into a 384-well plate, and incubated at room temperature for 15 minutes. 40 μL of MEDIA containing ˜5×10³ cells are then added to the siRNA mixture. Cells are incubated for 24 hours prior to RNA purification. Experiments are performed at 10 nM and 0.1 nM. Transfection experiments are performed in human hepatoma Hep3B cells (ATCC HB-8064) with EMEM (ATCC catalog no. 30-2003), human neuroblastoma Be(2)-C cells (ATCC CRL-2268) with EMEM:F12 media (Gibco catalog no. 11765054) and mouse neuroblastoma Neuro-2A cells (ATCC CCL-131) with EMEM media.

In Vitro Screening—Total RNA Isolation Using DYNABEADS mRNA Isolation Kit

RNA is isolated using an automated protocol on a BioTek-EL406 platform using DYNABEADs (Invitrogen, cat #61012). Briefly, 70 μL of Lysis/Binding Buffer and 10 μL of lysis buffer containing 3 μL of magnetic beads are added to the plate with cells. Plates are incubated on an electromagnetic shaker for 10 minutes at room temperature and then magnetic beads are captured and the supernatant is removed. Bead-bound RNA is then washed 2 times with 150 μL Wash Buffer A and once with Wash Buffer B. Beads are then washed with 150 μL Elution Buffer, re-captured and supernatant removed.

In Vitro Screening—cDNA Synthesis Using ABI High Capacity cDNA Reverse Transcription Kit (Applied Biosystems, Foster City, CA, Cat #4368813)

12 μL of a master mix containing 1.2 μL 10× Buffer, 0.48 μL 25×dNTPs, 1.2 μL 10× Random primers, 0.6 μL Reverse Transcriptase, 0.6 μL RNase inhibitor and 7.92 μL of H2O per reaction is added to the bead bound RNA isolated above. Plates are sealed, mixed, and incubated on an electromagnetic shaker for 10 minutes at room temperature, followed by 2 h incubation at 37° C.

### In Vitro Screening—Real Time PCR

2 μL of cDNA are added to a master mix containing 0.5 μL of human or mouse GAPDH TaqMan Probe (ThermoFisher cat 4352934E or 4351309) and 0.5 μL of appropriate APP probe (e.g., Thermo Fisher Taqman human: Hs00959010, mouse: Mm00436767) and 5 μL Lightcycler 480 probe master mix (Roche Cat #04887301001) per well in a 384 well plates (Roche cat #04887301001). Real time PCR is performed in a LightCycler480 Real Time PCR system (Roche). Each duplex is tested with N=4 and data are normalized to cells transfected with a non-targeting control siRNA. To calculate relative fold change, real time data are analyzed using the ΔΔCt method and normalized to assays performed with cells transfected with a non-targeting control siRNA.

In Vivo Evaluation of APP RNAi Agents in hAPP-IRES-gLuc-Expressing Mice

Female 6-8 week old C57BL/6 mice are injected intravenously with AAV harboring a Homo sapiens APP (hAPP)-IRES (internal ribosome entry site)-Gaussia luciferase (gLuc) construct. Viral titer is assessed, e.g., targeting approx. 1E+13 VP/mL (viral particles per mL), and total dose is determined (e.g., 2E+11 VP/mouse). 5 mg/kg siRNAs are injected subcutaneously (SC, at DO) two weeks after injection of virus, and serum is sampled at DO and at day 14 (D14), while mice are sacrificed and livers harvested at D14. gLuc assessment and qPCR are performed upon D14 liver samples. A Hs00959010_ml APP Tagman probe is used for qPCR evaluation, while a Mm00436767_ml control probe is also employed.

### APP Endogenous Rodent Pharamacodynamics (PD) Study

Mouse APP (mAPP) knockdown potency of various siRNA duplexes is evaluated. Animals (C57BL/6 female mice, 20-25 g weight, or Sprague Dawley® rats) are dosed with a single ICV injection (mice) or IT injection (rats) on day 0. On day 14, animals are euthanized. CNS tissue is collected (flash frozen in 15 ml grinding vials with 2 metal balls) for mRNA analysis via RT-qPCR for relative expression of mAPP in treated groups vs a PBS control group. Three mice are dosed per group.

### CVN Mouse Studies

Male and female CVN mice were bred at CRL Germany and genotyped, housed, dosed and analyzed (IHC, ¹H-MRS and open field test) at CRL Finland, as previously reported, under regular temperature, humidity and light/dark cycle conditions (Wilcock et al. J. Neurosci. 28: 1537-1545; Colton et al. J. Neuropathol. Exp. Neurol. 73: 752-769; Kohonen et al. Nat. Commun. 8: 15932). Homozygous Tg-hAPP^{SwDI}/mNos2^{-/-} (CVN) mice were produced by crossing mice expressing the vasculotropic Swedish K670N/M671L, Dutch E693Q and Iowa D694N human APP mutations under control of the Thy-1 promoter (Davis et al. J. Biol. Chem. 279: 20296-20306) with mNos2^{-/-} (B6 129PNos2 tau1Lau/J) mice (Laubach et al. Am. J. Physiol. 275: H2211-H2218). The study was divided into two phases. In the first phase, 15 CVN mice were dosed by ICV at 3 months of age, with terminal tissue sampling at 6 months of age and 9 months of age. In the second phase, 40 CVN mice and 20 WT mice were divided into three experimental groups, with terminal tissue sampling at 9 months of age and 12 months of age. Metabolic analyses using ¹H-MRS were at 12 months of age, respectively, as previously described (Colton et al. J. Neuropathol. Exp. Neurol. 73: 752-769). Open field test measurements were performed at approximately 9 months of age. Activity chambers (Med Associates, 27×27×20.3 cm) were equipped with infrared beams. Mice were placed in the center of the chamber, and their behaviors were recorded for 30 minutes. The following parameters were recorded and analyzed: distance traveled, number of vertical rearings and average velocity. IHC was performed as described (Wilcock et al. J. Neurosci. 28: 1537-1545; Kan et al. J Neurosci. 35: 5969-5982) using anti-amyloid-beta antibody WO2 (Sigma-Aldrich, MABN10, lot 3557956, 1:500) and anti-IBA1 antibody (Wako Pure Chemicals, 01-1941, lot LEP3218, 1:200). All data are presented as mean±standard deviation or standard error of the mean, and differences were statistically significant at the P<0.05 level. Statistical analyses were performed using the PRISM statistical program (GraphPad Software).

### Mouse Intracerebroventricular (ICV) Infusion

ICV administration was performed using stereotaxically guided Hamilton syringe and infusion system (Harvard Apparatus). A single 5-μl unilateral injection was performed in the right lateral ventricle at the following coordinates: AP=−0.5 mm (posterior from bregma), ML=+1.0 mm and DV=−1.75 mm, using standard aseptic surgical procedures. After midline skin incision on the scalp, a burr hole was generated at selected coordinates using stereotaxic coordinates and a dental drill. Thereafter, a small puncture in the dura was made at the epicenter of the burr hole, and the needle was lowered to specified depth to reach the lateral ventricle. A Hamilton syringe with a 28-gauge blunt needle was filled with a volume slightly greater than 5 μl. Administration volume of the test articles was at 5 μl and infusion rate at 1 μl min⁻¹ (over 5 minutes). After the infusion, the needle was left in place for a stabilization period of 5 minutes before withdrawal from the ventricle. Withdrawal was performed slowly and paused for 30 seconds when the needle tip was at the cortical area.

### Patient Derived Cell Sources

PSEN mutation carrier Neural Progenitor cells (NPCs) were obtained from Axol Bioscience (ax0112 (PSEN1 L286V) and ax0114 (PSEN1 A246E)). Healthy control NPCs were obtained from Axol Bioscience (ax0019). APP mutation carrier and isogenic control iPS cells were obtained by CRISPR engineering of the homozygous Swedish mutation into the WTC-11 line with a RAB5-GFP reporter (Allen Cell Institute, #40). Neuron differentiation into a mixed culture of both neurons and astrocytes is described below.

Generation of Human iPSC Derived Neurons and Astrocyte Co-Cultures

iPSCs were thawed in mTESR Plus basal media (StemCell Technologies, Cat #05826) supplemented with 1×mTESR Plus Supplement (StemCell Technologies, Cat #05827) plus 10 μM Rock inhibitor Y-27632 (Sigma, Cat #Y0503), and seeded in 6 well plates pre-coated with hESCqualified Matrigel (Corning, Cat #354277). Cells were maintained in mTESR Complete media. When iPSCs reached ˜75% confluence, cells were passaged 1:10 into a new Matrigel-coated 6 well plate. On the next day (Day 1), cells were fed by complete media exchange with fresh mTESR Complete media. On Day 2, cells were transduced with 20 μl NGN2 LV plus 0.5 μl Polybrene. On Day 3, cells were fed by complete media exchange with fresh mTESR Complete media. On Day 4-6, NGN2 LV transduced iPSCs were expanded and frozen in mTESR complete media supplemented with 10% DMSO. For differentiation into neural progenitor cells (NPCs), NGN2 LV transduced iPSCs were thawed as described above. On the next day (Day 1), the media was changed to N2B27 media (50:50 Neurobasal [Gibco, Cat #21103-049] and DMEM/F-12 [Gibco, Cat #11320033] base media supplemented with 1×B-27 without vitamin A [Gibco, Cat #12587010], N2 [Gibco, Cat #17502-048], GlutaMAX [Gibco, Cat #35050], Pen/Strep [Gibco, Cat #15140122. On Day 2, cells were switched to N2B27 media supplemented with 1 μg/ml puromycin to select for stably transduced cells. On Day 4, cells were either detached and plated in new Matrigel-coated plates or frozen down in NPC Media supplemented with 10% DMSO. For neuronal differentiation and subsequent experiments, NPCs were plated in Cell Carrier-96 optically clear imaging plates (Perkin Elmer, Cat #6005550) pre-coated with 1× Matrigel at a density of 25,000 cells per well. Cells were maintained in Complete NGN2 Neuron media (N2B27 Media supplemented with 1 μM dbcAMP [Sigma, Cat #D0260], 200 μM Ascorbic Acid [Sigma, Cat #A4403], 10 ng/mL BDNF [Tocris, Cat #2837], and 10 ng/ml GDNF [R&D Systems, Cat #212-GD-050]). Wells were replenished with 50% fresh NGN2 Neuron media daily until DIV 7. At DIV 7, cells were transfected with siRNAs as outlined below following a 50% media change fresh NGN2 Neuron media supplemented with 1× CultureOne Supplement [Gibco, Cat #A3320201]. Cells were treated with BACE inhibitor 4 days prior to take down at DIV 28.

Cell Treatment with siRNA or BACE Inhibitor

At DIV 7 following neuron differentiation as described above, cells were transfected with siRNA at various concentrations using Lipofectamine RNAiMax [ThermoFisher Scientific Cat #13778100] according to manufacturer's protocol with the following modifications. siRNA and RNAiMax are diluted in Opti-MEM I Reduced Serum Medium [Gibco Cat #31985062] and added to neurons with fresh Neuron media supplemented with 1× Culture One Supplement [Gibco, Cat #A3320201]. BACE inhibitor LY2886721 [AdooQ Bioscience Cat #A10543] was added to the cells 4 days prior to take down at DIV 28.

### Visualization of Early and Late Endosomes

For live-cell imaging experiments, cells were pre-incubated with the following cell permeant fluorescent dyes added to culture media at least 1 hr prior to visualization: NucBlue Hoechst 33342 [Invitrogen, Cat #R37605, 1:200 dilution] or with the following BacMam reporters to visualize either the early endosomes (Rab5) [Invitrogen, Cat #C10586] or late endosomes (Rab7) [Invitrogen, Cat #C10589] added to culture media at least 16 hrs prior to visualization according to manufacturer recommendations. Cells were imaged at 63× magnification at 37° C. using the Perkin Elmer Opera Phenix high-content confocal imager.

### Example 2: APP-Targeting RNAi Agents Exhibited Little Impact on Morphology of Spheroids in Culture

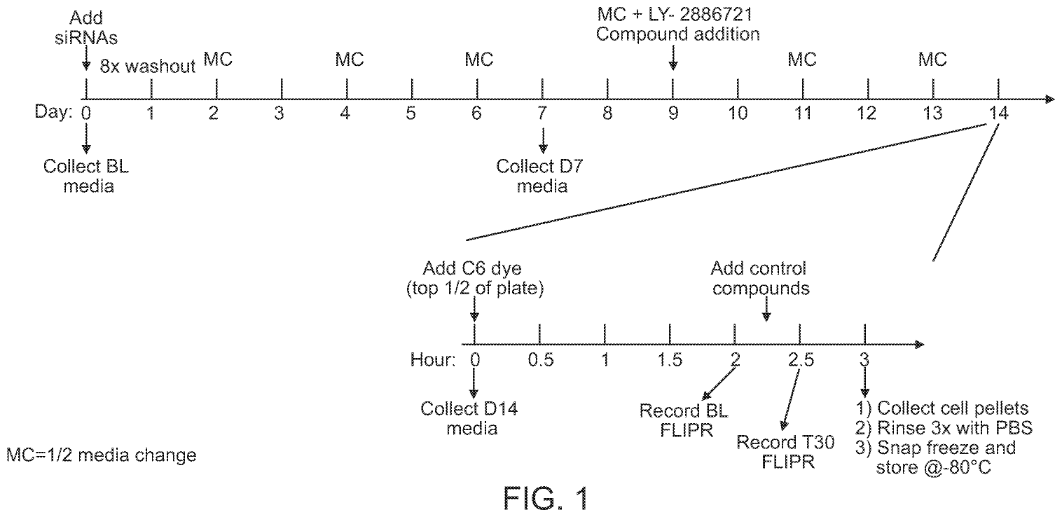

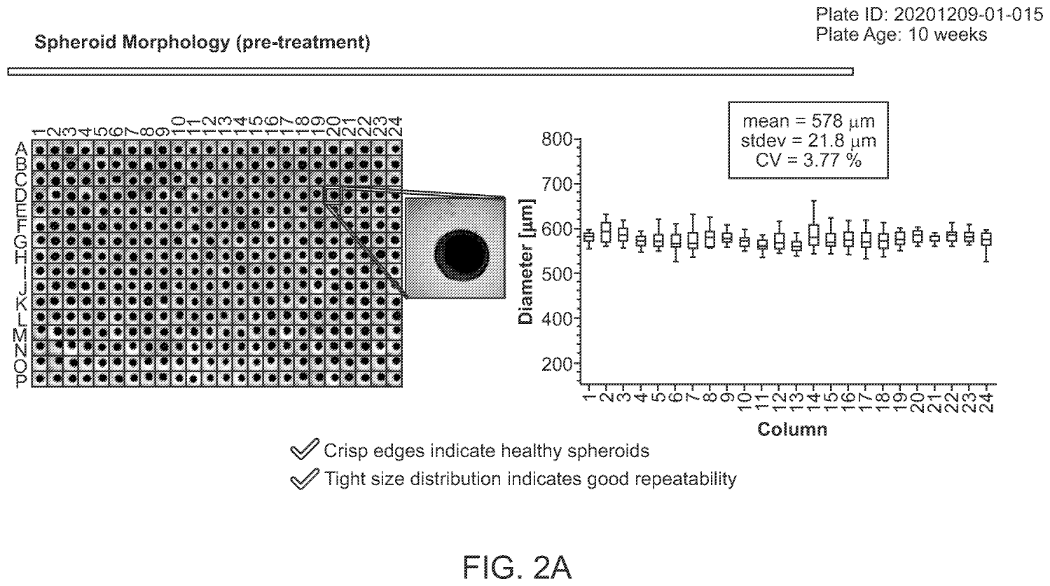

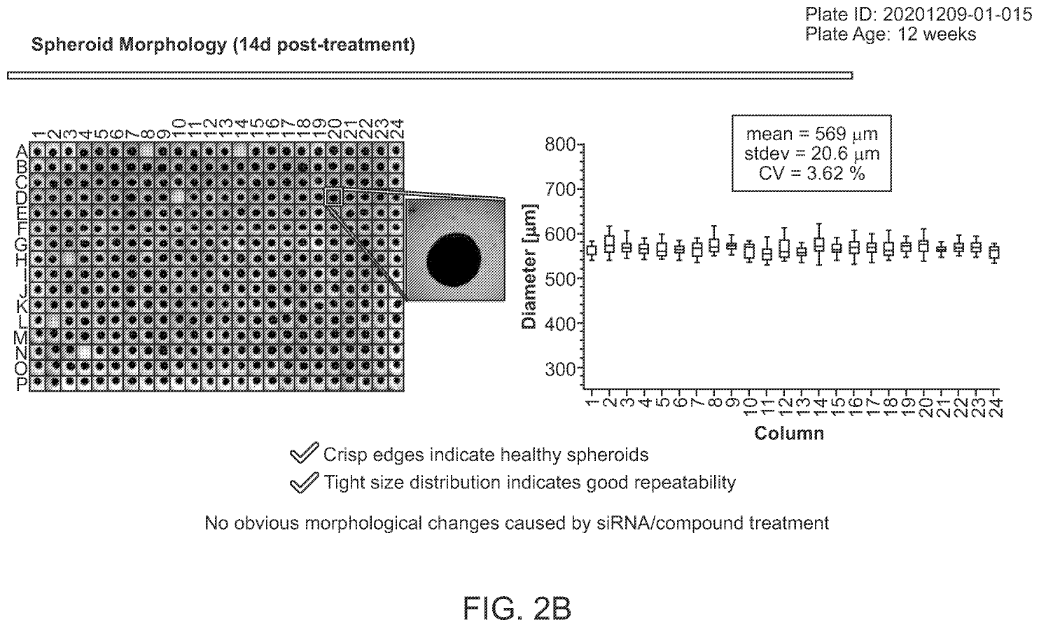

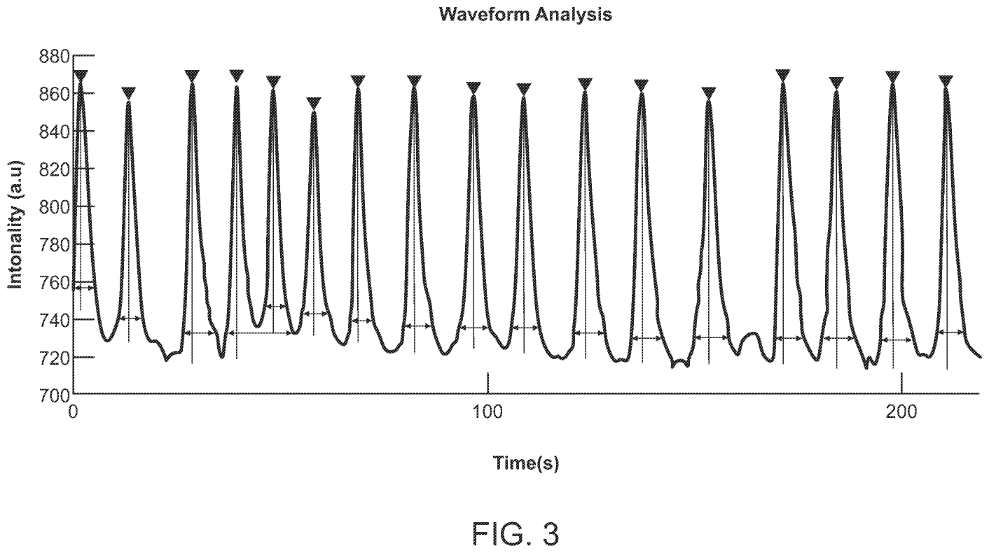

Amyloid lowering via administration of 3-Site Amyloid Precursor Protein Cleaving Enzyme (BACE) inhibitors was recently observed to impact calcium fluctuation in rat primary neurons (Satir et al. Alzheimer's Research & Therapy 12: 63). A study design for evaluation of APP-targeting RNAi agents in culture cells is shown in FIG. 1. When spheroids in culture were examined for morphology, no changes from pre-treatment morphology (FIG. 2A) were observed at a 14 day time point (FIG. 2B) after administration of APP-targeting siRNAs/compounds. These results were reinforced by waveform analysis (FIG. 3), in which parameters that described the number, size and shape of spontaneous calcium oscillations in each spheroid were extracted. In total, eight parameters were evaluated: peak count, peak height, peak height standard deviation (SD), peak width, peak spacing, peak spacing standard deviation (SD), peak rise time and peak decay time. All values were normalized to vehicle control in FIG. 3 and plotted as a percentage.

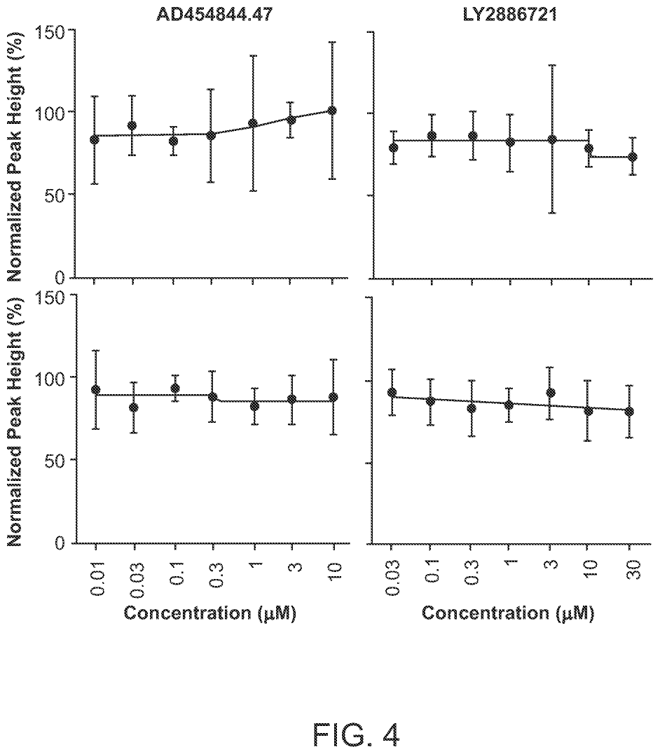

Minimal modulation of peak count and peak height was observed across treatment groups and concentrations (FIG. 4), regardless of whether an APP-targeting RNAi agent (ΔD454844.47) or the BACE inhibitor LY2886721 was administered. Further analysis of spheroid lysates and supernatants for levels of APP (total), amyloid species (e.g., Aβ38, Aβ40, Aβ42), as well as β-secretase-derived fragment (β-CTF) is also performed.

### Example 3: APP-Targeting RNAi Agents Robustly Reduced Both Soluble APPα (sAPPα) and Soluble APPβ (sAPPβ) Levels in Both Spheroid Cell Media and Intracellularly

Spheroids in media were assessed for the respective impacts of an APP-targeting siRNA and the BACE inhibitor LY2886721, upon both sAPPα and sAPPβ levels, both in cell media (therefore reflecting secreted levels of sAPPα and sAPPβ) and in cell lysates (to capture intracellular levels of sAPPα and sAPPβ).

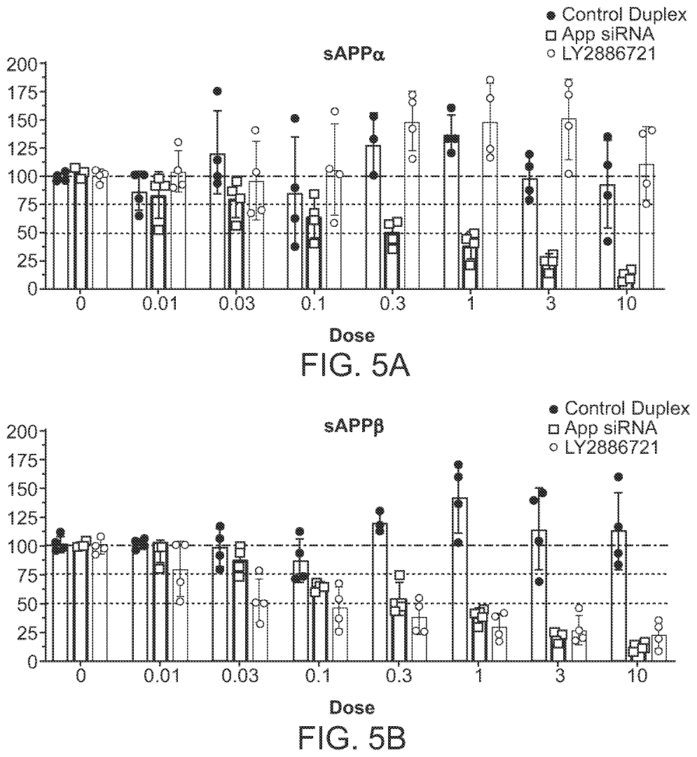

In spheroid cell media, the APP-targeting siRNA exhibited dose-dependent inhibition of sAPPα levels, whereas the BACE inhibitor LY2886721 exhibited no impact on sAPPα levels in cell media (FIG. 5A). When sAPPβ levels were assessed in spheroid cell media, both the APP-targeting siRNA and the BACE inhibitor LY2886721 exhibited dose-dependent inhibition of sAPPβ levels (FIG. 5B). Indeed, the APP-targeting siRNA showed comparable sAPPβ lowering to the BACE inhibitor in cell media. The APP-targeting siRNA was therefore observed to lower both sAPPα and sAPPβ levels in cell media, while the BACE inhibitor LY2886721 was shown to specifically lower sAPPβ levels in cell media.

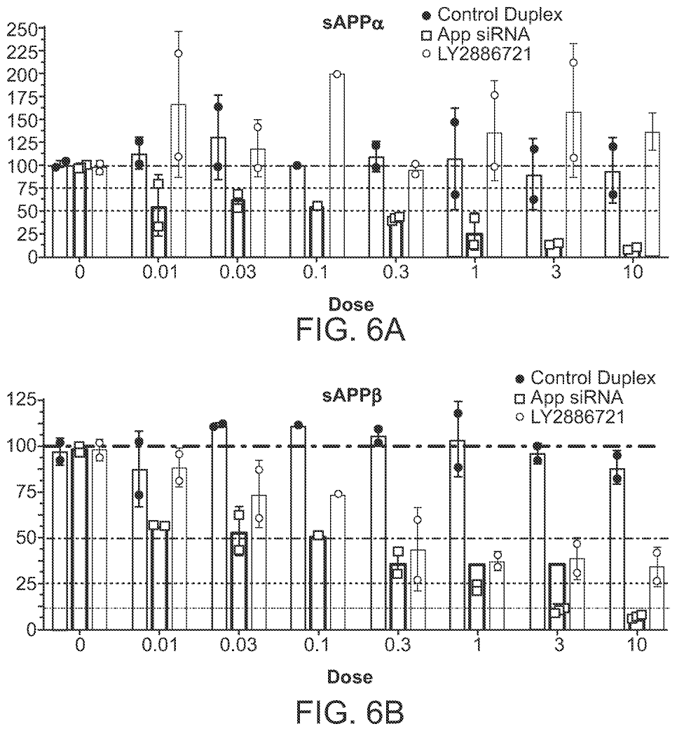

In spheroid cell lysates, the APP-targeting siRNA again exhibited dose-dependent inhibition of sAPPα levels, whereas the BACE inhibitor LY2886721 exhibited no impact on sAPPα levels in cell lysates (FIG. 6A). When sAPPβ levels were assessed in spheroid cell lysates, both the APP-targeting siRNA and the BACE inhibitor LY2886721 exhibited dose-dependent inhibition of sAPPβ levels (FIG. 6B). Indeed, the APP-targeting siRNA actually showed superior sAPPβ lowering to the BACE inhibitor in cell lysates. The APP-targeting siRNA was therefore observed to lower both sAPPα and sAPPβ levels in cell lysates, while the BACE inhibitor LY2886721 was shown to specifically lower sAPPβ levels in cell lysates. In addition, lowering of sAPPβ levels in cell lysates appeared to hit a floor following treatment with the BACE inhibitor LY2886721, whereas no such floor was observed for the APP-targeting siRNA.

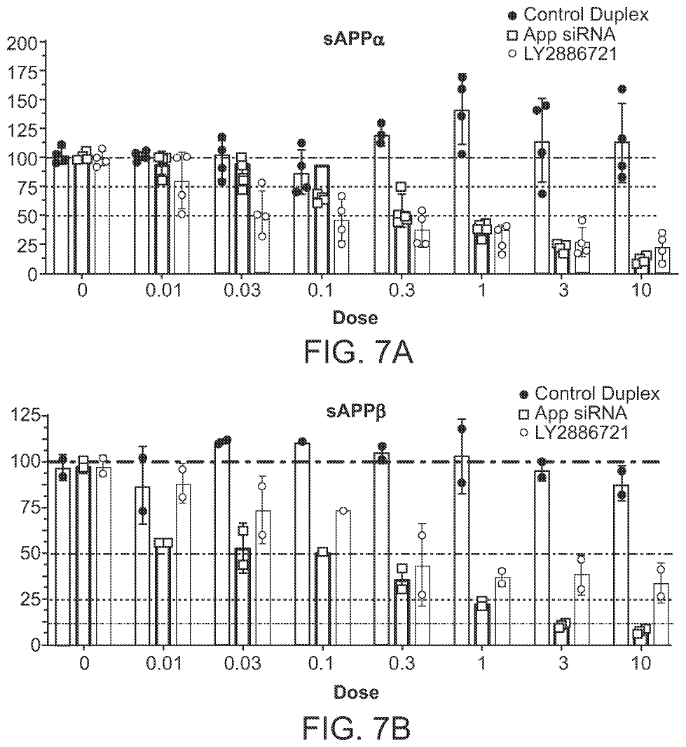

FIGS. 5B and 6B above are directly compared in FIG. 7A and FIG. 7B. This direct comparison reinforced that the APP-targeting siRNA showed comparable sAPPβ lowering to BACE inhibitor in cell media. The APP-targeting siRNA lowered both sAPPα and sAPPβ levels in cell lysates, while the BACE inhibitor LY2886721 specifically lowered sAPPβ levels in cell lysates and also hit a floor upon treatment with the BACE inhibitor LY2886721, which indicated that an intracellular reserve of sAPPβ was likely being liberated by the APP-targeting siRNA while not being accessible to the activity of BACE inhibitor LY2886721.

Notably, Amyloid beta (Aβ) assessments such as those of the current Example carry certain assay sensitivity limitations. For the above-described analysis of spheroid lysates and supernatant, the Aβ peptide panel showed all samples were at or below the lower limit of quantitation (LLOQ) in spheroid lysates. In the media, nearly all samples were LLOQ for A038. When Aβ40 and Aβ42 were assessed, most samples treated with 10 μM-0.3 μM APP-targeting siRNA ΔD454844 and BACE inhibitor LY2886721 were also LLOQ. At the lower doses (0.01-0.1 μM), Aβ40 and Aβ42 were detectable, but near the LLOQ. Samples treated with negative control siRNA exhibited detectable levels of Aβ40 and Aβ42 for all doses.

In sum, analysis of spheroid lysates and supernatants showed predicted extracellular changes of sAPP. The APP-targeting siRNA ΔD454844 showed superior intracellular sAPPβ lowering as compared to BACE inhibition. An Aβ panel showed LLOQ to near LLOQ for all APP siRNA and BACE inhibitor treatment conditions. No change in cell viability or spheroid quality measures were observed for any treatments. β-secretase-derived fragment ($-CTF) biomarker analysis is also performed.

### Example 4: APP Knockdown Decreased Endosome Size in Mutated PSEN1 Patient-Derived Neurons

Mutations in APP and PSEN1 have been observed to cause enlargement of Rab5+ endosomes in human induced pluripotent stem cell (iPSC)-derived neurons. (See Kwart et al., Neuron 2019, 104, 256-270.) In particular, Kwart et al. demonstrated that significantly enlarged Rab5+(early) endosomes can be observed in human iPSC-derived neurons (i) homozygous for the APP “Swedish mutation” (KM670/671NL); (ii) homozygous for the APP A692G mutation; (iii) homozygous for the APP V717G mutation; (iv) homozygous for the PSEN1 M146V mutation; (v) homozygous for the PSEN1 L166P mutation; (vi) having PSEN1 A246E/null mutations; and (vii) having double APP/PSEN1 mutations APP knockout has been shown to reduce mean Rab5+ endosome size in neurons, relative to wild-type and neurons harboring the APP A673T mutation.

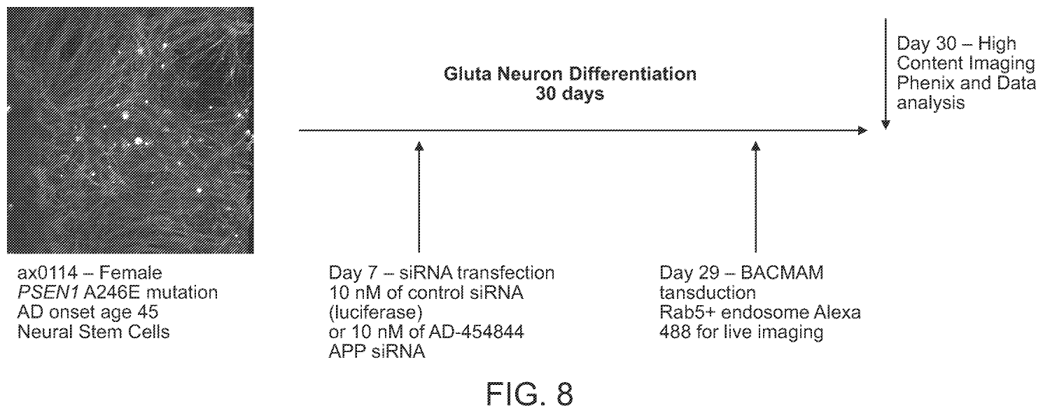

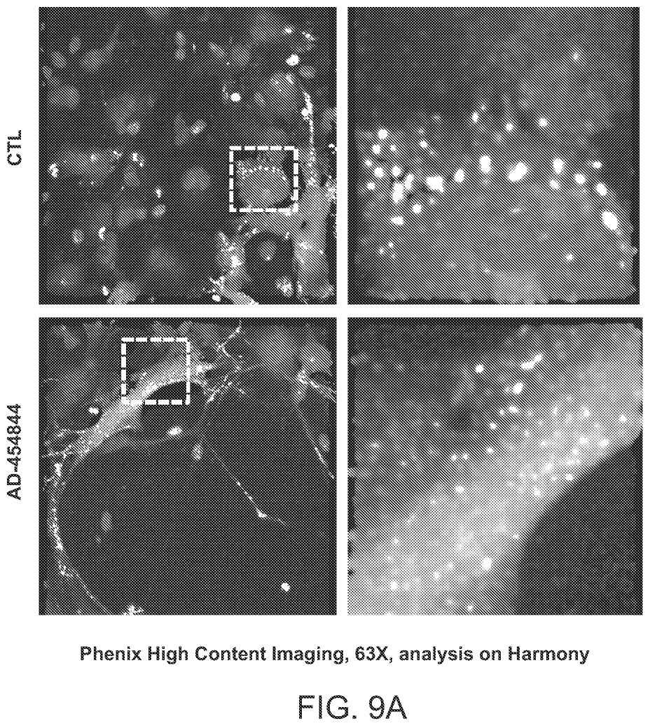

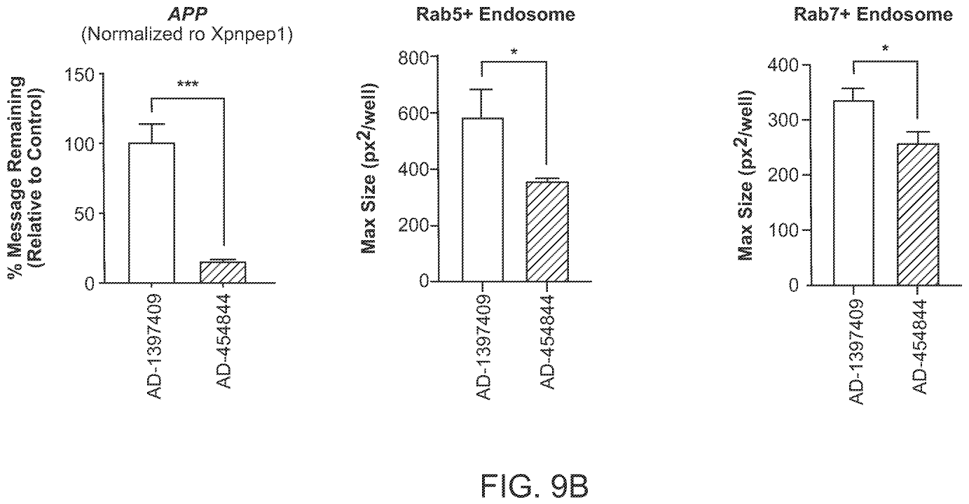

The impact of siRNA-mediated APP knockdown upon PSEN1 mutant neuronal cell endosome size was assessed. iPSC-derived neurons were generated from a PSEN1 A246E patient (cells are commercially available from Axol Bioscience Ltd.) and were then administered either a control (anti-luciferase) siRNA or the APP-targeting siRNA AD-454844 (FIG. 8). The significantly elevated Rab5+ endosome sizes observed in cells homozygous for PSEN1 A246E were previously identified. Immunofluorescent Rab5 imaging revealed that treatment of cells homozygous for PSEN1 A246E with the APP-targeting siRNA AD-454844 significantly reduced endosome size (FIGS. 9A and 9B), thereby confirming the association between APP levels and modulation of endosome size, as well as identifying APP as a molecular target for therapeutic treatment of diseases characterized by PSEN1 mutations and enlarged early endosomes. The effects of administering APP-targeting siRNA AD-454844 upon endosome size were further shown to be preferential for early endosomes, as significantly greater reduction of Rab5+(early) endosome size was observed with siRNA AD-454844 treatment, as compared to the more modest size reductions observed in the Rab7-associated endosomal population (an endosomal population that includes both early endosomes and late endosomes/multivesicular bodies (LEs/MVBs)) (FIG. 9B, right-hand panel).

### Example 5: siRNA-Mediated APP Knockdown Reduced Both APP β-CTF Levels and Early Endosome Size in PSEN1 Mutant Patient-Derived Neurons More Effectively than Small Molecule APP-Targeting BACE Inhibitor LY2886721

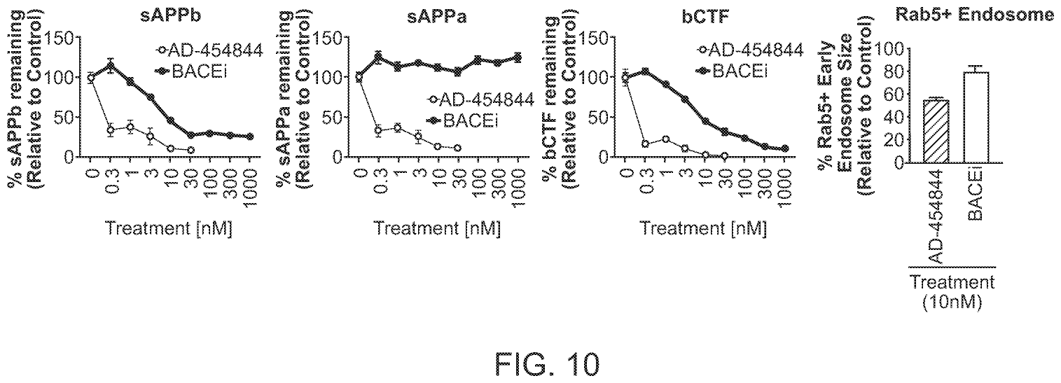

The relative efficacies of APP-targeting siRNA AD-454844 and the APP-targeting small molecule BACE inhibitor LY2886721 in reducing early endosomal APP forms and early endosome size in PSEN1^{A246E} patient iPSC-derived cortical neuron model cells were evaluated. Dose-dependent inhibition of sAPPβ levels was observed for both the AD-454844 siRNA and for the BACE inhibitor LY2886721, though AD-454844 exhibited enhanced potency of sAPPβ knockdown across all concentrations assessed (FIG. 10, left-hand panel). In contrast, dose-dependent inhibition of sAPPα levels was only observed for AD-454844 siRNA, whereas BACE inhibitor LY2886721 exhibited no sAPPα knockdown at any dose tested (FIG. 10, second panel from left). While dose-dependent reduction of APP β-C-terminal fragment (β-CTF) levels was again observed for both AD-454844 siRNA and small molecule BACE inhibitor LY2886721, significantly more potent reduction of β-CTF was observed for AD-454844 siRNA, as compared to LY2886721 (FIG. 10, third panel from left). Meanwhile, early endosome (RAB5+ endosome) size was significantly more reduced in PSEN1 A246E patient iPSC-derived cortical neuron model cells treated with AD-454844 siRNA at 10 nM (approx. 40-50% reduction in early endosome size), as compared to the more modest levels of early endosome size reduction observed for such cells treated with LY2886721 at 10 nM (approx. 20% reduction in early endosome size) (FIG. 10, right-hand panel). It was therefore demonstrated that APP-targeting siRNAs are capable of exerting a preferential reduction of APP forms found in early endosomes and upon early endosome size, with a molecular profile and magnitude of effect that was clearly different from that observed for the APP-targeting small molecule BACE inhibitor LY2886721, and which is also distinct from antibody-based therapies (as antibody-based therapeutics do not target intracellular β-CTF).

### Example 6: Preclinical Efficacy of C16-siRNA in a Mouse Model of Alzheimer's Disease

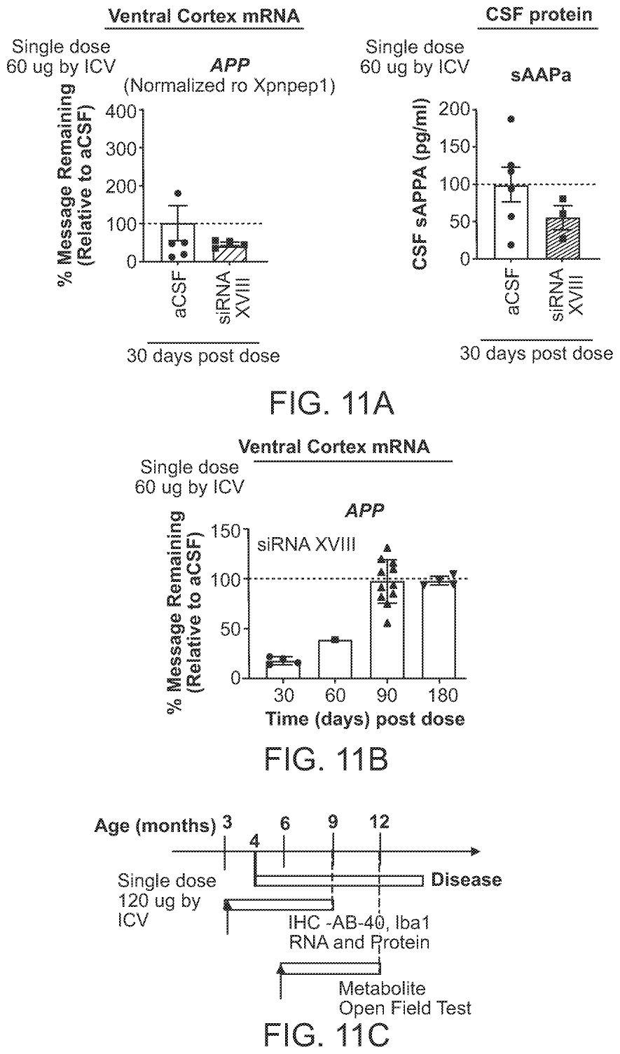

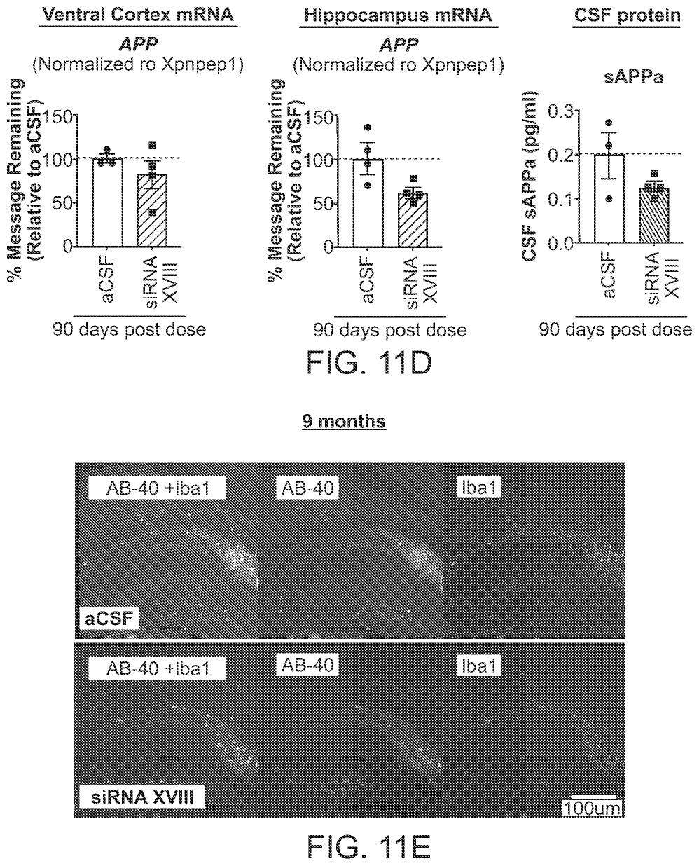

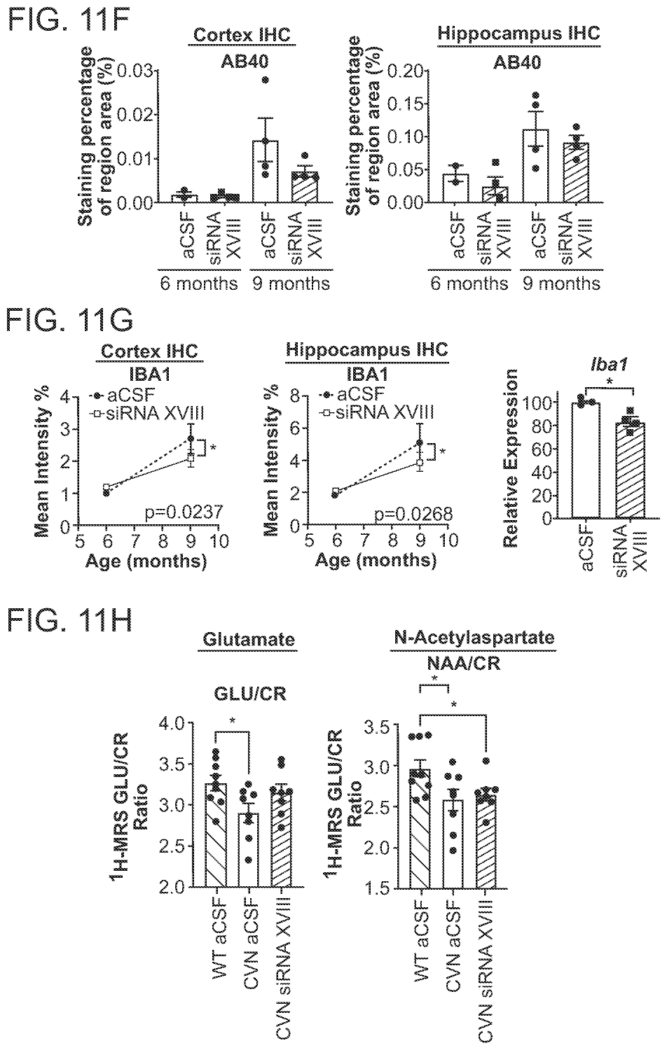

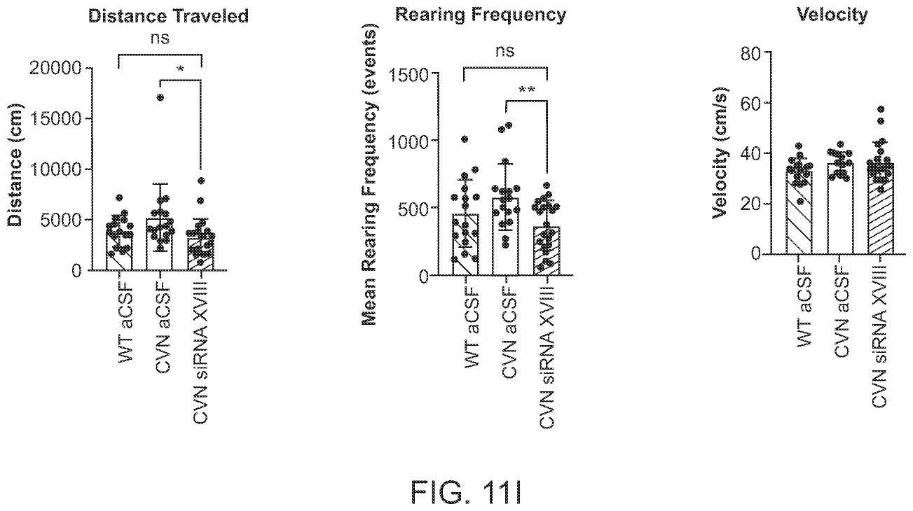

A Tg-hAPP^{SwDI}/mNos2^{-/-} (CVN) transgenic mouse expressing human APP harboring the Swedish K670N/M671L, Dutch E693Q and Iowa D694N mutations was crossed to a Nos2^{-/-} background (Wilcock et al. J. Neurosci. 28, 1537-1545), which accumulates Aβ in the brain parenchyma and vasculature (Colton et al. J. Neuropathol. Exp. Neurol. 73, 752-769), to evaluate preclinical efficacy of C16-siRNAs. Using an APP targeting C16-siRNA (AD-454972), a single 60 μg ICV administration was demonstrated to reduce APP mRNA and sAPPα protein levels by 50% in the ventral cortex (VC) and CSF (FIG. 11A). A higher, 120 μg dose exhibited an approximate 75% reduction of APP mRNA at 30 days and >50% reduction at 60 days, finally normalizing at 90 days in the VC (FIGS. 11B and 11D). When dosed at 3 months of age (FIG. 11C), hippocampal APP mRNA and CSF sAPPα protein levels remained reduced at approximately 40-50% after 90 days (FIG. 11D). It was further examined whether knockdown of APP was sufficient to reduce pathology in the CVN mice, and the study was accordingly divided into two categories: the first group, was dosed pre-symptomatically, and was assessed for Aβ40 (AB40) deposits as well as inflammation; the second group was dosed post-symptomatically, and was evaluated for changes in metabolites as well as behavior (FIG. 11C). Knockdown of APP lasted for 3 months in the VC (FIG. 11B); however, AB40 deposits were reduced by approximately 50% even after 6 months (FIGS. 11E and 11F), albeit not significantly due to the small sample size (n=2-4). In this model, ionized calcium-binding adaptor molecule 1 (Iba1) was upregulated at 4 months and continued to elevate at 9 months, concomitant with increases in inflammation when neurodegeneration occurred (Kan et al. J Neurosci. 35: 5969-5982). The AD-454972 siRNA-treated group showed a significantly slower rate of increase in IBA1 IHC (immunohistochemistry) intensity in the cortex and hippocampus (FIG. 11G). In addition, the expression of Iba1 mRNA was reduced by 20-30% in AD-454972 siRNA-treated animals after 6 months (FIG. 11G). To determine whether decreases in Aβ deposition and inflammation could be associated with restoration of neuronal function, proton magnetic resonance spectroscopy (Hall et al. Curr. Top. Behav. Neurosci. 11: 169-198; ¹H-MRS) was used to measure metabolites, such as glutamate (Glu) and N-acetylaspartate (NAA). These metabolites have been found to be reduced in patients with Alzheimer's disease, are reflective of neuronal integrity and are predictors of cognitive deficits (Su et al. Transl. Psychiatry 6: e877; Wang et al. J. Alzheimers Dis. 46: 1049-1070). Here, a significant reduction in both Glu and NAA levels was observed in the CVN mice at 12 months of age, as compared with wild-type (WT) (FIG. 11H). Knockdown of APP in the CVN mice normalized Glu levels to that of the WT; however, it had little effect on NAA levels (FIG. 11H). In the open field test, the CVN mice were observed to exhibit a measurable increase in total distance traveled and an increase in rearing frequency (FIG. 11I). Treatment with the APP-targeting AD-454972 siRNA normalized both measures to levels seen in the WT (FIG. 11I). Velocity measures in all groups were not significantly different from each other (FIG. 11I). Accordingly, C16-siRNAs (particularly the AD-454972 siRNA) exhibited preclinical efficacy in a mouse model of AD.

### Example 7: Preclinical Efficacy of C16-siRNA in Human iPSC-Derived Neural Stem Cells

The efficacy of administration of C16-siRNAs of the instant disclosure in decreasing endosome size and/or having therapeutic impact upon a treated subject is assessed in the following iPSC-derived neural stem cells: ax0112 fibroblasts (38 year old female; AD patient PSEN1 L286V) and ax0114 fibroblasts (31 year old female; AD patient PSEN1 A246E). Endosome size is expected to decrease in C16-siRNA-treated subjects, as compared to appropriate control(s). The “mRNA” sequences of the Informal Sequence Listing and certain of the “mRNA target” sequences listed herein may be noted as reciting thymine (T) residues rather than uracil (U) residues. As is apparent to one of ordinary skill in the art, such sequences reciting “T” residues rather than “U” residues can be derived from NCBI accession records that list, as “mRNA” sequences, the DNA sequences (not RNA sequences) that directly correspond to mRNA sequences. Such DNA sequences that directly correspond to mRNA sequences technically constitute the DNA sequence that is the complement of the cDNA (complementary DNA) sequence for an indicated mRNA. Thus, while the mRNA target sequence does, in fact, actually include uracil (U) rather than thymine (T), the NCBI record-derived “mRNA” sequence includes thymine (T) residues rather than uracil (U) residues.

TABLE 1
Abbreviations of nucleotide monomers used in nucleic acid sequence representation
It will be understood that these monomers, when present in an oligonucleotide, are mutually linked
by 5′-3′-phosphodiester bonds. The abbreviations of below Table 1 are understood to omit the 3′-
phosphate (i.e. they are 3′-OH) when placed at the 3′-terminal position of an oligonucleotide of
the instant disclosure.

| Abbreviation | Nucleotide(s) |
|---|---|
| A | Adenosine-3′-phosphate |
| Ab | beta-L-adenosine-3′-phosphate |
| Abs | beta-L-adenosine-3′-phosphorothioate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cb | beta-L-cytidine-3′-phosphate |
| Cbs | beta-L-cytidine-3′-phosphorothioate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gb | beta-L-guanosine-3′-phosphate |
| Gbs | beta-L-guanosine-3′-phosphorothioate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine-3′-phosphorothioate |
| Us | uridine-3′-phosphorothioate |
| N | any nucleotide, modified or unmodified |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| s | phosphorothioate linkage |
| L96 | N-[tris(GalNAc-alkyl)-amidodecanoyl)]-4-hydroxyprolinol Hyp-(GalNAc- |
| | alkyl)3 (Hyp-(GalNAc-alkyl)3) |
| | OR |
| | (2S,4R)-1-[29-[[2-(acetylamino)-2-deoxy-ß-D-galactopyranosyl]oxy]-14,14- |
| | bis[[3-[[3-[[5-[[2-(acetylamino)-2-deoxy-ß-D-galactopyranosyl]oxy]-1- |
| | oxopentyl]amino]propyl]amino]-3-oxopropoxy]methyl]-1,12,19,25-tetraoxo-16- |
| | oxa-13,20,24-triazanonacos-1-yl]-4-hydroxy-2-hydroxymethylpyrrolidine |
| | 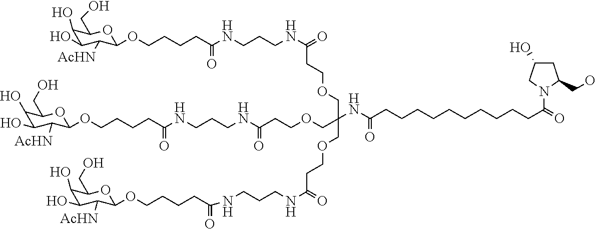 [SMILES: CC(=O)NC1C(OCCCCC(=O)NCCCNC(=O)CCOCC(COCCC(=O)NCCCNC(=O)CCCCOC2OC(CO)C(O)C(O)C2NC(C)=O)(COCCC(=O)NCCCNC(=O)CCCCOC2OC(CO)C(O)C(O)C2NC(C)=O)NC(=O)CCCCCCCCCCC(=O)N2C[C@H](O)C[C@H]2CO)OC(CO)C(O)C1O] |
| uL96 | 2′-O-methyluridine-3′-phosphate ((2S,4R)-1-[29-[[2-(acetylamino)-2-deoxy-ß- |
| | D-galactopyranosyl]oxy]-14,14-bis[[3-[[3-[[5-[[2-(acetylamino)-2-deoxy-ß-D- |
| | galactopyranosyl]oxy]-1-oxopentyl]amino]propyl]amino]-3- |
| | oxopropoxy]methyl]-1,12,19,25-tetraoxo-16-oxa-13,20,24-triazanonacos-1-yl]- |
| | 4-hydroxy-2-pyrrolidinyl)methyl ester |
| | 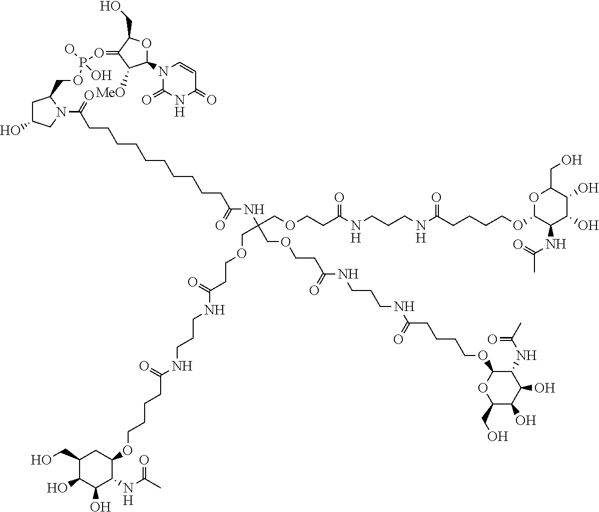 |
| aL96 | 2′-O-methyladenosine-3′-phosphate ((2S,4R)-1-[29-[2-(acetylamino)-2-deoxy- |
| | ß-D-galactopyranosyl]oxy]-14,14-bis[[3-[[3-[[5-[[2-(acetylamino)-2-deoxy-ß-D- |
| | galactopyranosyl]oxy]-1-oxopentyl]amino]propyl]amino]-3- |
| | oxopropoxy]methyl]-1,12,19,25-tetraoxo-16-oxa-13,20,24-triazanonacos-1-yl]- |
| | 4-hydroxy-2-pyrrolidinyl)methyl ester |
| cL96 | 2′-O-methylcytidine-3′-phosphate ((2S,4R)-1-[29-[[2-(acetylamino)-2-deoxy-ß- |
| | D-galactopyranosyl]oxy]-14,14-bis[[3-[[3-[[5-[[2-(acetylamino)-2-deoxy-ß-D- |
| | galactopyranosyl]oxy]-1-oxopentyl]amino]propyl]amino]-3- |
| | oxopropoxy]methyl]-1,12,19,25-tetraoxo-16-oxa-13,20,24-triazanonacos-1-yl]- |
| | 4-hydroxy-2-pyrrolidinyl)methyl ester |
| gL96 | 2′-O-methylguanosine-3′-phosphate ((2S,4R)-1-[29-[[2-(acetylamino)-2-deoxy- |
| | ß-D-galactopyranosyl]oxy]-14,14-bis[[3-[[3-[[5-[[2-(acetylamino)-2-deoxy-ß-D- |
| | galactopyranosyl]oxy]-1-oxopentyl]amino]propyl]amino]-3- |
| | oxopropoxy]methyl]-1,12,19,25-tetraoxo-16-oxa-13,20,24-triazanonacos-1-yl]- |
| | 4-hydroxy-2-pyrrolidinyl)methyl ester |
| Y34 | 2-hydroxymethyl-tetrahydrofurane-4-methoxy-3-phosphate (abasic 2′-OMe |
| | furanose) |
| Y44 | inverted abasic DNA (2-hydroxymethyl-tetrahydrofurane-5-phosphate) |
| (Agn) | Adenosine-glycol nucleic acid (GNA) |
| (Cgn) | Cytidine-glycol nucleic acid (GNA) |
| (Ggn) | Guanosine-glycol nucleic acid (GNA) |
| (Tgn) | Thymidine-glycol nucleic acid (GNA) S-Isomer |
| P | Phosphate |
| VP | Vinyl-phosphate |
| (Aam) | 2′-O-(N-methylacetamide)adenosine-3′-phosphate |
| (Aams) | 2′-O-(N-methylacetamide)adenosine-3′-phosphorothioate |
| (Gam) | 2′-O-(N-methylacetamide)guanosine-3′-phosphate |
| (Gams) | 2′-O-(N-methylacetamide)guanosine-3′-phosphorothioate |
| (Tam) | 2′-O-(N-methylacetamide)thymidine-3′-phosphate |
| (Tams) | 2′-O-(N-methylacetamide)thymidine-3′-phosphorothioate |
| dA | 2′-deoxyadenosine-3′-phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxythymidine-3′-phosphate |
| dTs | 2′-deoxythymidine-3′-phosphorothioate |
| dU | 2′-deoxyuridine |
| dUs | 2′-deoxyuridine-3-phosphorothioate |
| (Aeo) | 2′-O-methoxyethyladenosine-3′-phosphate |
| (Aeos) | 2′-O-methoxyethyladenosine-3′-phosphorothioate |
| (Geo) | 2′-O-methoxyethylguanosine-3′-phosphate |
| (Geos) | 2′-O-methoxyethylguanosine-3′-phosphorothioate |
| (Teo) | 2′-O-methoxyethyl-5-methyluridine-3′-phosphate |
| (Teos) | 2′-O-methoxyethyl-5-methyluridine-3′-phosphorothioate |
| (m5Ceo) | 2′-O-methoxyethyl-5-methylcytidine-3′-phosphate |
| (m5Ceos) | 2′-O-methoxyethyl-5-methylcytidine-3′-phosphorothioate |
| (A3m) | 3′-O-methyladenosine-2′-phosphate |
| (A3mx) | 3′-O-methyl-xylofuranosyladenosine-2′-phosphate |
| (G3m) | 3′-O-methylguanosine-2′-phosphate |
| (G3mx) | 3′-O-methyl-xylofuranosylguanosine-2′-phosphate |
| (C3m) | 3′-O-methylcytidine-2′-phosphate |
| (C3mx) | 3′-O-methyl-xylofuranosylcytidine-2′-phosphate |
| (U3m) | 3′-O-methyluridine-2′-phosphate |
| U3mx) | 3′-O-methyl-xylofuranosyluridine-2′-phosphate |
| (m5Cam) | 2′-O-(N-methylacetamide)-5-methylcytidine-3′-phosphate |
| (m5Cams) | 2′-O-(N-methylacetamide)-5-methylcytidine-3′-phosphorothioate |
| (Ahd) | 2′-O-hexadecyl-adenosine-3′-phosphate |
| (Ahds) | 2′-O-hexadecyl-adenosine-3′-phosphorothioate |
| (Ghd) | 2′-O-hexadecyl-guanosine-3′-phosphate |
| (Ghds) | 2′-O-hexadecyl-guanosine-3′-phosphorothioate |
| (Chd) | 2′-O-hexadecyl-cytidine-3′-phosphate |
| (Chds) | 2′-O-hexadecyl-cytidine-3′-phosphorothioate |
| (Uhd) | 2′-O-hexadecyl-uridine-3′-phosphate |
| (Uhds) | 2′-O-hexadecyl-uridine-3′-phosphorothioate |
| (pshe) | Hydroxyethylphosphorothioate |
| Q363 | N-(hexadecanoyl)-(S)-pyrrolidin-3-ol-phosphate |
| | 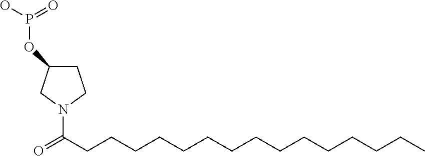 |
| (A2p) | adenosine-2′-phosphate |
| (C2p) | cytidine-2′-phosphate |
| (G2p) | guanosine-2′-phosphate |
| (U2p) | uridine-2′-phosphate |
| (A2ps) | adenosine-2′-phosphorothioate |
| (C2ps) | cytidine-2′-phosphorothioate |
| (G2ps) | guanosine-2′-phosphorothioate |
| (U2ps) | uridine-2′-phosphorothioate |

Exemplary APP-targeting iRNA agents expressly contemplated for use in the methods and compositions of the instant disclosure are presented in Tables 2-21 below.

TABLE 2
Exemplary Human APP-Targeting RNAi Agent Duplexes, Modified Sequences

| | | SEQ | Antisense | SEQ | | SEQ |
|---|---|---|---|---|---|---|
| Duplex | Sense Sequence | ID | Sequence | ID | mRNA target | ID |
| Name | (5′ to 3′) | NO | (5′ to 3′) | NO | sequence | NO |
| AD- | gsasccc(Ahd)Afu | 65 | asAfsagua(Ggn)g | 339 | GUGACCCAAUUAAGU | 613 |
| 392699 | UfAfAfguccuacuu | | acuuaAfuUfggguc | | CCUACUUU | |
| | uL96 | | sasc | | | |
| AD- | uscsucc(Uhd)Gfa | 66 | asUfsguga(Tgn)a | 340 | UCUCUCCUGAUUAUU | 614 |
| 392700 | UfUfAfuuuaucaca | | aauaaUfcAfggaga | | UAUCACAU | |
| | uL96 | | sgsa | | | |
| AD- | cscsuga(Ahd)Cfu | 67 | asUfsggau(Tgn)a | 341 | UGCCUGAACUUGAAU | 615 |
| 392703 | UfGfAfauuaaucca | | auucaAfgUfucagg | | UAAUCCAC | |
| | uL96 | | scsa | | | |
| AD- | gsgsuuc(Ahd)Afa | 68 | asAfsuugc(Agn)c | 342 | UGGGUUCAAACAAAG | 616 |
| 392704 | CfAfAfaggugcaau | | cuuugUfuUfgaacc | | GUGCAAUC | |
| | uL96 | | scsa | | | |
| AD- | ususuac(Uhd)Cfa | 69 | csAfsaaag(Ggn)c | 343 | GAUUUACUCAUUAUC | 617 |
| 392705 | UfUfAfucgccuuuu | | gauaaUfgAfguaaa | | GCCUUUUG | |
| | gL96 | | susc | | | |
| AD- | asusuua(Ghd)Cfu | 70 | asCfsuagu(Tgn)u | 344 | GAAUUUAGCUGUAUC | 618 |
| 392707 | GfUfAfucaaacuag | | gauacAfgCfuaaau | | AAACUAGU | |
| | uL96 | | susc | | | |
| AD- | asgsuau(Uhd)Cfc | 71 | asGfsugau(Cgn)a | 345 | UAAGUAUUCCUUUCC | 619 |
| 392708 | UfUfUfccugaucac | | ggaaaGfgAfauacu | | UGAUCACU | |
| | uL96 | | susa | | | |
| AD- | gscsuua(Uhd)Gfa | 72 | gsAfsaagc(Ggn)a | 346 | UUGCUUAUGACAUGA | 620 |
| 392709 | CfAfUfgaucgcuuu | | ucaugUfcAfuaagc | | UCGCUUUC | |
| | cL96 | | sasa | | | |
| AD- | asasgau(Ghd)Ufg | 73 | usAfscaaa(Tgn)u | 347 | UUAAGAUGUGUCUUC | 621 |
| 392710 | UfCfUfucaauuugu | | gaagaCfaCfaucuu | | AAUUUGUA | |
| | aL96 | | sasa | | | |
| AD- | gscsaaa(Ahd)Cfc | 74 | asUfsagug(Agn)a | 348 | CAGCAAAACCAUUGC | 622 |
| 392711 | AfUfUfgcuucacua | | gcaauGfgUfuuugc | | UUCACUAC | |
| | uL96 | | susg | | | |
| AD- | asusuua(Chd)Ufc | 75 | asAfsaagg(Cgn)g | 349 | UGAUUUACUCAUUAU | 623 |
| 392712 | AfUfUfaucgccuuu | | auaauGfaGfuaaau | | CGCCUUUU | |
| | uL96 | | scsa | | | |
| AD- | usascuc(Ahd)Ufu | 76 | asUfscaaa(Agn)g | 350 | UUUACUCAUUAUCGC | 624 |
| 392713 | AfUfCfgccuuuuga | | gcgauAfaUfgagua | | CUUUUGAC | |
| | uL96 | | sasa | | | |
| AD- | usgsccu(Ghd)Afa | 77 | asGfsauua(Agn)u | 351 | GAUGCCUGAACUUGA | 625 |
| 392714 | CfUfUfgaauuaauc | | ucaagUfuCfaggca | | AUUAAUCC | |
| | uL96 | | susc | | | |
| AD- | csusgaa(Chd)Ufu | 78 | usGfsugga(Tgn)u | 352 | GCCUGAACUUGAAUU | 626 |
| 392715 | GfAfAfuuaauccac | | aauucAfaGfuucag | | AAUCCACA | |
| | aL96 | | sgsc | | | |
| AD- | ususuag(Chd)Ufg | 79 | asAfscuag(Tgn)u | 353 | AAUUUAGCUGUAUCA | 627 |
| 392716 | UfAfUfcaaacuagu | | ugauaCfaGfcuaaa | | AACUAGUG | |
| | uL96 | | susu | | | |
| AD- | gsasaua(Ghd)Afu | 80 | usAfsauca(Ggn)g | 354 | AUGAAUAGAUUCUCU | 628 |
| 392717 | UfCfUfcuccugauu | | agagaAfuCfuauuc | | CCUGAUUA | |
| | aL96 | | sasu | | | |
| AD- | uscscug(Ahd)Ufu | 81 | asUfsaugu(Ggn)a | 355 | UCUCCUGAUUAUUUA | 629 |
| 392718 | AfUfUfuaucacaua | | uaaauAfaUfcagga | | UCACAUAG | |
| | uL96 | | sgsa | | | |
| AD- | cscscaa(Uhd)Ufa | 82 | asUfsaaag(Tgn)a | 356 | GACCCAAUUAAGUCC | 630 |
| 392719 | AfGfUfccuacuuua | | ggacuUfaAfuuggg | | UACUUUAC | |
| | uL96 | | susc | | | |
| AD- | csasuau(Ghd)Cfu | 83 | asAfsucga(Tgn)u | 357 | UACAUAUGCUUUAAG | 631 |
| 392720 | UfUfAfagaaucgau | | cuuaaAfgCfauaug | | AAUCGAUG | |
| | uL96 | | susa | | | |
| AD- | csusucu(Chd)Ufu | 84 | asGfsaaua(Cgn)u | 358 | UGCUUCUCUUGCCUA | 632 |
| 392721 | GfCfCfuaaguauuc | | uaggcAfaGfagaag | | AGUAUUCC | |
| | uL96 | | scsa | | | |
| AD- | csasuug(Chd)Ufu | 85 | asCfsgauc(Agn)u | 359 | AUCAUUGCUUAUGAC | 633 |
| 392722 | AfUfGfacaugaucg | | gucauAfaGfcaaug | | AUGAUCGC | |
| | uL96 | | sasu | | | |
| AD- | csusuau(Ghd)Afc | 86 | asGfsaaag(Cgn)g | 360 | UGCUUAUGACAUGAU | 634 |
| 392723 | AfUfGfaucgcuuuc | | aucauGfuCfauaag | | CGCUUUCU | |
| | uL96 | | scsa | | | |
| AD- | usasuga(Chd)Afu | 87 | asUfsagaa(Agn)g | 361 | CUUAUGACAUGAUCG | 635 |
| 392724 | GfAfUfcgcuuucua | | cgaucAfuGfucaua | | CUUUCUAC | |
| | uL96 | | sasg | | | |
| AD- | usgsaca(Uhd)Gfa | 88 | asUfsguag(Agn)a | 362 | UAUGACAUGAUCGCU | 636 |
| 392725 | UfCfGfcuuucuaca | | agcgaUfcAfuguca | | UUCUACAC | |
| | uL96 | | susa | | | |
| AD- | gsasucg(Chd)Ufu | 89 | asAfsuaca(Ggn)u | 363 | AUGAUCGCUUUCUAC | 637 |
| 392726 | UfCfUfacacuguau | | guagaAfaGfcgauc | | ACUGUAUU | |
| | uL96 | | sasu | | | |
| AD- | asasaac(Uhd)Afu | 90 | asGfsacgu(Cgn)a | 364 | GCAAAACUAUUCAGA | 638 |
| 392727 | UfCfAfgaugacguc | | ucugaAfuAfguuuu | | UGACGUCU | |
| | uL96 | | sgsc | | | |
| AD- | asasacu(Ahd)Ufu | 91 | asAfsgacg(Tgn)c | 365 | CAAAACUAUUCAGAU | 639 |
| 392728 | CfAfGfaugacgucu | | aucugAfaUfaguuu | | GACGUCUU | |
| | uL96 | | susg | | | |
| AD- | ascsgaa(Ahd)Afu | 92 | asCfsuugu(Agn)g | 366 | CUACGAAAAUCCAAC | 640 |
| 392729 | CfCfAfaccuacaag | | guuggAfuUfuucgu | | CUACAAGU | |
| | uL96 | | sasg | | | |
| AD- | usgscuu(Chd)Ufc | 93 | asAfsuacu(Tgn)a | 367 | GCUGCUUCUCUUGCC | 641 |
| 392730 | UfUfGfccuaaguau | | ggcaaGfaGfaagca | | UAAGUAUU | |
| | uL96 | | sgsc | | | |
| AD- | usgscuu(Ahd)Ufg | 94 | asAfsagcg(Agn)u | 368 | AUUGCUUAUGACAUG | 642 |
| 392731 | AfCfAfugaucgcuu | | cauguCfaUfaagca | | AUCGCUUU | |
| | uL96 | | sasu | | | |
| AD- | usgsauc(Ghd)Cfu | 95 | asUfsacag(Tgn)g | 369 | CAUGAUCGCUUUCUA | 643 |
| 392732 | UfUfCfuacacugua | | uagaaAfgCfgauca | | CACUGUAU | |
| | uL96 | | susg | | | |
| AD- | asuscgc(Uhd)Ufu | 96 | usAfsauac(Agn)g | 370 | UGAUCGCUUUCUACA | 644 |
| 392733 | CfUfAfcacuguauu | | uguagAfaAfgcgau | | CUGUAUUA | |
| | aL96 | | scsa | | | |
| AD- | uscsuuu(Ghd)Afc | 97 | asGfsuuuu(Cgn)g | 371 | CAUCUUUGACCGAAA | 645 |
| 392734 | CfGfAfaacgaaaac | | uuucgGfuCfaaaga | | CGAAAACC | |
| | uL96 | | susg | | | |
| AD- | gsusucu(Ghd)Gfg | 98 | usGfsauau(Tgn)u | 372 | AGGUUCUGGGUUGAC | 646 |
| 392735 | UfUfGfacaaauauc | | gucaaCfcCfagaac | | AAAUAUCA | |
| | aL96 | | scsu | | | |
| AD- | usgsggu(Uhd)Gfa | 99 | asUfscuug(Agn)u | 373 | UCUGGGUUGACAAAU | 647 |
| 392736 | CfAfAfauaucaaga | | auuugUfcAfaccca | | AUCAAGAC | |
| | uL96 | | sgsa | | | |
| AD- | gsasuuu(Ahd)Cfu | 100 | asAfsaggc(Ggn)a | 374 | AUGAUUUACUCAUUA | 648 |
| 392737 | CfAfUfuaucgccuu | | uaaugAfgUfaaauc | | UCGCCUUU | |
| | uL96 | | sasu | | | |
| AD- | uscscuu(Uhd)Cfc | 101 | usGfscaua(Ggn)u | 375 | AUUCCUUUCCUGAUC | 649 |
| 392738 | UfGfAfucacuaugc | | gaucaGfgAfaagga | | ACUAUGCA | |
| | aL96 | | sasu | | | |
| AD- | csusuuc(Chd)Ufg | 102 | asAfsugca(Tgn)a | 376 | UCCUUUCCUGAUCAC | 650 |
| 392739 | AfUfCfacuaugcau | | gugauCfaGfgaaag | | UAUGCAUU | |
| | uL96 | | sgsa | | | |
| AD- | asusugc(Uhd)Ufa | 103 | asGfscgau(Cgn)a | 377 | UCAUUGCUUAUGACA | 651 |
| 392740 | UfGfAfcaugaucgc | | ugucaUfaAfgcaau | | UGAUCGCU | |
| | uL96 | | sgsa | | | |
| AD- | uscsuuu(Ahd)Afc | 104 | asAfsacuu(Cgn)a | 378 | UUUCUUUAACCAGUC | 652 |
| 392741 | CfAfGfucugaaguu | | gacugGfuUfaaaga | | UGAAGUUU | |
| | uL96 | | sasa | | | |
| AD- | gsgsauc(Ahd)Gfu | 105 | asAfsucgu(Tgn)u | 379 | AAGGAUCAGUUACGG | 653 |
| 392742 | UfAfCfggaaacgau | | ccguaAfcUfgaucc | | AAACGAUG | |
| | uL96 | | susu | | | |
| AD- | csusggg(Uhd)Ufg | 106 | usCfsuuga(Tgn)a | 380 | UUCUGGGUUGACAAA | 654 |
| 392743 | AfCfAfaauaucaag | | uuuguCfaAfcccag | | UAUCAAGA | |
| | aL96 | | sasa | | | |
| AD- | asusgau(Uhd)Ufa | 107 | asGfsgcga(Tgn)a | 381 | UUAUGAUUUACUCAU | 655 |
| 392744 | CfUfCfauuaucgcc | | augagUfaAfaucau | | UAUCGCCU | |
| | uL96 | | sasa | | | |
| AD- | csusugu(Ghd)Gfu | 108 | asAfsuugg(Ggn)u | 382 | UUCUUGUGGUUUGUG | 656 |
| 392745 | UfUfGfugacccaau | | cacaaAfcCfacaag | | ACCCAAUU | |
| | uL96 | | sasa | | | |
| AD- | asusaug(Chd)Ufu | 109 | asCfsaucg(Agn)u | 383 | ACAUAUGCUUUAAGA | 657 |
| 392746 | UfAfAfgaaucgaug | | ucuuaAfaGfcauau | | AUCGAUGG | |
| | uL96 | | sgsu | | | |
| AD- | ususugu(Chd)Cfa | 110 | asCfsccaa(Agn)g | 384 | UUUUUGUCCACGUAU | 658 |
| 392747 | CfGfUfaucuuuggg | | auacgUfgGfacaaa | | CUUUGGGU | |
| | uL96 | | sasa | | | |
| AD- | uscsauu(Ghd)Ufa | 111 | asCfsguaa(Agn)a | 385 | GUUCAUUGUAAGCAC | 659 |
| 392748 | AfGfCfacuuuuacg | | gugcuUfaCfaauga | | UUUUACGG | |
| | uL96 | | sasc | | | |
| AD- | gsgscca(Ahd)Cfa | 112 | asGfsuuca(Cgn)u | 386 | UUGGCCAACAUGAUU | 660 |
| 392749 | UfGfAfuuagugaac | | aaucaUfgUfuggcc | | AGUGAACC | |
| | uL96 | | sasa | | | |
| AD- | gsasuca(Ghd)Ufu | 113 | asCfsaucg(Tgn)u | 387 | AGGAUCAGUUACGGA | 661 |
| 392750 | AfCfGfgaaacgaug | | uccguAfaCfugauc | | AACGAUGC | |
| | uL96 | | scsu | | | |
| AD- | usascgg(Ahd)Afa | 114 | asAfsugag(Agn)g | 388 | GUUACGGAAACGAUG | 662 |
| 392751 | CfGfAfugcucucau | | caucgUfuUfccgua | | CUCUCAUG | |
| | uL96 | | sasc | | | |
| AD- | usgsauu(Uhd)Afc | 115 | asAfsggcg(Agn)u | 389 | UAUGAUUUACUCAUU | 663 |
| 392752 | UfCfAfuuaucgccu | | aaugaGfuAfaauca | | AUCGCCUU | |
| | uL96 | | susa | | | |
| AD- | gsusaga(Uhd)Gfc | 116 | asAfsuuca(Agn)g | 390 | AAGUAGAUGCCUGAA | 664 |
| 392753 | CfUfGfaacuugaau | | uucagGfcAfucuac | | CUUGAAUU | |
| | uL96 | | susu | | | |
| AD- | ususgua(Uhd)Afu | 117 | asAfsccac(Agn)a | 391 | AGUUGUAUAUUAUUC | 665 |
| 392754 | UfAfUfucuuguggu | | gaauaAfuAfuacaa | | UUGUGGUU | |
| | uL96 | | scsu | | | |
| AD- | asusugc(Uhd)Gfc | 118 | asAfsauau(Agn)g | 392 | AGAUUGCUGCUUCUG | 666 |
| 392755 | UfUfCfugcuauauu | | cagaaGfcAfgcaau | | CUAUAUUU | |
| | uL96 | | scsu | | | |
| AD- | usgscua(Uhd)Afu | 119 | usCfscuau(Agn)u | 393 | UCUGCUAUAUUUGUG | 667 |
| 392756 | UfUfGfugauauagg | | cacaaAfuAfuagca | | AUAUAGGA | |
| | aL96 | | sgsa | | | |
| AD- | ascsaca(Uhd)Ufa | 120 | asAfsgucu(Cgn)a | 394 | GCACACAUUAGGCAU | 668 |
| 392757 | GfGfCfauugagacu | | augccUfaAfugugu | | UGAGACUU | |
| | uL96 | | sgsc | | | |
| AD- | asasgaa(Uhd)Cfc | 121 | usUfsacaa(Tgn)g | 395 | AAAAGAAUCCCUGUU | 669 |
| 392758 | CfUfGfuucauugua | | aacagGfgAfuucuu | | CAUUGUAA | |
| | aL96 | | susu | | | |
| AD- | csasuug(Uhd)Afa | 122 | asCfscgua(Agn)a | 396 | UUCAUUGUAAGCACU | 670 |
| 392759 | GfCfAfcuuuuacgg | | agugcUfuAfcaaug | | UUUACGGG | |
| | uL96 | | sasa | | | |
| AD- | ususgcu(Uhd)Afu | 123 | asAfsgcga(Tgn)c | 397 | CAUUGCUUAUGACAU | 671 |
| 392760 | GfAfCfaugaucgcu | | augucAfuAfagcaa | | GAUCGCUU | |
| | uL96 | | susg | | | |
| AD- | csasagg(Ahd)Ufc | 124 | asGfsuuuc(Cgn)g | 398 | ACCAAGGAUCAGUUA | 672 |
| 392761 | AfGfUfuacggaaac | | uaacuGfaUfccuug | | CGGAAACG | |
| | uL96 | | sgsu | | | |
| AD- | asgsguu(Chd)Ufg | 125 | asUfsauuu(Ggn)u | 399 | CCAGGUUCUGGGUUG | 673 |
| 392762 | GfGfUfugacaaaua | | caaccCfaGfaaccu | | ACAAAUAU | |
| | uL96 | | sgsg | | | |
| AD- | asasgau(Ghd)Ufg | 126 | asUfsuugu(Tgn)u | 400 | AGAAGAUGUGGGUUC | 674 |
| 392763 | GfGfUfucaaacaaa | | gaaccCfaCfaucuu | | AAACAAAG | |
| | uL96 | | scsu | | | |
| AD- | csusgaa(Ghd)Afa | 127 | usGfsugua(Cgn)u | 401 | UGCUGAAGAAGAAAC | 675 |
| 392764 | GfAfAfacaguacac | | guuucUfuCfuucag | | AGUACACA | |
| | aL96 | | scsa | | | |
| AD- | asasguu(Ghd)Gfa | 128 | asAfsuggu(Tgn)u | 402 | UGAAGUUGGACAGCA | 676 |
| 392765 | CfAfGfcaaaaccau | | ugcugUfcCfaacuu | | AAACCAUU | |
| | uL96 | | scsa | | | |
| AD- | asuscgg(Uhd)Gfu | 129 | asUfsucua(Tgn)a | 403 | CCAUCGGUGUCCAUU | 677 |
| 392766 | CfCfAfuuuauagaa | | aauggAfcAfccgau | | UAUAGAAU | |
| | uL96 | | sgsg | | | |
| AD- | uscsggu(Ghd)Ufc | 130 | usAfsuucu(Agn)u | 404 | CAUCGGUGUCCAUUU | 678 |
| 392767 | CfAfUfuuauagaau | | aaaugGfaCfaccga | | AUAGAAUA | |
| | aL96 | | susg | | | |
| AD- | gscsugu(Ahd)Afc | 131 | asGfscauc(Tgn)a | 405 | GUGCUGUAACACAAG | 679 |
| 392768 | AfCfAfaguagaugc | | cuuguGfuUfacagc | | UAGAUGCC | |
| | uL96 | | sasc | | | |
| AD- | asasgua(Ghd)Afu | 132 | usUfscaag(Tgn)u | 406 | ACAAGUAGAUGCCUG | 680 |
| 392769 | GfCfCfugaacuuga | | caggcAfuCfuacuu | | AACUUGAA | |
| | aL96 | | sgsu | | | |
| AD- | ususgug(Ghd)Ufu | 133 | usAfsauug(Ggn)g | 407 | UCUUGUGGUUUGUGA | 681 |
| 392770 | UfGfUfgacccaauu | | ucacaAfaCfcacaa | | CCCAAUUA | |
| | aL96 | | sgsa | | | |
| AD- | gsusuug(Uhd)Gfa | 134 | asGfsacuu(Agn)a | 408 | UGGUUUGUGACCCAA | 682 |
| 392771 | CfCfCfaauuaaguc | | uugggUfcAfcaaac | | UUAAGUCC | |
| | uL96 | | scsa | | | |
| AD- | gsusgac(Chd)Cfa | 135 | asGfsuagg(Agn)c | 409 | UUGUGACCCAAUUAA | 683 |
| 392772 | AfUfUfaaguccuac | | uuaauUfgGfgucac | | GUCCUACU | |
| | uL96 | | sasa | | | |
| AD- | usasugc(Uhd)Ufu | 136 | asCfscauc(Ggn)a | 410 | CAUAUGCUUUAAGAA | 684 |
| 392773 | AfAfGfaaucgaugg | | uucuuAfaAfgcaua | | UCGAUGGG | |
| | uL96 | | susg | | | |
| AD- | ususugu(Ghd)Afu | 137 | usCfsuuaa(Tgn)u | 411 | UAUUUGUGAUAUAGG | 685 |
| 392774 | AfUfAfggaauuaag | | ccuauAfuCfacaaa | | AAUUAAGA | |
| | aL96 | | susa | | | |
| AD- | asasaga(Ahd)Ufc | 138 | usAfscaau(Ggn)a | 412 | GAAAAGAAUCCCUGU | 686 |
| 392775 | CfCfUfguucauugu | | acaggGfaUfucuuu | | UCAUUGUA | |
| | aL96 | | susc | | | |
| AD- | usgsauu(Ghd)Ufa | 139 | asGfscaau(Ggn)a | 413 | GAUGAUUGUACAGAA | 687 |
| 392776 | CfAfGfaaucauugc | | uucugUfaCfaauca | | UCAUUGCU | |
| | uL96 | | susc | | | |
| AD- | usgsccu(Ghd)Gfa | 140 | asAfsagaa(Ggn)g | 414 | CAUGCCUGGACAAAC | 688 |
| 392777 | CfAfAfacccuucuu | | guuugUfcCfaggca | | CCUUCUUU | |
| | uL96 | | susg | | | |
| AD- | gsasgca(Ahd)Afa | 141 | asUfscauc(Tgn)g | 415 | AAGAGCAAAACUAUU | 689 |
| 392778 | CfUfAfuucagauga | | aauagUfuUfugcuc | | CAGAUGAC | |
| | uL96 | | susu | | | |
| AD- | asgsuga(Ahd)Cfc | 142 | asUfsaacu(Ggn)a | 416 | UUAGUGAACCAAGGA | 690 |
| 392779 | AfAfGfgaucaguua | | uccuuGfgUfucacu | | UCAGUUAC | |
| | uL96 | | sasa | | | |
| AD- | usgsaac(Chd)Afa | 143 | asCfsguaa(Cgn)u | 417 | AGUGAACCAAGGAUC | 691 |
| 392780 | GfGfAfucaguuacg | | gauccUfuGfguuca | | AGUUACGG | |
| | uL96 | | scsu | | | |
| AD- | csasguu(Ahd)Cfg | 144 | asGfsagca(Tgn)c | 418 | AUCAGUUACGGAAAC | 692 |
| 392781 | GfAfAfacgaugcuc | | guuucCfgUfaacug | | GAUGCUCU | |
| | uL96 | | sasu | | | |
| AD- | asgsaag(Ahd)Ufg | 145 | usUfsguuu(Ggn)a | 419 | GCAGAAGAUGUGGGU | 693 |
| 392782 | UfGfGfguucaaaca | | acccaCfaUfcuucu | | UCAAACAA | |
| | aL96 | | sgsc | | | |
| AD- | cscsucu(Ghd)Afa | 146 | usUfsugcu(Ggn)u | 420 | AGCCUCUGAAGUUGG | 694 |
| 392783 | GfUfUfggacagcaa | | ccaacUfuCfagagg | | ACAGCAAA | |
| | aL96 | | scsu | | | |
| AD- | ususaug(Ahd)Ufu | 147 | asCfsgaua(Agn)u | 421 | UUUUAUGAUUUACUC | 695 |
| 392784 | UfAfCfucauuaucg | | gaguaAfaUfcauaa | | AUUAUCGC | |
| | uL96 | | sasa | | | |
| AD- | ascsagc(Uhd)Gfu | 148 | asUfsugug(Tgn)u | 422 | UGACAGCUGUGCUGU | 696 |
| 392785 | GfCfUfguaacacaa | | acagcAfcAfgcugu | | AACACAAG | |
| | uL96 | | scsa | | | |
| AD- | usgsuga(Chd)Cfc | 149 | asUfsagga(Cgn)u | 423 | UUUGUGACCCAAUUA | 697 |
| 392786 | AfAfUfuaaguccua | | uaauuGfgGfucaca | | AGUCCUAC | |
| | uL96 | | sasa | | | |
| AD- | usascau(Ahd)Ufg | 150 | usCfsgauu(Cgn)u | 424 | UUUACAUAUGCUUUA | 698 |
| 392787 | CfUfUfuaagaaucg | | uaaagCfaUfaugua | | AGAAUCGA | |
| | aL96 | | sasa | | | |
| AD- | gsusaaa(Uhd)Afa | 151 | usCfscaag(Agn)a | 425 | AUGUAAAUAAAUACA | 699 |
| 392788 | AfUfAfcauucuugg | | uguauUfuAfuuuac | | UUCUUGGA | |
| | aL96 | | sasu | | | |
| AD- | uscsagu(Uhd)Afc | 152 | asAfsgcau(Cgn)g | 426 | GAUCAGUUACGGAAA | 700 |
| 392789 | GfGfAfaacgaugcu | | uuuccGfuAfacuga | | CGAUGCUC | |
| | uL96 | | susc | | | |
| AD- | csusucc(Chd)Gfu | 153 | asAfsacuc(Tgn)c | 427 | UCCUUCCCGUGAAUG | 701 |
| 392790 | GfAfAfuggagaguu | | cauucAfcGfggaag | | GAGAGUUC | |
| | uL96 | | sgsa | | | |
| AD- | asgsuug(Ghd)Afc | 154 | asAfsaugg(Tgn)u | 428 | GAAGUUGGACAGCAA | 702 |
| 392791 | AfGfCfaaaaccauu | | uugcuGfuCfcaacu | | AACCAUUG | |
| | uL96 | | susc | | | |
| AD- | cscscau(Chd)Gfg | 155 | asUfsauaa(Agn)u | 429 | UACCCAUCGGUGUCC | 703 |
| 392792 | UfGfUfccauuuaua | | ggacaCfcGfauggg | | AUUUAUAG | |
| | uL96 | | susa | | | |
| AD- | usgscac(Ahd)Cfa | 156 | usCfsucaa(Tgn)g | 430 | UGUGCACACAUUAGG | 704 |
| 392793 | UfUfAfggcauugag | | ccuaaUfgUfgugca | | CAUUGAGA | |
| | aL96 | | scsa | | | |
| AD- | cscsaac(Ahd)Ufg | 157 | usUfsgguu(Cgn)a | 431 | GGCCAACAUGAUUAG | 705 |
| 392794 | AfUfUfagugaacca | | cuaauCfaUfguugg | | UGAACCAA | |
| | aL96 | | scsc | | | |
| AD- | asusgau(Uhd)Afg | 158 | asAfsuccu(Tgn)g | 432 | ACAUGAUUAGUGAAC | 706 |
| 392795 | UfGfAfaccaaggau | | guucaCfuAfaucau | | CAAGGAUC | |
| | uL96 | | sgsu | | | |
| AD- | ususagu(Ghd)Afa | 159 | asAfscuga(Tgn)c | 433 | GAUUAGUGAACCAAG | 707 |
| 392796 | CfCfAfaggaucagu | | cuuggUfuCfacuaa | | GAUCAGUU | |
| | uL96 | | susc | | | |
| AD- | asascca(Ahd)Gfg | 160 | usUfsccgu(Agn)a | 434 | UGAACCAAGGAUCAG | 708 |
| 392797 | AfUfCfaguuacgga | | cugauCfcUfugguu | | UUACGGAA | |
| | aL96 | | scsa | | | |
| AD- | gsusuac(Ghd)Gfa | 161 | usGfsagag(Cgn)a | 435 | CAGUUACGGAAACGA | 709 |
| 392798 | AfAfCfgaugcucuc | | ucguuUfcCfguaac | | UGCUCUCA | |
| | aL96 | | susg | | | |
| AD- | gsasugc(Ahd)Gfa | 162 | asUfscaug(Tgn)c | 436 | UGGAUGCAGAAUUCC | 710 |
| 392799 | AfUfUfccgacauga | | ggaauUfcUfgcauc | | GACAUGAC | |
| | uL96 | | scsa | | | |
| AD- | ususgga(Chd)Afg | 163 | asGfscaau(Ggn)g | 437 | AGUUGGACAGCAAAA | 711 |
| 392800 | CfAfAfaaccauugc | | uuuugCfuGfuccaa | | CCAUUGCU | |
| | uL96 | | scsu | | | |
| AD- | asasacc(Ahd)Ufu | 164 | usGfsggua(Ggn)u | 438 | CAAAACCAUUGCUUC | 712 |
| 392801 | GfCfUfucacuaccc | | gaagcAfaUfgguuu | | ACUACCCA | |
| | aL96 | | susg | | | |
| AD- | cscsauc(Ghd)Gfu | 165 | usCfsuaua(Agn)a | 439 | ACCCAUCGGUGUCCA | 713 |
| 392802 | GfUfCfcauuuauag | | uggacAfcCfgaugg | | UUUAUAGA | |
| | aL96 | | sgsu | | | |
| AD- | ususauc(Ghd)Cfc | 166 | asCfsagcu(Ggn)u | 440 | CAUUAUCGCCUUUUG | 714 |
| 392803 | UfUfUfugacagcug | | caaaaGfgCfgauaa | | ACAGCUGU | |
| | uL96 | | susg | | | |
| AD- | asuscgc(Chd)Ufu | 167 | asCfsacag(Cgn)u | 441 | UUAUCGCCUUUUGAC | 715 |
| 392804 | UfUfGfacagcugug | | gucaaAfaGfgcgau | | AGCUGUGC | |
| | uL96 | | sasa | | | |
| AD- | ascsaca(Ahd)Gfu | 168 | asGfsuuca(Ggn)g | 442 | UAACACAAGUAGAUG | 716 |
| 392805 | AfGfAfugccugaac | | caucuAfcUfugugu | | CCUGAACU | |
| | uL96 | | susa | | | |
| AD- | usgsugg(Uhd)Ufu | 169 | usUfsaauu(Ggn)g | 443 | CUUGUGGUUUGUGAC | 717 |
| 392806 | GfUfGfacccaauua | | gucacAfaAfccaca | | CCAAUUAA | |
| | aL96 | | sasg | | | |
| AD- | gsgsgau(Ghd)Cfu | 170 | asAfscguu(Cgn)a | 444 | GGGGGAUGCUUCAUG | 718 |
| 392807 | UfCfAfugugaacgu | | caugaAfgCfauccc | | UGAACGUG | |
| | uL96 | | scsc | | | |
| AD- | usgsugc(Ahd)Cfa | 171 | usCfsaaug(Cgn)c | 445 | UAUGUGCACACAUUA | 719 |
| 392808 | CfAfUfuaggcauug | | uaaugUfgUfgcaca | | GGCAUUGA | |
| | aL96 | | susa | | | |
| AD- | asasaug(Ghd)Afa | 172 | asUfsuaua(Tgn)u | 446 | GAAAAUGGAAGUGGC | 720 |
| 392809 | GfUfGfgcaauauaa | | gccacUfuCfcauuu | | AAUAUAAG | |
| | uL96 | | susc | | | |
| AD- | asusgga(Ahd)Gfu | 173 | asCfscuua(Tgn)a | 447 | AAAUGGAAGUGGCAA | 721 |
| 392810 | GfGfCfaauauaagg | | uugccAfcUfuccau | | UAUAAGGG | |
| | uL96 | | susu | | | |
| AD- | usgsccc(Ghd)Afg | 174 | asGfsuuua(Agn)c | 448 | CUUGCCCGAGAUCCU | 722 |
| 392811 | AfUfCfcuguuaaac | | aggauCfuCfgggca | | GUUAAACU | |
| | uL96 | | sasg | | | |
| AD- | asusuag(Uhd)Gfa | 175 | asCfsugau(Cgn)c | 449 | UGAUUAGUGAACCAA | 723 |
| 392812 | AfCfCfaaggaucag | | uugguUfcAfcuaau | | GGAUCAGU | |
| | uL96 | | scsa | | | |
| AD- | gsasacc(Ahd)Afg | 176 | usCfscgua(Agn)c | 450 | GUGAACCAAGGAUCA | 724 |
| 392813 | GfAfUfcaguuacgg | | ugaucCfuUfgguuc | | GUUACGGA | |
| | aL96 | | sasc | | | |
| AD- | asasgga(Uhd)Cfa | 177 | usCfsguuu(Cgn)c | 451 | CCAAGGAUCAGUUAC | 725 |
| 392814 | GfUfUfacggaaacg | | guaacUfgAfuccuu | | GGAAACGA | |
| | aL96 | | sgsg | | | |
| AD- | csasaca(Chd)Afg | 178 | usCfsaacu(Tgn)c | 452 | GCCAACACAGAAAAC | 726 |
| 392815 | AfAfAfacgaaguug | | guuuuCfuGfuguug | | GAAGUUGA | |
| | aL96 | | sgsc | | | |
| AD- | usgsggu(Uhd)Cfa | 179 | usUfsgcac(Cgn)u | 453 | UGUGGGUUCAAACAA | 727 |
| 392816 | AfAfCfaaaggugca | | uuguuUfgAfaccca | | AGGUGCAA | |
| | aL96 | | scsa | | | |
| AD- | csasgug(Ahd)Ufc | 180 | asCfsaagg(Tgn)g | 454 | GACAGUGAUCGUCAU | 728 |
| 392817 | GfUfCfaucaccuug | | augacGfaUfcacug | | CACCUUGG | |
| | uL96 | | susc | | | |
| AD- | ascscca(Uhd)Cfg | 181 | usAfsuaaa(Tgn)g | 455 | CUACCCAUCGGUGUC | 729 |
| 392818 | GfUfGfuccauuuau | | gacacCfgAfugggu | | CAUUUAUA | |
| | aL96 | | sasg | | | |
| AD- | uscsuug(Uhd)Gfg | 182 | asUfsuggg(Tgn)c | 456 | AUUCUUGUGGUUUGU | 730 |
| 392819 | UfUfUfgugacccaa | | acaaaCfcAfcaaga | | GACCCAAU | |
| | uL96 | | sasu | | | |
| AD- | ususugu(Ghd)Afc | 183 | asGfsgacu(Tgn)a | 457 | GGUUUGUGACCCAAU | 731 |
| 392820 | CfCfAfauuaagucc | | auuggGfuCfacaaa | | UAAGUCCU | |
| | uL96 | | scsc | | | |
| AD- | ususgug(Ahd)Cfc | 184 | usAfsggac(Tgn)u | 458 | GUUUGUGACCCAAUU | 732 |
| 392821 | CfAfAfuuaaguccu | | aauugGfgUfcacaa | | AAGUCCUA | |
| | aL96 | | sasc | | | |
| AD- | ususcag(Ahd)Ufg | 185 | usUfsggcc(Agn)a | 459 | UAUUCAGAUGACGUC | 733 |
| 392822 | AfCfGfucuuggcca | | gacguCfaUfcugaa | | UUGGCCAA | |
| | aL96 | | susa | | | |
| AD- | asuscag(Uhd)Ufa | 186 | asGfscauc(Ggn)u | 460 | GGAUCAGUUACGGAA | 734 |
| 392823 | CfGfGfaaacgaugc | | uuccgUfaAfcugau | | ACGAUGCU | |
| | uL96 | | scsc | | | |
| AD- | usgsgau(Ghd)Cfa | 187 | asAfsuguc(Ggn)g | 461 | GAUGGAUGCAGAAUU | 735 |
| 392824 | GfAfAfuuccgacau | | aauucUfgCfaucca | | CCGACAUG | |
| | uL96 | | susc | | | |
| AD- | gsuscca(Ahd)Gfa | 188 | asCfsguuc(Tgn)g | 462 | CUGUCCAAGAUGCAG | 736 |
| 392825 | UfGfCfagcagaacg | | cugcaUfcUfuggac | | CAGAACGG | |
| | uL96 | | sasg | | | |
| AD- | usasccc(Ahd)Ufc | 189 | asUfsaaau(Ggn)g | 463 | ACUACCCAUCGGUGU | 737 |
| 392826 | GfGfUfguccauuua | | acaccGfaUfgggua | | CCAUUUAU | |
| | uL96 | | sgsu | | | |
| AD- | ususuug(Ahd)Cfa | 190 | asUfsuaca(Ggn)c | 464 | CCUUUUGACAGCUGU | 738 |
| 392827 | GfCfUfgugcuguaa | | acagcUfgUfcaaaa | | GCUGUAAC | |
| | uL96 | | sgsg | | | |
| AD- | ususgac(Ahd)Gfc | 191 | asUfsguua(Cgn)a | 465 | UUUUGACAGCUGUGC | 739 |
| 392828 | UfGfUfgcuguaaca | | gcacaGfcUfgucaa | | UGUAACAC | |
| | uL96 | | sasa | | | |
| AD- | asgscug(Uhd)Gfc | 192 | usAfscuug(Tgn)g | 466 | ACAGCUGUGCUGUAA | 740 |
| 392829 | UfGfUfaacacaagu | | uuacaGfcAfcagcu | | CACAAGUA | |
| | aL96 | | sgsu | | | |
| AD- | gsusuuu(Ahd)Ufg | 193 | asCfsuaau(Ggn)u | 467 | CUGUUUUAUGUGCAC | 741 |
| 392830 | UfGfCfacacauuag | | gugcaCfaUfaaaac | | ACAUUAGG | |
| | uL96 | | sasg | | | |
| AD- | ususcaa(Uhd)Ufa | 194 | asGfsagaa(Tgn)u | 468 | UCUUCAAUUACCAAG | 742 |
| 392831 | CfCfAfagaauucuc | | cuuggUfaAfuugaa | | AAUUCUCC | |
| | uL96 | | sgsa | | | |
| AD- | csascac(Ahd)Ufc | 195 | asGfsaaua(Cgn)a | 469 | UCCACACAUCAGUAA | 743 |
| 392832 | AfGfUfaauguauuc | | uuacuGfaUfgugug | | UGUAUUCU | |
| | uL96 | | sgsa | | | |
| AD- | usgsguc(Uhd)Cfu | 196 | asAfsuaau(Ggn)u | 470 | UUUGGUCUCUAUACU | 744 |
| 392833 | AfUfAfcuacauuau | | aguauAfgAfgacca | | ACAUUAUU | |
| | uL96 | | sasa | | | |
| AD- | ascsccg(Uhd)Ufu | 197 | usGfsagua(Agn)a | 471 | AAACCCGUUUUAUGA | 745 |
| 392834 | UfAfUfgauuuacuc | | ucauaAfaAfcgggu | | UUUACUCA | |
| | aL96 | | susu | | | |
| AD- | usascga(Ahd)Afa | 198 | asUfsugua(Ggn)g | 472 | GCUACGAAAAUCCAA | 746 |
| 392835 | UfCfCfaaccuacaa | | uuggaUfuUfucgua | | CCUACAAG | |
| | uL96 | | sgsc | | | |
| AD- | uscscac(Ahd)Cfa | 199 | asAfsuaca(Tgn)u | 473 | AAUCCACACAUCAGU | 747 |
| 392836 | UfCfAfguaauguau | | acugaUfgUfgugga | | AAUGUAUU | |
| | uL96 | | susu | | | |
| AD- | csusggu(Chd)Ufu | 200 | usUfscuug(Ggn)u | 474 | UGCUGGUCUUCAAUU | 748 |
| 392837 | CfAfAfuuaccaaga | | aauugAfaGfaccag | | ACCAAGAA | |
| | aL96 | | scsa | | | |
| AD- | gscscau(Chd)Ufu | 201 | usUfscguu(Tgn)c | 475 | AUGCCAUCUUUGACC | 749 |
| 392838 | UfGfAfccgaaacga | | ggucaAfaGfauggc | | GAAACGAA | |
| | aL96 | | sasu | | | |
| AD- | cscsauc(Uhd)Ufu | 202 | usUfsucgu(Tgn)u | 476 | UGCCAUCUUUGACCG | 750 |
| 392839 | GfAfCfcgaaacgaa | | cggucAfaAfgaugg | | AAACGAAA | |
| | aL96 | | scsa | | | |
| AD- | csusacg(Ahd)Afa | 203 | usUfsguag(Ggn)u | 477 | GGCUACGAAAAUCCA | 751 |
| 392840 | AfUfCfcaaccuaca | | uggauUfuUfcguag | | ACCUACAA | |
| | aL96 | | scsc | | | |
| AD- | asuscca(Chd)Afc | 204 | asUfsacau(Tgn)a | 478 | UAAUCCACACAUCAG | 752 |
| 392841 | AfUfCfaguaaugua | | cugauGfuGfuggau | | UAAUGUAU | |
| | uL96 | | susa | | | |
| AD- | csasugc(Chd)Afu | 205 | asUfsuucg(Ggn)u | 479 | CUCAUGCCAUCUUUG | 753 |
| 392842 | CfUfUfugaccgaaa | | caaagAfuGfgcaug | | ACCGAAAC | |
| | uL96 | | sasg | | | |
| AD- | gsgscua(Chd)Gfa | 206 | asUfsaggu(Tgn)g | 480 | ACGGCUACGAAAAUC | 754 |
| 392843 | AfAfAfuccaaccua | | gauuuUfcGfuagcc | | CAACCUAC | |
| | uL96 | | sgsu | | | |
| AD- | uscsaug(Chd)Cfa | 207 | usUfsucgg(Tgn)c | 481 | UCUCAUGCCAUCUUU | 755 |
| 392844 | UfCfUfuugaccgaa | | aaagaUfgGfcauga | | GACCGAAA | |
| | aL96 | | sgsa | | | |
| AD- | csasgua(Chd)Afc | 208 | asUfsgaug(Agn)a | 482 | AACAGUACACAUCCA | 756 |
| 392845 | AfUfCfcauucauca | | uggauGfuGfuacug | | UUCAUCAU | |
| | uL96 | | susu | | | |
| AD- | asascgg(Chd)Ufa | 209 | asGfsuugg(Agn)u | 483 | AGAACGGCUACGAAA | 757 |
| 392846 | CfGfAfaaauccaac | | uuucgUfaGfccguu | | AUCCAACC | |
| | uL96 | | scsu | | | |
| AD- | gsasagu(Uhd)Ufc | 210 | usUfsguau(Cgn)a | 484 | CUGAAGUUUCAUUUA | 758 |
| 392847 | AfUfUfuaugauaca | | uaaauGfaAfacuuc | | UGAUACAA | |
| | aL96 | | sasg | | | |
| AD- | asusgcc(Ahd)Ufc | 211 | asGfsuuuc(Ggn)g | 485 | UCAUGCCAUCUUUGA | 759 |
| 392848 | UfUfUfgaccgaaac | | ucaaaGfaUfggcau | | CCGAAACG | |
| | uL96 | | sgsa | | | |
| AD- | gsasacg(Ghd)Cfu | 212 | asUfsugga(Tgn)u | 486 | CAGAACGGCUACGAA | 760 |
| 392849 | AfCfGfaaaauccaa | | uucguAfgCfcguuc | | AAUCCAAC | |
| | uL96 | | susg | | | |
| AD- | uscsuuc(Ghd)Ufg | 213 | asAfscaua(Agn)a | 487 | UUUCUUCGUGCCUGU | 761 |
| 392850 | CfCfUfguuuuaugu | | acaggCfaCfgaaga | | UUUAUGUG | |
| | uL96 | | sasa | | | |
| AD- | ususgcc(Chd)Gfa | 214 | asUfsuuaa(Cgn)a | 488 | UCUUGCCCGAGAUCC | 762 |
| 392851 | GfAfUfccuguuaaa | | ggaucUfcGfggcaa | | UGUUAAAC | |
| | uL96 | | sgsa | | | |
| AD- | csusucg(Uhd)Gfc | 215 | asCfsacau(Agn)a | 489 | UUCUUCGUGCCUGUU | 763 |
| 392852 | CfUfGfuuuuaugug | | aacagGfcAfcgaag | | UUAUGUGC | |
| | uL96 | | sasa | | | |
| AD- | gscsgcc(Ahd)Ufg | 216 | asUfsaaac(Tgn)u | 490 | CAGCGCCAUGUCCCA | 764 |
| 392853 | UfCfCfcaaaguuua | | ugggaCfaUfggcgc | | AAGUUUAC | |
| | uL96 | | susg | | | |
| AD- | gsuscau(Ahd)Gfc | 217 | asAfscgau(Cgn)a | 491 | UUGUCAUAGCGACAG | 765 |
| 392854 | GfAfCfagugaucgu | | cugucGfcUfaugac | | UGAUCGUC | |
| | uL96 | | sasa | | | |
| AD- | gscsuac(Ghd)Afa | 218 | usGfsuagg(Tgn)u | 492 | CGGCUACGAAAAUCC | 766 |
| 392855 | AfAfUfccaaccuac | | ggauuUfuCfguagc | | AACCUACA | |
| | aL96 | | scsg | | | |
| AD- | asusagc(Ghd)Afc | 219 | asAfsugac(Ggn)a | 493 | UCAUAGCGACAGUGA | 767 |
| 392856 | AfGfUfgaucgucau | | ucacuGfuCfgcuau | | UCGUCAUC | |
| | uL96 | | sgsa | | | |
| AD- | csusugc(Chd)Cfg | 220 | usUfsuaac(Agn)g | 494 | CUCUUGCCCGAGAUC | 768 |
| 392857 | AfGfAfuccuguuaa | | gaucuCfgGfgcaag | | CUGUUAAA | |
| | aL96 | | sasg | | | |
| AD- | csuscau(Ghd)Cfc | 221 | usUfscggu(Cgn)a | 495 | CUCUCAUGCCAUCUU | 769 |
| 392858 | AfUfCfuuugaccga | | aagauGfgCfaugag | | UGACCGAA | |
| | aL96 | | sasg | | | |
| AD- | ascsggc(Uhd)Afc | 222 | asGfsguug(Ggn)a | 496 | GAACGGCUACGAAAA | 770 |
| 392859 | GfAfAfaauccaacc | | uuuucGfuAfgccgu | | UCCAACCU | |
| | uL96 | | susc | | | |
| AD- | csasuca(Ahd)Afa | 223 | asAfsagaa(Cgn)a | 497 | AUCAUCAAAAAUUGG | 771 |
| 392860 | AfUfUfgguguucuu | | ccaauUfuUfugaug | | UGUUCUUU | |
| | uL96 | | sasu | | | |
| AD- | asuscca(Ahd)Cfc | 224 | csAfsaaga(Agn)c | 498 | AAAUCCAACCUACAA | 772 |
| 392861 | UfAfCfaaguucuuu | | uuguaGfgUfuggau | | GUUCUUUG | |
| | gL96 | | susu | | | |
| AD- | csgscuu(Uhd)Cfu | 225 | usGfsuaau(Agn)c | 499 | AUCGCUUUCUACACU | 773 |
| 392862 | AfCfAfcuguauuac | | aguguAfgAfaagcg | | GUAUUACA | |
| | aL96 | | sasu | | | |
| AD- | uscscaa(Chd)Cfu | 226 | usCfsaaag(Agn)a | 500 | AAUCCAACCUACAAG | 774 |
| 392863 | AfCfAfaguucuuug | | cuuguAfgGfuugga | | UUCUUUGA | |
| | aL96 | | susu | | | |
| AD- | uscsucu(Chd)Ufu | 227 | asGfsacca(Agn)a | 501 | UAUCUCUCUUUACAU | 775 |
| 392864 | UfAfCfauuuugguc | | auguaAfaGfagaga | | UUUGGUCU | |
| | uL96 | | susa | | | |
| AD- | csuscuc(Uhd)Ufu | 228 | asAfsgacc(Agn)a | 502 | AUCUCUCUUUACAUU | 776 |
| 392865 | AfCfAfuuuuggucu | | aauguAfaAfgagag | | UUGGUCUC | |
| | uL96 | | sasu | | | |
| AD- | ususugu(Ghd)Ufa | 229 | asAfsauuc(Tgn)u | 503 | GUUUUGUGUACUGUA | 777 |
| 392866 | CfUfGfuaaagaauu | | uacagUfaCfacaaa | | AAGAAUUU | |
| | uL96 | | sasc | | | |
| AD- | gsusgua(Chd)Ufg | 230 | asCfsuaaa(Tgn)u | 504 | UUGUGUACUGUAAAG | 778 |
| 392867 | UfAfAfagaauuuag | | cuuuaCfaGfuacac | | AAUUUAGC | |
| | uL96 | | sasa | | | |
| AD- | ascscca(Ahd)Ufu | 231 | usAfsaagu(Agn)g | 505 | UGACCCAAUUAAGUC | 779 |
| 392868 | AfAfGfuccuacuuu | | gacuuAfaUfugggu | | CUACUUUA | |
| | aL96 | | scsa | | | |
| AD- | uscscua(Chd)Ufu | 232 | usAfsaagc(Agn)u | 506 | AGUCCUACUUUACAU | 780 |
| 392869 | UfAfCfauaugcuuu | | auguaAfaGfuagga | | AUGCUUUA | |
| | aL96 | | scsu | | | |
| AD- | cscsuac(Uhd)Ufu | 233 | usUfsaaag(Cgn)a | 507 | GUCCUACUUUACAUA | 781 |
| 392870 | AfCfAfuaugcuuua | | uauguAfaAfguagg | | UGCUUUAA | |
| | aL96 | | sasc | | | |
| AD- | ususcua(Chd)Afc | 234 | usUfsuaug(Tgn)a | 508 | CUUUCUACACUGUAU | 782 |
| 392871 | UfGfUfauuacauaa | | auacaGfuGfuagaa | | UACAUAAA | |
| | aL96 | | sasg | | | |
| AD- | uscsuac(Ahd)Cfu | 235 | asUfsuuau(Ggn)u | 509 | UUUCUACACUGUAUU | 783 |
| 392872 | GfUfAfuuacauaaa | | aauacAfgUfguaga | | ACAUAAAU | |
| | uL96 | | sasa | | | |
| AD- | csusuuu(Ahd)Afg | 236 | asUfsugaa(Ggn)a | 510 | UUCUUUUAAGAUGUG | 784 |
| 392873 | AfUfGfugucuucaa | | cacauCfuUfaaaag | | UCUUCAAU | |
| | uL96 | | sasa | | | |
| AD- | asusgug(Uhd)Cfu | 237 | usUfsauac(Agn)a | 511 | AGAUGUGUCUUCAAU | 785 |
| 392874 | UfCfAfauuuguaua | | auugaAfgAfcacau | | UUGUAUAA | |
| | aL96 | | scsu | | | |
| AD- | asuscaa(Ahd)Afa | 238 | csAfsaaga(Agn)c | 512 | UCAUCAAAAAUUGGU | 786 |
| 392875 | UfUfGfguguucuuu | | accaaUfuUfuugau | | GUUCUUUG | |
| | gL96 | | sgsa | | | |
| AD- | asasauc(Chd)Afa | 239 | asAfsgaac(Tgn)u | 513 | GAAAAUCCAACCUAC | 787 |
| 392876 | CfCfUfacaaguucu | | guaggUfuGfgauuu | | AAGUUCUU | |
| | uL96 | | susc | | | |
| AD- | gsusacu(Ghd)Ufa | 240 | asAfsgcua(Agn)a | 514 | GUGUACUGUAAAGAA | 788 |
| 392877 | AfAfGfaauuuagcu | | uucuuUfaCfaguac | | UUUAGCUG | |
| | uL96 | | sasc | | | |
| AD- | csusccu(Ghd)Afu | 241 | usAfsugug(Agn)u | 515 | CUCUCCUGAUUAUUU | 789 |
| 392878 | UfAfUfuuaucacau | | aaauaAfuCfaggag | | AUCACAUA | |
| | aL96 | | sasg | | | |
| AD- | gscscag(Uhd)Ufg | 242 | asAfsagaa(Tgn)a | 516 | UAGCCAGUUGUAUAU | 790 |
| 392879 | UfAfUfauuauucuu | | auauaCfaAfcuggc | | UAUUCUUG | |
| | uL96 | | susa | | | |
| AD- | asasuua(Ahd)Gfu | 243 | usAfsugua(Agn)a | 517 | CCAAUUAAGUCCUAC | 791 |
| 392880 | CfCfUfacuuuacau | | guaggAfcUfuaauu | | UUUACAUA | |
| | aL96 | | sgsg | | | |
| AD- | csusugc(Chd)Ufa | 244 | asGfsaaag(Ggn)a | 518 | CUCUUGCCUAAGUAU | 792 |
| 392881 | AfGfUfauuccuuuc | | auacuUfaGfgcaag | | UCCUUUCC | |
| | uL96 | | sasg | | | |
| AD- | asusucc(Uhd)Ufu | 245 | asAfsuagu(Ggn)a | 519 | GUAUUCCUUUCCUGA | 793 |
| 392882 | CfCfUfgaucacuau | | ucaggAfaAfggaau | | UCACUAUG | |
| | uL96 | | sasc | | | |
| AD- | ascsuau(Ghd)Cfa | 246 | usUfsuaac(Tgn)u | 520 | UCACUAUGCAUUUUA | 794 |
| 392883 | UfUfUfuaaaguuaa | | uaaaaUfgCfauagu | | AAGUUAAA | |
| | aL96 | | sgsa | | | |
| AD- | usgsuuc(Ahd)Ufu | 247 | usAfsaaag(Tgn)g | 521 | CCUGUUCAUUGUAAG | 795 |
| 392884 | GfUfAfagcacuuuu | | cuuacAfaUfgaaca | | CACUUUUA | |
| | aL96 | | sgsg | | | |
| AD- | asasuua(Chd)Cfa | 248 | usUfsugga(Ggn)a | 522 | UCAAUUACCAAGAAU | 796 |
| 392885 | AfGfAfauucuccaa | | auucuUfgGfuaauu | | UCUCCAAA | |
| | aL96 | | sgsa | | | |
| AD- | ususacc(Ahd)Afg | 249 | asUfsuuug(Ggn)a | 523 | AAUUACCAAGAAUUC | 797 |
| 392886 | AfAfUfucuccaaaa | | gaauuCfuUfgguaa | | UCCAAAAC | |
| | uL96 | | susu | | | |
| AD- | uscsauu(Ghd)Cfu | 250 | asGfsauca(Tgn)g | 524 | AAUCAUUGCUUAUGA | 798 |
| 392887 | UfAfUfgacaugauc | | ucauaAfgCfaauga | | CAUGAUCG | |
| | uL96 | | susu | | | |
| AD- | ususuua(Ahd)Gfa | 251 | asAfsuuga(Agn)g | 525 | UCUUUUAAGAUGUGU | 799 |
| 392889 | UfGfUfgucuucaau | | acacaUfcUfuaaaa | | CUUCAAUU | |
| | uL96 | | sgsa | | | |
| AD- | asusccu(Ghd)Ufu | 252 | usUfsguag(Ggn)a | 526 | AGAUCCUGUUAAACU | 800 |
| 392890 | AfAfAfcuuccuaca | | aguuuAfaCfaggau | | UCCUACAA | |
| | aL96 | | scsu | | | |
| AD- | ascsuau(Uhd)Cfa | 253 | asCfsaaga(Cgn)g | 527 | AAACUAUUCAGAUGA | 801 |
| 392891 | GfAfUfgacgucuug | | ucaucUfgAfauagu | | CGUCUUGG | |
| | uL96 | | susu | | | |
| AD- | gsusuca(Uhd)Cfa | 254 | asAfsccaa(Tgn)u | 528 | AAGUUCAUCAUCAAA | 802 |
| 392892 | UfCfAfaaaauuggu | | uuugaUfgAfugaac | | AAUUGGUG | |
| | uL96 | | susu | | | |
| AD- | usasucu(Chd)Ufc | 255 | asCfscaaa(Agn)u | 529 | UCUAUCUCUCUUUAC | 803 |
| 392893 | UfUfUfacauuuugg | | guaaaGfaGfagaua | | AUUUUGGU | |
| | uL96 | | sgsa | | | |
| AD- | asuscuc(Uhd)Cfu | 256 | asAfsccaa(Agn)a | 530 | CUAUCUCUCUUUACA | 804 |
| 392894 | UfUfAfcauuuuggu | | uguaaAfgAfgagau | | UUUUGGUC | |
| | uL96 | | sasg | | | |
| AD- | usgsugu(Ahd)Cfu | 257 | asUfsaaau(Tgn)c | 531 | UUUGUGUACUGUAAA | 805 |
| 392895 | GfUfAfaagaauuua | | uuuacAfgUfacaca | | GAAUUUAG | |
| | uL96 | | sasa | | | |
| AD- | csusacu(Uhd)Ufa | 258 | asUfsuaaa(Ggn)c | 532 | UCCUACUUUACAUAU | 806 |
| 392896 | CfAfUfaugcuuuaa | | auaugUfaAfaguag | | GCUUUAAG | |
| | uL96 | | sgsa | | | |
| AD- | usgsccu(Ahd)Afg | 259 | asAfsggaa(Agn)g | 533 | CUUGCCUAAGUAUUC | 807 |
| 392897 | UfAfUfuccuuuccu | | gaauaCfuUfaggca | | CUUUCCUG | |
| | uL96 | | sasg | | | |
| AD- | asasgua(Uhd)Ufc | 260 | asUfsgauc(Agn)g | 534 | CUAAGUAUUCCUUUC | 808 |
| 392898 | CfUfUfuccugauca | | gaaagGfaAfuacuu | | CUGAUCAC | |
| | uL96 | | sasg | | | |
| AD- | gsusauu(Chd)Cfu | 261 | usAfsguga(Tgn)c | 535 | AAGUAUUCCUUUCCU | 809 |
| 392899 | UfUfCfcugaucacu | | aggaaAfgGfaauac | | GAUCACUA | |
| | aL96 | | susu | | | |
| AD- | ususccu(Ghd)Afu | 262 | asAfsaaug(Cgn)a | 536 | CUUUCCUGAUCACUA | 810 |
| 392900 | CfAfCfuaugcauuu | | uagugAfuCfaggaa | | UGCAUUUU | |
| | uL96 | | sasg | | | |
| AD- | csusgau(Chd)Afc | 263 | usUfsuaaa(Agn)u | 537 | UCCUGAUCACUAUGC | 811 |
| 392901 | UfAfUfgcauuuuaa | | gcauaGfuGfaucag | | AUUUUAAA | |
| | aL96 | | sgsa | | | |
| AD- | csascgu(Ahd)Ufc | 264 | usCfsaaag(Agn)c | 538 | UCCACGUAUCUUUGG | 812 |
| 392902 | UfUfUfgggucuuug | | ccaaaGfaUfacgug | | GUCUUUGA | |
| | aL96 | | sgsa | | | |
| AD- | usgsggu(Chd)Ufu | 265 | asUfsuuuc(Tgn)u | 539 | UUUGGGUCUUUGAUA | 813 |
| 392903 | UfGfAfuaaagaaaa | | uaucaAfaGfaccca | | AAGAAAAG | |
| | uL96 | | sasa | | | |
| AD- | uscsaau(Uhd)Afc | 266 | usGfsgaga(Agn)u | 540 | CUUCAAUUACCAAGA | 814 |
| 392904 | CfAfAfgaauucucc | | ucuugGfuAfauuga | | AUUCUCCA | |
| | aL96 | | sasg | | | |
| AD- | uscsgcu(Uhd)Ufc | 267 | asUfsaaua(Cgn)a | 541 | GAUCGCUUUCUACAC | 815 |
| 392906 | UfAfCfacuguauua | | guguaGfaAfagcga | | UGUAUUAC | |
| | uL96 | | susc | | | |
| AD- | asusuuu(Chd)Ufu | 268 | usUfscaga(Cgn)u | 542 | CAAUUUUCUUUAACC | 816 |
| 392907 | UfAfAfccagucuga | | gguuaAfaGfaaaau | | AGUCUGAA | |
| | aL96 | | susg | | | |
| AD- | csusuua(Ahd)Cfc | 269 | gsAfsaacu(Tgn)c | 543 | UUCUUUAACCAGUCU | 817 |
| 392908 | AfGfUfcugaaguuu | | agacuGfgUfuaaag | | GAAGUUUC | |
| | cL96 | | sasa | | | |
| AD- | usasaga(Uhd)Gfu | 270 | asCfsaaau(Tgn)g | 544 | UUUAAGAUGUGUCUU | 818 |
| 392909 | GfUfCfuucaauuug | | aagacAfcAfucuua | | CAAUUUGU | |
| | uL96 | | sasa | | | |
| AD- | gsasucc(Uhd)Gfu | 271 | usGfsuagg(Agn)a | 545 | GAGAUCCUGUUAAAC | 819 |
| 392910 | UfAfAfacuuccuac | | guuuaAfcAfggauc | | UUCCUACA | |
| | aL96 | | susc | | | |
| AD- | csusgcu(Uhd)Cfa | 272 | asUfsuuug(Cgn)u | 546 | AGCUGCUUCAGAAAG | 820 |
| 392911 | GfAfAfagagcaaaa | | cuuucUfgAfagcag | | AGCAAAAC | |
| | uL96 | | scsu | | | |
| AD- | csasgaa(Ahd)Gfa | 273 | usGfsaaua(Ggn)u | 547 | UUCAGAAAGAGCAAA | 821 |
| 392912 | GfCfAfaaacuauuc | | uuugcUfcUfuucug | | ACUAUUCA | |
| | aL96 | | sasa | | | |
| AD- | usasuga(Ahd)Gfu | 274 | usUfsuuug(Agn)u | 548 | GAUAUGAAGUUCAUC | 822 |
| 392913 | UfCfAfucaucaaaa | | gaugaAfcUfucaua | | AUCAAAAA | |
| | aL96 | | susc | | | |
| AD- | csasuca(Uhd)Cfa | 275 | asAfsacac(Cgn)a | 549 | UUCAUCAUCAAAAAU | 823 |
| 392914 | AfAfAfauugguguu | | auuuuUfgAfugaug | | UGGUGUUC | |
| | uL96 | | sasa | | | |
| AD- | uscsaaa(Ahd)Afu | 276 | asCfsaaag(Agn)a | 550 | CAUCAAAAAUUGGUG | 824 |
| 392915 | UfGfGfuguucuuug | | caccaAfuUfuuuga | | UUCUUUGC | |
| | uL96 | | susg | | | |
| AD- | asasaau(Chd)Cfa | 277 | asGfsaacu(Tgn)g | 551 | CGAAAAUCCAACCUA | 825 |
| 392916 | AfCfCfuacaaguuc | | uagguUfgGfauuuu | | CAAGUUCU | |
| | uL96 | | scsg | | | |
| AD- | cscsaac(Chd)Ufa | 278 | asUfscaaa(Ggn)a | 552 | AUCCAACCUACAAGU | 826 |
| 392917 | CfAfAfguucuuuga | | acuugUfaGfguugg | | UCUUUGAG | |
| | uL96 | | sasu | | | |
| AD- | ascsuca(Uhd)Ufa | 279 | usGfsucaa(Agn)a | 553 | UUACUCAUUAUCGCC | 827 |
| 392918 | UfCfGfccuuuugac | | ggcgaUfaAfugagu | | UUUUGACA | |
| | aL96 | | sasa | | | |
| AD- | csuscau(Uhd)Afu | 280 | asUfsguca(Agn)a | 554 | UACUCAUUAUCGCCU | 828 |
| 392919 | CfGfCfcuuuugaca | | aggcgAfuAfaugag | | UUUGACAG | |
| | uL96 | | susa | | | |
| AD- | usgsugc(Uhd)Gfu | 281 | asUfscuac(Tgn)u | 555 | GCUGUGCUGUAACAC | 829 |
| 392920 | AfAfCfacaaguaga | | guguuAfcAfgcaca | | AAGUAGAU | |
| | uL96 | | sgsc | | | |
| AD- | gsusgcu(Ghd)Ufa | 282 | asAfsucua(Cgn)u | 556 | CUGUGCUGUAACACA | 830 |
| 392921 | AfCfAfcaaguagau | | uguguUfaCfagcac | | AGUAGAUG | |
| | uL96 | | sasg | | | |
| AD- | uscsuuu(Ahd)Cfa | 283 | asUfsagag(Agn)c | 557 | UCUCUUUACAUUUUG | 831 |
| 392922 | UfUfUfuggucucua | | caaaaUfgUfaaaga | | GUCUCUAU | |
| | uL96 | | sgsa | | | |
| AD- | asusggg(Uhd)Ufu | 284 | usUfsuaca(Ggn)u | 558 | UAAUGGGUUUUGUGU | 832 |
| 392923 | UfGfUfguacuguaa | | acacaAfaAfcccau | | ACUGUAAA | |
| | aL96 | | susa | | | |
| AD- | ususgug(Uhd)Afc | 285 | usAfsaauu(Cgn)u | 559 | UUUUGUGUACUGUAA | 833 |
| 392924 | UfGfUfaaagaauuu | | uuacaGfuAfcacaa | | AGAAUUUA | |
| | aL96 | | sasa | | | |
| AD- | gscsugu(Ahd)Ufc | 286 | asAfsugca(Cgn)u | 560 | UAGCUGUAUCAAACU | 834 |
| 392925 | AfAfAfcuagugcau | | aguuuGfaUfacagc | | AGUGCAUG | |
| | uL96 | | susa | | | |
| AD- | csusagu(Ghd)Cfa | 287 | asAfsgaau(Cgn)u | 561 | AACUAGUGCAUGAAU | 835 |
| 392926 | UfGfAfauagauucu | | auucaUfgCfacuag | | AGAUUCUC | |
| | uL96 | | susu | | | |
| AD- | usasgug(Chd)Afu | 288 | asGfsagaa(Tgn)c | 562 | ACUAGUGCAUGAAUA | 836 |
| 392927 | GfAfAfuagauucuc | | uauucAfuGfcacua | | GAUUCUCU | |
| | uL96 | | sgsu | | | |
| AD- | csuscuc(Chd)Ufg | 289 | usGfsugau(Agn)a | 563 | UUCUCUCCUGAUUAU | 837 |
| 392928 | AfUfUfauuuaucac | | auaauCfaGfgagag | | UUAUCACA | |
| | aL96 | | sasa | | | |
| AD- | cscsuga(Uhd)Ufa | 290 | asCfsuaug(Tgn)g | 564 | CUCCUGAUUAUUUAU | 838 |
| 392929 | UfUfUfaucacauag | | auaaaUfaAfucagg | | CACAUAGC | |
| | uL96 | | sasg | | | |
| AD- | usasagu(Chd)Cfu | 291 | asCfsauau(Ggn)u | 565 | AUUAAGUCCUACUUU | 839 |
| 392930 | AfCfUfuuacauaug | | aaaguAfgGfacuua | | ACAUAUGC | |
| | uL96 | | sasu | | | |
| AD- | asgsucc(Uhd)Afc | 292 | asAfsgcau(Agn)u | 566 | UAAGUCCUACUUUAC | 840 |
| 392931 | UfUfUfacauaugcu | | guaaaGfuAfggacu | | AUAUGCUU | |
| | uL96 | | susa | | | |
| AD- | gsusccu(Ahd)Cfu | 293 | asAfsagca(Tgn)a | 567 | AAGUCCUACUUUACA | 841 |
| 392932 | UfUfAfcauaugcuu | | uguaaAfgUfaggac | | UAUGCUUU | |
| | uL96 | | susu | | | |
| AD- | ususcuc(Uhd)Ufg | 294 | asGfsgaau(Agn)c | 568 | GCUUCUCUUGCCUAA | 842 |
| 392933 | CfCfUfaaguauucc | | uuaggCfaAfgagaa | | GUAUUCCU | |
| | uL96 | | sgsc | | | |
| AD- | csuscuu(Ghd)Cfc | 295 | asAfsagga(Agn)u | 569 | UUCUCUUGCCUAAGU | 843 |
| 392934 | UfAfAfguauuccuu | | acuuaGfgCfaagag | | AUUCCUUU | |
| | uL96 | | sasa | | | |
| AD- | usasuuc(Chd)Ufu | 296 | asUfsagug(Agn)u | 570 | AGUAUUCCUUUCCUG | 844 |
| 392935 | UfCfCfugaucacua | | caggaAfaGfgaaua | | AUCACUAU | |
| | uL96 | | scsu | | | |
| AD- | ususucc(Uhd)Gfa | 297 | asAfsaugc(Agn)u | 571 | CCUUUCCUGAUCACU | 845 |
| 392936 | UfCfAfcuaugcauu | | agugaUfcAfggaaa | | AUGCAUUU | |
| | uL96 | | sgsg | | | |
| AD- | csascua(Uhd)Gfc | 298 | usUfsaacu(Tgn)u | 572 | AUCACUAUGCAUUUU | 846 |
| 392937 | AfUfUfuuaaaguua | | aaaauGfcAfuagug | | AAAGUUAA | |
| | aL96 | | sasu | | | |
| AD- | csusgca(Uhd)Ufu | 299 | asAfsucug(Tgn)a | 573 | GACUGCAUUUUACUG | 847 |
| 392938 | UfAfCfuguacagau | | caguaAfaAfugcag | | UACAGAUU | |
| | uL96 | | susc | | | |
| AD- | ususcug(Chd)Ufa | 300 | usAfsuauc(Agn)c | 574 | GCUUCUGCUAUAUUU | 848 |
| 392939 | UfAfUfuugugauau | | aaauaUfaGfcagaa | | GUGAUAUA | |
| | aL96 | | sgsc | | | |
| AD- | uscsugc(Uhd)Afu | 301 | asUfsauau(Cgn)a | 575 | CUUCUGCUAUAUUUG | 849 |
| 392940 | AfUfUfugugauaua | | caaauAfuAfgcaga | | UGAUAUAG | |
| | uL96 | | sasg | | | |
| AD- | ascsgua(Uhd)Cfu | 302 | asUfscaaa(Ggn)a | 576 | CCACGUAUCUUUGGG | 850 |
| 392941 | UfUfGfggucuuuga | | cccaaAfgAfuacgu | | UCUUUGAU | |
| | uL96 | | sgsg | | | |
| AD- | uscsuuu(Ghd)Gfg | 303 | usCfsuuua(Tgn)c | 577 | UAUCUUUGGGUCUUU | 851 |
| 392942 | UfCfUfuugauaaag | | aaagaCfcCfaaaga | | GAUAAAGA | |
| | aL96 | | susa | | | |
| AD- | csusuug(Ghd)Gfu | 304 | usUfscuuu(Agn)u | 578 | AUCUUUGGGUCUUUG | 852 |
| 392943 | CfUfUfugauaaaga | | caaagAfcCfcaaag | | AUAAAGAA | |
| | aL96 | | sasu | | | |
| AD- | ususggg(Uhd)Cfu | 305 | usUfsuucu(Tgn)u | 579 | CUUUGGGUCUUUGAU | 853 |
| 392944 | UfUfGfauaaagaaa | | aucaaAfgAfcccaa | | AAAGAAAA | |
| | aL96 | | sasg | | | |
| AD- | asgsaau(Chd)Cfc | 306 | asUfsuaca(Agn)u | 580 | AAAGAAUCCCUGUUC | 854 |
| 392945 | UfGfUfucauuguaa | | gaacaGfgGfauucu | | AUUGUAAG | |
| | uL96 | | susu | | | |
| AD- | gsasauc(Chd)Cfu | 307 | asCfsuuac(Agn)a | 581 | AAGAAUCCCUGUUCA | 855 |
| 392946 | GfUfUfcauuguaag | | ugaacAfgGfgauuc | | UUGUAAGC | |
| | uL96 | | susu | | | |
| AD- | gsusuca(Uhd)Ufg | 308 | asUfsaaaa(Ggn)u | 582 | CUGUUCAUUGUAAGC | 856 |
| 392947 | UfAfAfgcacuuuua | | gcuuaCfaAfugaac | | ACUUUUAC | |
| | uL96 | | sasg | | | |
| AD- | ususaug(Ahd)Cfa | 309 | usAfsgaaa(Ggn)c | 583 | GCUUAUGACAUGAUC | 857 |
| 392948 | UfGfAfucgcuuucu | | gaucaUfgUfcauaa | | GCUUUCUA | |
| | aL96 | | sgsc | | | |
| AD- | asusgac(Ahd)Ufg | 310 | usGfsuaga(Agn)a | 584 | UUAUGACAUGAUCGC | 858 |
| 392949 | AfUfCfgcuuucuac | | gcgauCfaUfgucau | | UUUCUACA | |
| | aL96 | | sasa | | | |
| AD- | csasuga(Uhd)Cfg | 311 | asCfsagug(Tgn)a | 585 | GACAUGAUCGCUUUC | 859 |
| 392950 | CfUfUfucuacacug | | gaaagCfgAfucaug | | UACACUGU | |
| | uL96 | | susc | | | |
| AD- | csusuuc(Uhd)Afc | 312 | usAfsugua(Agn)u | 586 | CGCUUUCUACACUGU | 860 |
| 392951 | AfCfUfguauuacau | | acaguGfuAfgaaag | | AUUACAUA | |
| | aL96 | | scsg | | | |
| AD- | gsasuuc(Ahd)Afu | 313 | asUfsgguu(Agn)a | 587 | CAGAUUCAAUUUUCU | 861 |
| 392952 | UfUfUfcuuuaacca | | agaaaAfuUfgaauc | | UUAACCAG | |
| | uL96 | | susg | | | |
| AD- | ususucu(Uhd)Ufa | 314 | asCfsuuca(Ggn)a | 588 | AUUUUCUUUAACCAG | 862 |
| 392953 | AfCfCfagucugaag | | cugguUfaAfagaaa | | UCUGAAGU | |
| | uL96 | | sasu | | | |
| AD- | ususuaa(Ghd)Afu | 315 | asAfsauug(Agn)a | 589 | CUUUUAAGAUGUGUC | 863 |
| 392954 | GfUfGfucuucaauu | | gacacAfuCfuuaaa | | UUCAAUUU | |
| | uL96 | | sasg | | | |
| AD- | ususaag(Ahd)Ufg | 316 | csAfsaauu(Ggn)a | 590 | UUUUAAGAUGUGUCU | 864 |
| 392955 | UfGfUfcuucaauuu | | agacaCfaUfcuuaa | | UCAAUUUG | |
| | gL96 | | sasa | | | |
| AD- | asgsaug(Uhd)Gfu | 317 | asUfsacaa(Agn)u | 591 | UAAGAUGUGUCUUCA | 865 |
| 392956 | CfUfUfcaauuugua | | ugaagAfcAfcaucu | | AUUUGUAU | |
| | uL96 | | susa | | | |
| AD- | usgsucu(Uhd)Cfa | 318 | asUfsuuua(Tgn)a | 592 | UGUGUCUUCAAUUUG | 866 |
| 392957 | AfUfUfuguauaaaa | | caaauUfgAfagaca | | UAUAAAAU | |
| | uL96 | | scsa | | | |
| AD- | csusuca(Ahd)Ufu | 319 | asCfscauu(Tgn)u | 593 | GUCUUCAAUUUGUAU | 867 |
| 392958 | UfGfUfauaaaaugg | | auacaAfaUfugaag | | AAAAUGGU | |
| | uL96 | | sasc | | | |
| AD- | asusggu(Ghd)Ufu | 320 | usUfsauuu(Agn)c | 594 | AAAUGGUGUUUUCAU | 868 |
| 392959 | UfUfCfauguaaaua | | augaaAfaCfaccau | | GUAAAUAA | |
| | aL96 | | susu | | | |
| AD- | ususcuu(Uhd)Ufa | 321 | usGfsaaga(Cgn)a | 595 | CCUUCUUUUAAGAUG | 869 |
| 392960 | AfGfAfugugucuuc | | caucuUfaAfaagaa | | UGUCUUCA | |
| | aL96 | | sgsg | | | |
| AD- | usgsuau(Uhd)Cfu | 322 | usGfsuaaa(Ggn)a | 596 | AAUGUAUUCUAUCUC | 870 |
| 392961 | AfUfCfucucuuuac | | gagauAfgAfauaca | | UCUUUACA | |
| | aL96 | | susu | | | |
| AD- | gsuscuc(Uhd)Afu | 323 | usUfsaaua(Agn)u | 597 | UGGUCUCUAUACUAC | 871 |
| 392962 | AfCfUfacauuauua | | guaguAfuAfgagac | | AUUAUUAA | |
| | aL96 | | scsa | | | |
| AD- | uscsucu(Ahd)Ufa | 324 | asUfsuaau(Agn)a | 598 | GGUCUCUAUACUACA | 872 |
| 392963 | CfUfAfcauuauuaa | | uguagUfaUfagaga | | UUAUUAAU | |
| | uL96 | | scsc | | | |
| AD- | csuscua(Uhd)Afc | 325 | asAfsuuaa(Tgn)a | 599 | GUCUCUAUACUACAU | 873 |
| 392964 | UfAfCfauuauuaau | | auguaGfuAfuagag | | UAUUAAUG | |
| | uL96 | | sasc | | | |
| AD- | csusuca(Ahd)Ufu | 326 | asAfsgaau(Tgn)c | 600 | GUCUUCAAUUACCAA | 874 |
| 392965 | AfCfCfaagaauucu | | uugguAfaUfugaag | | GAAUUCUC | |
| | uL96 | | sasc | | | |
| AD- | cscsaca(Chd)Afu | 327 | asAfsauac(Agn)u | 601 | AUCCACACAUCAGUA | 875 |
| 392966 | CfAfGfuaauguauu | | uacugAfuGfugugg | | AUGUAUUC | |
| | uL96 | | sasu | | | |
| AD- | csusauc(Uhd)Cfu | 328 | asCfsaaaa(Tgn)g | 602 | UUCUAUCUCUCUUUA | 876 |
| 392967 | CfUfUfuacauuuug | | uaaagAfgAfgauag | | CAUUUUGG | |
| | uL96 | | sasa | | | |
| AD- | gsgsucu(Chd)Ufa | 329 | usAfsauaa(Tgn)g | 603 | UUGGUCUCUAUACUA | 877 |
| 392968 | UfAfCfuacauuauu | | uaguaUfaGfagacc | | CAUUAUUA | |
| | aL96 | | sasa | | | |
| AD- | uscsuau(Ahd)Cfu | 330 | asCfsauua(Agn)u | 604 | UCUCUAUACUACAUU | 878 |
| 392969 | AfCfAfuuauuaaug | | aauguAfgUfauaga | | AUUAAUGG | |
| | uL96 | | sgsa | | | |
| AD- | gsgsucu(Uhd)Cfa | 331 | asAfsuucu(Tgn)g | 605 | CUGGUCUUCAAUUAC | 879 |
| 392970 | AfUfUfaccaagaau | | guaauUfgAfagacc | | CAAGAAUU | |
| | uL96 | | sasg | | | |
| AD- | csasgga(Uhd)Afu | 332 | asAfsugau(Ggn)a | 606 | CUCAGGAUAUGAAGU | 880 |
| 392971 | GfAfAfguucaucau | | acuucAfuAfuccug | | UCAUCAUC | |
| | uL96 | | sasg | | | |
| AD- | ascsaca(Uhd)Cfa | 333 | usAfsgaau(Agn)c | 607 | CCACACAUCAGUAAU | 881 |
| 392972 | GfUfAfauguauucu | | auuacUfgAfugugu | | GUAUUCUA | |
| | aL96 | | sgsg | | | |
| AD- | csusaua(Chd)Ufa | 334 | asCfscauu(Agn)a | 608 | CUCUAUACUACAUUA | 882 |
| 392973 | CfAfUfuauuaaugg | | uaaugUfaGfuauag | | UUAAUGGG | |
| | uL96 | | sasg | | | |
| AD- | cscscgu(Uhd)Ufu | 335 | asUfsgagu(Agn)a | 609 | AACCCGUUUUAUGAU | 883 |
| 392974 | AfUfGfauuuacuca | | aucauAfaAfacggg | | UUACUCAU | |
| | uL96 | | susu | | | |
| AD- | ususcca(Uhd)Gfa | 336 | asAfsguaa(Agn)a | 610 | UUUUCCAUGACUGCA | 884 |
| 392975 | CfUfGfcauuuuacu | | ugcagUfcAfuggaa | | UUUUACUG | |
| | uL96 | | sasa | | | |
| AD- | uscsuuc(Ahd)Afu | 337 | asGfsaauu(Cgn)u | 611 | GGUCUUCAAUUACCA | 885 |
| 392976 | UfAfCfcaagaauuc | | ugguaAfuUfgaaga | | AGAAUUCU | |
| | uL96 | | scsc | | | |
| AD- | csusgaa(Ghd)Ufu | 338 | asUfsauca(Tgn)a | 612 | GUCUGAAGUUUCAUU | 886 |
| 392977 | UfCfAfuuuaugaua | | aaugaAfaCfuucag | | UAUGAUAC | |
| | uL96 | | sasc | | | |

TABLE 3
Exemplary Human APP-Targeting RNAi Agent Modified Duplexes, No “L96” Linker

| | Sense | SEQ | Antisense | SEQ | | SEQ |
|---|---|---|---|---|---|---|
| Duplex | Sequence | ID | Sequence | ID | mRNA target | ID |
| Name | (5′ to 3′) | NO | (5′ to 3′) | NO | sequence | NO |
| AD- | gsasccc(Ahd)Afu | 65 | asAfsagua(Ggn)g | 339 | GUGACCCAAUUAAGU | 613 |
| 392699 | UfAfAfguccuacuu | | acuuaAfuUfggguc | | CCUACUUU | |
| | u | | sasc | | | |
| AD- | uscsucc(Uhd)Gfa | 66 | asUfsguga(Tgn)a | 340 | UCUCUCCUGAUUAUU | 614 |
| 392700 | UfUfAfuuuaucaca | | aauaaUfcAfggaga | | UAUCACAU | |
| | u | | sgsa | | | |
| AD- | cscsuga(Ahd)Cfu | 67 | asUfsggau(Tgn)a | 341 | UGCCUGAACUUGAAU | 615 |
| 392703 | UfGfAfauuaaucca | | auucaAfgUfucagg | | UAAUCCAC | |
| | u | | scsa | | | |
| AD- | gsgsuuc(Ahd)Afa | 68 | asAfsuugc(Agn)c | 342 | UGGGUUCAAACAAAG | 616 |
| 392704 | CfAfAfaggugcaau | | cuuugUfuUfgaacc | | GUGCAAUC | |
| | u | | scsa | | | |
| AD- | ususuac(Uhd)Cfa | 69 | csAfsaaag(Ggn)c | 343 | GAUUUACUCAUUAUC | 617 |
| 392705 | UfUfAfucgccuuuu | | gauaaUfgAfguaaa | | GCCUUUUG | |
| | g | | susc | | | |
| AD- | asusuua(Ghd)Cfu | 70 | asCfsuagu(Tgn)u | 344 | GAAUUUAGCUGUAUC | 618 |
| 392707 | GfUfAfucaaacuag | | gauacAfgCfuaaau | | AAACUAGU | |
| | u | | susc | | | |
| AD- | asgsuau(Uhd)Cfc | 71 | asGfsugau(Cgn)a | 345 | UAAGUAUUCCUUUCC | 619 |
| 392708 | UfUfUfccugaucac | | ggaaaGfgAfauacu | | UGAUCACU | |
| | u | | susa | | | |
| AD- | gscsuua(Uhd)Gfa | 72 | gsAfsaagc(Ggn)a | 346 | UUGCUUAUGACAUGA | 620 |
| 392709 | CfAfUfgaucgcuuu | | ucaugUfcAfuaagc | | UCGCUUUC | |
| | c | | sasa | | | |
| AD- | asasgau(Ghd)Ufg | 73 | usAfscaaa(Tgn)u | 347 | UUAAGAUGUGUCUUC | 621 |
| 392710 | UfCfUfucaauuugu | | gaagaCfaCfaucuu | | AAUUUGUA | |
| | a | | sasa | | | |
| AD- | gscsaaa(Ahd)Cfc | 74 | asUfsagug(Agn)a | 348 | CAGCAAAACCAUUGC | 622 |
| 392711 | AfUfUfgcuucacua | | gcaauGfgUfuuugc | | UUCACUAC | |
| | u | | susg | | | |
| AD- | asusuua(Chd)Ufc | 75 | asAfsaagg(Cgn)g | 349 | UGAUUUACUCAUUAU | 623 |
| 392712 | AfUfUfaucgccuuu | | auaauGfaGfuaaau | | CGCCUUUU | |
| | u | | scsa | | | |
| AD- | usascuc(Ahd)Ufu | 76 | asUfscaaa(Agn)g | 350 | UUUACUCAUUAUCGC | 624 |
| 392713 | AfUfCfgccuuuuga | | gcgauAfaUfgagua | | CUUUUGAC | |
| | u | | sasa | | | |
| AD. | usgsccu(Ghd)Afa | 77 | asGfsauua(Agn)u | 351 | GAUGCCUGAACUUGA | 625 |
| 392714 | CfUfUfgaauuaauc | | ucaagUfuCfaggca | | AUUAAUCC | |
| | u | | susc | | | |
| AD- | csusgaa(Chd)Ufu | 78 | usGfsugga(Tgn)u | 352 | GCCUGAACUUGAAUU | 626 |
| 392715 | GfAfAfuuaauccac | | aauucAfaGfuucag | | AAUCCACA | |
| | a | | sgsc | | | |
| AD- | ususuag(Chd)Ufg | 79 | asAfscuag(Tgn)u | 353 | AAUUUAGCUGUAUCA | 627 |
| 392716 | UfAfUfcaaacuagu | | ugauaCfaGfcuaaa | | AACUAGUG | |
| | u | | susu | | | |
| AD- | gsasaua(Ghd)Afu | 80 | usAfsauca(Ggn)g | 354 | AUGAAUAGAUUCUCU | 628 |
| 392717 | UfCfUfcuccugauu | | agagaAfuCfuauuc | | CCUGAUUA | |
| | a | | sasu | | | |
| AD | uscscug(Ahd)Ufu | 81 | asUfsaugu(Ggn)a | 355 | UCUCCUGAUUAUUUA | 629 |
| 392718 | AfUfUfuaucacaua | | uaaauAfaUfcagga | | UCACAUAG | |
| | u | | sgsa | | | |
| AD- | cscscaa(Uhd)Ufa | 82 | asUfsaaag(Tgn)a | 356 | GACCCAAUUAAGUCC | 630 |
| 392719 | AfGfUfccuacuuua | | ggacuUfaAfuuggg | | UACUUUAC | |
| | u | | susc | | | |
| AD- | csasuau(Ghd)Cfu | 83 | asAfsucga(Tgn)u | 357 | UACAUAUGCUUUAAG | 631 |
| 392720 | UfUfAfagaaucgau | | cuuaaAfgCfauaug | | AAUCGAUG | |
| | u | | susa | | | |
| AD- | csusucu(Chd)Ufu | 84 | asGfsaaua(Cgn)u | 358 | UGCUUCUCUUGCCUA | 632 |
| 392721 | GfCfCfuaaguauuc | | uaggcAfaGfagaag | | AGUAUUCC | |
| | u | | scsa | | | |
| AD- | csasuug(Chd)Ufu | 85 | asCfsgauc(Agn)u | 359 | AUCAUUGCUUAUGAC | 633 |
| 392722 | AfUfGfacaugaucg | | gucauAfaGfcaaug | | AUGAUCGC | |
| | u | | sasu | | | |
| AD | csusuau(Ghd)Afc | 86 | asGfsaaag(Cgn)g | 360 | UGCUUAUGACAUGAU | 634 |
| 392723 | AfUfGfaucgcuuuc | | aucauGfuCfauaag | | CGCUUUCU | |
| | u | | scsa | | | |
| AD- | usasuga(Chd)Afu | 87 | asUfsagaa(Agn)g | 361 | CUUAUGACAUGAUCG | 635 |
| 392724 | GfAfUfcgcuuucua | | cgaucAfuGfucaua | | CUUUCUAC | |
| | u | | sasg | | | |
| AD | usgsaca(Uhd)Gfa | 88 | asUfsguag(Agn)a | 362 | UAUGACAUGAUCGCU | 636 |
| 392725 | UfCfGfcuuucuaca | | agcgaUfcAfuguca | | UUCUACAC | |
| | u | | susa | | | |
| AD- | gsasucg(Chd)Ufu | 89 | asAfsuaca(Ggn)u | 363 | AUGAUCGCUUUCUAC | 637 |
| 392726 | UfCfUfacacuguau | | guagaAfaGfcgauc | | ACUGUAUU | |
| | u | | sasu | | | |
| AD- | asasaac(Uhd)Afu | 90 | asGfsacgu(Cgn)a | 364 | GCAAAACUAUUCAGA | 638 |
| 392727 | UfCfAfgaugacguc | | ucugaAfuAfguuuu | | UGACGUCU | |
| | u | | sgsc | | | |
| AD | asasacu(Ahd)Ufu | 91 | asAfsgacg(Tgn)c | 365 | CAAAACUAUUCAGAU | 639 |
| 392728 | CfAfGfaugacgucu | | aucugAfaUfaguuu | | GACGUCUU | |
| | u | | susg | | | |
| AD- | ascsgaa(Ahd)Afu | 92 | asCfsuugu(Agn)g | 366 | CUACGAAAAUCCAAC | 640 |
| 392729 | CfCfAfaccuacaag | | guuggAfuUfuucgu | | CUACAAGU | |
| | u | | sasg | | | |
| AD- | usgscuu(Chd)Ufc | 93 | asAfsuacu(Tgn)a | 367 | GCUGCUUCUCUUGCC | 641 |
| 392730 | UfUfGfccuaaguau | | ggcaaGfaGfaagca | | UAAGUAUU | |
| | u | | sgsc | | | |
| AD- | usgscuu(Ahd)Ufg | 94 | asAfsagcg(Agn)u | 368 | AUUGCUUAUGACAUG | 642 |
| 392731 | AfCfAfugaucgcuu | | cauguCfaUfaagca | | AUCGCUUU | |
| | u | | sasu | | | |
| AD- | usgsauc(Ghd)Cfu | 95 | asUfsacag(Tgn)g | 369 | CAUGAUCGCUUUCUA | 643 |
| 392732 | UfUfCfuacacugua | | uagaaAfgCfgauca | | CACUGUAU | |
| | u | | susg | | | |
| AD- | asuscgc(Uhd)Ufu | 96 | usAfsauac(Agn)g | 370 | UGAUCGCUUUCUACA | 644 |
| 392733 | CfUfAfcacuguauu | | uguagAfaAfgcgau | | CUGUAUUA | |
| | a | | scsa | | | |
| AD- | uscsuuu(Ghd)Afc | 97 | asGfsuuuu(Cgn)g | 371 | CAUCUUUGACCGAAA | 645 |
| 392734 | CfGfAfaacgaaaac | | uuucgGfuCfaaaga | | CGAAAACC | |
| | u | | susg | | | |
| AD- | gsusucu(Ghd)Gfg | 98 | usGfsauau(Tgn)u | 372 | AGGUUCUGGGUUGAC | 646 |
| 392735 | UfUfGfacaaauauc | | gucaaCfcCfagaac | | AAAUAUCA | |
| | a | | scsu | | | |
| AD- | usgsggu(Uhd)Gfa | 99 | asUfscuug(Agn)u | 373 | UCUGGGUUGACAAAU | 647 |
| 392736 | CfAfAfauaucaaga | | auuugUfcAfaccca | | AUCAAGAC | |
| | u | | sgsa | | | |
| AD- | gsasuuu(Ahd)Cfu | 100 | asAfsaggc(Ggn)a | 374 | AUGAUUUACUCAUUA | 648 |
| 392737 | CfAfUfuaucgccuu | | uaaugAfgUfaaauc | | UCGCCUUU | |
| | u | | sasu | | | |
| AD- | uscscuu(Uhd)Cfc | 101 | usGfscaua(Ggn)u | 375 | AUUCCUUUCCUGAUC | 649 |
| 392738 | UfGfAfucacuaugc | | gaucaGfgAfaagga | | ACUAUGCA | |
| | a | | sasu | | | |
| AD- | csusuuc(Chd)Ufg | 102 | asAfsugca(Tgn)a | 376 | UCCUUUCCUGAUCAC | 650 |
| 392739 | AfUfCfacuaugcau | | gugauCfaGfgaaag | | UAUGCAUU | |
| | u | | sgsa | | | |
| AD- | asusugc(Uhd)Ufa | 103 | asGfscgau(Cgn)a | 377 | UCAUUGCUUAUGACA | 651 |
| 392740 | UfGfAfcaugaucgc | | ugucaUfaAfgcaau | | UGAUCGCU | |
| | u | | sgsa | | | |
| AD- | uscsuuu(Ahd)Afc | 104 | asAfsacuu(Cgn)a | 378 | UUUCUUUAACCAGUC | 652 |
| 392741 | CfAfGfucugaaguu | | gacugGfuUfaaaga | | UGAAGUUU | |
| | u | | sasa | | | |
| AD- | gsgsauc(Ahd)Gfu | 105 | asAfsucgu(Tgn)u | 379 | AAGGAUCAGUUACGG | 653 |
| 392742 | UfAfCfggaaacgau | | ccguaAfcUfgaucc | | AAACGAUG | |
| | u | | susu | | | |
| AD- | csusggg(Uhd)Ufg | 106 | usCfsuuga(Tgn)a | 380 | UUCUGGGUUGACAAA | 654 |
| 392743 | AfCfAfaauaucaag | | uuuguCfaAfcccag | | UAUCAAGA | |
| | a | | sasa | | | |
| AD- | asusgau(Uhd)Ufa | 107 | asGfsgcga(Tgn)a | 381 | UUAUGAUUUACUCAU | 655 |
| 392744 | CfUfCfauuaucgcc | | augagUfaAfaucau | | UAUCGCCU | |
| | u | | sasa | | | |
| AD- | csusugu(Ghd)Gfu | 108 | asAfsuugg(Ggn)u | 382 | UUCUUGUGGUUUGUG | 656 |
| 392745 | UfUfGfugacccaau | | cacaaAfcCfacaag | | ACCCAAUU | |
| | u | | sasa | | | |
| AD- | asusaug(Chd)Ufu | 109 | asCfsaucg(Agn)u | 383 | ACAUAUGCUUUAAGA | 657 |
| 392746 | UfAfAfgaaucgaug | | ucuuaAfaGfcauau | | AUCGAUGG | |
| | u | | sgsu | | | |
| AD- | ususugu(Chd)Cfa | 110 | asCfsccaa(Agn)g | 384 | UUUUUGUCCACGUAU | 658 |
| 392747 | CfGfUfaucuuuggg | | auacgUfgGfacaaa | | CUUUGGGU | |
| | u | | sasa | | | |
| AD- | uscsauu(Ghd)Ufa | 111 | asCfsguaa(Agn)a | 385 | GUUCAUUGUAAGCAC | 659 |
| 392748 | AfGfCfacuuuuacg | | gugcuUfaCfaauga | | UUUUACGG | |
| | u | | sasc | | | |
| AD- | gsgscca(Ahd)Cfa | 112 | asGfsuuca(Cgn)u | 386 | UUGGCCAACAUGAUU | 660 |
| 392749 | UfGfAfuuagugaac | | aaucaUfgUfuggcc | | AGUGAACC | |
| | u | | sasa | | | |
| AD- | gsasuca(Ghd)Ufu | 113 | asCfsaucg(Tgn)u | 387 | AGGAUCAGUUACGGA | 661 |
| 392750 | AfCfGfgaaacgaug | | uccguAfaCfugauc | | AACGAUGC | |
| | u | | scsu | | | |
| AD. | usascgg(Ahd)Afa | 114 | asAfsugag(Agn)g | 388 | GUUACGGAAACGAUG | 662 |
| 392751 | CfGfAfugcucucau | | caucgUfuUfccgua | | CUCUCAUG | |
| | u | | sasc | | | |
| AD | usgsauu(Uhd)Afc | 115 | asAfsggcg(Agn)u | 389 | UAUGAUUUACUCAUU | 663 |
| 392752 | UfCfAfuuaucgccu | | aaugaGfuAfaauca | | AUCGCCUU | |
| | u | | susa | | | |
| AD- | gsusaga(Uhd)Gfc | 116 | asAfsuuca(Agn)g | 390 | AAGUAGAUGCCUGAA | 664 |
| 392753 | CfUfGfaacuugaau | | uucagGfcAfucuac | | CUUGAAUU | |
| | u | | susu | | | |
| AD- | ususgua(Uhd)Afu | 117 | asAfsccac(Agn)a | 391 | AGUUGUAUAUUAUUC | 665 |
| 392754 | UfAfUfucuuguggu | | gaauaAfuAfuacaa | | UUGUGGUU | |
| | u | | scsu | | | |
| AD- | asusugc(Uhd)Gfc | 118 | asAfsauau(Agn)g | 392 | AGAUUGCUGCUUCUG | 666 |
| 392755 | UfUfCfugcuauauu | | cagaaGfcAfgcaau | | CUAUAUUU | |
| | u | | scsu | | | |
| AD- | usgscua(Uhd)Afu | 119 | usCfscuau(Agn)u | 393 | UCUGCUAUAUUUGUG | 667 |
| 392756 | UfUfGfugauauagg | | cacaaAfuAfuagca | | AUAUAGGA | |
| | a | | sgsa | | | |
| AD- | ascsaca(Uhd)Ufa | 120 | asAfsgucu(Cgn)a | 394 | GCACACAUUAGGCAU | 668 |
| 392757 | GfGfCfauugagacu | | augccUfaAfugugu | | UGAGACUU | |
| | u | | sgsc | | | |
| AD- | asasgaa(Uhd)Cfc | 121 | usUfsacaa(Tgn)g | 395 | AAAAGAAUCCCUGUU | 669 |
| 392758 | CfUfGfuucauugua | | aacagGfgAfuucuu | | CAUUGUAA | |
| | a | | susu | | | |
| AD | csasuug(Uhd)Afa | 122 | asCfscgua(Agn)a | 396 | UUCAUUGUAAGCACU | 670 |
| 392759 | GfCfAfcuuuuacgg | | agugcUfuAfcaaug | | UUUACGGG | |
| | u | | sasa | | | |
| AD- | ususgcu(Uhd)Afu | 123 | asAfsgcga(Tgn)c | 397 | CAUUGCUUAUGACAU | 671 |
| 392760 | GfAfCfaugaucgcu | | augucAfuAfagcaa | | GAUCGCUU | |
| | u | | susg | | | |
| AD | csasagg(Ahd)Ufc | 124 | asGfsuuuc(Cgn)g | 398 | ACCAAGGAUCAGUUA | 672 |
| 392761 | AfGfUfuacggaaac | | uaacuGfaUfccuug | | CGGAAACG | |
| | u | | sgsu | | | |
| AD. | asgsguu(Chd)Ufg | 125 | asUfsauuu(Ggn)u | 399 | CCAGGUUCUGGGUUG | 673 |
| 392762 | GfGfUfugacaaaua | | caaccCfaGfaaccu | | ACAAAUAU | |
| | u | | sgsg | | | |
| AD- | asasgau(Ghd)Ufg | 126 | asUfsuugu(Tgn)u | 400 | AGAAGAUGUGGGUUC | 674 |
| 392763 | GfGfUfucaaacaaa | | gaaccCfaCfaucuu | | AAACAAAG | |
| | u | | scsu | | | |
| AD- | csusgaa(Ghd)Afa | 127 | usGfsugua(Cgn)u | 401 | UGCUGAAGAAGAAAC | 675 |
| 392764 | GfAfAfacaguacac | | guuucUfuCfuucag | | AGUACACA | |
| | a | | scsa | | | |
| AD- | asasguu(Ghd)Gfa | 128 | asAfsuggu(Tgn)u | 402 | UGAAGUUGGACAGCA | 676 |
| 392765 | CfAfGfcaaaaccau | | ugcugUfcCfaacuu | | AAACCAUU | |
| | u | | scsa | | | |
| AD- | asuscgg(Uhd)Gfu | 129 | asUfsucua(Tgn)a | 403 | CCAUCGGUGUCCAUU | 677 |
| 392766 | CfCfAfuuuauagaa | | aauggAfcAfccgau | | UAUAGAAU | |
| | u | | sgsg | | | |
| AD- | uscsggu(Ghd)Ufc | 130 | usAfsuucu(Agn)u | 404 | CAUCGGUGUCCAUUU | 678 |
| 392767 | CfAfUfuuauagaau | | aaaugGfaCfaccga | | AUAGAAUA | |
| | a | | susg | | | |
| AD- | gscsugu(Ahd)Afc | 131 | asGfscauc(Tgn)a | 405 | GUGCUGUAACACAAG | 679 |
| 392768 | AfCfAfaguagaugc | | cuuguGfuUfacagc | | UAGAUGCC | |
| | u | | sasc | | | |
| AD- | asasgua(Ghd)Afu | 132 | usUfscaag(Tgn)u | 406 | ACAAGUAGAUGCCUG | 680 |
| 392769 | GfCfCfugaacuuga | | caggcAfuCfuacuu | | AACUUGAA | |
| | a | | sgsu | | | |
| AD- | ususgug(Ghd)Ufu | 133 | usAfsauug(Ggn)g | 407 | UCUUGUGGUUUGUGA | 681 |
| 392770 | UfGfUfgacccaauu | | ucacaAfaCfcacaa | | CCCAAUUA | |
| | a | | sgsa | | | |
| AD | gsusuug(Uhd)Gfa | 134 | asGfsacuu(Agn)a | 408 | UGGUUUGUGACCCAA | 682 |
| 392771 | CfCfCfaauuaaguc | | uugggUfcAfcaaac | | UUAAGUCC | |
| | u | | scsa | | | |
| AD- | gsusgac(Chd)Cfa | 135 | asGfsuagg(Agn)c | 409 | UUGUGACCCAAUUAA | 683 |
| 392772 | AfUfUfaaguccuac | | uuaauUfgGfgucac | | GUCCUACU | |
| | u | | sasa | | | |
| AD- | usasugc(Uhd)Ufu | 136 | asCfscauc(Ggn)a | 410 | CAUAUGCUUUAAGAA | 684 |
| 392773 | AfAfGfaaucgaugg | | uucuuAfaAfgcaua | | UCGAUGGG | |
| | u | | susg | | | |
| AD- | ususugu(Ghd)Afu | 137 | usCfsuuaa(Tgn)u | 411 | UAUUUGUGAUAUAGG | 685 |
| 392774 | AfUfAfggaauuaag | | ccuauAfuCfacaaa | | AAUUAAGA | |
| | a | | susa | | | |
| AD- | asasaga(Ahd)Ufc | 138 | usAfscaau(Ggn)a | 412 | GAAAAGAAUCCCUGU | 686 |
| 392775 | CfCfUfguucauugu | | acaggGfaUfucuuu | | UCAUUGUA | |
| | a | | susc | | | |
| AD- | usgsauu(Ghd)Ufa | 139 | asGfscaau(Ggn)a | 413 | GAUGAUUGUACAGAA | 687 |
| 392776 | CfAfGfaaucauugc | | uucugUfaCfaauca | | UCAUUGCU | |
| | u | | susc | | | |
| AD- | usgsccu(Ghd)Gfa | 140 | asAfsagaa(Ggn)g | 414 | CAUGCCUGGACAAAC | 688 |
| 392777 | CfAfAfacccuucuu | | guuugUfcCfaggca | | CCUUCUUU | |
| | u | | susg | | | |
| AD- | gsasgca(Ahd)Afa | 141 | asUfscauc(Tgn)g | 415 | AAGAGCAAAACUAUU | 689 |
| 392778 | CfUfAfuucagauga | | aauagUfuUfugcuc | | CAGAUGAC | |
| | u | | susu | | | |
| AD- | asgsuga(Ahd)Cfc | 142 | asUfsaacu(Ggn)a | 416 | UUAGUGAACCAAGGA | 690 |
| 392779 | AfAfGfgaucaguua | | uccuuGfgUfucacu | | UCAGUUAC | |
| | u | | sasa | | | |
| AD- | usgsaac(Chd)Afa | 143 | asCfsguaa(Cgn)u | 417 | AGUGAACCAAGGAUC | 691 |
| 392780 | GfGfAfucaguuacg | | gauccUfuGfguuca | | AGUUACGG | |
| | u | | scsu | | | |
| AD- | csasguu(Ahd)Cfg | 144 | asGfsagca(Tgn)c | 418 | AUCAGUUACGGAAAC | 692 |
| 392781 | GfAfAfacgaugcuc | | guuucCfgUfaacug | | GAUGCUCU | |
| | u | | sasu | | | |
| AD- | asgsaag(Ahd)Ufg | 145 | usUfsguuu(Ggn)a | 419 | GCAGAAGAUGUGGGU | 693 |
| 392782 | UfGfGfguucaaaca | | acccaCfaUfcuucu | | UCAAACAA | |
| | a | | sgsc | | | |
| AD- | cscsucu(Ghd)Afa | 146 | usUfsugcu(Ggn)u | 420 | AGCCUCUGAAGUUGG | 694 |
| 392783 | GfUfUfggacagcaa | | ccaacUfuCfagagg | | ACAGCAAA | |
| | a | | scsu | | | |
| AD- | ususaug(Ahd)Ufu | 147 | asCfsgaua(Agn)u | 421 | UUUUAUGAUUUACUC | 695 |
| 392784 | UfAfCfucauuaucg | | gaguaAfaUfcauaa | | AUUAUCGC | |
| | u | | sasa | | | |
| AD- | ascsagc(Uhd)Gfu | 148 | asUfsugug(Tgn)u | 422 | UGACAGCUGUGCUGU | 696 |
| 392785 | GfCfUfguaacacaa | | acagcAfcAfgcugu | | AACACAAG | |
| | u | | scsa | | | |
| AD- | usgsuga(Chd)Cfc | 149 | asUfsagga(Cgn)u | 423 | UUUGUGACCCAAUUA | 697 |
| 392786 | AfAfUfuaaguccua | | uaauuGfgGfucaca | | AGUCCUAC | |
| | u | | sasa | | | |
| AD- | usascau(Ahd)Ufg | 150 | usCfsgauu(Cgn)u | 424 | UUUACAUAUGCUUUA | 698 |
| 392787 | CfUfUfuaagaaucg | | uaaagCfaUfaugua | | AGAAUCGA | |
| | a | | sasa | | | |
| AD- | gsusaaa(Uhd)Afa | 151 | usCfscaag(Agn)a | 425 | AUGUAAAUAAAUACA | 699 |
| 392788 | AfUfAfcauucuugg | | uguauUfuAfuuuac | | UUCUUGGA | |
| | a | | sasu | | | |
| AD- | uscsagu(Uhd)Afc | 152 | asAfsgcau(Cgn)g | 426 | GAUCAGUUACGGAAA | 700 |
| 392789 | GfGfAfaacgaugcu | | uuuccGfuAfacuga | | CGAUGCUC | |
| | u | | susc | | | |
| AD- | csusucc(Chd)Gfu | 153 | asAfsacuc(Tgn)c | 427 | UCCUUCCCGUGAAUG | 701 |
| 392790 | GfAfAfuggagaguu | | cauucAfcGfggaag | | GAGAGUUC | |
| | u | | sgsa | | | |
| AD- | asgsuug(Ghd)Afc | 154 | asAfsaugg(Tgn)u | 428 | GAAGUUGGACAGCAA | 702 |
| 392791 | AfGfCfaaaaccauu | | uugcuGfuCfcaacu | | AACCAUUG | |
| | u | | susc | | | |
| AD- | cscscau(Chd)Gfg | 155 | asUfsauaa(Agn)u | 429 | UACCCAUCGGUGUCC | 703 |
| 392792 | UfGfUfccauuuaua | | ggacaCfcGfauggg | | AUUUAUAG | |
| | u | | susa | | | |
| AD- | usgscac(Ahd)Cfa | 156 | usCfsucaa(Tgn)g | 430 | UGUGCACACAUUAGG | 704 |
| 392793 | UfUfAfggcauugag | | ccuaaUfgUfgugca | | CAUUGAGA | |
| | a | | scsa | | | |
| AD- | cscsaac(Ahd)Ufg | 157 | usUfsgguu(Cgn)a | 431 | GGCCAACAUGAUUAG | 705 |
| 392794 | AfUfUfagugaacca | | cuaauCfaUfguugg | | UGAACCAA | |
| | a | | scsc | | | |
| AD- | asusgau(Uhd)Afg | 158 | asAfsuccu(Tgn)g | 432 | ACAUGAUUAGUGAAC | 706 |
| 392795 | UfGfAfaccaaggau | | guucaCfuAfaucau | | CAAGGAUC | |
| | u | | sgsu | | | |
| AD- | ususagu(Ghd)Afa | 159 | asAfscuga(Tgn)c | 433 | GAUUAGUGAACCAAG | 707 |
| 392796 | CfCfAfaggaucagu | | cuuggUfuCfacuaa | | GAUCAGUU | |
| | u | | susc | | | |
| AD- | asascca(Ahd)Gfg | 160 | usUfsccgu(Agn)a | 434 | UGAACCAAGGAUCAG | 708 |
| 392797 | AfUfCfaguuacgga | | cugauCfcUfugguu | | UUACGGAA | |
| | a | | scsa | | | |
| AD- | gsusuac(Ghd)Gfa | 161 | usGfsagag(Cgn)a | 435 | CAGUUACGGAAACGA | 709 |
| 392798 | AfAfCfgaugcucuc | | ucguuUfcCfguaac | | UGCUCUCA | |
| | a | | susg | | | |
| AD- | gsasugc(Ahd)Gfa | 162 | asUfscaug(Tgn)c | 436 | UGGAUGCAGAAUUCC | 710 |
| 392799 | AfUfUfccgacauga | | ggaauUfcUfgcauc | | GACAUGAC | |
| | u | | scsa | | | |
| AD- | ususgga(Chd)Afg | 163 | asGfscaau(Ggn)g | 437 | AGUUGGACAGCAAAA | 711 |
| 392800 | CfAfAfaaccauugc | | uuuugCfuGfuccaa | | CCAUUGCU | |
| | u | | scsu | | | |
| AD- | asasacc(Ahd)Ufu | 164 | usGfsggua(Ggn)u | 438 | CAAAACCAUUGCUUC | 712 |
| 392801 | GfCfUfucacuaccc | | gaagcAfaUfgguuu | | ACUACCCA | |
| | a | | susg | | | |
| AD- | cscsauc(Ghd)Gfu | 165 | usCfsuaua(Agn)a | 439 | ACCCAUCGGUGUCCA | 713 |
| 392802 | GfUfCfcauuuauag | | uggacAfcCfgaugg | | UUUAUAGA | |
| | a | | sgsu | | | |
| AD- | ususauc(Ghd)Cfc | 166 | asCfsagcu(Ggn)u | 440 | CAUUAUCGCCUUUUG | 714 |
| 392803 | UfUfUfugacagcug | | caaaaGfgCfgauaa | | ACAGCUGU | |
| | u | | susg | | | |
| AD | asuscgc(Chd)Ufu | 167 | asCfsacag(Cgn)u | 441 | UUAUCGCCUUUUGAC | 715 |
| 392804 | UfUfGfacagcugug | | gucaaAfaGfgcgau | | AGCUGUGC | |
| | u | | sasa | | | |
| AD- | ascsaca(Ahd)Gfu | 168 | asGfsuuca(Ggn)g | 442 | UAACACAAGUAGAUG | 716 |
| 392805 | AfGfAfugccugaac | | caucuAfcUfugugu | | CCUGAACU | |
| | u | | susa | | | |
| AD- | usgsugg(Uhd)Ufu | 169 | usUfsaauu(Ggn)g | 443 | CUUGUGGUUUGUGAC | 717 |
| 392806 | GfUfGfacccaauua | | gucacAfaAfccaca | | CCAAUUAA | |
| | a | | sasg | | | |
| AD- | gsgsgau(Ghd)Cfu | 170 | asAfscguu(Cgn)a | 444 | GGGGGAUGCUUCAUG | 718 |
| 392807 | UfCfAfugugaacgu | | caugaAfgCfauccc | | UGAACGUG | |
| | u | | scsc | | | |
| AD | usgsugc(Ahd)Cfa | 171 | usCfsaaug(Cgn)c | 445 | UAUGUGCACACAUUA | 719 |
| 392808 | CfAfUfuaggcauug | | uaaugUfgUfgcaca | | GGCAUUGA | |
| | a | | susa | | | |
| AD- | asasaug(Ghd)Afa | 172 | asUfsuaua(Tgn)u | 446 | GAAAAUGGAAGUGGC | 720 |
| 392809 | GfUfGfgcaauauaa | | gccacUfuCfcauuu | | AAUAUAAG | |
| | u | | susc | | | |
| AD- | asusgga(Ahd)Gfu | 173 | asCfscuua(Tgn)a | 447 | AAAUGGAAGUGGCAA | 721 |
| 392810 | GfGfCfaauauaagg | | uugccAfcUfuccau | | UAUAAGGG | |
| | u | | susu | | | |
| AD- | usgsccc(Ghd)Afg | 174 | asGfsuuua(Agn)c | 448 | CUUGCCCGAGAUCCU | 722 |
| 392811 | AfUfCfcuguuaaac | | aggauCfuCfgggca | | GUUAAACU | |
| | u | | sasg | | | |
| AD- | asusuag(Uhd)Gfa | 175 | asCfsugau(Cgn)c | 449 | UGAUUAGUGAACCAA | 723 |
| 392812 | AfCfCfaaggaucag | | uugguUfcAfcuaau | | GGAUCAGU | |
| | u | | scsa | | | |
| AD- | gsasacc(Ahd)Afg | 176 | usCfscgua(Agn)c | 450 | GUGAACCAAGGAUCA | 724 |
| 392813 | GfAfUfcaguuacgg | | ugaucCfuUfgguuc | | GUUACGGA | |
| | a | | sasc | | | |
| AD- | asasgga(Uhd)Cfa | 177 | usCfsguuu(Cgn)c | 451 | CCAAGGAUCAGUUAC | 725 |
| 392814 | GfUfUfacggaaacg | | guaacUfgAfuccuu | | GGAAACGA | |
| | a | | sgsg | | | |
| AD- | csasaca(Chd)Afg | 178 | usCfsaacu(Tgn)c | 452 | GCCAACACAGAAAAC | 726 |
| 392815 | AfAfAfacgaaguug | | guuuuCfuGfuguug | | GAAGUUGA | |
| | a | | sgsc | | | |
| AD- | usgsggu(Uhd)Cfa | 179 | usUfsgcac(Cgn)u | 453 | UGUGGGUUCAAACAA | 727 |
| 392816 | AfAfCfaaaggugca | | uuguuUfgAfaccca | | AGGUGCAA | |
| | a | | scsa | | | |
| AD- | csasgug(Ahd)Ufc | 180 | asCfsaagg(Tgn)g | 454 | GACAGUGAUCGUCAU | 728 |
| 392817 | GfUfCfaucaccuug | | augacGfaUfcacug | | CACCUUGG | |
| | u | | susc | | | |
| AD- | ascscca(Uhd)Cfg | 181 | usAfsuaaa(Tgn)g | 455 | CUACCCAUCGGUGUC | 729 |
| 392818 | GfUfGfuccauuuau | | gacacCfgAfugggu | | CAUUUAUA | |
| | a | | sasg | | | |
| AD- | uscsuug(Uhd)Gfg | 182 | asUfsuggg(Tgn)c | 456 | AUUCUUGUGGUUUGU | 730 |
| 392819 | UfUfUfgugacccaa | | acaaaCfcAfcaaga | | GACCCAAU | |
| | u | | sasu | | | |
| AD- | ususugu(Ghd)Afc | 183 | asGfsgacu(Tgn)a | 457 | GGUUUGUGACCCAAU | 731 |
| 392820 | CfCfAfauuaagucc | | auuggGfuCfacaaa | | UAAGUCCU | |
| | u | | scsc | | | |
| AD- | ususgug(Ahd)Cfc | 184 | usAfsggac(Tgn)u | 458 | GUUUGUGACCCAAUU | 732 |
| 392821 | CfAfAfuuaaguccu | | aauugGfgUfcacaa | | AAGUCCUA | |
| | a | | sasc | | | |
| AD- | ususcag(Ahd)Ufg | 185 | usUfsggcc(Agn)a | 459 | UAUUCAGAUGACGUC | 733 |
| 392822 | AfCfGfucuuggcca | | gacguCfaUfcugaa | | UUGGCCAA | |
| | a | | susa | | | |
| AD- | asuscag(Uhd)Ufa | 186 | asGfscauc(Ggn)u | 460 | GGAUCAGUUACGGAA | 734 |
| 392823 | CfGfGfaaacgaugc | | uuccgUfaAfcugau | | ACGAUGCU | |
| | u | | scsc | | | |
| AD- | usgsgau(Ghd)Cfa | 187 | asAfsuguc(Ggn)g | 461 | GAUGGAUGCAGAAUU | 735 |
| 392824 | GfAfAfuuccgacau | | aauucUfgCfaucca | | CCGACAUG | |
| | u | | susc | | | |
| AD | gsuscca(Ahd)Gfa | 188 | asCfsguuc(Tgn)g | 462 | CUGUCCAAGAUGCAG | 736 |
| 392825 | UfGfCfagcagaacg | | cugcaUfcUfuggac | | CAGAACGG | |
| | u | | sasg | | | |
| AD- | usasccc(Ahd)Ufc | 189 | asUfsaaau(Ggn)g | 463 | ACUACCCAUCGGUGU | 737 |
| 392826 | GfGfUfguccauuua | | acaccGfaUfgggua | | CCAUUUAU | |
| | u | | sgsu | | | |
| AD- | ususuug(Ahd)Cfa | 190 | asUfsuaca(Ggn)c | 464 | CCUUUUGACAGCUGU | 738 |
| 392827 | GfCfUfgugcuguaa | | acagcUfgUfcaaaa | | GCUGUAAC | |
| | u | | sgsg | | | |
| AD- | ususgac(Ahd)Gfc | 191 | asUfsguua(Cgn)a | 465 | UUUUGACAGCUGUGC | 739 |
| 392828 | UfGfUfgcuguaaca | | gcacaGfcUfgucaa | | UGUAACAC | |
| | u | | sasa | | | |
| AD- | asgscug(Uhd)Gfc | 192 | usAfscuug(Tgn)g | 466 | ACAGCUGUGCUGUAA | 740 |
| 392829 | UfGfUfaacacaagu | | uuacaGfcAfcagcu | | CACAAGUA | |
| | a | | sgsu | | | |
| AD- | gsusuuu(Ahd)Ufg | 193 | asCfsuaau(Ggn)u | 467 | CUGUUUUAUGUGCAC | 741 |
| 392830 | UfGfCfacacauuag | | gugcaCfaUfaaaac | | ACAUUAGG | |
| | u | | sasg | | | |
| AD | ususcaa(Uhd)Ufa | 194 | asGfsagaa(Tgn)u | 468 | UCUUCAAUUACCAAG | 742 |
| 392831 | CfCfAfagaauucuc | | cuuggUfaAfuugaa | | AAUUCUCC | |
| | u | | sgsa | | | |
| AD- | csascac(Ahd)Ufc | 195 | asGfsaaua(Cgn)a | 469 | UCCACACAUCAGUAA | 743 |
| 392832 | AfGfUfaauguauuc | | uuacuGfaUfgugug | | UGUAUUCU | |
| | u | | sgsa | | | |
| AD- | usgsguc(Uhd)Cfu | 196 | asAfsuaau(Ggn)u | 470 | UUUGGUCUCUAUACU | 744 |
| 392833 | AfUfAfcuacauuau | | aguauAfgAfgacca | | ACAUUAUU | |
| | u | | sasa | | | |
| AD- | ascsccg(Uhd)Ufu | 197 | usGfsagua(Agn)a | 471 | AAACCCGUUUUAUGA | 745 |
| 392834 | UfAfUfgauuuacuc | | ucauaAfaAfcgggu | | UUUACUCA | |
| | a | | susu | | | |
| AD- | usascga(Ahd)Afa | 198 | asUfsugua(Ggn)g | 472 | GCUACGAAAAUCCAA | 746 |
| 392835 | UfCfCfaaccuacaa | | uuggaUfuUfucgua | | CCUACAAG | |
| | u | | sgsc | | | |
| AD- | uscscac(Ahd)Cfa | 199 | asAfsuaca(Tgn)u | 473 | AAUCCACACAUCAGU | 747 |
| 392836 | UfCfAfguaauguau | | acugaUfgUfgugga | | AAUGUAUU | |
| | u | | susu | | | |
| AD- | csusggu(Chd)Ufu | 200 | usUfscuug(Ggn)u | 474 | UGCUGGUCUUCAAUU | 748 |
| 392837 | CfAfAfuuaccaaga | | aauugAfaGfaccag | | ACCAAGAA | |
| | a | | scsa | | | |
| AD- | gscscau(Chd)Ufu | 201 | usUfscguu(Tgn)c | 475 | AUGCCAUCUUUGACC | 749 |
| 392838 | UfGfAfccgaaacga | | ggucaAfaGfauggc | | GAAACGAA | |
| | a | | sasu | | | |
| AD- | cscsauc(Uhd)Ufu | 202 | usUfsucgu(Tgn)u | 476 | UGCCAUCUUUGACCG | 750 |
| 392839 | GfAfCfcgaaacgaa | | cggucAfaAfgaugg | | AAACGAAA | |
| | a | | scsa | | | |
| AD- | csusacg(Ahd)Afa | 203 | usUfsguag(Ggn)u | 477 | GGCUACGAAAAUCCA | 751 |
| 392840 | AfUfCfcaaccuaca | | uggauUfuUfcguag | | ACCUACAA | |
| | a | | scsc | | | |
| AD- | asuscca(Chd)Afc | 204 | asUfsacau(Tgn)a | 478 | UAAUCCACACAUCAG | 752 |
| 392841 | AfUfCfaguaaugua | | cugauGfuGfuggau | | UAAUGUAU | |
| | u | | susa | | | |
| AD- | csasugc(Chd)Afu | 205 | asUfsuucg(Ggn)u | 479 | CUCAUGCCAUCUUUG | 753 |
| 392842 | CfUfUfugaccgaaa | | caaagAfuGfgcaug | | ACCGAAAC | |
| | u | | sasg | | | |
| AD- | gsgscua(Chd)Gfa | 206 | asUfsaggu(Tgn)g | 480 | ACGGCUACGAAAAUC | 754 |
| 392843 | AfAfAfuccaaccua | | gauuuUfcGfuagcc | | CAACCUAC | |
| | u | | sgsu | | | |
| AD- | uscsaug(Chd)Cfa | 207 | usUfsucgg(Tgn)c | 481 | UCUCAUGCCAUCUUU | 755 |
| 392844 | UfCfUfuugaccgaa | | aaagaUfgGfcauga | | GACCGAAA | |
| | a | | sgsa | | | |
| AD- | csasgua(Chd)Afc | 208 | asUfsgaug(Agn)a | 482 | AACAGUACACAUCCA | 756 |
| 392845 | AfUfCfcauucauca | | uggauGfuGfuacug | | UUCAUCAU | |
| | u | | susu | | | |
| AD- | asascgg(Chd)Ufa | 209 | asGfsuugg(Agn)u | 483 | AGAACGGCUACGAAA | 757 |
| 392846 | CfGfAfaaauccaac | | uuucgUfaGfccguu | | AUCCAACC | |
| | u | | scsu | | | |
| AD- | gsasagu(Uhd)Ufc | 210 | usUfsguau(Cgn)a | 484 | CUGAAGUUUCAUUUA | 758 |
| 392847 | AfUfUfuaugauaca | | uaaauGfaAfacuuc | | UGAUACAA | |
| | a | | sasg | | | |
| AD- | asusgcc(Ahd)Ufc | 211 | asGfsuuuc(Ggn)g | 485 | UCAUGCCAUCUUUGA | 759 |
| 392848 | UfUfUfgaccgaaac | | ucaaaGfaUfggcau | | CCGAAACG | |
| | u | | sgsa | | | |
| AD- | gsasacg(Ghd)Cfu | 212 | asUfsugga(Tgn)u | 486 | CAGAACGGCUACGAA | 760 |
| 392849 | AfCfGfaaaauccaa | | uucguAfgCfcguuc | | AAUCCAAC | |
| | u | | susg | | | |
| AD- | uscsuuc(Ghd)Ufg | 213 | asAfscaua(Agn)a | 487 | UUUCUUCGUGCCUGU | 761 |
| 392850 | CfCfUfguuuuaugu | | acaggCfaCfgaaga | | UUUAUGUG | |
| | u | | sasa | | | |
| AD- | ususgcc(Chd)Gfa | 214 | asUfsuuaa(Cgn)a | 488 | UCUUGCCCGAGAUCC | 762 |
| 392851 | GfAfUfccuguuaaa | | ggaucUfcGfggcaa | | UGUUAAAC | |
| | u | | sgsa | | | |
| AD- | csusucg(Uhd)Gfc | 215 | asCfsacau(Agn)a | 489 | UUCUUCGUGCCUGUU | 763 |
| 392852 | CfUfGfuuuuaugug | | aacagGfcAfcgaag | | UUAUGUGC | |
| | u | | sasa | | | |
| AD- | gscsgcc(Ahd)Ufg | 216 | asUfsaaac(Tgn)u | 490 | CAGCGCCAUGUCCCA | 764 |
| 392853 | UfCfCfcaaaguuua | | ugggaCfaUfggcgc | | AAGUUUAC | |
| | u | | susg | | | |
| AD- | gsuscau(Ahd)Gfc | 217 | asAfscgau(Cgn)a | 491 | UUGUCAUAGCGACAG | 765 |
| 392854 | GfAfCfagugaucgu | | cugucGfcUfaugac | | UGAUCGUC | |
| | u | | sasa | | | |
| AD- | gscsuac(Ghd)Afa | 218 | usGfsuagg(Tgn)u | 492 | CGGCUACGAAAAUCC | 766 |
| 392855 | AfAfUfccaaccuac | | ggauuUfuCfguagc | | AACCUACA | |
| | a | | scsg | | | |
| AD- | asusagc(Ghd)Afc | 219 | asAfsugac(Ggn)a | 493 | UCAUAGCGACAGUGA | 767 |
| 392856 | AfGfUfgaucgucau | | ucacuGfuCfgcuau | | UCGUCAUC | |
| | u | | sgsa | | | |
| AD- | csusugc(Chd)Cfg | 220 | usUfsuaac(Agn)g | 494 | CUCUUGCCCGAGAUC | 768 |
| 392857 | AfGfAfuccuguuaa | | gaucuCfgGfgcaag | | CUGUUAAA | |
| | a | | sasg | | | |
| AD- | csuscau(Ghd)Cfc | 221 | usUfscggu(Cgn)a | 495 | CUCUCAUGCCAUCUU | 769 |
| 392858 | AfUfCfuuugaccga | | aagauGfgCfaugag | | UGACCGAA | |
| | a | | sasg | | | |
| AD- | ascsggc(Uhd)Afc | 222 | asGfsguug(Ggn)a | 496 | GAACGGCUACGAAAA | 770 |
| 392859 | GfAfAfaauccaacc | | uuuucGfuAfgccgu | | UCCAACCU | |
| | u | | susc | | | |
| AD- | csasuca(Ahd)Afa | 223 | asAfsagaa(Cgn)a | 497 | AUCAUCAAAAAUUGG | 771 |
| 392860 | AfUfUfgguguucuu | | ccaauUfuUfugaug | | UGUUCUUU | |
| | u | | sasu | | | |
| AD | asuscca(Ahd)Cfc | 224 | csAfsaaga(Agn)c | 498 | AAAUCCAACCUACAA | 772 |
| 392861 | UfAfCfaaguucuuu | | uuguaGfgUfuggau | | GUUCUUUG | |
| | g | | susu | | | |
| AD- | csgscuu(Uhd)Cfu | 225 | usGfsuaau(Agn)c | 499 | AUCGCUUUCUACACU | 773 |
| 392862 | AfCfAfcuguauuac | | aguguAfgAfaagcg | | GUAUUACA | |
| | a | | sasu | | | |
| AD- | uscscaa(Chd)Cfu | 226 | usCfsaaag(Agn)a | 500 | AAUCCAACCUACAAG | 774 |
| 392863 | AfCfAfaguucuuug | | cuuguAfgGfuugga | | UUCUUUGA | |
| | a | | susu | | | |
| AD- | uscsucu(Chd)Ufu | 227 | asGfsacca(Agn)a | 501 | UAUCUCUCUUUACAU | 775 |
| 392864 | UfAfCfauuuugguc | | auguaAfaGfagaga | | UUUGGUCU | |
| | u | | susa | | | |
| AD- | csuscuc(Uhd)Ufu | 228 | asAfsgacc(Agn)a | 502 | AUCUCUCUUUACAUU | 776 |
| 392865 | AfCfAfuuuuggucu | | aauguAfaAfgagag | | UUGGUCUC | |
| | u | | sasu | | | |
| AD- | ususugu(Ghd)Ufa | 229 | asAfsauuc(Tgn)u | 503 | GUUUUGUGUACUGUA | 777 |
| 392866 | CfUfGfuaaagaauu | | uacagUfaCfacaaa | | AAGAAUUU | |
| | u | | sasc | | | |
| AD- | gsusgua(Chd)Ufg | 230 | asCfsuaaa(Tgn)u | 504 | UUGUGUACUGUAAAG | 778 |
| 392867 | UfAfAfagaauuuag | | cuuuaCfaGfuacac | | AAUUUAGC | |
| | u | | sasa | | | |
| AD- | ascscca(Ahd)Ufu | 231 | usAfsaagu(Agn)g | 505 | UGACCCAAUUAAGUC | 779 |
| 392868 | AfAfGfuccuacuuu | | gacuuAfaUfugggu | | CUACUUUA | |
| | a | | scsa | | | |
| AD- | uscscua(Chd)Ufu | 232 | usAfsaagc(Agn)u | 506 | AGUCCUACUUUACAU | 780 |
| 392869 | UfAfCfauaugcuuu | | auguaAfaGfuagga | | AUGCUUUA | |
| | a | | scsu | | | |
| AD- | cscsuac(Uhd)Ufu | 233 | usUfsaaag(Cgn)a | 507 | GUCCUACUUUACAUA | 781 |
| 392870 | AfCfAfuaugcuuua | | uauguAfaAfguagg | | UGCUUUAA | |
| | a | | sasc | | | |
| AD- | ususcua(Chd)Afc | 234 | usUfsuaug(Tgn)a | 508 | CUUUCUACACUGUAU | 782 |
| 392871 | UfGfUfauuacauaa | | auacaGfuGfuagaa | | UACAUAAA | |
| | a | | sasg | | | |
| AD- | uscsuac(Ahd)Cfu | 235 | asUfsuuau(Ggn)u | 509 | UUUCUACACUGUAUU | 783 |
| 392872 | GfUfAfuuacauaaa | | aauacAfgUfguaga | | ACAUAAAU | |
| | u | | sasa | | | |
| AD- | csusuuu(Ahd)Afg | 236 | asUfsugaa(Ggn)a | 510 | UUCUUUUAAGAUGUG | 784 |
| 392873 | AfUfGfugucuucaa | | cacauCfuUfaaaag | | UCUUCAAU | |
| | u | | sasa | | | |
| AD- | asusgug(Uhd)Cfu | 237 | usUfsauac(Agn)a | 511 | AGAUGUGUCUUCAAU | 785 |
| 392874 | UfCfAfauuuguaua | | auugaAfgAfcacau | | UUGUAUAA | |
| | a | | scsu | | | |
| AD- | asuscaa(Ahd)Afa | 238 | csAfsaaga(Agn)c | 512 | UCAUCAAAAAUUGGU | 786 |
| 392875 | UfUfGfguguucuuu | | accaaUfuUfuugau | | GUUCUUUG | |
| | g | | sgsa | | | |
| AD- | asasauc(Chd)Afa | 239 | asAfsgaac(Tgn)u | 513 | GAAAAUCCAACCUAC | 787 |
| 392876 | CfCfUfacaaguucu | | guaggUfuGfgauuu | | AAGUUCUU | |
| | u | | susc | | | |
| AD- | gsusacu(Ghd)Ufa | 240 | asAfsgcua(Agn)a | 514 | GUGUACUGUAAAGAA | 788 |
| 392877 | AfAfGfaauuuagcu | | uucuuUfaCfaguac | | UUUAGCUG | |
| | u | | sasc | | | |
| AD- | csusccu(Ghd)Afu | 241 | usAfsugug(Agn)u | 515 | CUCUCCUGAUUAUUU | 789 |
| 392878 | UfAfUfuuaucacau | | aaauaAfuCfaggag | | AUCACAUA | |
| | a | | sasg | | | |
| AD | gscscag(Uhd)Ufg | 242 | asAfsagaa(Tgn)a | 516 | UAGCCAGUUGUAUAU | 790 |
| 392879 | UfAfUfauuauucuu | | auauaCfaAfcuggc | | UAUUCUUG | |
| | u | | susa | | | |
| AD- | asasuua(Ahd)Gfu | 243 | usAfsugua(Agn)a | 517 | CCAAUUAAGUCCUAC | 791 |
| 392880 | CfCfUfacuuuacau | | guaggAfcUfuaauu | | UUUACAUA | |
| | a | | sgsg | | | |
| AD- | csusugc(Chd)Ufa | 244 | asGfsaaag(Ggn)a | 518 | CUCUUGCCUAAGUAU | 792 |
| 392881 | AfGfUfauuccuuuc | | auacuUfaGfgcaag | | UCCUUUCC | |
| | u | | sasg | | | |
| AD- | asusucc(Uhd)Ufu | 245 | asAfsuagu(Ggn)a | 519 | GUAUUCCUUUCCUGA | 793 |
| 392882 | CfCfUfgaucacuau | | ucaggAfaAfggaau | | UCACUAUG | |
| | u | | sasc | | | |
| AD- | ascsuau(Ghd)Cfa | 246 | usUfsuaac(Tgn)u | 520 | UCACUAUGCAUUUUA | 794 |
| 392883 | UfUfUfuaaaguuaa | | uaaaaUfgCfauagu | | AAGUUAAA | |
| | a | | sgsa | | | |
| AD- | usgsuuc(Ahd)Ufu | 247 | usAfsaaag(Tgn)g | 521 | CCUGUUCAUUGUAAG | 795 |
| 392884 | GfUfAfagcacuuuu | | cuuacAfaUfgaaca | | CACUUUUA | |
| | a | | sgsg | | | |
| AD- | asasuua(Chd)Cfa | 248 | usUfsugga(Ggn)a | 522 | UCAAUUACCAAGAAU | 796 |
| 392885 | AfGfAfauucuccaa | | auucuUfgGfuaauu | | UCUCCAAA | |
| | a | | sgsa | | | |
| AD- | ususacc(Ahd)Afg | 249 | asUfsuuug(Ggn)a | 523 | AAUUACCAAGAAUUC | 797 |
| 392886 | AfAfUfucuccaaaa | | gaauuCfuUfgguaa | | UCCAAAAC | |
| | u | | susu | | | |
| AD- | uscsauu(Ghd)Cfu | 250 | asGfsauca(Tgn)g | 524 | AAUCAUUGCUUAUGA | 798 |
| 392887 | UfAfUfgacaugauc | | ucauaAfgCfaauga | | CAUGAUCG | |
| | u | | susu | | | |
| AD- | ususuua(Ahd)Gfa | 251 | asAfsuuga(Agn)g | 525 | UCUUUUAAGAUGUGU | 799 |
| 392889 | UfGfUfgucuucaau | | acacaUfcUfuaaaa | | CUUCAAUU | |
| | u | | sgsa | | | |
| AD- | asusccu(Ghd)Ufu | 252 | usUfsguag(Ggn)a | 526 | AGAUCCUGUUAAACU | 800 |
| 392890 | AfAfAfcuuccuaca | | aguuuAfaCfaggau | | UCCUACAA | |
| | a | | scsu | | | |
| AD- | ascsuau(Uhd)Cfa | 253 | asCfsaaga(Cgn)g | 527 | AAACUAUUCAGAUGA | 801 |
| 392891 | GfAfUfgacgucuug | | ucaucUfgAfauagu | | CGUCUUGG | |
| | u | | susu | | | |
| AD- | gsusuca(Uhd)Cfa | 254 | asAfsccaa(Tgn)u | 528 | AAGUUCAUCAUCAAA | 802 |
| 392892 | UfCfAfaaaauuggu | | uuugaUfgAfugaac | | AAUUGGUG | |
| | u | | susu | | | |
| AD- | usasucu(Chd)Ufc | 255 | asCfscaaa(Agn)u | 529 | UCUAUCUCUCUUUAC | 803 |
| 392893 | UfUfUfacauuuugg | | guaaaGfaGfagaua | | AUUUUGGU | |
| | u | | sgsa | | | |
| AD- | asuscuc(Uhd)Cfu | 256 | asAfsccaa(Agn)a | 530 | CUAUCUCUCUUUACA | 804 |
| 392894 | UfUfAfcauuuuggu | | uguaaAfgAfgagau | | UUUUGGUC | |
| | u | | sasg | | | |
| AD- | usgsugu(Ahd)Cfu | 257 | asUfsaaau(Tgn)c | 531 | UUUGUGUACUGUAAA | 805 |
| 392895 | GfUfAfaagaauuua | | uuuacAfgUfacaca | | GAAUUUAG | |
| | u | | sasa | | | |
| AD- | csusacu(Uhd)Ufa | 258 | asUfsuaaa(Ggn)c | 532 | UCCUACUUUACAUAU | 806 |
| 392896 | CfAfUfaugcuuuaa | | auaugUfaAfaguag | | GCUUUAAG | |
| | u | | sgsa | | | |
| AD- | usgsccu(Ahd)Afg | 259 | asAfsggaa(Agn)g | 533 | CUUGCCUAAGUAUUC | 807 |
| 392897 | UfAfUfuccuuuccu | | gaauaCfuUfaggca | | CUUUCCUG | |
| | u | | sasg | | | |
| AD- | asasgua(Uhd)Ufc | 260 | asUfsgauc(Agn)g | 534 | CUAAGUAUUCCUUUC | 808 |
| 392898 | CfUfUfuccugauca | | gaaagGfaAfuacuu | | CUGAUCAC | |
| | u | | sasg | | | |
| AD- | gsusauu(Chd)Cfu | 261 | usAfsguga(Tgn)c | 535 | AAGUAUUCCUUUCCU | 809 |
| 392899 | UfUfCfcugaucacu | | aggaaAfgGfaauac | | GAUCACUA | |
| | a | | susu | | | |
| AD- | ususccu(Ghd)Afu | 262 | asAfsaaug(Cgn)a | 536 | CUUUCCUGAUCACUA | 810 |
| 392900 | CfAfCfuaugcauuu | | uagugAfuCfaggaa | | UGCAUUUU | |
| | u | | sasg | | | |
| AD- | csusgau(Chd)Afc | 263 | usUfsuaaa(Agn)u | 537 | UCCUGAUCACUAUGC | 811 |
| 392901 | UfAfUfgcauuuuaa | | gcauaGfuGfaucag | | AUUUUAAA | |
| | a | | sgsa | | | |
| AD- | csascgu(Ahd)Ufc | 264 | usCfsaaag(Agn)c | 538 | UCCACGUAUCUUUGG | 812 |
| 392902 | UfUfUfgggucuuug | | ccaaaGfaUfacgug | | GUCUUUGA | |
| | a | | sgsa | | | |
| AD- | usgsggu(Chd)Ufu | 265 | asUfsuuuc(Tgn)u | 539 | UUUGGGUCUUUGAUA | 813 |
| 392903 | UfGfAfuaaagaaaa | | uaucaAfaGfaccca | | AAGAAAAG | |
| | u | | sasa | | | |
| AD- | uscsaau(Uhd)Afc | 266 | usGfsgaga(Agn)u | 540 | CUUCAAUUACCAAGA | 814 |
| 392904 | CfAfAfgaauucucc | | ucuugGfuAfauuga | | AUUCUCCA | |
| | a | | sasg | | | |
| AD- | uscsgcu(Uhd)Ufc | 267 | asUfsaaua(Cgn)a | 541 | GAUCGCUUUCUACAC | 815 |
| 392906 | UfAfCfacuguauua | | guguaGfaAfagcga | | UGUAUUAC | |
| | u | | susc | | | |
| AD- | asusuuu(Chd)Ufu | 268 | usUfscaga(Cgn)u | 542 | CAAUUUUCUUUAACC | 816 |
| 392907 | UfAfAfccagucuga | | gguuaAfaGfaaaau | | AGUCUGAA | |
| | a | | susg | | | |
| AD- | csusuua(Ahd)Cfc | 269 | gsAfsaacu(Tgn)c | 543 | UUCUUUAACCAGUCU | 817 |
| 392908 | AfGfUfcugaaguuu | | agacuGfgUfuaaag | | GAAGUUUC | |
| | c | | sasa | | | |
| AD- | usasaga(Uhd)Gfu | 270 | asCfsaaau(Tgn)g | 544 | UUUAAGAUGUGUCUU | 818 |
| 392909 | GfUfCfuucaauuug | | aagacAfcAfucuua | | CAAUUUGU | |
| | u | | sasa | | | |
| AD- | gsasucc(Uhd)Gfu | 271 | usGfsuagg(Agn)a | 545 | GAGAUCCUGUUAAAC | 819 |
| 392910 | UfAfAfacuuccuac | | guuuaAfcAfggauc | | UUCCUACA | |
| | a | | susc | | | |
| AD- | csusgcu(Uhd)Cfa | 272 | asUfsuuug(Cgn)u | 546 | AGCUGCUUCAGAAAG | 820 |
| 392911 | GfAfAfagagcaaaa | | cuuucUfgAfagcag | | AGCAAAAC | |
| | u | | scsu | | | |
| AD- | csasgaa(Ahd)Gfa | 273 | usGfsaaua(Ggn)u | 547 | UUCAGAAAGAGCAAA | 821 |
| 392912 | GfCfAfaaacuauuc | | uuugcUfcUfuucug | | ACUAUUCA | |
| | a | | sasa | | | |
| AD- | usasuga(Ahd)Gfu | 274 | usUfsuuug(Agn)u | 548 | GAUAUGAAGUUCAUC | 822 |
| 392913 | UfCfAfucaucaaaa | | gaugaAfcUfucaua | | AUCAAAAA | |
| | a | | susc | | | |
| AD- | csasuca(Uhd)Cfa | 275 | asAfsacac(Cgn)a | 549 | UUCAUCAUCAAAAAU | 823 |
| 392914 | AfAfAfauugguguu | | auuuuUfgAfugaug | | UGGUGUUC | |
| | u | | sasa | | | |
| AD- | uscsaaa(Ahd)Afu | 276 | asCfsaaag(Agn)a | 550 | CAUCAAAAAUUGGUG | 824 |
| 392915 | UfGfGfuguucuuug | | caccaAfuUfuuuga | | UUCUUUGC | |
| | u | | susg | | | |
| AD- | asasaau(Chd)Cfa | 277 | asGfsaacu(Tgn)g | 551 | CGAAAAUCCAACCUA | 825 |
| 392916 | AfCfCfuacaaguuc | | uagguUfgGfauuuu | | CAAGUUCU | |
| | u | | scsg | | | |
| AD- | cscsaac(Chd)Ufa | 278 | asUfscaaa(Ggn)a | 552 | AUCCAACCUACAAGU | 826 |
| 392917 | CfAfAfguucuuuga | | acuugUfaGfguugg | | UCUUUGAG | |
| | u | | sasu | | | |
| AD- | ascsuca(Uhd)Ufa | 279 | usGfsucaa(Agn)a | 553 | UUACUCAUUAUCGCC | 827 |
| 392918 | UfCfGfccuuuugac | | ggcgaUfaAfugagu | | UUUUGACA | |
| | a | | sasa | | | |
| AD- | csuscau(Uhd)Afu | 280 | asUfsguca(Agn)a | 554 | UACUCAUUAUCGCCU | 828 |
| 392919 | CfGfCfcuuuugaca | | aggcgAfuAfaugag | | UUUGACAG | |
| | u | | susa | | | |
| AD- | usgsugc(Uhd)Gfu | 281 | asUfscuac(Tgn)u | 555 | GCUGUGCUGUAACAC | 829 |
| 392920 | AfAfCfacaaguaga | | guguuAfcAfgcaca | | AAGUAGAU | |
| | u | | sgsc | | | |
| AD- | gsusgcu(Ghd)Ufa | 282 | asAfsucua(Cgn)u | 556 | CUGUGCUGUAACACA | 830 |
| 392921 | AfCfAfcaaguagau | | uguguUfaCfagcac | | AGUAGAUG | |
| | u | | sasg | | | |
| AD- | uscsuuu(Ahd)Cfa | 283 | asUfsagag(Agn)c | 557 | UCUCUUUACAUUUUG | 831 |
| 392922 | UfUfUfuggucucua | | caaaaUfgUfaaaga | | GUCUCUAU | |
| | u | | sgsa | | | |
| AD- | asusggg(Uhd)Ufu | 284 | usUfsuaca(Ggn)u | 558 | UAAUGGGUUUUGUGU | 832 |
| 392923 | UfGfUfguacuguaa | | acacaAfaAfcccau | | ACUGUAAA | |
| | a | | susa | | | |
| AD- | ususgug(Uhd)Afc | 285 | usAfsaauu(Cgn)u | 559 | UUUUGUGUACUGUAA | 833 |
| 392924 | UfGfUfaaagaauuu | | uuacaGfuAfcacaa | | AGAAUUUA | |
| | a | | sasa | | | |
| AD- | gscsugu(Ahd)Ufc | 286 | asAfsugca(Cgn)u | 560 | UAGCUGUAUCAAACU | 834 |
| 392925 | AfAfAfcuagugcau | | aguuuGfaUfacagc | | AGUGCAUG | |
| | u | | susa | | | |
| AD- | csusagu(Ghd)Cfa | 287 | asAfsgaau(Cgn)u | 561 | AACUAGUGCAUGAAU | 835 |
| 392926 | UfGfAfauagauucu | | auucaUfgCfacuag | | AGAUUCUC | |
| | u | | susu | | | |
| AD- | usasgug(Chd)Afu | 288 | asGfsagaa(Tgn)c | 562 | ACUAGUGCAUGAAUA | 836 |
| 392927 | GfAfAfuagauucuc | | uauucAfuGfcacua | | GAUUCUCU | |
| | u | | sgsu | | | |
| AD- | csuscuc(Chd)Ufg | 289 | usGfsugau(Agn)a | 563 | UUCUCUCCUGAUUAU | 837 |
| 392928 | AfUfUfauuuaucac | | auaauCfaGfgagag | | UUAUCACA | |
| | a | | sasa | | | |
| AD- | cscsuga(Uhd)Ufa | 290 | asCfsuaug(Tgn)g | 564 | CUCCUGAUUAUUUAU | 838 |
| 392929 | UfUfUfaucacauag | | auaaaUfaAfucagg | | CACAUAGC | |
| | u | | sasg | | | |
| AD- | usasagu(Chd)Cfu | 291 | asCfsauau(Ggn)u | 565 | AUUAAGUCCUACUUU | 839 |
| 392930 | AfCfUfuuacauaug | | aaaguAfgGfacuua | | ACAUAUGC | |
| | u | | sasu | | | |
| AD- | asgsucc(Uhd)Afc | 292 | asAfsgcau(Agn)u | 566 | UAAGUCCUACUUUAC | 840 |
| 392931 | UfUfUfacauaugcu | | guaaaGfuAfggacu | | AUAUGCUU | |
| | u | | susa | | | |
| AD- | gsusccu(Ahd)Cfu | 293 | asAfsagca(Tgn)a | 567 | AAGUCCUACUUUACA | 841 |
| 392932 | UfUfAfcauaugcuu | | uguaaAfgUfaggac | | UAUGCUUU | |
| | u | | susu | | | |
| AD- | ususcuc(Uhd)Ufg | 294 | asGfsgaau(Agn)c | 568 | GCUUCUCUUGCCUAA | 842 |
| 392933 | CfCfUfaaguauucc | | uuaggCfaAfgagaa | | GUAUUCCU | |
| | u | | sgsc | | | |
| AD- | csuscuu(Ghd)Cfc | 295 | asAfsagga(Agn)u | 569 | UUCUCUUGCCUAAGU | 843 |
| 392934 | UfAfAguauuccuuu | | acuuaGfgCfaagag | | AUUCCUUU | |
| | | | sasa | | | |
| AD- | usasuuc(Chd)Ufu | 296 | asUfsagug(Agn)u | 570 | AGUAUUCCUUUCCUG | 844 |
| 392935 | UfCfCfugaucacua | | caggaAfaGfgaaua | | AUCACUAU | |
| | u | | scsu | | | |
| AD- | ususucc(Uhd)Gfa | 297 | asAfsaugc(Agn)u | 571 | CCUUUCCUGAUCACU | 845 |
| 392936 | UfCfAfcuaugcauu | | agugaUfcAfggaaa | | AUGCAUUU | |
| | u | | sgsg | | | |
| AD- | csascua(Uhd)Gfc | 298 | usUfsaacu(Tgn)u | 572 | AUCACUAUGCAUUUU | 846 |
| 392937 | AfUfUfuuaaaguua | | aaaauGfcAfuagug | | AAAGUUAA | |
| | a | | sasu | | | |
| AD- | csusgca(Uhd)Ufu | 299 | asAfsucug(Tgn)a | 573 | GACUGCAUUUUACUG | 847 |
| 392938 | UfAfCfuguacagau | | caguaAfaAfugcag | | UACAGAUU | |
| | u | | susc | | | |
| AD- | ususcug(Chd)Ufa | 300 | usAfsuauc(Agn)c | 574 | GCUUCUGCUAUAUUU | 848 |
| 392939 | UfAfUfuugugauau | | aaauaUfaGfcagaa | | GUGAUAUA | |
| | a | | sgsc | | | |
| AD- | uscsugc(Uhd)Afu | 301 | asUfsauau(Cgn)a | 575 | CUUCUGCUAUAUUUG | 849 |
| 392940 | AfUfUfugugauaua | | caaauAfuAfgcaga | | UGAUAUAG | |
| | u | | sasg | | | |
| AD- | ascsgua(Uhd)Cfu | 302 | asUfscaaa(Ggn)a | 576 | CCACGUAUCUUUGGG | 850 |
| 392941 | UfUfGfggucuuuga | | cccaaAfgAfuacgu | | UCUUUGAU | |
| | u | | sgsg | | | |
| AD- | uscsuuu(Ghd)Gfg | 303 | usCfsuuua(Tgn)c | 577 | UAUCUUUGGGUCUUU | 851 |
| 392942 | UfCfUfuugauaaag | | aaagaCfcCfaaaga | | GAUAAAGA | |
| | a | | susa | | | |
| AD- | csusuug(Ghd)Gfu | 304 | usUfscuuu(Agn)u | 578 | AUCUUUGGGUCUUUG | 852 |
| 392943 | CfUfUfugauaaaga | | caaagAfcCfcaaag | | AUAAAGAA | |
| | a | | sasu | | | |
| AD- | ususggg(Uhd)Cfu | 305 | usUfsuucu(Tgn)u | 579 | CUUUGGGUCUUUGAU | 853 |
| 392944 | UfUfGfauaaagaaa | | aucaaAfgAfcccaa | | AAAGAAAA | |
| | a | | sasg | | | |
| AD- | asgsaau(Chd)Cfc | 306 | asUfsuaca(Agn)u | 580 | AAAGAAUCCCUGUUC | 854 |
| 392945 | UfGfUfucauuguaa | | gaacaGfgGfauucu | | AUUGUAAG | |
| | u | | susu | | | |
| AD- | gsasauc(Chd)Cfu | 307 | asCfsuuac(Agn)a | 581 | AAGAAUCCCUGUUCA | 855 |
| 392946 | GfUfUfcauuguaag | | ugaacAfgGfgauuc | | UUGUAAGC | |
| | u | | susu | | | |
| AD- | gsusuca(Uhd)Ufg | 308 | asUfsaaaa(Ggn)u | 582 | CUGUUCAUUGUAAGC | 856 |
| 392947 | UfAfAfgcacuuuua | | gcuuaCfaAfugaac | | ACUUUUAC | |
| | u | | sasg | | | |
| AD- | ususaug(Ahd)Cfa | 309 | usAfsgaaa(Ggn)c | 583 | GCUUAUGACAUGAUC | 857 |
| 392948 | UfGfAfucgcuuucu | | gaucaUfgUfcauaa | | GCUUUCUA | |
| | a | | sgsc | | | |
| AD- | asusgac(Ahd)Ufg | 310 | usGfsuaga(Agn)a | 584 | UUAUGACAUGAUCGC | 858 |
| 392949 | AfUfCfgcuuucuac | | gcgauCfaUfgucau | | UUUCUACA | |
| | a | | sasa | | | |
| AD- | csasuga(Uhd)Cfg | 311 | asCfsagug(Tgn)a | 585 | GACAUGAUCGCUUUC | 859 |
| 392950 | CfUfUfucuacacug | | gaaagCfgAfucaug | | UACACUGU | |
| | u | | susc | | | |
| AD- | csusuuc(Uhd)Afc | 312 | usAfsugua(Agn)u | 586 | CGCUUUCUACACUGU | 860 |
| 392951 | AfCfUfguauuacau | | acaguGfuAfgaaag | | AUUACAUA | |
| | a | | scsg | | | |
| AD- | gsasuuc(Ahd)Afu | 313 | asUfsgguu(Agn)a | 587 | CAGAUUCAAUUUUCU | 861 |
| 392952 | UfUfUfcuuuaacca | | agaaaAfuUfgaauc | | UUAACCAG | |
| | u | | susg | | | |
| AD- | ususucu(Uhd)Ufa | 314 | asCfsuuca(Ggn)a | 588 | AUUUUCUUUAACCAG | 862 |
| 392953 | AfCfCfagucugaag | | cugguUfaAfagaaa | | UCUGAAGU | |
| | u | | sasu | | | |
| AD- | ususuaa(Ghd)Afu | 315 | asAfsauug(Agn)a | 589 | CUUUUAAGAUGUGUC | 863 |
| 392954 | GfUfGfucuucaauu | | gacacAfuCfuuaaa | | UUCAAUUU | |
| | u | | sasg | | | |
| AD- | ususaag(Ahd)Ufg | 316 | csAfsaauu(Ggn)a | 590 | UUUUAAGAUGUGUCU | 864 |
| 392955 | UfGfUfcuucaauuu | | agacaCfaUfcuuaa | | UCAAUUUG | |
| | g | | sasa | | | |
| AD- | asgsaug(Uhd)Gfu | 317 | asUfsacaa(Agn)u | 591 | UAAGAUGUGUCUUCA | 865 |
| 392956 | CfUfUfcaauuugua | | ugaagAfcAfcaucu | | AUUUGUAU | |
| | u | | susa | | | |
| AD- | usgsucu(Uhd)Cfa | 318 | asUfsuuua(Tgn)a | 592 | UGUGUCUUCAAUUUG | 866 |
| 392957 | AfUfUfuguauaaaa | | caaauUfgAfagaca | | UAUAAAAU | |
| | u | | scsa | | | |
| AD- | csusuca(Ahd)Ufu | 319 | asCfscauu(Tgn)u | 593 | GUCUUCAAUUUGUAU | 867 |
| 392958 | UfGfUfauaaaaugg | | auacaAfaUfugaag | | AAAAUGGU | |
| | u | | sasc | | | |
| AD- | asusggu(Ghd)Ufu | 320 | usUfsauuu(Agn)c | 594 | AAAUGGUGUUUUCAU | 868 |
| 392959 | UfUfCfauguaaaua | | augaaAfaCfaccau | | GUAAAUAA | |
| | a | | susu | | | |
| AD- | ususcuu(Uhd)Ufa | 321 | usGfsaaga(Cgn)a | 595 | CCUUCUUUUAAGAUG | 869 |
| 392960 | AfGfAfugugucuuc | | caucuUfaAfaagaa | | UGUCUUCA | |
| | a | | sgsg | | | |
| AD- | usgsuau(Uhd)Cfu | 322 | usGfsuaaa(Ggn)a | 596 | AAUGUAUUCUAUCUC | 870 |
| 392961 | AfUfCfucucuuuac | | gagauAfgAfauaca | | UCUUUACA | |
| | a | | susu | | | |
| AD- | gsuscuc(Uhd)Afu | 323 | usUfsaaua(Agn)u | 597 | UGGUCUCUAUACUAC | 871 |
| 392962 | AfCfUfacauuauua | | guaguAfuAfgagac | | AUUAUUAA | |
| | a | | scsa | | | |
| AD- | uscsucu(Ahd)Ufa | 324 | asUfsuaau(Agn)a | 598 | GGUCUCUAUACUACA | 872 |
| 392963 | CfUfAfcauuauuaa | | uguagUfaUfagaga | | UUAUUAAU | |
| | u | | scsc | | | |
| AD | csuscua(Uhd)Afc | 325 | asAfsuuaa(Tgn)a | 599 | GUCUCUAUACUACAU | 873 |
| 392964 | UfAfCfauuauuaau | | auguaGfuAfuagag | | UAUUAAUG | |
| | u | | sasc | | | |
| AD- | csusuca(Ahd)Ufu | 326 | asAfsgaau(Tgn)c | 600 | GUCUUCAAUUACCAA | 874 |
| 392965 | AfCfCfaagaauucu | | uugguAfaUfugaag | | GAAUUCUC | |
| | u | | sasc | | | |
| AD- | cscsaca(Chd)Afu | 327 | asAfsauac(Agn)u | 601 | AUCCACACAUCAGUA | 875 |
| 392966 | CfAfGfuaauguauu | | uacugAfuGfugugg | | AUGUAUUC | |
| | u | | sasu | | | |
| AD- | csusauc(Uhd)Cfu | 328 | asCfsaaaa(Tgn)g | 602 | UUCUAUCUCUCUUUA | 876 |
| 392967 | CfUfUfuacauuuug | | uaaagAfgAfgauag | | CAUUUUGG | |
| | u | | sasa | | | |
| AD- | gsgsucu(Chd)Ufa | 329 | usAfsauaa(Tgn)g | 603 | UUGGUCUCUAUACUA | 877 |
| 392968 | UfAfCfuacauuauu | | uaguaUfaGfagacc | | CAUUAUUA | |
| | a | | sasa | | | |
| AD- | uscsuau(Ahd)Cfu | 330 | asCfsauua(Agn)u | 604 | UCUCUAUACUACAUU | 878 |
| 392969 | AfCfAfuuauuaaug | | aauguAfgUfauaga | | AUUAAUGG | |
| | u | | sgsa | | | |
| AD- | gsgsucu(Uhd)Cfa | 331 | asAfsuucu(Tgn)g | 605 | CUGGUCUUCAAUUAC | 879 |
| 392970 | AfUfUfaccaagaau | | guaauUfgAfagacc | | CAAGAAUU | |
| | u | | sasg | | | |
| AD- | csasgga(Uhd)Afu | 332 | asAfsugau(Ggn)a | 606 | CUCAGGAUAUGAAGU | 880 |
| 392971 | GfAfAfguucaucau | | acuucAfuAfuccug | | UCAUCAUC | |
| | u | | sasg | | | |
| AD- | ascsaca(Uhd)Cfa | 333 | usAfsgaau(Agn)c | 607 | CCACACAUCAGUAAU | 881 |
| 392972 | GfUfAfauguauucu | | auuacUfgAfugugu | | GUAUUCUA | |
| | a | | sgsg | | | |
| AD- | csusaua(Chd)Ufa | 334 | asCfscauu(Agn)a | 608 | CUCUAUACUACAUUA | 882 |
| 392973 | CfAfUfuauuaaugg | | uaaugUfaGfuauag | | UUAAUGGG | |
| | u | | sasg | | | |
| AD- | cscscgu(Uhd)Ufu | 335 | asUfsgagu(Agn)a | 609 | AACCCGUUUUAUGAU | 883 |
| 392974 | AfUfGfauuuacuca | | aucauAfaAfacggg | | UUACUCAU | |
| | u | | susu | | | |
| AD- | ususcca(Uhd)Gfa | 336 | asAfsguaa(Agn)a | 610 | UUUUCCAUGACUGCA | 884 |
| 392975 | CfUfGfcauuuuacu | | ugcagUfcAfuggaa | | UUUUACUG | |
| | u | | sasa | | | |
| AD- | uscsuuc(Ahd)Afu | 337 | asGfsaauu(Cgn)u | 611 | GGUCUUCAAUUACCA | 885 |
| 392976 | UfAfCfcaagaauuc | | ugguaAfuUfgaaga | | AGAAUUCU | |
| | u | | scsc | | | |
| AD- | csusgaa(Ghd)Ufu | 338 | asUfsauca(Tgn)a | 612 | GUCUGAAGUUUCAUU | 886 |
| 392977 | UfCfAfuuuaugaua | | aaugaAfaCfuucag | | UAUGAUAC | |
| | u | | sasc | | | |

TABLE 4
Additional Exemplary Human APP-Targeting RNAi Duplexes, Unmodified Sequences
(Human NM_000484 Targeting)

| | | SEQ | | | SEQ | |
|---|---|---|---|---|---|---|
| Duplex | Sense Sequence | ID | Position in | Antisense Sequence | ID | Position in |
| Name | (5′ to 3′) | NO | NM_000484 | (5′ to 3′) | NO | NM_000484 |
| AD-392853 | GCGCCAUGUCCCAAAGUUUAU | 887 | 1228-1248 | AUAAACTUUGGGACAUGGCGCUG | 1161 | 1226-1248 |
| AD-392857 | CUUGCCCGAGAUCCUGUUAAA | 888 | 1269-1289 | UUUAACAGGAUCUCGGGCAAGAG | 1162 | 1267-1289 |
| AD-392851 | UUGCCCGAGAUCCUGUUAAAU | 889 | 1270-1290 | AUUUAACAGGAUCUCGGGCAAGA | 1163 | 1268-1290 |
| AD-392811 | UGCCCGAGAUCCUGUUAAACU | 890 | 1271-1291 | AGUUUAACAGGAUCUCGGGCAAG | 1164 | 1269-1291 |
| AD-392910 | GAUCCUGUUAAACUUCCUACA | 891 | 1278-1298 | UGUAGGAAGUUUAACAGGAUCUC | 1165 | 1276-1298 |
| AD-392890 | AUCCUGUUAAACUUCCUACAA | 892 | 1279-1299 | UUGUAGGAAGUUUAACAGGAUCU | 1166 | 1277-1299 |
| AD-392911 | CUGCUUCAGAAAGAGCAAAAU | 893 | 1893-1913 | AUUUUGCUCUUUCUGAAGCAGCU | 1167 | 1891-1913 |
| AD-392912 | CAGAAAGAGCAAAACUAUUCA | 894 | 1899-1919 | UGAAUAGUUUUGCUCUUUCUGAA | 1168 | 1897-1919 |
| AD-392778 | GAGCAAAACUAUUCAGAUGAU | 895 | 1905-1925 | AUCAUCTGAAUAGUUUUGCUCUU | 1169 | 1903-1925 |
| AD-392727 | AAAACUAUUCAGAUGACGUCU | 896 | 1909-1929 | AGACGUCAUCUGAAUAGUUUUGC | 1170 | 1907-1929 |
| AD-392728 | AAACUAUUCAGAUGACGUCUU | 897 | 1910-1930 | AAGACGTCAUCUGAAUAGUUUUG | 1171 | 1908-1930 |
| AD-392891 | ACUAUUCAGAUGACGUCUUGU | 898 | 1912-1932 | ACAAGACGUCAUCUGAAUAGUUU | 1172 | 1910-1932 |
| AD-392822 | UUCAGAUGACGUCUUGGCCAA | 899 | 1916-1936 | UUGGCCAAGACGUCAUCUGAAUA | 1173 | 1914-1936 |
| AD-392749 | GGCCAACAUGAUUAGUGAACU | 900 | 1931-1951 | AGUUCACUAAUCAUGUUGGCCAA | 1174 | 1929-1951 |
| AD-392794 | CCAACAUGAUUAGUGAACCAA | 901 | 1933-1953 | UUGGUUCACUAAUCAUGUUGGCC | 1175 | 1931-1953 |
| AD-392795 | AUGAUUAGUGAACCAAGGAUU | 902 | 1938-1958 | AAUCCUTGGUUCACUAAUCAUGU | 1176 | 1936-1958 |
| AD-392812 | AUUAGUGAACCAAGGAUCAGU | 903 | 1941-1961 | ACUGAUCCUUGGUUCACUAAUCA | 1177 | 1939-1961 |
| AD-392796 | UUAGUGAACCAAGGAUCAGUU | 904 | 1942-1962 | AACUGATCCUUGGUUCACUAAUC | 1178 | 1940-1962 |
| AD-392779 | AGUGAACCAAGGAUCAGUUAU | 905 | 1944-1964 | AUAACUGAUCCUUGGUUCACUAA | 1179 | 1942-1964 |
| AD-392780 | UGAACCAAGGAUCAGUUACGU | 906 | 1946-1966 | ACGUAACUGAUCCUUGGUUCACU | 1180 | 1944-1966 |
| AD-392813 | GAACCAAGGAUCAGUUACGGA | 907 | 1947-1967 | UCCGUAACUGAUCCUUGGUUCAC | 1181 | 1945-1967 |
| AD-392797 | AACCAAGGAUCAGUUACGGAA | 908 | 1948-1968 | UUCCGUAACUGAUCCUUGGUUCA | 1182 | 1946-1968 |
| AD-392761 | CAAGGAUCAGUUACGGAAACU | 909 | 1951-1971 | AGUUUCCGUAACUGAUCCUUGGU | 1183 | 1949-1971 |
| AD-392814 | AAGGAUCAGUUACGGAAACGA | 910 | 1952-1972 | UCGUUUCCGUAACUGAUCCUUGG | 1184 | 1950-1972 |
| AD-392742 | GGAUCAGUUACGGAAACGAUU | 911 | 1954-1974 | AAUCGUTUCCGUAACUGAUCCUU | 1185 | 1952-1974 |
| AD-392750 | GAUCAGUUACGGAAACGAUGU | 912 | 1955-1975 | ACAUCGTUUCCGUAACUGAUCCU | 1186 | 1953-1975 |
| AD-392823 | AUCAGUUACGGAAACGAUGCU | 913 | 1956-1976 | AGCAUCGUUUCCGUAACUGAUCC | 1187 | 1954-1976 |
| AD-392789 | UCAGUUACGGAAACGAUGCUU | 914 | 1957-1977 | AAGCAUCGUUUCCGUAACUGAUC | 1188 | 1955-1977 |
| AD-392781 | CAGUUACGGAAACGAUGCUCU | 915 | 1958-1978 | AGAGCATCGUUUCCGUAACUGAU | 1189 | 1956-1978 |
| AD-392798 | GUUACGGAAACGAUGCUCUCA | 916 | 1960-1980 | UGAGAGCAUCGUUUCCGUAACUG | 1190 | 1958-1980 |
| AD-392751 | UACGGAAACGAUGCUCUCAUU | 917 | 1962-1982 | AAUGAGAGCAUCGUUUCCGUAAC | 1191 | 1960-1982 |
| AD-392858 | CUCAUGCCAUCUUUGACCGAA | 918 | 1977-1997 | UUCGGUCAAAGAUGGCAUGAGAG | 1192 | 1975-1997 |
| AD-392844 | UCAUGCCAUCUUUGACCGAAA | 919 | 1978-1998 | UUUCGGTCAAAGAUGGCAUGAGA | 1193 | 1976-1998 |
| AD-392842 | CAUGCCAUCUUUGACCGAAAU | 920 | 1979-1999 | AUUUCGGUCAAAGAUGGCAUGAG | 1194 | 1977-1999 |
| AD-392848 | AUGCCAUCUUUGACCGAAACU | 921 | 1980-2000 | AGUUUCGGUCAAAGAUGGCAUGA | 1195 | 1978-2000 |
| AD-392838 | GCCAUCUUUGACCGAAACGAA | 922 | 1982-2002 | UUCGUUTCGGUCAAAGAUGGCAU | 1196 | 1980-2002 |
| AD-392839 | CCAUCUUUGACCGAAACGAAA | 923 | 1983-2003 | UUUCGUTUCGGUCAAAGAUGGCA | 1197 | 1981-2003 |
| AD-392734 | UCUUUGACCGAAACGAAAACU | 924 | 1986-2006 | AGUUUUCGUUUCGGUCAAAGAUG | 1198 | 1984-2006 |
| AD-392790 | CUUCCCGUGAAUGGAGAGUUU | 925 | 2019-2039 | AAACUCTCCAUUCACGGGAAGGA | 1199 | 2017-2039 |
| AD-392815 | CAACACAGAAAACGAAGUUGA | 926 | 2093-2113 | UCAACUTCGUUUUCUGUGUUGGC | 1200 | 2091-2113 |
| AD-392762 | AGGUUCUGGGUUGACAAAUAU | 927 | 2162-2182 | AUAUUUGUCAACCCAGAACCUGG | 1201 | 2160-2182 |
| AD-392735 | GUUCUGGGUUGACAAAUAUCA | 928 | 2164-2184 | UGAUAUTUGUCAACCCAGAACCU | 1202 | 2162-2184 |
| AD-392743 | CUGGGUUGACAAAUAUCAAGA | 929 | 2167-2187 | UCUUGATAUUUGUCAACCCAGAA | 1203 | 2165-2187 |
| AD-392736 | UGGGUUGACAAAUAUCAAGAU | 930 | 2168-2188 | AUCUUGAUAUUUGUCAACCCAGA | 1204 | 2166-2188 |
| AD-392824 | UGGAUGCAGAAUUCCGACAUU | 931 | 2212-2232 | AAUGUCGGAAUUCUGCAUCCAUC | 1205 | 2210-2232 |
| AD-392799 | GAUGCAGAAUUCCGACAUGAU | 932 | 2214-2234 | AUCAUGTCGGAAUUCUGCAUCCA | 1206 | 2212-2234 |
| AD-392971 | CAGGAUAUGAAGUUCAUCAUU | 933 | 2236-2256 | AAUGAUGAACUUCAUAUCCUGAG | 1207 | 2234-2256 |
| AD-392913 | UAUGAAGUUCAUCAUCAAAAA | 934 | 2241-2261 | UUUUUGAUGAUGAACUUCAUAUC | 1208 | 2239-2261 |
| AD-392892 | GUUCAUCAUCAAAAAUUGGUU | 935 | 2247-2267 | AACCAATUUUUGAUGAUGAACUU | 1209 | 2245-2267 |
| AD-392914 | CAUCAUCAAAAAUUGGUGUUU | 936 | 2250-2270 | AAACACCAAUUUUUGAUGAUGAA | 1210 | 2248-2270 |
| AD-392860 | CAUCAAAAAUUGGUGUUCUUU | 937 | 2253-2273 | AAAGAACACCAAUUUUUGAUGAU | 1211 | 2251-2273 |
| AD-392875 | AUCAAAAAUUGGUGUUCUUUG | 938 | 2254-2274 | CAAAGAACACCAAUUUUUGAUGA | 1212 | 2252-2274 |
| AD-392915 | UCAAAAAUUGGUGUUCUUUGU | 939 | 2255-2275 | ACAAAGAACACCAAUUUUUGAUG | 1213 | 2253-2275 |
| AD-392782 | AGAAGAUGUGGGUUCAAACAA | 940 | 2276-2296 | UUGUUUGAACCCACAUCUUCUGC | 1214 | 2274-2296 |
| AD-392763 | AAGAUGUGGGUUCAAACAAAU | 941 | 2278-2298 | AUUUGUTUGAACCCACAUCUUCU | 1215 | 2276-2298 |
| AD-392816 | UGGGUUCAAACAAAGGUGCAA | 942 | 2284-2304 | UUGCACCUUUGUUUGAACCCACA | 1216 | 2282-2304 |
| AD-392704 | GGUUCAAACAAAGGUGCAAUU | 943 | 2286-2306 | AAUUGCACCUUUGUUUGAACCCA | 1217 | 2284-2306 |
| AD-392854 | GUCAUAGCGACAGUGAUCGUU | 944 | 2331-2351 | AACGAUCACUGUCGCUAUGACAA | 1218 | 2329-2351 |
| AD-392856 | AUAGCGACAGUGAUCGUCAUU | 945 | 2334-2354 | AAUGACGAUCACUGUCGCUAUGA | 1219 | 2332-2354 |
| AD-392817 | CAGUGAUCGUCAUCACCUUGU | 946 | 2341-2361 | ACAAGGTGAUGACGAUCACUGUC | 1220 | 2339-2361 |
| AD-392764 | CUGAAGAAGAAACAGUACACA | 947 | 2367-2387 | UGUGUACUGUUUCUUCUUCAGCA | 1221 | 2365-2387 |
| AD-392845 | CAGUACACAUCCAUUCAUCAU | 948 | 2379-2399 | AUGAUGAAUGGAUGUGUACUGUU | 1222 | 2377-2399 |
| AD-392825 | GUCCAAGAUGCAGCAGAACGU | 949 | 2447-2467 | ACGUUCTGCUGCAUCUUGGACAG | 1223 | 2445-2467 |
| AD-392849 | GAACGGCUACGAAAAUCCAAU | 950 | 2462-2482 | AUUGGATUUUCGUAGCCGUUCUG | 1224 | 2460-2482 |
| AD-392846 | AACGGCUACGAAAAUCCAACU | 951 | 2463-2483 | AGUUGGAUUUUCGUAGCCGUUCU | 1225 | 2461-2483 |
| AD-392859 | ACGGCUACGAAAAUCCAACCU | 952 | 2464-2484 | AGGUUGGAUUUUCGUAGCCGUUC | 1226 | 2462-2484 |
| AD-392843 | GGCUACGAAAAUCCAACCUAU | 953 | 2466-2486 | AUAGGUTGGAUUUUCGUAGCCGU | 1227 | 2464-2486 |
| AD-392855 | GCUACGAAAAUCCAACCUACA | 954 | 2467-2487 | UGUAGGTUGGAUUUUCGUAGCCG | 1228 | 2465-2487 |
| AD-392840 | CUACGAAAAUCCAACCUACAA | 955 | 2468-2488 | UUGUAGGUUGGAUUUUCGUAGCC | 1229 | 2466-2488 |
| AD-392835 | UACGAAAAUCCAACCUACAAU | 956 | 2469-2489 | AUUGUAGGUUGGAUUUUCGUAGC | 1230 | 2467-2489 |
| AD-392729 | ACGAAAAUCCAACCUACAAGU | 957 | 2470-2490 | ACUUGUAGGUUGGAUUUUCGUAG | 1231 | 2468-2490 |
| AD-392916 | AAAAUCCAACCUACAAGUUCU | 958 | 2473-2493 | AGAACUTGUAGGUUGGAUUUUCG | 1232 | 2471-2493 |
| AD-392876 | AAAUCCAACCUACAAGUUCUU | 959 | 2474-2494 | AAGAACTUGUAGGUUGGAUUUUC | 1233 | 2472-2494 |
| AD-392861 | AUCCAACCUACAAGUUCUUUG | 960 | 2476-2496 | CAAAGAACUUGUAGGUUGGAUUU | 1234 | 2474-2496 |
| AD-392863 | UCCAACCUACAAGUUCUUUGA | 961 | 2477-2497 | UCAAAGAACUUGUAGGUUGGAUU | 1235 | 2475-2497 |
| AD-392917 | CCAACCUACAAGUUCUUUGAU | 962 | 2478-2498 | AUCAAAGAACUUGUAGGUUGGAU | 1236 | 2476-2498 |
| AD-392783 | CCUCUGAAGUUGGACAGCAAA | 963 | 2530-2550 | UUUGCUGUCCAACUUCAGAGGCU | 1237 | 2528-2550 |
| AD-392765 | AAGUUGGACAGCAAAACCAUU | 964 | 2536-2556 | AAUGGUTUUGCUGUCCAACUUCA | 1238 | 2534-2556 |
| AD-392791 | AGUUGGACAGCAAAACCAUUU | 965 | 2537-2557 | AAAUGGTUUUGCUGUCCAACUUC | 1239 | 2535-2557 |
| AD-392800 | UUGGACAGCAAAACCAUUGCU | 966 | 2539-2559 | AGCAAUGGUUUUGCUGUCCAACU | 1240 | 2537-2559 |
| AD-392711 | GCAAAACCAUUGCUUCACUAU | 967 | 2546-2566 | AUAGUGAAGCAAUGGUUUUGCUG | 1241 | 2544-2566 |
| AD-392801 | AAACCAUUGCUUCACUACCCA | 968 | 2549-2569 | UGGGUAGUGAAGCAAUGGUUUUG | 1242 | 2547-2569 |
| AD-392826 | UACCCAUCGGUGUCCAUUUAU | 969 | 2564-2584 | AUAAAUGGACACCGAUGGGUAGU | 1243 | 2562-2584 |
| AD-392818 | ACCCAUCGGUGUCCAUUUAUA | 970 | 2565-2585 | UAUAAATGGACACCGAUGGGUAG | 1244 | 2563-2585 |
| AD-392792 | CCCAUCGGUGUCCAUUUAUAU | 971 | 2566-2586 | AUAUAAAUGGACACCGAUGGGUA | 1245 | 2564-2586 |
| AD-392802 | CCAUCGGUGUCCAUUUAUAGA | 972 | 2567-2587 | UCUAUAAAUGGACACCGAUGGGU | 1246 | 2565-2587 |
| AD-392766 | AUCGGUGUCCAUUUAUAGAAU | 973 | 2569-2589 | AUUCUATAAAUGGACACCGAUGG | 1247 | 2567-2589 |
| AD-392767 | UCGGUGUCCAUUUAUAGAAUA | 974 | 2570-2590 | UAUUCUAUAAAUGGACACCGAUG | 1248 | 2568-2590 |
| AD-392834 | ACCCGUUUUAUGAUUUACUCA | 975 | 2607-2627 | UGAGUAAAUCAUAAAACGGGUUU | 1249 | 2605-2627 |
| AD-392974 | CCCGUUUUAUGAUUUACUCAU | 976 | 2608-2628 | AUGAGUAAAUCAUAAAACGGGUU | 1250 | 2606-2628 |
| AD-392784 | UUAUGAUUUACUCAUUAUCGU | 977 | 2614-2634 | ACGAUAAUGAGUAAAUCAUAAAA | 1251 | 2612-2634 |
| AD-392744 | AUGAUUUACUCAUUAUCGCCU | 978 | 2616-2636 | AGGCGATAAUGAGUAAAUCAUAA | 1252 | 2614-2636 |
| AD-392752 | UGAUUUACUCAUUAUCGCCUU | 979 | 2617-2637 | AAGGCGAUAAUGAGUAAAUCAUA | 1253 | 2615-2637 |
| AD-392737 | GAUUUACUCAUUAUCGCCUUU | 980 | 2618-2638 | AAAGGCGAUAAUGAGUAAAUCAU | 1254 | 2616-2638 |
| AD-392712 | AUUUACUCAUUAUCGCCUUUU | 981 | 2619-2639 | AAAAGGCGAUAAUGAGUAAAUCA | 1255 | 2617-2639 |
| AD-392705 | UUUACUCAUUAUCGCCUUUUG | 982 | 2620-2640 | CAAAAGGCGAUAAUGAGUAAAUC | 1256 | 2618-2640 |
| AD-392713 | UACUCAUUAUCGCCUUUUGAU | 983 | 2622-2642 | AUCAAAAGGCGAUAAUGAGUAAA | 1257 | 2620-2642 |
| AD-392918 | ACUCAUUAUCGCCUUUUGACA | 984 | 2623-2643 | UGUCAAAAGGCGAUAAUGAGUAA | 1258 | 2621-2643 |
| AD-392919 | CUCAUUAUCGCCUUUUGACAU | 985 | 2624-2644 | AUGUCAAAAGGCGAUAAUGAGUA | 1259 | 2622-2644 |
| AD-392803 | UUAUCGCCUUUUGACAGCUGU | 986 | 2628-2648 | ACAGCUGUCAAAAGGCGAUAAUG | 1260 | 2626-2648 |
| AD-392804 | AUCGCCUUUUGACAGCUGUGU | 987 | 2630-2650 | ACACAGCUGUCAAAAGGCGAUAA | 1261 | 2628-2650 |
| AD-392827 | UUUUGACAGCUGUGCUGUAAU | 988 | 2636-2656 | AUUACAGCACAGCUGUCAAAAGG | 1262 | 2634-2656 |
| AD-392828 | UUGACAGCUGUGCUGUAACAU | 989 | 2638-2658 | AUGUUACAGCACAGCUGUCAAAA | 1263 | 2636-2658 |
| AD-392785 | ACAGCUGUGCUGUAACACAAU | 990 | 2641-2661 | AUUGUGTUACAGCACAGCUGUCA | 1264 | 2639-2661 |
| AD-392829 | AGCUGUGCUGUAACACAAGUA | 991 | 2643-2663 | UACUUGTGUUACAGCACAGCUGU | 1265 | 2641-2663 |
| AD-392920 | UGUGCUGUAACACAAGUAGAU | 992 | 2646-2666 | AUCUACTUGUGUUACAGCACAGC | 1266 | 2644-2666 |
| AD-392921 | GUGCUGUAACACAAGUAGAUU | 993 | 2647-2667 | AAUCUACUUGUGUUACAGCACAG | 1267 | 2645-2667 |
| AD-392768 | GCUGUAACACAAGUAGAUGCU | 994 | 2649-2669 | AGCAUCTACUUGUGUUACAGCAC | 1268 | 2647-2669 |
| AD-392805 | ACACAAGUAGAUGCCUGAACU | 995 | 2655-2675 | AGUUCAGGCAUCUACUUGUGUUA | 1269 | 2653-2675 |
| AD-392769 | AAGUAGAUGCCUGAACUUGAA | 996 | 2659-2679 | UUCAAGTUCAGGCAUCUACUUGU | 1270 | 2657-2679 |
| AD-392753 | GUAGAUGCCUGAACUUGAAUU | 997 | 2661-2681 | AAUUCAAGUUCAGGCAUCUACUU | 1271 | 2659-2681 |
| AD-392714 | UGCCUGAACUUGAAUUAAUCU | 998 | 2666-2686 | AGAUUAAUUCAAGUUCAGGCAUC | 1272 | 2664-2686 |
| AD-392703 | CCUGAACUUGAAUUAAUCCAU | 999 | 2668-2688 | AUGGAUTAAUUCAAGUUCAGGCA | 1273 | 2666-2688 |
| AD-392715 | CUGAACUUGAAUUAAUCCACA | 1000 | 2669-2689 | UGUGGATUAAUUCAAGUUCAGGC | 1274 | 2667-2689 |
| AD-392841 | AUCCACACAUCAGUAAUGUAU | 1001 | 2683-2703 | AUACAUTACUGAUGUGUGGAUUA | 1275 | 2681-2703 |
| AD-392836 | UCCACACAUCAGUAAUGUAUU | 1002 | 2684-2704 | AAUACATUACUGAUGUGUGGAUU | 1276 | 2682-2704 |
| AD-392966 | CCACACAUCAGUAAUGUAUUU | 1003 | 2685-2705 | AAAUACAUUACUGAUGUGUGGAU | 1277 | 2683-2705 |
| AD-392832 | CACACAUCAGUAAUGUAUUCU | 1004 | 2686-2706 | AGAAUACAUUACUGAUGUGUGGA | 1278 | 2684-2706 |
| AD-392972 | ACACAUCAGUAAUGUAUUCUA | 1005 | 2687-2707 | UAGAAUACAUUACUGAUGUGUGG | 1279 | 2685-2707 |
| AD-392961 | UGUAUUCUAUCUCUCUUUACA | 1006 | 2699-2719 | UGUAAAGAGAGAUAGAAUACAUU | 1280 | 2697-2719 |
| AD-392967 | CUAUCUCUCUUUACAUUUUGU | 1007 | 2705-2725 | ACAAAATGUAAAGAGAGAUAGAA | 1281 | 2703-2725 |
| AD-392893 | UAUCUCUCUUUACAUUUUGGU | 1008 | 2706-2726 | ACCAAAAUGUAAAGAGAGAUAGA | 1282 | 2704-2726 |
| AD-392894 | AUCUCUCUUUACAUUUUGGUU | 1009 | 2707-2727 | AACCAAAAUGUAAAGAGAGAUAG | 1283 | 2705-2727 |
| AD-392864 | UCUCUCUUUACAUUUUGGUCU | 1010 | 2708-2728 | AGACCAAAAUGUAAAGAGAGAUA | 1284 | 2706-2728 |
| AD-392865 | CUCUCUUUACAUUUUGGUCUU | 1011 | 2709-2729 | AAGACCAAAAUGUAAAGAGAGAU | 1285 | 2707-2729 |
| AD-392922 | UCUUUACAUUUUGGUCUCUAU | 1012 | 2712-2732 | AUAGAGACCAAAAUGUAAAGAGA | 1286 | 2710-2732 |
| AD-392833 | UGGUCUCUAUACUACAUUAUU | 1013 | 2723-2743 | AAUAAUGUAGUAUAGAGACCAAA | 1287 | 2721-2743 |
| AD-392968 | GGUCUCUAUACUACAUUAUUA | 1014 | 2724-2744 | UAAUAATGUAGUAUAGAGACCAA | 1288 | 2722-2744 |
| AD-392962 | GUCUCUAUACUACAUUAUUAA | 1015 | 2725-2745 | UUAAUAAUGUAGUAUAGAGACCA | 1289 | 2723-2745 |
| AD-392963 | UCUCUAUACUACAUUAUUAAU | 1016 | 2726-2746 | AUUAAUAAUGUAGUAUAGAGACC | 1290 | 2724-2746 |
| AD-392964 | CUCUAUACUACAUUAUUAAUU | 1017 | 2727-2747 | AAUUAATAAUGUAGUAUAGAGAC | 1291 | 2725-2747 |
| AD-392969 | UCUAUACUACAUUAUUAAUGU | 1018 | 2728-2748 | ACAUUAAUAAUGUAGUAUAGAGA | 1292 | 2726-2748 |
| AD-392973 | CUAUACUACAUUAUUAAUGGU | 1019 | 2729-2749 | ACCAUUAAUAAUGUAGUAUAGAG | 1293 | 2727-2749 |
| AD-392923 | AUGGGUUUUGUGUACUGUAAA | 1020 | 2745-2765 | UUUACAGUACACAAAACCCAUUA | 1294 | 2743-2765 |
| AD-392866 | UUUGUGUACUGUAAAGAAUUU | 1021 | 2751-2771 | AAAUUCTUUACAGUACACAAAAC | 1295 | 2749-2771 |
| AD-392924 | UUGUGUACUGUAAAGAAUUUA | 1022 | 2752-2772 | UAAAUUCUUUACAGUACACAAAA | 1296 | 2750-2772 |
| AD-392895 | UGUGUACUGUAAAGAAUUUAU | 1023 | 2753-2773 | AUAAAUTCUUUACAGUACACAAA | 1297 | 2751-2773 |
| AD-392867 | GUGUACUGUAAAGAAUUUAGU | 1024 | 2754-2774 | ACUAAATUCUUUACAGUACACAA | 1298 | 2752-2774 |
| AD-392877 | GUACUGUAAAGAAUUUAGCUU | 1025 | 2756-2776 | AAGCUAAAUUCUUUACAGUACAC | 1299 | 2754-2776 |
| AD-392707 | AUUUAGCUGUAUCAAACUAGU | 1026 | 2768-2788 | ACUAGUTUGAUACAGCUAAAUUC | 1300 | 2766-2788 |
| AD-392716 | UUUAGCUGUAUCAAACUAGUU | 1027 | 2769-2789 | AACUAGTUUGAUACAGCUAAAUU | 1301 | 2767-2789 |
| AD-392925 | GCUGUAUCAAACUAGUGCAUU | 1028 | 2773-2793 | AAUGCACUAGUUUGAUACAGCUA | 1302 | 2771-2793 |
| AD-392926 | CUAGUGCAUGAAUAGAUUCUU | 1029 | 2784-2804 | AAGAAUCUAUUCAUGCACUAGUU | 1303 | 2782-2804 |
| AD-392927 | UAGUGCAUGAAUAGAUUCUCU | 1030 | 2785-2805 | AGAGAATCUAUUCAUGCACUAGU | 1304 | 2783-2805 |
| AD-392717 | GAAUAGAUUCUCUCCUGAUUA | 1031 | 2793-2813 | UAAUCAGGAGAGAAUCUAUUCAU | 1305 | 2791-2813 |
| AD-392928 | CUCUCCUGAUUAUUUAUCACA | 1032 | 2802-2822 | UGUGAUAAAUAAUCAGGAGAGAA | 1306 | 2800-2822 |
| AD-392700 | UCUCCUGAUUAUUUAUCACAU | 1033 | 2803-2823 | AUGUGATAAAUAAUCAGGAGAGA | 1307 | 2801-2823 |
| AD-392878 | CUCCUGAUUAUUUAUCACAUA | 1034 | 2804-2824 | UAUGUGAUAAAUAAUCAGGAGAG | 1308 | 2802-2824 |
| AD-392718 | UCCUGAUUAUUUAUCACAUAU | 1035 | 2805-2825 | AUAUGUGAUAAAUAAUCAGGAGA | 1309 | 2803-2825 |
| AD-392929 | CCUGAUUAUUUAUCACAUAGU | 1036 | 2806-2826 | ACUAUGTGAUAAAUAAUCAGGAG | 1310 | 2804-2826 |
| AD-392879 | GCCAGUUGUAUAUUAUUCUUU | 1037 | 2833-2853 | AAAGAATAAUAUACAACUGGCUA | 1311 | 2831-2853 |
| AD-392754 | UUGUAUAUUAUUCUUGUGGUU | 1038 | 2838-2858 | AACCACAAGAAUAAUAUACAACU | 1312 | 2836-2858 |
| AD-392819 | UCUUGUGGUUUGUGACCCAAU | 1039 | 2849-2869 | AUUGGGTCACAAACCACAAGAAU | 1313 | 2847-2869 |
| AD-392745 | CUUGUGGUUUGUGACCCAAUU | 1040 | 2850-2870 | AAUUGGGUCACAAACCACAAGAA | 1314 | 2848-2870 |
| AD-392770 | UUGUGGUUUGUGACCCAAUUA | 1041 | 2851-2871 | UAAUUGGGUCACAAACCACAAGA | 1315 | 2849-2871 |
| AD-392806 | UGUGGUUUGUGACCCAAUUAA | 1042 | 2852-2872 | UUAAUUGGGUCACAAACCACAAG | 1316 | 2850-2872 |
| AD-392771 | GUUUGUGACCCAAUUAAGUCU | 1043 | 2856-2876 | AGACUUAAUUGGGUCACAAACCA | 1317 | 2854-2876 |
| AD-392820 | UUUGUGACCCAAUUAAGUCCU | 1044 | 2857-2877 | AGGACUTAAUUGGGUCACAAACC | 1318 | 2855-2877 |
| AD-392821 | UUGUGACCCAAUUAAGUCCUA | 1045 | 2858-2878 | UAGGACTUAAUUGGGUCACAAAC | 1319 | 2856-2878 |
| AD-392786 | UGUGACCCAAUUAAGUCCUAU | 1046 | 2859-2879 | AUAGGACUUAAUUGGGUCACAAA | 1320 | 2857-2879 |
| AD-392772 | GUGACCCAAUUAAGUCCUACU | 1047 | 2860-2880 | AGUAGGACUUAAUUGGGUCACAA | 1321 | 2858-2880 |
| AD-392699 | GACCCAAUUAAGUCCUACUUU | 1048 | 2862-2882 | AAAGUAGGACUUAAUUGGGUCAC | 1322 | 2860-2882 |
| AD-392868 | ACCCAAUUAAGUCCUACUUUA | 1049 | 2863-2883 | UAAAGUAGGACUUAAUUGGGUCA | 1323 | 2861-2883 |
| AD-392719 | CCCAAUUAAGUCCUACUUUAU | 1050 | 2864-2884 | AUAAAGTAGGACUUAAUUGGGUC | 1324 | 2862-2884 |
| AD-392880 | AAUUAAGUCCUACUUUACAUA | 1051 | 2867-2887 | UAUGUAAAGUAGGACUUAAUUGG | 1325 | 2865-2887 |
| AD-392930 | UAAGUCCUACUUUACAUAUGU | 1052 | 2870-2890 | ACAUAUGUAAAGUAGGACUUAAU | 1326 | 2868-2890 |
| AD-392931 | AGUCCUACUUUACAUAUGCUU | 1053 | 2872-2892 | AAGCAUAUGUAAAGUAGGACUUA | 1327 | 2870-2892 |
| AD-392932 | GUCCUACUUUACAUAUGCUUU | 1054 | 2873-2893 | AAAGCATAUGUAAAGUAGGACUU | 1328 | 2871-2893 |
| AD-392869 | UCCUACUUUACAUAUGCUUUA | 1055 | 2874-2894 | UAAAGCAUAUGUAAAGUAGGACU | 1329 | 2872-2894 |
| AD-392870 | CCUACUUUACAUAUGCUUUAA | 1056 | 2875-2895 | UUAAAGCAUAUGUAAAGUAGGAC | 1330 | 2873-2895 |
| AD-392896 | CUACUUUACAUAUGCUUUAAU | 1057 | 2876-2896 | AUUAAAGCAUAUGUAAAGUAGGA | 1331 | 2874-2896 |
| AD-392787 | UACAUAUGCUUUAAGAAUCGA | 1058 | 2882-2902 | UCGAUUCUUAAAGCAUAUGUAAA | 1332 | 2880-2902 |
| AD-392720 | CAUAUGCUUUAAGAAUCGAUU | 1059 | 2884-2904 | AAUCGATUCUUAAAGCAUAUGUA | 1333 | 2882-2904 |
| AD-392746 | AUAUGCUUUAAGAAUCGAUGU | 1060 | 2885-2905 | ACAUCGAUUCUUAAAGCAUAUGU | 1334 | 2883-2905 |
| AD-392773 | UAUGCUUUAAGAAUCGAUGGU | 1061 | 2886-2906 | ACCAUCGAUUCUUAAAGCAUAUG | 1335 | 2884-2906 |
| AD-392807 | GGGAUGCUUCAUGUGAACGUU | 1062 | 2906-2926 | AACGUUCACAUGAAGCAUCCCCC | 1336 | 2904-2926 |
| AD-392730 | UGCUUCUCUUGCCUAAGUAUU | 1063 | 2937-2957 | AAUACUTAGGCAAGAGAAGCAGC | 1337 | 2935-2957 |
| AD-392721 | CUUCUCUUGCCUAAGUAUUCU | 1064 | 2939-2959 | AGAAUACUUAGGCAAGAGAAGCA | 1338 | 2937-2959 |
| AD-392933 | UUCUCUUGCCUAAGUAUUCCU | 1065 | 2940-2960 | AGGAAUACUUAGGCAAGAGAAGC | 1339 | 2938-2960 |
| AD-392934 | CUCUUGCCUAAGUAUUCCUUU | 1066 | 2942-2962 | AAAGGAAUACUUAGGCAAGAGAA | 1340 | 2940-2962 |
| AD-392881 | CUUGCCUAAGUAUUCCUUUCU | 1067 | 2944-2964 | AGAAAGGAAUACUUAGGCAAGAG | 1341 | 2942-2964 |
| AD-392897 | UGCCUAAGUAUUCCUUUCCUU | 1068 | 2946-2966 | AAGGAAAGGAAUACUUAGGCAAG | 1342 | 2944-2966 |
| AD-392898 | AAGUAUUCCUUUCCUGAUCAU | 1069 | 2951-2971 | AUGAUCAGGAAAGGAAUACUUAG | 1343 | 2949-2971 |
| AD-392708 | AGUAUUCCUUUCCUGAUCACU | 1070 | 2952-2972 | AGUGAUCAGGAAAGGAAUACUUA | 1344 | 2950-2972 |
| AD-392899 | GUAUUCCUUUCCUGAUCACUA | 1071 | 2953-2973 | UAGUGATCAGGAAAGGAAUACUU | 1345 | 2951-2973 |
| AD-392935 | UAUUCCUUUCCUGAUCACUAU | 1072 | 2954-2974 | AUAGUGAUCAGGAAAGGAAUACU | 1346 | 2952-2974 |
| AD-392882 | AUUCCUUUCCUGAUCACUAUU | 1073 | 2955-2975 | AAUAGUGAUCAGGAAAGGAAUAC | 1347 | 2953-2975 |
| AD-392738 | UCCUUUCCUGAUCACUAUGCA | 1074 | 2957-2977 | UGCAUAGUGAUCAGGAAAGGAAU | 1348 | 2955-2977 |
| AD-392739 | CUUUCCUGAUCACUAUGCAUU | 1075 | 2959-2979 | AAUGCATAGUGAUCAGGAAAGGA | 1349 | 2957-2979 |
| AD-392936 | UUUCCUGAUCACUAUGCAUUU | 1076 | 2960-2980 | AAAUGCAUAGUGAUCAGGAAAGG | 1350 | 2958-2980 |
| AD-392900 | UUCCUGAUCACUAUGCAUUUU | 1077 | 2961-2981 | AAAAUGCAUAGUGAUCAGGAAAG | 1351 | 2959-2981 |
| AD-392901 | CUGAUCACUAUGCAUUUUAAA | 1078 | 2964-2984 | UUUAAAAUGCAUAGUGAUCAGGA | 1352 | 2962-2984 |
| AD-392937 | CACUAUGCAUUUUAAAGUUAA | 1079 | 2969-2989 | UUAACUTUAAAAUGCAUAGUGAU | 1353 | 2967-2989 |
| AD-392883 | ACUAUGCAUUUUAAAGUUAAA | 1080 | 2970-2990 | UUUAACTUUAAAAUGCAUAGUGA | 1354 | 2968-2990 |
| AD-392975 | UUCCAUGACUGCAUUUUACUU | 1081 | 3029-3049 | AAGUAAAAUGCAGUCAUGGAAAA | 1355 | 3027-3049 |
| AD-392938 | CUGCAUUUUACUGUACAGAUU | 1082 | 3037-3057 | AAUCUGTACAGUAAAAUGCAGUC | 1356 | 3035-3057 |
| AD-392755 | AUUGCUGCUUCUGCUAUAUUU | 1083 | 3055-3075 | AAAUAUAGCAGAAGCAGCAAUCU | 1357 | 3053-3075 |
| AD-392939 | UUCUGCUAUAUUUGUGAUAUA | 1084 | 3063-3083 | UAUAUCACAAAUAUAGCAGAAGC | 1358 | 3061-3083 |
| AD-392940 | UCUGCUAUAUUUGUGAUAUAU | 1085 | 3064-3084 | AUAUAUCACAAAUAUAGCAGAAG | 1359 | 3062-3084 |
| AD-392756 | UGCUAUAUUUGUGAUAUAGGA | 1086 | 3066-3086 | UCCUAUAUCACAAAUAUAGCAGA | 1360 | 3064-3086 |
| AD-392774 | UUUGUGAUAUAGGAAUUAAGA | 1087 | 3073-3093 | UCUUAATUCCUAUAUCACAAAUA | 1361 | 3071-3093 |
| AD-392850 | UCUUCGUGCCUGUUUUAUGUU | 1088 | 3111-3131 | AACAUAAAACAGGCACGAAGAAA | 1362 | 3109-3131 |
| AD-392852 | CUUCGUGCCUGUUUUAUGUGU | 1089 | 3112-3132 | ACACAUAAAACAGGCACGAAGAA | 1363 | 3110-3132 |
| AD-392830 | GUUUUAUGUGCACACAUUAGU | 1090 | 3122-3142 | ACUAAUGUGUGCACAUAAAACAG | 1364 | 3120-3142 |
| AD-392808 | UGUGCACACAUUAGGCAUUGA | 1091 | 3128-3148 | UCAAUGCCUAAUGUGUGCACAUA | 1365 | 3126-3148 |
| AD-392793 | UGCACACAUUAGGCAUUGAGA | 1092 | 3130-3150 | UCUCAATGCCUAAUGUGUGCACA | 1366 | 3128-3150 |
| AD-392757 | ACACAUUAGGCAUUGAGACUU | 1093 | 3133-3153 | AAGUCUCAAUGCCUAAUGUGUGC | 1367 | 3131-3153 |
| AD-392747 | UUUGUCCACGUAUCUUUGGGU | 1094 | 3168-3188 | ACCCAAAGAUACGUGGACAAAAA | 1368 | 3166-3188 |
| AD-392902 | CACGUAUCUUUGGGUCUUUGA | 1095 | 3174-3194 | UCAAAGACCCAAAGAUACGUGGA | 1369 | 3172-3194 |
| AD-392941 | ACGUAUCUUUGGGUCUUUGAU | 1096 | 3175-3195 | AUCAAAGACCCAAAGAUACGUGG | 1370 | 3173-3195 |
| AD-392942 | UCUUUGGGUCUUUGAUAAAGA | 1097 | 3180-3200 | UCUUUATCAAAGACCCAAAGAUA | 1371 | 3178-3200 |
| AD-392943 | CUUUGGGUCUUUGAUAAAGAA | 1098 | 3181-3201 | UUCUUUAUCAAAGACCCAAAGAU | 1372 | 3179-3201 |
| AD-392944 | UUGGGUCUUUGAUAAAGAAAA | 1099 | 3183-3203 | UUUUCUTUAUCAAAGACCCAAAG | 1373 | 3181-3203 |
| AD-392903 | UGGGUCUUUGAUAAAGAAAAU | 1100 | 3184-3204 | AUUUUCTUUAUCAAAGACCCAAA | 1374 | 3182-3204 |
| AD-392775 | AAAGAAUCCCUGUUCAUUGUA | 1101 | 3201-3221 | UACAAUGAACAGGGAUUCUUUUC | 1375 | 3199-3221 |
| AD-392758 | AAGAAUCCCUGUUCAUUGUAA | 1102 | 3202-3222 | UUACAATGAACAGGGAUUCUUUU | 1376 | 3200-3222 |
| AD-392945 | AGAAUCCCUGUUCAUUGUAAU | 1103 | 3203-3223 | AUUACAAUGAACAGGGAUUCUUU | 1377 | 3201-3223 |
| AD-392946 | GAAUCCCUGUUCAUUGUAAGU | 1104 | 3204-3224 | ACUUACAAUGAACAGGGAUUCUU | 1378 | 3202-3224 |
| AD-392884 | UGUUCAUUGUAAGCACUUUUA | 1105 | 3211-3231 | UAAAAGTGCUUACAAUGAACAGG | 1379 | 3209-3231 |
| AD-392947 | GUUCAUUGUAAGCACUUUUAU | 1106 | 3212-3232 | AUAAAAGUGCUUACAAUGAACAG | 1380 | 3210-3232 |
| AD-392748 | UCAUUGUAAGCACUUUUACGU | 1107 | 3214-3234 | ACGUAAAAGUGCUUACAAUGAAC | 1381 | 3212-3234 |
| AD-392759 | CAUUGUAAGCACUUUUACGGU | 1108 | 3215-3235 | ACCGUAAAAGUGCUUACAAUGAA | 1382 | 3213-3235 |
| AD-392837 | CUGGUCUUCAAUUACCAAGAA | 1109 | 3258-3278 | UUCUUGGUAAUUGAAGACCAGCA | 1383 | 3256-3278 |
| AD-392970 | GGUCUUCAAUUACCAAGAAUU | 1110 | 3260-3280 | AAUUCUTGGUAAUUGAAGACCAG | 1384 | 3258-3280 |
| AD-392976 | UCUUCAAUUACCAAGAAUUCU | 1111 | 3262-3282 | AGAAUUCUUGGUAAUUGAAGACC | 1385 | 3260-3282 |
| AD-392965 | CUUCAAUUACCAAGAAUUCUU | 1112 | 3263-3283 | AAGAAUTCUUGGUAAUUGAAGAC | 1386 | 3261-3283 |
| AD-392831 | UUCAAUUACCAAGAAUUCUCU | 1113 | 3264-3284 | AGAGAATUCUUGGUAAUUGAAGA | 1387 | 3262-3284 |
| AD-392904 | UCAAUUACCAAGAAUUCUCCA | 1114 | 3265-3285 | UGGAGAAUUCUUGGUAAUUGAAG | 1388 | 3263-3285 |
| AD-392885 | AAUUACCAAGAAUUCUCCAAA | 1115 | 3267-3287 | UUUGGAGAAUUCUUGGUAAUUGA | 1389 | 3265-3287 |
| AD-392886 | UUACCAAGAAUUCUCCAAAAU | 1116 | 3269-3289 | AUUUUGGAGAAUUCUUGGUAAUU | 1390 | 3267-3289 |
| AD-392776 | UGAUUGUACAGAAUCAUUGCU | 1117 | 3304-3324 | AGCAAUGAUUCUGUACAAUCAUC | 1391 | 3302-3324 |
| AD-392887 | UCAUUGCUUAUGACAUGAUCU | 1118 | 3317-3337 | AGAUCATGUCAUAAGCAAUGAUU | 1392 | 3315-3337 |
| AD-392722 | CAUUGCUUAUGACAUGAUCGU | 1119 | 3318-3338 | ACGAUCAUGUCAUAAGCAAUGAU | 1393 | 3316-3338 |
| AD-392740 | AUUGCUUAUGACAUGAUCGCU | 1120 | 3319-3339 | AGCGAUCAUGUCAUAAGCAAUGA | 1394 | 3317-3339 |
| AD-392760 | UUGCUUAUGACAUGAUCGCUU | 1121 | 3320-3340 | AAGCGATCAUGUCAUAAGCAAUG | 1395 | 3318-3340 |
| AD-392731 | UGCUUAUGACAUGAUCGCUUU | 1122 | 3321-3341 | AAAGCGAUCAUGUCAUAAGCAAU | 1396 | 3319-3341 |
| AD-392709 | GCUUAUGACAUGAUCGCUUUC | 1123 | 3322-3342 | GAAAGCGAUCAUGUCAUAAGCAA | 1397 | 3320-3342 |
| AD-392723 | CUUAUGACAUGAUCGCUUUCU | 1124 | 3323-3343 | AGAAAGCGAUCAUGUCAUAAGCA | 1398 | 3321-3343 |
| AD-392948 | UUAUGACAUGAUCGCUUUCUA | 1125 | 3324-3344 | UAGAAAGCGAUCAUGUCAUAAGC | 1399 | 3322-3344 |
| AD-392724 | UAUGACAUGAUCGCUUUCUAU | 1126 | 3325-3345 | AUAGAAAGCGAUCAUGUCAUAAG | 1400 | 3323-3345 |
| AD-392949 | AUGACAUGAUCGCUUUCUACA | 1127 | 3326-3346 | UGUAGAAAGCGAUCAUGUCAUAA | 1401 | 3324-3346 |
| AD-392725 | UGACAUGAUCGCUUUCUACAU | 1128 | 3327-3347 | AUGUAGAAAGCGAUCAUGUCAUA | 1402 | 3325-3347 |
| AD-392950 | CAUGAUCGCUUUCUACACUGU | 1129 | 3330-3350 | ACAGUGTAGAAAGCGAUCAUGUC | 1403 | 3328-3350 |
| AD-392732 | UGAUCGCUUUCUACACUGUAU | 1130 | 3332-3352 | AUACAGTGUAGAAAGCGAUCAUG | 1404 | 3330-3352 |
| AD-392726 | GAUCGCUUUCUACACUGUAUU | 1131 | 3333-3353 | AAUACAGUGUAGAAAGCGAUCAU | 1405 | 3331-3353 |
| AD-392733 | AUCGCUUUCUACACUGUAUUA | 1132 | 3334-3354 | UAAUACAGUGUAGAAAGCGAUCA | 1406 | 3332-3354 |
| AD-392906 | UCGCUUUCUACACUGUAUUAU | 1133 | 3335-3355 | AUAAUACAGUGUAGAAAGCGAUC | 1407 | 3333-3355 |
| AD-392862 | CGCUUUCUACACUGUAUUACA | 1134 | 3336-3356 | UGUAAUACAGUGUAGAAAGCGAU | 1408 | 3334-3356 |
| AD-392951 | CUUUCUACACUGUAUUACAUA | 1135 | 3338-3358 | UAUGUAAUACAGUGUAGAAAGCG | 1409 | 3336-3358 |
| AD-392871 | UUCUACACUGUAUUACAUAAA | 1136 | 3340-3360 | UUUAUGTAAUACAGUGUAGAAAG | 1410 | 3338-3360 |
| AD-392872 | UCUACACUGUAUUACAUAAAU | 1137 | 3341-3361 | AUUUAUGUAAUACAGUGUAGAAA | 1411 | 3339-3361 |
| AD-392952 | GAUUCAAUUUUCUUUAACCAU | 1138 | 3456-3476 | AUGGUUAAAGAAAAUUGAAUCUG | 1412 | 3454-3476 |
| AD-392907 | AUUUUCUUUAACCAGUCUGAA | 1139 | 3462-3482 | UUCAGACUGGUUAAAGAAAAUUG | 1413 | 3460-3482 |
| AD-392953 | UUUCUUUAACCAGUCUGAAGU | 1140 | 3464-3484 | ACUUCAGACUGGUUAAAGAAAAU | 1414 | 3462-3484 |
| AD-392741 | UCUUUAACCAGUCUGAAGUUU | 1141 | 3466-3486 | AAACUUCAGACUGGUUAAAGAAA | 1415 | 3464-3486 |
| AD-392908 | CUUUAACCAGUCUGAAGUUUC | 1142 | 3467-3487 | GAAACUTCAGACUGGUUAAAGAA | 1416 | 3465-3487 |
| AD-392977 | CUGAAGUUUCAUUUAUGAUAU | 1143 | 3478-3498 | AUAUCATAAAUGAAACUUCAGAC | 1417 | 3476-3498 |
| AD-392847 | GAAGUUUCAUUUAUGAUACAA | 1144 | 3480-3500 | UUGUAUCAUAAAUGAAACUUCAG | 1418 | 3478-3500 |
| AD-392809 | AAAUGGAAGUGGCAAUAUAAU | 1145 | 3511-3531 | AUUAUATUGCCACUUCCAUUUUC | 1419 | 3509-3531 |
| AD-392810 | AUGGAAGUGGCAAUAUAAGGU | 1146 | 3513-3533 | ACCUUATAUUGCCACUUCCAUUU | 1420 | 3511-3533 |
| AD-392777 | UGCCUGGACAAACCCUUCUUU | 1147 | 3547-3567 | AAAGAAGGGUUUGUCCAGGCAUG | 1421 | 3545-3567 |
| AD-392960 | UUCUUUUAAGAUGUGUCUUCA | 1148 | 3562-3582 | UGAAGACACAUCUUAAAAGAAGG | 1422 | 3560-3582 |
| AD-392873 | CUUUUAAGAUGUGUCUUCAAU | 1149 | 3564-3584 | AUUGAAGACACAUCUUAAAAGAA | 1423 | 3562-3584 |
| AD-392889 | UUUUAAGAUGUGUCUUCAAUU | 1150 | 3565-3585 | AAUUGAAGACACAUCUUAAAAGA | 1424 | 3563-3585 |
| AD-392954 | UUUAAGAUGUGUCUUCAAUUU | 1151 | 3566-3586 | AAAUUGAAGACACAUCUUAAAAG | 1425 | 3564-3586 |
| AD-392955 | UUAAGAUGUGUCUUCAAUUUG | 1152 | 3567-3587 | CAAAUUGAAGACACAUCUUAAAA | 1426 | 3565-3587 |
| AD-392909 | UAAGAUGUGUCUUCAAUUUGU | 1153 | 3568-3588 | ACAAAUTGAAGACACAUCUUAAA | 1427 | 3566-3588 |
| AD-392710 | AAGAUGUGUCUUCAAUUUGUA | 1154 | 3569-3589 | UACAAATUGAAGACACAUCUUAA | 1428 | 3567-3589 |
| AD-392956 | AGAUGUGUCUUCAAUUUGUAU | 1155 | 3570-3590 | AUACAAAUUGAAGACACAUCUUA | 1429 | 3568-3590 |
| AD-392874 | AUGUGUCUUCAAUUUGUAUAA | 1156 | 3572-3592 | UUAUACAAAUUGAAGACACAUCU | 1430 | 3570-3592 |
| AD-392957 | UGUCUUCAAUUUGUAUAAAAU | 1157 | 3575-3595 | AUUUUATACAAAUUGAAGACACA | 1431 | 3573-3595 |
| AD-392958 | CUUCAAUUUGUAUAAAAUGGU | 1158 | 3578-3598 | ACCAUUTUAUACAAAUUGAAGAC | 1432 | 3576-3598 |
| AD-392959 | AUGGUGUUUUCAUGUAAAUAA | 1159 | 3594-3614 | UUAUUUACAUGAAAACACCAUUU | 1433 | 3592-3614 |
| AD-392788 | GUAAAUAAAUACAUUCUUGGA | 1160 | 3607-3627 | UCCAAGAAUGUAUUUAUUUACAU | 1434 | 3605-3627 |

TABLE 5
Further Exemplary Human APP-Targeting RNAi Agent Duplexes, Modified Sequences

| | | SEQ | | SEQ | | SEQ |
|---|---|---|---|---|---|---|
| | Sense Strand | ID | Antisense Strand | ID | mRNA target | ID |
| Agent | Oligo Sequence | NO: | Oligo Sequence | NO: | sequence | NO: |
| AD- | usasuga(Ahd)GfuUfCf | 1435 | VPusUfsuuug(Agn)ugauga | 1440 | GAUAUGAAGUUCAUCAUCAAAAA | 1445 |
| 454972 | Afucaucaaasasa | | AfcUfucauasusc | | | |
| AD- | gsgscua(Chd)GfaAfAf | 1436 | VPusUfsaggu(Tgn)ggauuu | 1441 | ACGGCUACGAAAAUCCAACCUAA | 1446 |
| 454973 | Afuccaaccusasa | | UfcGfuagccsgsu | | | |
| AD- | ususugu(Ghd)UfaCfUf | 1437 | VPusAfsauuc(Tgn)uuacag | 1442 | GUUUUGUGUACUGUAAAGAAUUA | 1447 |
| 454842 | Gfuaaagaaususa | | UfaCfacaaasasc | | | |
| AD- | usasgug(Chd)AfuGfAf | 1438 | VPusGfsagaa(Tgn)cuauuc | 1443 | ACUAGUGCAUGAAUAGAUUCUCA | 1448 |
| 454843 | Afuagauucuscsa | | AfuGfcacuasgsu | | | |
| AD- | asasaau(Chd)CfaAfCf | 1439 | VPusGfsaacu(Tgn)guaggu | 1444 | CGAAAAUCCAACCUACAAGUUCA | 1449 |
| 454844 | Cfuacaaguuscsa | | UfgGfauuuuscsg | | | |

Table 5 key: U = uridine-3′-phosphate, u = 2′-O-methyluridine-3′-phosphate, us = 2′-O-methyluridine-3′-phosphorothioate, a = 2′-O-methyladenosine-3′-phosphate, A = adenosine-3′-phosphate, as = 2′-O-methyladenosine-3′-phosphorothioate, (Ahd) = 2′-O-hexadecyl-adenosine-3′-phosphate, Gf = 2′ -fluoroguanosine-3′-phosphate,
Uf = 2′-fluorouridine-3′-phosphate, Cf = 2′-fluorocytidine-3′-phosphate, Af = 2′-fluoroadenosine-3′-phosphate, cs = 2′-O-methylcytidine-3′-phosphate , VP = Vinylphosphate 5′, (Agn) = Adenosine-glycol nucleic acid (GNA), gs = 2′-O-methylguanosine-3-phosphorothioate, (Chd) = 2′-O-hexadecyl-cytidine-3′-phosphate,
(Tgn) = Thymidine-glycol nucleic acid (GNA) S-Isomer,
(Ghd) = 2′-O-hexadecyl-guanosine-3′-phosphate, and cs = 2′-O-methylcytidine-3′-phosphorothioate.

TABLE 6
Further Exemplary Human APP-Targeting RNAi Agent Duplexes, Unmodified Sequences

| | | SEQ | | SEQ | | SEQ |
|---|---|---|---|---|---|---|
| | Sense Strand Oligo | ID | Antisense Strand Oligo | ID | | ID |
| Agent | Sequence | NO: | Sequence | NO: | mRNA target sequence | NO: |
| AD- | UAUGAAGUUCAUCAUCAAAAA | 1450 | UUUUUGAUGAUGAACUUCAUAUC | 1455 | GAUAUGAAGUUCAUCAUCAAAAA | 1445 |
| 454972 | | | | | | |
| AD- | GGCUACGAAAAUCCAACCUAA | 1451 | UUAGGUTGGAUUUUCGUAGCCGU | 1456 | ACGGCUACGAAAAUCCAACCUAA | 1446 |
| 454973 | | | | | | |
| AD- | UUUGUGUACUGUAAAGAAUUA | 1452 | UAAUUCTUUACAGUACACAAAAC | 1457 | GUUUUGUGUACUGUAAAGAAUUA | 1447 |
| 454842 | | | | | | |
| AD- | UAGUGCAUGAAUAGAUUCUCA | 1453 | UGAGAATCUAUUCAUGCACUAGU | 1458 | ACUAGUGCAUGAAUAGAUUCUCA | 1448 |
| 454843 | | | | | | |
| AD- | AAAAUCCAACCUACAAGUUCA | 1454 | UGAACUTGUAGGUUGGAUUUUCG | 1459 | CGAAAAUCCAACCUACAAGUUCA | 1449 |
| 454844 | | | | | | |

TABLE 7
More Exemplary Human APP-Targeting RNAi Agent Duplexes, Modified Sequences

| | | SEQ | | SEQ | | SEQ |
|---|---|---|---|---|---|---|
| Duplex | Sense Sequence | ID | Antisense Sequence | ID | | ID |
| Name | (5′ to 3′) | NO | (5′ to 3′) | NO | mRNA target sequence | NO |
| AD- | asasagagCfaAfAfAf | 1460 | asUfscugAfaUfAfguuuUfg | 1471 | AGAAAGAGCAAAACUAUUCAGAU | 1482 |
| 506935.2 | cuauucagauL96 | | Cfucuuuscsu | | | |
| AD- | ususggccAfaCfAfUf | 1461 | asUfscacUfaAfUfcaugUfu | 1472 | UCUUGGCCAACAUGAUUAGUGAA | 1483 |
| 507065.2 | gauuagugauL96 | | Gfgccaasgsa | | | |
| AD- | uscsugggUfuGfAfCf | 1462 | asUfsugaUfaUfUfugucAfa | 1473 | GUUCUGGGUUGACAAAUAUCAAG | 1484 |
| 507159.2 | aaauaucaauL96 | | Cfccagasas | | | |
| AD- | ususuaugAfuUfUfAf | 1463 | asGfsauaAfuGfAfguaaAfu | 1474 | GUUUUAUGAUUUACUCAUUAUCG | 1485 |
| 507538.2 | cucauuaucuL96 | | Cfauaaasasc | | | |
| AD- | asusgccuGfaAfCfUf | 1464 | asAfsuuaAfuUfCfaaguUfc | 1475 | AGAUGCCUGAACUUGAAUUAAUC | 1486 |
| 507624.2 | ugaauuaauuL96 | | Afggcauscsu | | | |
| AD- | asgsaugcCfuGfAfAf | 1465 | asUfsaauUfcAfAfguucAfg | 1476 | GUAGAUGCCUGAACUUGAAUUAA | 1487 |
| 507724.2 | cuugaauuauL96 | | Gfcaucusasc | | | |
| AD- | gscscugaAfcUfUfGf | 1466 | asGfsgauUfaAfUfucaaGfu | 1477 | AUGCCUGAACUUGAAUUAAUCCA | 1488 |
| 507725.2 | aauuaauccuL96 | | Ufcaggcsasu | | | |
| AD- | gsusgguuUfgUfGfAf | 1467 | asUfsuaaUfuGfGfgucaCfa | 1478 | UUGUGGUUUGUGACCCAAUUAAG | 1489 |
| 507789.2 | cccaauuaauL96 | | Afaccacsasa | | | |
| AD- | csasgaugCfuUfUfAf | 1468 | asAfsaauCfuCfUfcuaaAfg | 1479 | UUCAGAUGCUUUAGAGAGAUUUU | 1490 |
| 507874.2 | gagagauuuuL96 | | Cfaucugsasa | | | |
| AD- | uscsuugcCfuAfAfGf | 1469 | asAfsaagGfaAfUfacuuAfg | 1480 | UCUCUUGCCUAAGUAUUCCUUUC | 1491 |
| 507928.2 | uauuccuuuuL96 | | Gfcaagasgsa | | | |
| AD- | ususgcugCfuUfCfUf | 1470 | asAfsaauAfuAfGfcagaAfg | 1481 | GAUUGCUGCUUCUGCUAUAUUUG | 1492 |
| 507949.2 | gcuauauuuuL96 | | Cfagcaasusc | | | |

Table 10 key: U = uridine-3′-phosphate, u = 2′-O-methyluridine-3′-phosphate, us = 2′-O-methyluridine-3′-phosphorothioate, a = 2′-O-methyladenosine-3′-phosphate, A = adenosine-3′-phosphate, as = 2′-O-methyladenosine-3′-phosphorothioate, (Ahd) = 2′-O-hexadecyl-adenosine-3′-phosphate, Gf = 2′-fluoroguanosine-3′-phosphate,
Uf = 2′-fluorouridine-3′-phosphate, Cf = 2′-fluorocytidine-3′-phosphate, Af = 2′-fluoroadenosine-3′-phosphate, cs = 2′-O-methylcytidine-3′-phosphate , VP = Vinylphosphate 5′, (Agn) = Adenosine-glycol nucleic acid (GNA), gs = 2′-O-methylguanosine-3′-phosphorothioate, (Chd) = 2′-O-hexadecyl-cytidine-3′-phosphate,
(Tgn) = Thymidine-glycol nucleic acid (GNA) S-Isomer, (Ghd) = 2′-O-hexadecyl-guanosine-3′-phosphate, and cs = 2′-O-methylcytidine-3′-phosphorothioate.

TABLE 8
Additional APP-Targeting RNAi Agents, Unmodified Sequences; NM_000484.3 and
NM_201414.2 Targeting

| | | SEQ | | Antisense | SEQ | | Cross |
|---|---|---|---|---|---|---|---|
| Duplex | Sense Sequence | ID | Source Name | Sequence | ID | Source Name | Spe- |
| Name | (5′ to 3′) | NO | (Range) | (5′ to 3′) | NO | (Range) | cies |
| AD- | AAAGAGCAAAACUA | 1493 | NM_000484.3_1902- | AUCUGAAUAGUUUU | 1504 | NM_201414.2_1675- | UNK |
| 506935.2 | UUCAGAU | | 1922_s | GCUCUUUCU | | 1697_as | |
| | | | (1902-1922) | | | (1900-1922) | |
| AD- | UUGGCCAACAUGAU | 1494 | NM_201414.2_1704- | AUCACUAAUCAUGU | 1505 | NM_201414.2_1702- | UNK |
| 507065.2 | UAGUGAU | | 1724_A21U_s | UGGCCAAGA | | 1724_U1A_as | |
| | | | (1704-1724) | | | (1702-1724) | |
| AD- | UCUGGGUUGACAAA | 1495 | NM_000484.3_2166- | AUUGAUAUUUGUCA | 1506 | NM_201414.2_1939- | UNK |
| 507159.2 | UAUCAAU | | 2186_G21U_s | ACCCAGAAC | | 1961_C1A_as | |
| | | | (2166-2186) | | | (2164-2186) | |
| AD- | UUUAUGAUUUACUC | 1496 | NM_000484.3_2613- | AGAUAAUGAGUAAA | 1507 | NM_201414.2_2386- | UNK |
| 507538.2 | AUUAUCU | | 2633_G21U_s | UCAUAAAAC | | 2408_C1A_as | |
| | | | (2613-2633) | | | (2611-2633) | |
| AD- | AUGCCUGAACUUGA | 1497 | NM_000484.3_2665- | AAUUAAUUCAAGUU | 1508 | NM_201414.2_2438- | UNK |
| 507624.2 | AUUAAUU | | 2685_C21U_s | CAGGCAUCU | | 2460_G1A_as | |
| | | | (2665-2685) | | | (2663-2685) | |
| AD- | AGAUGCCUGAACUU | 1498 | NM_201414.2_2438- | AUAAUUCAAGUUCA | 1509 | NM_201414.2_2436- | UNK |
| 507724.2 | GAAUUAU | | 2458_A21U_s | GGCAUCUAC | | 2458_U1A_as | |
| | | | (2438-2458) | | | (2436-2458) | |
| AD- | GCCUGAACUUGAAU | 1499 | NM_201414.2_2442- | AGGAUUAAUUCAAG | 1510 | NM_201414.2_2440- | UNK |
| 507725.2 | UAAUCCU | | 2462_A21U_s | UUCAGGCAU | | 2462_U1A_as | |
| | | | (2442-2462) | | | (2440-2462) | |
| AD- | GUGGUUUGUGACCC | 1500 | NM_000484.3_2853- | AUUAAUUGGGUCAC | 1511 | NM_201414.2_2626- | UNK |
| 507789.2 | AAUUAAU | | 2873_G21U_s | AAACCACAA | | 2648_C1A_as | |
| | | | (2853-2873) | | | (2851-2873) | |
| AD- | CAGAUGCUUUAGAG | 1501 | NM_000484.3_3006- | AAAAUCUCUCUAAA | 1512 | NM_201414.2_2779- | UNK |
| 507874.2 | AGAUUUU | | 3026_s | GCAUCUGAA | | 2801_as | |
| | | | (3006-3026) | | | (3004-3026) | |
| AD- | UCUUGCCUAAGUAU | 1502 | NM_201414.2_2718- | AAAAGGAAUACUUA | 1513 | NM_201414.2_2716- | UNK |
| 507928.2 | UCCUUUU | | 2738_C21U_s | GGCAAGAGA | | 2738_G1A_as | |
| | | | (2718-2738) | | | (2716-2738) | |
| AD- | UUGCUGCUUCUGCU | 1503 | NM_201414.2_2831- | AAAAUAUAGCAGAA | 1514 | NM_201414.2_2829- | UNK |
| 507949.2 | AUAUUUU | | 2851_G21U_s | GCAGCAAUC | | 2851_C1A_as | |
| | | | (2831-2851) | | | (2829-2851) | |

TABLE 9
Additional Human APP-Targeting RNAi Agents, Modified Sequences.

| | | SEQ | | SEQ |
|---|---|---|---|---|
| Duplex | | ID | | ID |
| Name | Sense Sequence (5′ to 3′) | NO | Antisense Sequence (5′ to 3′) | NO |
| AD-738012.1 | csgscuu(Uhd)CfuAfCfAfcuguauuacaL96 | 1515 | VPusGfsuaaUfaCfAfguguAfgAfaagcgsasu | 1605 |
| AD-738013.1 | gscsuuu(Chd)UfaCfAfCfuguauuacaaL96 | 1516 | VPusUfsguaAfuAfCfagugUfaGfaaagcsgsa | 1606 |
| AD-738014.1 | ususcua(Chd)AfcUfGfUfauuacauaaaL96 | 1517 | VPusUfsuauGfuAfAfuacaGfuGfuagaasasg | 1607 |
| AD-738015.1 | ususucu(Ahd)CfaCfUfGfuauuacauaaL96 | 1518 | VPusUfsaugUfaAfUfacagUfgUfagaaasgsc | 1608 |
| AD-738016.1 | asusuua(Ghd)CfuGfUfAfucaaacuagaL96 | 1519 | VPusCfsuagUfuUfGfauacAfgCfuaaaususc | 1609 |
| AD-738017.1 | ususccu(Ghd)AfuCfAfCfuaugcauuuaL96 | 1520 | VPusAfsaauGfcAfUfagugAfuCfaggaasasg | 1610 |
| AD-738018.1 | gsusgcu(Ghd)UfaAfCfAfcaaguagauaL96 | 1521 | VPusAfsucuAfcUfUfguguUfaCfagcacsasg | 1611 |
| AD-738019.1 | ususuag(Chd)UfgUfAfUfcaaacuaguaL96 | 1522 | VPusAfscuaGfuUfUfgauaCfaGfcuaaasusu | 1612 |
| AD-738020.1 | ususucc(Uhd)GfaUfCfAfcuaugcauuaL96 | 1523 | VPusAfsaugCfaUfAfgugaUfcAfggaaasgsg | 1613 |
| AD-738021.1 | asasugg(Ghd)UfuUfUfGfuguacuguaaL96 | 1524 | VPusUfsacaGfuAfCfacaaAfaCfccauusasa | 1614 |
| AD-738022.1 | asusugu(Ahd)CfaGfAfAfucauugcuuaL96 | 1525 | VPusAfsagcAfaUfGfauucUfgUfacaauscsa | 1615 |
| AD-738023.1 | ususgua(Chd)AfgAfAfUfcauugcuuaaL96 | 1526 | VPusUfsaagCfaAfUfgauuCfuGfuacaasusc | 1616 |
| AD-738024.1 | ususacu(Ghd)UfaCfAfGfauugcugcuaL96 | 1527 | VPusAfsgcaGfcAfAfucugUfaCfaguaasasa | 1617 |
| AD-738025.1 | asusaug(Chd)UfgAfAfGfaaguacgucaL96 | 1528 | VPusGfsacgUfaCfUfucuuCfaGfcauaususg | 1618 |
| AD-738026.1 | ascscau(Uhd)GfcUfUfCfacuacccauaL96 | 1529 | VPusAfsuggGfuAfGfugaaGfcAfauggususu | 1619 |
| AD-738027.1 | csusgug(Chd)UfgUfAfAfcacaaguagaL96 | 1530 | VPusCfsuacUfuGfUfguuaCfaGfcacagscsu | 1620 |
| AD-738028.1 | usgscug(Uhd)AfaCfAfCfaaguagaugaL96 | 1531 | VPusCfsaucUfaCfUfugugUfuAfcagcascsa | 1621 |
| AD-738029.1 | ascsagc(Uhd)GfuGfCfUfguaacacaaaL96 | 1532 | VPusUfsuguGfuUfAfcagcAfcAfgcuguscsa | 1622 |
| AD-738030.1 | gscsugu(Ahd)AfcAfCfAfaguagaugcaL96 | 1533 | VPusGfscauCfuAfCfuuguGfuUfacagcsasc | 1623 |
| AD-738031.1 | uscsaaa(Chd)UfaGfUfGfcaugaauagaL96 | 1534 | VPusCfsuauUfcAfUfgcacUfaGfuuugasusa | 1624 |
| AD-738032.1 | csasaac(Uhd)AfgUfGfCfaugaauagaaL96 | 1535 | VPusUfscuaUfuCfAfugcaCfuAfguuugsasu | 1625 |
| AD-738033.1 | usgscag(Ghd)AfuGfAfUfuguacagaaaL96 | 1536 | VPusUfsucuGfuAfCfaaucAfuCfcugcasgsa | 1626 |
| AD-738034.1 | gscsagg(Ahd)UfgAfUfUfguacagaauaL96 | 1537 | VPusAfsuucUfgUfAfcaauCfaUfccugcsasg | 1627 |
| AD-738035.1 | csasgga(Uhd)GfaUfUfGfuacagaaucaL96 | 1538 | VPusGfsauuCfuGfUfacaaUfcAfuccugscsa | 1628 |
| AD-738036.1 | usasuca(Ahd)AfcUfAfGfugcaugaauaL96 | 1539 | VPusAfsuucAfuGfCfacuaGfuUfugauascsa | 1629 |
| AD-738037.1 | ususugu(Ghd)CfcUfGfUfuuuaugugcaL96 | 1540 | VPusGfscacAfuAfAfaacaGfgCfacaaasgsa | 1630 |
| AD-738038.1 | ususgug(Chd)CfuGfUfUfuuaugugcaaL96 | 1541 | VPusUfsgcaCfaUfAfaaacAfgGfcacaasasg | 1631 |
| AD-738039.1 | csusgca(Ghd)GfaUfGfAfuuguacagaaL96 | 1542 | VPusUfscugUfaCfAfaucaUfcCfugcagsasa | 1632 |
| AD-738040.1 | csasggu(Chd)AfuGfAfGfagaaugggaaL96 | 1543 | VPusUfscccAfuUfCfucucAfuGfaccugsgsg | 1633 |
| AD-738041.1 | usasugu(Ghd)CfaCfAfCfauuaggcauaL96 | 1544 | VPusAfsugcCfuAfAfugugUfgCfacauasasa | 1634 |
| AD-738042.1 | usgsugc(Ahd)CfaCfAfUfuaggcauugaL96 | 1545 | VPusCfsaauGfcCfUfaaugUfgUfgcacasusa | 1635 |
| AD-738043.1 | gsgsaug(Ahd)UfuGfUfAfcagaaucauaL96 | 1546 | VPusAfsugaUfuCfUfguacAfaUfcauccsusg | 1636 |
| AD-738044.1 | ascscau(Chd)CfaGfAfAfcuggugcaaaL96 | 1547 | VPusUfsugcAfcCfAfguucUfgGfaugguscsa | 1637 |
| AD-738045.1 | usasugc(Uhd)GfaAfGfAfaguacguccaL96 | 1548 | VPusGfsgacGfuAfCfuucuUfcAfgcauasusu | 1638 |
| AD-738046.1 | asusgcu(Ghd)AfaGfAfAfguacguccgaL96 | 1549 | VPusCfsggaCfgUfAfcuucUfuCfagcausasu | 1639 |
| AD-738047.1 | asasacc(Ahd)UfuGfCfUfucacuacccaL96 | 1550 | VPusGfsgguAfgUfGfaagcAfaUfgguuususg | 1640 |
| AD-738048.1 | asascca(Uhd)UfgCfUfUfcacuacccaaL96 | 1551 | VPusUfsgggUfaGfUfgaagCfaAfugguususu | 1641 |
| AD-397217.2 | csasccg(Ahd)GfaGfAfGfaaugucccaaL96 | 1552 | VPusUfsgggAfcAfUfucucUfcUfcggugscsu | 1642 |
| AD-738049.1 | gsusugu(Ahd)UfaUfUfAfuucuuguggaL96 | 1553 | VPusCfscacAfaGfAfauaaUfaUfacaacsusg | 1643 |
| AD-738050.1 | ususaug(Uhd)GfcAfCfAfcauuaggcaaL96 | 1554 | VPusUfsgccUfaAfUfguguGfcAfcauaasasa | 1644 |
| AD-738051.1 | asusgug(Chd)AfcAfCfAfuuaggcauuaL96 | 1555 | VPusAfsaugCfcUfAfauguGfuGfcacausasa | 1645 |
| AD-738052.1 | gsusgca(Chd)AfcAfUfUfaggcauugaaL96 | 1556 | VPusUfscaaUfgCfCfuaauGfuGfugcacsasu | 1646 |
| AD-738053.1 | usgsauu(Ghd)UfaCfAfGfaaucauugcaL96 | 1557 | VPusGfscaaUfgAfUfucugUfaCfaaucasusc | 1647 |
| AD-738054.1 | gscsuuc(Ahd)CfuAfCfCfcaucgguguaL96 | 1558 | VPusAfscacCfgAfUfggguAfgUfgaagcsasa | 1648 |
| AD-738055.1 | ususuua(Uhd)GfuGfCfAfcacauuaggaL96 | 1559 | VPusCfscuaAfuGfUfgugcAfcAfuaaaascsa | 1649 |
| AD-738056.1 | csgscuu(Uhd)CfuAfCfAfcuguauuacaL96 | 1560 | VPusGfsuaau(Agn)caguguAfgAfaagcgsasu | 1650 |
| AD-738057.1 | gscsuuu(Chd)UfaCfAfCfuguauuacaaL96 | 1561 | VPusUfsguaa(Tgn)acagugUfaGfaaagcsgsa | 1651 |
| AD-738058.1 | ususcua(Chd)AfcUfGfUfauuacauaaaL96 | 1562 | VPusUfsuaug(Tgn)aauacaGfuGfuagaasasg | 1652 |
| AD-738059.1 | ususucu(Ahd)CfaCfUfGfuauuacauaaL96 | 1563 | VPusUfsaugu(Agn)auacagUfgUfagaaasgsc | 1653 |
| AD-738060.1 | asusuua(Ghd)CfuGfUfAfucaaacuagaL96 | 1564 | VPusCfsuagu(Tgn)ugauacAfgCfuaaaususc | 1654 |
| AD-738061.1 | ususccu(Ghd)AfuCfAfCfuaugcauuuaL96 | 1565 | VPusAfsaaug(Cgn)auagugAfuCfaggaasasg | 1655 |
| AD-738062.1 | gsusgcu(Ghd)UfaAfCfAfcaaguagauaL96 | 1566 | VPusAfsucua(Cgn)uuguguUfaCfagcacsasg | 1656 |
| AD-738063.1 | ususuag(Chd)UfgUfAfUfcaaacuaguaL96 | 1567 | VPusAfscuag(Tgn)uugauaCfaGfcuaaasusu | 1657 |
| AD-738064.1 | ususucc(Uhd)GfaUfCfAfcuaugcauuaL96 | 1568 | VPusAfsaugc(Agn)uagugaUfcAfggaaasgsg | 1658 |
| AD-738065.1 | asasugg(Ghd)UfuUfUfGfuguacuguaaL96 | 1569 | VPusUfsacag(Tgn)acacaaAfaCfccauusasa | 1659 |
| AD-738066.1 | ususacu(Ghd)UfaCfAfGfauugcugcuaL96 | 1570 | VPusAfsgcag(Cgn)aaucugUfaCfaguaasasa | 1660 |
| AD-738067.1 | asusugu(Ahd)CfaGfAfAfucauugcuuaL96 | 1571 | VPusAfsagca(Agn)ugauucUfgUfacaauscsa | 1661 |
| AD-738068.1 | ususgua(Chd)AfgAfAfUfcauugcuuaaL96 | 1572 | VPusUfsaagc(Agn)augauuCfuGfuacaasusc | 1662 |
| AD-738069.1 | asusaug(Chd)UfgAfAfGfaaguacgucaL96 | 1573 | VPusGfsacgu(Agn)cuucuuCfaGfcauaususg | 1663 |
| AD-738070.1 | ascscau(Uhd)GfcUfUfCfacuacccauaL96 | 1574 | VPusAfsuggg(Tgn)agugaaGfcAfauggususu | 1664 |
| AD-738071.1 | csusgug(Chd)UfgUfAfAfcacaaguagaL96 | 1575 | VPusCfsuacu(Tgn)guguuaCfaGfcacagscsu | 1665 |
| AD-738072.1 | usgscug(Uhd)AfaCfAfCfaaguagaugaL96 | 1576 | VPusCfsaucu(Agn)cuugugUfuAfcagcascsa | 1666 |
| AD-738073.1 | ascsagc(Uhd)GfuGfCfUfguaacacaaaL96 | 1577 | VPusUfsugug(Tgn)uacagcAfcAfgcuguscsa | 1667 |
| AD-738074.1 | gscsugu(Ahd)AfcAfCfAfaguagaugcaL96 | 1578 | VPusGfscauc(Tgn)acuuguGfuUfacagcsasc | 1668 |
| AD-738075.1 | uscsaaa(Chd)UfaGfUfGfcaugaauagaL96 | 1579 | VPusCfsuauu(Cgn)augcacUfaGfuuugasusa | 1669 |
| AD-738076.1 | csasaac(Uhd)AfgUfGfCfaugaauagaaL96 | 1580 | VPusUfscuau(Tgn)caugcaCfuAfguuugsasu | 1670 |
| AD-738077.1 | usgscag(Ghd)AfuGfAfUfuguacagaaaL96 | 1581 | VPusUfsucug(Tgn)acaaucAfuCfcugcasgsa | 1671 |
| AD-738078.1 | gscsagg(Ahd)UfgAfUfUfguacagaauaL96 | 1582 | VPusAfsuucu(Ggn)uacaauCfaUfccugcsasg | 1672 |
| AD-738079.1 | csasgga(Uhd)GfaUfUfGfuacagaaucaL96 | 1583 | VPusGfsauuc(Tgn)guacaaUfcAfuccugscsa | 1673 |
| AD-738080.1 | usasuca(Ahd)AfcUfAfGfugcaugaauaL96 | 1584 | VPusAfsuuca(Tgn)gcacuaGfuUfugauascsa | 1674 |
| AD-738081.1 | ususugu(Ghd)CfcUfGfUfuuuaugugcaL96 | 1585 | VPusGfscaca(Tgn)aaaacaGfgCfacaaasgsa | 1675 |
| AD-738082.1 | ususgug(Chd)CfuGfUfUfuuaugugcaaL96 | 1586 | VPusUfsgcac(Agn)uaaaacAfgGfcacaasasg | 1676 |
| AD-738083.1 | csusgca(Ghd)GfaUfGfAfuuguacagaaL96 | 1587 | VPusUfscugu(Agn)caaucaUfcCfugcagsasa | 1677 |
| AD-738084.1 | csasggu(Chd)AfuGfAfGfagaaugggaaL96 | 1588 | VPusUfsccca(Tgn)ucucucAfuGfaccugsgsg | 1678 |
| AD-738085.1 | usasugc(Uhd)GfaAfGfAfaguacguccaL96 | 1589 | VPusGfsgacg(Tgn)acuucuUfcAfgcauasusu | 1679 |
| AD-738086.1 | asusgcu(Ghd)AfaGfAfAfguacguccgaL96 | 1590 | VPusCfsggac(Ggn)uacuucUfuCfagcausasu | 1680 |
| AD-738087.1 | asasacc(Ahd)UfuGfCfUfucacuacccaL96 | 1591 | VPusGfsggua(Ggn)ugaagcAfaUfgguuususg | 1681 |
| AD-738088.1 | asascca(Uhd)UfgCfUfUfcacuacccaaL96 | 1592 | VPusUfsgggu(Agn)gugaagCfaAfugguususu | 1682 |
| AD-738089.1 | usasugu(Ghd)CfaCfAfCfauuaggcauaL96 | 1593 | VPusAfsugcc(Tgn)aaugugUfgCfacauasasa | 1683 |
| AD-738090.1 | usgsugc(Ahd)CfaCfAfUfuaggcauugaL96 | 1594 | VPusCfsaaug(Cgn)cuaaugUfgUfgcacasusa | 1684 |
| AD-738091.1 | gsgsaug(Ahd)UfuGfUfAfcagaaucauaL96 | 1595 | VPusAfsugau(Tgn)cuguacAfaUfcauccsusg | 1685 |
| AD-738092.1 | ascscau(Chd)CfaGfAfAfcuggugcaaaL96 | 1596 | VPusUfsugca(Cgn)caguucUfgGfaugguscsa | 1686 |
| AD-738093.1 | csasccg(Ahd)GfaGfAfGfaaugucccaaL96 | 1597 | VPusUfsggga(Cgn)auucucUfcUfcggugscsu | 1687 |
| AD-738094.1 | gsusugu(Ahd)UfaUfUfAfuucuuguggaL96 | 1598 | VPusCfscaca(Agn)gaauaaUfaUfacaacsusg | 1688 |
| AD-738095.1 | ususaug(Uhd)GfcAfCfAfcauuaggcaaL96 | 1599 | VPusUfsgccu(Agn)auguguGfcAfcauaasasa | 1689 |
| AD-738096.1 | asusgug(Chd)AfcAfCfAfuuaggcauuaL96 | 1600 | VPusAfsaugc(Cgn)uaauguGfuGfcacausasa | 1690 |
| AD-738097.1 | gsusgca(Chd)AfcAfUfUfaggcauugaaL96 | 1601 | VPusUfscaau(Ggn)ccuaauGfuGfugcacsasu | 1691 |
| AD-738098.1 | usgsauu(Ghd)UfaCfAfGfaaucauugcaL96 | 1602 | VPusGfscaau(Ggn)auucugUfaCfaaucasusc | 1692 |
| AD-738099.1 | gscsuuc(Ahd)CfuAfCfCfcaucgguguaL96 | 1603 | VPusAfscacc(Ggn)auggguAfgUfgaagcsasa | 1693 |
| AD-738100.1 | ususuua(Uhd)GfuGfCfAfcacauuaggaL96 | 1604 | VPusCfscuaa(Tgn)gugugcAfcAfuaaaascsa | 1694 |

Table 9 key: U = uridine-3′-phosphate, u = 2′-O-methyluridine-3′-phosphate, us = 2′-O-methyluridine-3′-phosphorothioate, a = 2′-O-methyladenosine-3′-phosphate, A = adenosine-3′-phosphate, as = 2′-O-methyladenosine-3′-phosphorothioate, (Ahd) = 2′-O-hexadecyl-adenosine-3′-phosphate, Gf = 2′-fluoroguanosine-3′-phosphate, Uf-2′-fluorouridine-3′-phosphate,
Cf = 2′-fluorocytidine-3′-phosphate, Af = 2′-fluoroadenosine-3′-phosphate, cs = 2′-O-methylcytidine-3′-phosphate, VP = Vinylphosphate 5′, (Agn) = Adenosine-glycol nucleic acid (GNA), gs = 2′-O-methylguanosine-3′-phosphorothioate, (Chd) = 2′-O-hexadecyl-cytidine-3′-phosphate, (Tgn) = Thymidine-glycol nucleic acid (GNA) S-Isomer, (Ghd) = 2′-O-hexadecyl-guanosine-3′-phosphate, and cs = 2′-O-methylcytidine-3′-phosphorothioate.

TABLE 10
Additional Human APP-Targeting RNAi Agents, Unmodified Sequences;
XM_005548887.2 and NM_001198823.1 Targeting.

| | | SEQ | | SEQ | |
|---|---|---|---|---|---|
| Duplex | Sense Sequence | ID | Antisense Sequence | ID | |
| Name | (5′ to 3′) | NO | (5′ to 3′) | NO | Source Name |
| AD- | CGCUUUCUACACUGUAUUACA | 1695 | UGUAAUACAGUGUAGAAAGCGAU | 1785 | XM_005548887.2_3401- |
| 738012.1 | | | | | 3423_as |
| AD- | GCUUUCUACACUGUAUUACAA | 1696 | UUGUAAUACAGUGUAGAAAGCGA | 1786 | XM_005548887.2_3402- |
| 738013.1 | | | | | 3424_as |
| AD- | UUCUACACUGUAUUACAUAAA | 1697 | UUUAUGUAAUACAGUGUAGAAAG | 1787 | NM_001198823.1_3306- |
| 738014.1 | | | | | 3328_as |
| AD- | UUUCUACACUGUAUUACAUAA | 1698 | UUAUGUAAUACAGUGUAGAAAGC | 1788 | NM_001198823.1_3305- |
| 738015.1 | | | | | 3327_as |
| AD- | AUUUAGCUGUAUCAAACUAGA | 1699 | UCUAGUUUGAUACAGCUAAAUUC | 1789 | XM_005548887.2_2837- |
| 738016.1 | | | | | 2859_as |
| AD- | UUCCUGAUCACUAUGCAUUUA | 1700 | UAAAUGCAUAGUGAUCAGGAAAG | 1790 | XM_005548887.2_3030- |
| 738017.1 | | | | | 3052_as |
| AD- | GUGCUGUAACACAAGUAGAUA | 1701 | UAUCUACUUGUGUUACAGCACAG | 1791 | NM_001198823.1_2602- |
| 738018.1 | | | | | 2624_C1A_as |
| AD- | UUUAGCUGUAUCAAACUAGUA | 1702 | UACUAGUUUGAUACAGCUAAAUU | 1792 | XM_005548887.2_2838- |
| 738019.1 | | | | | 2860_as |
| AD- | UUUCCUGAUCACUAUGCAUUA | 1703 | UAAUGCAUAGUGAUCAGGAAAGG | 1793 | XM_005548887.2_3029- |
| 738020.1 | | | | | 3051_as |
| AD- | AAUGGGUUUUGUGUACUGUAA | 1704 | UUACAGUACACAAAACCCAUUAA | 1794 | XM_005548887.2_2813- |
| 738021.1 | | | | | 2835_as |
| AD- | AUUGUACAGAAUCAUUGCUUA | 1705 | UAAGCAAUGAUUCUGUACAAUCA | 1795 | NM_001198823.1_3272- |
| 738022.1 | | | | | 3294_as |
| AD- | UUGUACAGAAUCAUUGCUUAA | 1706 | UUAAGCAAUGAUUCUGUACAAUC | 1796 | NM_001198823.1_3273- |
| 738023.1 | | | | | 3295_as |
| AD- | UUACUGUACAGAUUGCUGCUA | 1707 | UAGCAGCAAUCUGUACAGUAAAA | 1797 | XM_005548887.2_3113- |
| 738024.1 | | | | | 3135_as |
| AD- | AUAUGCUGAAGAAGUACGUCA | 1708 | UGACGUACUUCUUCAGCAUAUUG | 1798 | XM_005548887.2_1740- |
| 738025.1 | | | | | 1762_as |
| AD- | ACCAUUGCUUCACUACCCAUA | 1709 | UAUGGGUAGUGAAGCAAUGGUUU | 1799 | NM_001198823.1_2506- |
| 738026.1 | | | | | 2528_G1A_as |
| AD- | CUGUGCUGUAACACAAGUAGA | 1710 | UCUACUUGUGUUACAGCACAGCU | 1800 | NM_001198823.1_2600- |
| 738027.1 | | | | | 2622_as |
| AD- | UGCUGUAACACAAGUAGAUGA | 1711 | UCAUCUACUUGUGUUACAGCACA | 1801 | NM_001198823.1_2603- |
| 738028.1 | | | | | 2625_G1A_as |
| AD- | ACAGCUGUGCUGUAACACAAA | 1712 | UUUGUGUUACAGCACAGCUGUCA | 1802 | NM_001198823.1_2596- |
| 738029.1 | | | | | 2618_C1A_as |
| AD- | GCUGUAACACAAGUAGAUGCA | 1713 | UGCAUCUACUUGUGUUACAGCAC | 1803 | NM_001198823.1_2604- |
| 738030.1 | | | | | 2626_G1A_as |
| AD- | UCAAACUAGUGCAUGAAUAGA | 1714 | UCUAUUCAUGCACUAGUUUGAUA | 1804 | NM_001198823.1_2742- |
| 738031.1 | | | | | 2764_as |
| AD- | CAAACUAGUGCAUGAAUAGAA | 1715 | UUCUAUUCAUGCACUAGUUUGAU | 1805 | NM_001198823.1_2743- |
| 738032.1 | | | | | 2765_as |
| AD- | UGCAGGAUGAUUGUACAGAAA | 1716 | UUUCUGUACAAUCAUCCUGCAGA | 1806 | NM_001198823.1_3263- |
| 738033.1 | | | | | 3285_as |
| AD- | GCAGGAUGAUUGUACAGAAUA | 1717 | UAUUCUGUACAAUCAUCCUGCAG | 1807 | NM_001198823.1_3264- |
| 738034.1 | | | | | 3286_G1A_as |
| AD- | CAGGAUGAUUGUACAGAAUCA | 1718 | UGAUUCUGUACAAUCAUCCUGCA | 1808 | NM_001198823.1_3265- |
| 738035.1 | | | | | 3287_as |
| AD- | UAUCAAACUAGUGCAUGAAUA | 1719 | UAUUCAUGCACUAGUUUGAUACA | 1809 | NM_001198823.1_2740- |
| 738036.1 | | | | | 2762_as |
| AD- | UUUGUGCCUGUUUUAUGUGCA | 1720 | UGCACAUAAAACAGGCACAAAGA | 1810 | NM_001198823.1_3070- |
| 738037.1 | | | | | 3092_as |
| AD- | UUGUGCCUGUUUUAUGUGCAA | 1721 | UUGCACAUAAAACAGGCACAAAG | 1811 | NM_001198823.1_3071- |
| 738038.1 | | | | | 3093_G1A_as |
| AD- | CUGCAGGAUGAUUGUACAGAA | 1722 | UUCUGUACAAUCAUCCUGCAGAA | 1812 | NM_001198823.1_3262- |
| 738039.1 | | | | | 3284_as |
| AD- | CAGGUCAUGAGAGAAUGGGAA | 1723 | UUCCCAUUCUCUCAUGACCUGGG | 1813 | NM_001198823.1_1369- |
| 738040.1 | | | | | 1391_as |
| AD- | UAUGUGCACACAUUAGGCAUA | 1724 | UAUGCCUAAUGUGUGCACAUAAA | 1814 | NM_001198823.1_3083- |
| 738041.1 | | | | | 3105_as |
| AD- | UGUGCACACAUUAGGCAUUGA | 1725 | UCAAUGCCUAAUGUGUGCACAUA | 1815 | NM_001198823.1_3085- |
| 738042.1 | | | | | 3107_as |
| AD- | GGAUGAUUGUACAGAAUCAUA | 1726 | UAUGAUUCUGUACAAUCAUCCUG | 1816 | NM_001198823.1_3267- |
| 738043.1 | | | | | 3289_as |
| AD- | ACCAUCCAGAACUGGUGCAAA | 1727 | UUUGCACCAGUUCUGGAUGGUCA | 1817 | NM_001198823.1_424- |
| 738044.1 | | | | | 446_C1A_as |
| AD- | UAUGCUGAAGAAGUACGUCCA | 1728 | UGGACGUACUUCUUCAGCAUAUU | 1818 | XM_005548887.2_1741- |
| 738045.1 | | | | | 1763_as |
| AD- | AUGCUGAAGAAGUACGUCCGA | 1729 | UCGGACGUACUUCUUCAGCAUAU | 1819 | XM_005548887.2_1742- |
| 738046.1 | | | | | 1764_as |
| AD- | AAACCAUUGCUUCACUACCCA | 1730 | UGGGUAGUGAAGCAAUGGUUUUG | 1820 | XM_005548887.2_2614- |
| 738047.1 | | | | | 2636_as |
| AD- | AACCAUUGCUUCACUACCCAA | 1731 | UUGGGUAGUGAAGCAAUGGUUUU | 1821 | XM_005548887.2_2615- |
| 738048.1 | | | | | 2637_as |
| AD- | CACCGAGAGAGAAUGUCCCAA | 1732 | UUGGGACAUUCUCUCUCGGUGCU | 1822 | NM_001198823.1_1351- |
| 397217.2 | | | | | 1373_C1A_as |
| AD- | GUUGUAUAUUAUUCUUGUGGA | 1733 | UCCACAAGAAUAAUAUACAACUG | 1823 | XM_005548887.2_2906- |
| 738049.1 | | | | | 2928_as |
| AD- | UUAUGUGCACACAUUAGGCAA | 1734 | UUGCCUAAUGUGUGCACAUAAAA | 1824 | NM_001198823.1_3082- |
| 738050.1 | | | | | 3104_as |
| AD- | AUGUGCACACAUUAGGCAUUA | 1735 | UAAUGCCUAAUGUGUGCACAUAA | 1825 | NM_001198823.1_3084- |
| 738051.1 | | | | | 3106_C1A_as |
| AD- | GUGCACACAUUAGGCAUUGAA | 1736 | UUCAAUGCCUAAUGUGUGCACAU | 1826 | NM_001198823.1_3086- |
| 738052.1 | | | | | 3108_C1A_as |
| AD- | UGAUUGUACAGAAUCAUUGCA | 1737 | UGCAAUGAUUCUGUACAAUCAUC | 1827 | NM_001198823.1_3270- |
| 738053.1 | | | | | 3292_as |
| AD- | GCUUCACUACCCAUCGGUGUA | 1738 | UACACCGAUGGGUAGUGAAGCAA | 1828 | NM_001198823.1_2512- |
| 738054.1 | | | | | 2534_as |
| AD- | UUUUAUGUGCACACAUUAGGA | 1739 | UCCUAAUGUGUGCACAUAAAACA | 1829 | NM_001198823.1_3080- |
| 738055.1 | | | | | 3102_G1A_as |
| AD- | CGCUUUCUACACUGUAUUACA | 1740 | UGUAAUACAGUGUAGAAAGCGAU | 1830 | XM_005548887.2_3401- |
| 738056.1 | | | | | 3423_as |
| AD- | GCUUUCUACACUGUAUUACAA | 1741 | UUGUAATACAGUGUAGAAAGCGA | 1831 | XM_005548887.2_3402- |
| 738057.1 | | | | | 3424_as |
| AD- | UUCUACACUGUAUUACAUAAA | 1742 | UUUAUGTAAUACAGUGUAGAAAG | 1832 | XM_005548887.2_3405- |
| 738058.1 | | | | | 3427_as |
| AD- | UUUCUACACUGUAUUACAUAA | 1743 | UUAUGUAAUACAGUGUAGAAAGC | 1833 | XM_005548887.2_3404- |
| 738059.1 | | | | | 3426_as |
| AD- | AUUUAGCUGUAUCAAACUAGA | 1744 | UCUAGUTUGAUACAGCUAAAUUC | 1834 | XM_005548887.2_2837- |
| 738060.1 | | | | | 2859_as |
| AD- | UUCCUGAUCACUAUGCAUUUA | 1745 | UAAAUGCAUAGUGAUCAGGAAAG | 1835 | XM_005548887.2_3030- |
| 738061.1 | | | | | 3052_as |
| AD- | GUGCUGUAACACAAGUAGAUA | 1746 | UAUCUACUUGUGUUACAGCACAG | 1836 | XM_005548887.2_2716- |
| 738062.1 | | | | | 2738_as |
| AD- | UUUAGCUGUAUCAAACUAGUA | 1747 | UACUAGTUUGAUACAGCUAAAUU | 1837 | XM_005548887.2_2838- |
| 738063.1 | | | | | 2860_as |
| AD- | UUUCCUGAUCACUAUGCAUUA | 1748 | UAAUGCAUAGUGAUCAGGAAAGG | 1838 | XM_005548887.2_3029- |
| 738064.1 | | | | | 3051_as |
| AD- | AAUGGGUUUUGUGUACUGUAA | 1749 | UUACAGTACACAAAACCCAUUAA | 1839 | XM_005548887.2_2813- |
| 738065.1 | | | | | 2835_as |
| AD- | UUACUGUACAGAUUGCUGCUA | 1750 | UAGCAGCAAUCUGUACAGUAAAA | 1840 | XM_005548887.2_3113- |
| 738066.1 | | | | | 3135_as |
| AD- | AUUGUACAGAAUCAUUGCUUA | 1751 | UAAGCAAUGAUUCUGUACAAUCA | 1841 | XM_005548887.2_3371- |
| 738067.1 | | | | | 3393_as |
| AD- | UUGUACAGAAUCAUUGCUUAA | 1752 | UUAAGCAAUGAUUCUGUACAAUC | 1842 | XM_005548887.2_3372- |
| 738068.1 | | | | | 3394_as |
| AD- | AUAUGCUGAAGAAGUACGUCA | 1753 | UGACGUACUUCUUCAGCAUAUUG | 1843 | XM_005548887.2_1740- |
| 738069.1 | | | | | 1762_as |
| AD- | ACCAUUGCUUCACUACCCAUA | 1754 | UAUGGGTAGUGAAGCAAUGGUUU | 1844 | XM_005548887.2_2616- |
| 738070.1 | | | | | 2638_as |
| AD- | CUGUGCUGUAACACAAGUAGA | 1755 | UCUACUTGUGUUACAGCACAGCU | 1845 | XM_005548887.2_2714- |
| 738071.1 | | | | | 2736_as |
| AD- | UGCUGUAACACAAGUAGAUGA | 1756 | UCAUCUACUUGUGUUACAGCACA | 1846 | XM_005548887.2_2717- |
| 738072.1 | | | | | 2739_as |
| AD- | ACAGCUGUGCUGUAACACAAA | 1757 | UUUGUGTUACAGCACAGCUGUCA | 1847 | XM_005548887.2_2710- |
| 738073.1 | | | | | 2732_as |
| AD- | GCUGUAACACAAGUAGAUGCA | 1758 | UGCAUCTACUUGUGUUACAGCAC | 1848 | XM_005548887.2_2718- |
| 738074.1 | | | | | 2740_as |
| AD- | UCAAACUAGUGCAUGAAUAGA | 1759 | UCUAUUCAUGCACUAGUUUGAUA | 1849 | XM_005548887.2_2848- |
| 738075.1 | | | | | 2870_as |
| AD- | CAAACUAGUGCAUGAAUAGAA | 1760 | UUCUAUTCAUGCACUAGUUUGAU | 1850 | XM_005548887.2_2849- |
| 738076.1 | | | | | 2871_as |
| AD- | UGCAGGAUGAUUGUACAGAAA | 1761 | UUUCUGTACAAUCAUCCUGCAGA | 1851 | XM_005548887.2_3362- |
| 738077.1 | | | | | 3384_as |
| AD- | GCAGGAUGAUUGUACAGAAUA | 1762 | UAUUCUGUACAAUCAUCCUGCAG | 1852 | XM_005548887.2_3363- |
| 738078.1 | | | | | 3385_as |
| AD- | CAGGAUGAUUGUACAGAAUCA | 1763 | UGAUUCTGUACAAUCAUCCUGCA | 1853 | XM_005548887.2_3364- |
| 738079.1 | | | | | 3386_as |
| AD- | UAUCAAACUAGUGCAUGAAUA | 1764 | UAUUCATGCACUAGUUUGAUACA | 1854 | XM_005548887.2_2846- |
| 738080.1 | | | | | 2868_as |
| AD- | UUUGUGCCUGUUUUAUGUGCA | 1765 | UGCACATAAAACAGGCACAAAGA | 1855 | XM_005548887.2_3180- |
| 738081.1 | | | | | 3202_as |
| AD- | UUGUGCCUGUUUUAUGUGCAA | 1766 | UUGCACAUAAAACAGGCACAAAG | 1856 | XM_005548887.2_3181- |
| 738082.1 | | | | | 3203_as |
| AD- | CUGCAGGAUGAUUGUACAGAA | 1767 | UUCUGUACAAUCAUCCUGCAGAA | 1857 | XM_005548887.2_3361- |
| 738083.1 | | | | | 3383_as |
| AD- | CAGGUCAUGAGAGAAUGGGAA | 1768 | UUCCCATUCUCUCAUGACCUGGG | 1858 | XM 005548887.2_1487- |
| 738084.1 | | | | | 1509_as |
| AD- | UAUGCUGAAGAAGUACGUCCA | 1769 | UGGACGTACUUCUUCAGCAUAUU | 1859 | XM_005548887.2_1741- |
| 738085.1 | | | | | 1763_as |
| AD- | AUGCUGAAGAAGUACGUCCGA | 1770 | UCGGACGUACUUCUUCAGCAUAU | 1860 | XM_005548887.2_1742- |
| 738086.1 | | | | | 1764_as |
| AD- | AAACCAUUGCUUCACUACCCA | 1771 | UGGGUAGUGAAGCAAUGGUUUUG | 1861 | XM_005548887.2_2614- |
| 738087.1 | | | | | 2636_as |
| AD- | AACCAUUGCUUCACUACCCAA | 1772 | UUGGGUAGUGAAGCAAUGGUUUU | 1862 | XM_005548887.2_2615- |
| 738088.1 | | | | | 2637_as |
| AD- | UAUGUGCACACAUUAGGCAUA | 1773 | UAUGCCTAAUGUGUGCACAUAAA | 1863 | XM_005548887.2_3193- |
| 738089.1 | | | | | 3215_as |
| AD- | UGUGCACACAUUAGGCAUUGA | 1774 | UCAAUGCCUAAUGUGUGCACAUA | 1864 | XM_005548887.2_3195- |
| 738090.1 | | | | | 3217_as |
| AD- | GGAUGAUUGUACAGAAUCAUA | 1775 | UAUGAUTCUGUACAAUCAUCCUG | 1865 | XM_005548887.2_3366- |
| 738091.1 | | | | | 3388_as |
| AD- | ACCAUCCAGAACUGGUGCAAA | 1776 | UUUGCACCAGUUCUGGAUGGUCA | 1866 | XM_005548887.2_767- |
| 738092.1 | | | | | 789_as |
| AD- | CACCGAGAGAGAAUGUCCCAA | 1777 | UUGGGACAUUCUCUCUCGGUGCU | 1867 | XM_005548887.2_1469- |
| 738093.1 | | | | | 1491_as |
| AD- | GUUGUAUAUUAUUCUUGUGGA | 1778 | UCCACAAGAAUAAUAUACAACUG | 1868 | XM_005548887.2_2906- |
| 738094.1 | | | | | 2928_as |
| AD- | UUAUGUGCACACAUUAGGCAA | 1779 | UUGCCUAAUGUGUGCACAUAAAA | 1869 | XM_005548887.2_3192- |
| 738095.1 | | | | | 3214_as |
| AD- | AUGUGCACACAUUAGGCAUUA | 1780 | UAAUGCCUAAUGUGUGCACAUAA | 1870 | XM_005548887.2_3194- |
| 738096.1 | | | | | 3216_as |
| AD- | GUGCACACAUUAGGCAUUGAA | 1781 | UUCAAUGCCUAAUGUGUGCACAU | 1871 | XM_005548887.2_3196- |
| 738097.1 | | | | | 3218_as |
| AD- | UGAUUGUACAGAAUCAUUGCA | 1782 | UGCAAUGAUUCUGUACAAUCAUC | 1872 | XM_005548887.2_3369- |
| 738098.1 | | | | | 3391_as |
| AD- | GCUUCACUACCCAUCGGUGUA | 1783 | UACACCGAUGGGUAGUGAAGCAA | 1873 | XM_005548887.2_2622- |
| 738099.1 | | | | | 2644_as |
| AD- | UUUUAUGUGCACACAUUAGGA | 1784 | UCCUAATGUGUGCACAUAAAACA | 1874 | XM_005548887.2_3190- |
| 738100.1 | | | | | 3212_as |

TABLE 11
Additional Human APP-Targeting RNAi Agents, Modified Sequences.

| | | | SEQ | | SEQ |
|---|---|---|---|---|---|
| | Duplex | | ID | | ID |
| Target | Name | Sense Sequence (5′ to 3′) | NO | Antisense Sequence (5′ to 3′) | NO |
| APP | AD- | usasgug(Chd)AfuGfAfAfuagauucucaL96 | 1875 | VPusGfsagaa(Tgn)cuauucAfuGfcacuasgsu | 1984 |
| | 886823.1 | | | | |
| APP | AD- | usasgug(Chd)AfugAfAfuagauucucaL96 | 1876 | VPusGfsagaa(Tgn)cuauucAfuGfcacuasgsu | 1985 |
| | 886824.1 | | | | |
| APP | AD- | usasgug(Chd)AfudGAfAfuagauucucaL96 | 1877 | VPusGfsagaa(Tgn)cuauucAfuGfcacuasgsu | 1986 |
| | 886825.1 | | | | |
| APP | AD- | usasgug(Chd)AfudGadAuagauucucaL96 | 1878 | VPusGfsagaa(Tgn)cuauUfcAfuGfcacuasgsu | 1987 |
| | 886826.1 | | | | |
| APP | AD- | usasgug(Chd)AfuGfAfAfuagauucucaL96 | 1879 | VPusdGsagaa(Tgn)cuauucAfuGfcacuasgsu | 1988 |
| | 886827.1 | | | | |
| APP | AD- | usasgug(Chd)AfuGfAfAfuagauucucaL96 | 1880 | VPusGfsagaa(Tgn)cuauucAfugcacuasgsu | 1989 |
| | 886828.1 | | | | |
| APP | AD- | usasgug(Chd)AfuGfAfAfuagauucucaL96 | 1881 | VPusGfsagaa(Tgn)cuauucAfudGcacuasgsu | 1990 |
| | 886829.1 | | | | |
| APP | AD- | usasgug(Chd)AfuGfaAfuagauucucaL96 | 1882 | VPusGfsagaa(Tgn)cuaudTcAfudGcacuasgsu | 1991 |
| | 886830.1 | | | | |
| APP | AD- | usasgug(Chd)AfuGfaAfuagauucucaL96 | 1883 | VPusGfsagaa(Tgn)cuaudTcAfugcacuasgsu | 1992 |
| | 886831.1 | | | | |
| APP | AD- | usasgug(Chd)AfuGfAfAfuagauucucaL96 | 1884 | VPusdGsagaa(Tgn)cuauucAfudGcacuasgsu | 1993 |
| | 886832.1 | | | | |
| APP | AD- | usasgug(Chd)AfuGfaAfuagauucucaL96 | 1885 | VPusdGsagaa(Tgn)cuaudTcAfudGcacuasgsu | 1994 |
| | 886833.1 | | | | |
| APP | AD- | usasgug(Chd)AfudGAfAfuagauucucaL96 | 1886 | VPusdGsagaa(Tgn)cuauucAfuGfcacuasgsu | 1995 |
| | 886834.1 | | | | |
| APP | AD- | usasgug(Chd)AfudGAfAfuagauucucaL96 | 1887 | VPusGfsagaa(Tgn)cuauucAfudGcacuasgsu | 1996 |
| | 886836.1 | | | | |
| APP | AD- | usasgug(Chd)AfudGaAfuagauucucaL96 | 1888 | VPusGfsagaa(Tgn)cuaudTcAfudGcacuasgsu | 1997 |
| | 886837.1 | | | | |
| APP | AD- | usasgug(Chd)AfudGaAfuagauucucaL96 | 1889 | VPusGfsagaa(Tgn)cuaudTcAfugcacuasgsu | 1998 |
| | 886838.1 | | | | |
| APP | AD- | usasgug(Chd)AfudGAfAfuagauucucaL96 | 1890 | VPusdGsagaa(Tgn)cuauucAfudGcacuasgsu | 1999 |
| | 886839.1 | | | | |
| APP | AD- | usasgug(Chd)AfudGAfAfuagauucucaL96 | 1891 | VPusdGsagaa(Tgn)cuauucAfudGcacuasgsu | 2000 |
| | 886839.2 | | | | |
| APP | AD- | usasgug(Chd)AfudGAfAfuagauucucaL96 | 1892 | VPusdGsagaa(Tgn)cuaudTcAfudGcacuasgsu | 2001 |
| | 886840.1 | | | | |
| APP | AD- | usasgug(Chd)AfudGaAfuagauucucaL96 | 1893 | VPusdGsagaa(Tgn)cuaudTcAfudGcacuasgsu | 2002 |
| | 886841.1 | | | | |
| APP | AD- | usasgug(Chd)AfudGadAuagauucucaL96 | 1894 | VPusdGsagaa(Tgn)cuaudTcAfudGcacuasgsu | 2003 |
| | 886842.1 | | | | |
| APP | AD- | usasgug(Chd)audGadAuagauucucaL96 | 1895 | VPusdGsagaa(Tgn)cuaudTcAfudGcacuasgsu | 2004 |
| | 886843.1 | | | | |
| APP | AD- | usasgug(Chd)audGadAuagauucucaL96 | 1896 | VPudGagaa(Tgn)cuaudTcAfudGcacuasgsu | 2005 |
| | 886844.1 | | | | |
| APP | AD- | usasgug(Chd)audGadAuagauucucaL96 | 1897 | VPudGagaa(Tgn)cuauUfcAfudGcacuasgsu | 2006 |
| | 886845.1 | | | | |
| APP | AD- | usasgug(Chd)audGadAuagauucucaL96 | 1898 | VPudGadGadAucuauUfcAfudGcacuasgsu | 2007 |
| | 886846.1 | | | | |
| APP | AD- | usasgug(Chd)AfudGAfAfuagauucucaL96 | 1899 | VPudGagaa(Tgn)cuauucAfudGcacuasgsu | 2008 |
| | 886847.1 | | | | |
| APP | AD- | usasgug(Chd)AfudGAfAfuagauucucaL96 | 1900 | VPusdGsagadA(Tgn)cuauucAfudGcacuasgsu | 2009 |
| | 886848.1 | | | | |
| APP | AD- | usasgug(Chd)AfudGAfAfuagauucucaL96 | 1901 | VPusdGsagdAa(Tgn)cuauucAfudGcacuasgsu | 2010 |
| | 886849.1 | | | | |
| APP | AD- | usasgug(Chd)AfudGAfAfuagauucucaL96 | 1902 | VPusdGsagadA(Tgn)cuaudTcAfudGcacuasgsu | 2011 |
| | 886850.1 | | | | |
| APP | AD- | usasgug(Chd)AfudGAfAfuagauucucaL96 | 1903 | VPusdGsagdAa(Tgn)cuaudTcAfudGcacuasgsu | 2012 |
| | 886851.1 | | | | |
| APP | AD- | usasgug(Chd)AfudGAfAfuagauucucaL96 | 1904 | VPusdGsagadA(Tgn)cuaudTcAfugcacuasgsu | 2013 |
| | 886852.1 | | | | |
| APP | AD- | usasgug(Chd)AfudGAfAfuagauucucaL96 | 1905 | VPusdGsagdAa(Tgn)cuaudTcAfugcacuasgsu | 2014 |
| | 886853.1 | | | | |
| APP | AD- | usasgug(Chd)AfudGadAuagauucucaL96 | 1906 | VPusdGsagdAa(Tgn)cuaudTcAfugcacuasgsu | 2015 |
| | 886854.1 | | | | |
| APP | AD- | usasgug(Chd)AfudGAfAfuagauucucaL96 | 1907 | VPudGagadA(Tgn)cuauucAfudGcacuasgsu | 2016 |
| | 886855.1 | | | | |
| APP | AD- | usasgug(Chd)AfudGAfAfuagauucucaL96 | 1908 | VPudGagdAa(Tgn)cuauucAfudGcacuasgsu | 2017 |
| | 886856.1 | | | | |
| APP | AD- | usasgug(Chd)AfudGAfAfuagauucucaL96 | 1909 | VPudGagadA(Tgn)cuaudTcAfudGcacuasgsu | 2018 |
| | 886857.1 | | | | |
| APP | AD- | usasgug(Chd)AfudGAfAfuagauucucaL96 | 1910 | VPudGagdAa(Tgn)cuaudTcAfudGcacuasgsu | 2019 |
| | 886858.1 | | | | |
| APP | AD- | usasgug(Chd)AfudGAfAfuagauucucaL96 | 1911 | VPudGagadA(Tgn)cuaudTcAfugcacuasgsu | 2020 |
| | 886859.1 | | | | |
| APP | AD- | usasgug(Chd)AfudGAfAfuagauucucaL96 | 1912 | VPudGagdAa(Tgn)cuaudTcAfugcacuasgsu | 2021 |
| | 886860.1 | | | | |
| APP | AD- | usasgug(Chd)AfuGfAfAfuagauucucaL96 | 1913 | VPusGfsagaa(Tgn)cuauucAfuGfcacuasusg | 2022 |
| | 886861.1 | | | | |
| APP | AD- | usasgug(Chd)AfudGAfAfuagauucucaL96 | 1914 | VPusdGsagadA(Tgn)cuaudTcAfudGcacuasusg | 2023 |
| | 886862.1 | | | | |
| APP | AD- | usasgug(Chd)AfudGAfAfuagauucucaL96 | 1915 | VPusdGsagdAa(Tgn)cuaudTcAfudGcacuasusg | 2024 |
| | 886863.1 | | | | |
| APP | AD- | gsgscua(Chd)GfaAfAfAfuccaaccuaaL96 | 1916 | VPusUfsaggu(Tgn)ggauuuUfcGfuagccsgsu | 2025 |
| | 886864.1 | | | | |
| APP | AD- | gsgscua(Chd)gaAfAfAfuccaaccuaaL96 | 1917 | VPusUfsaggu(Tgn)ggauuuUfcGfuagccsgsu | 2026 |
| | 886865.1 | | | | |
| APP | AD- | gsgscua(Chd)dGaAfAfAfuccaaccuaaL96 | 1918 | VPusUfsaggu(Tgn)ggauuuUfcGfuagccsgsu | 2027 |
| | 886866.1 | | | | |
| APP | AD- | gsgscua(Chd)GfaAfAfAfuccaaccuaaL96 | 1919 | VPuUfaggu(Tgn)ggauuuUfcGfuagccsgsu | 2028 |
| | 886867.1 | | | | |
| APP | AD- | gsgscua(Chd)GfaAfAfAfuccaaccuaaL96 | 1920 | VPusUfsaggu(Tgn)ggauuuUfcguagccsgsu | 2029 |
| | 886868.1 | | | | |
| APP | AD- | gsgscua(Chd)GfaAfAfAfuccaaccuaaL96 | 1921 | VPusUfsaggu(Tgn)ggauuuUfcdGuagccsgsu | 2030 |
| | 886869.1 | | | | |
| APP | AD- | gsgscua(Chd)GfaAfAfAfuccaaccuaaL96 | 1922 | VPusUfsaggu(Tgn)ggaudTuUfcdGuagccsgsu | 2031 |
| | 886870.1 | | | | |
| APP | AD- | gsgscua(Chd)gaAfaAfuccaaccuaaL96 | 1923 | VPusUfsaggu(Tgn)ggaudTuUfcdGuagccsgsu | 2032 |
| | 886871.1 | | | | |
| APP | AD- | gsgscua(Chd)gaAfaAfuccaaccuaaL96 | 1924 | VPusUfsaggu(Tgn)ggauUfuUfcdGuagccsgsu | 2033 |
| | 886872.1 | | | | |
| APP | AD- | gsgscua(Chd)gadAadAuccaaccuaaL96 | 1925 | VPusUfsaggu(Tgn)ggaudTuUfcdGuagccsgsu | 2034 |
| | 886873.1 | | | | |
| APP | AD- | gsgscua(Chd)GfaAfAfAfuccaaccuaaL96 | 1926 | VPusUfsaggu(Tgn)ggaudTuUfcguagccsgsu | 2035 |
| | 886874.1 | | | | |
| APP | AD- | gsgscua(Chd)gaAfaAfuccaaccuaaL96 | 1927 | VPusUfsaggu(Tgn)ggaudTuUfcguagccsgsu | 2036 |
| | 886875.1 | | | | |
| APP | AD- | gsgscua(Chd)gaAfaAfuccaaccuaaL96 | 1928 | VPusUfsaggu(Tgn)ggauUfuUfcguagccsgsu | 2037 |
| | 886876.1 | | | | |
| APP | AD- | gsgscua(Chd)gadAadAuccaaccuaaL96 | 1929 | VPusUfsaggu(Tgn)ggaudTuUfcguagccsgsu | 2038 |
| | 886877.1 | | | | |
| APP | AD- | gsgscua(Chd)gaAfAfAfuccaaccuaaL96 | 1930 | VPusUfsaggu(Tgn)ggauuuUfcguagccsgsu | 2039 |
| | 886878.1 | | | | |
| APP | AD- | gsgscua(Chd)gaAfAfAfuccaaccuaaL96 | 1931 | VPusUfsaggu(Tgn)ggauuuUfcdGuagccsgsu | 2040 |
| | 886879.1 | | | | |
| APP | AD- | gsgscua(Chd)gaAfAfAfuccaaccuaaL96 | 1932 | VPusUfsaggu(Tgn)ggaudTuUfcdGuagccsgsu | 2041 |
| | 886880.1 | | | | |
| APP | AD- | gsgscua(Chd)gaAfAfAfuccaaccuaaL96 | 1933 | VPusUfsaggu(Tgn)ggaudTuUfcguagccsgsu | 2042 |
| | 886881.1 | | | | |
| APP | AD- | gsgscua(Chd)gaAfAfAfuccaaccuaaL96 | 1934 | VPuUfaggdT(Tgn)ggauuuUfcguagccsgsu | 2043 |
| | 886882.1 | | | | |
| APP | AD- | gsgscua(Chd)gaAfAfAfuccaaccuaaL96 | 1935 | VPuUfaggdT(Tgn)ggauuuUfcdGuagccsgsu | 2044 |
| | 886883.1 | | | | |
| APP | AD- | gsgscua(Chd)gaAfAfAfuccaaccuaaL96 | 1936 | VPuUfaggdT(Tgn)ggaudTuUfcdGuagccsgsu | 2045 |
| | 886884.1 | | | | |
| APP | AD- | gsgscua(Chd)gaAfAfAfuccaaccuaaL96 | 1937 | VPuUfaggdT(Tgn)ggaudTuUfcguagccsgsu | 2046 |
| | 886885.1 | | | | |
| APP | AD- | gsgscua(Chd)gaAfAfAfuccaaccuaaL96 | 1938 | VPuUfagdGu(Tgn)ggauuuUfcguagccsgsu | 2047 |
| | 886886.1 | | | | |
| APP | AD- | gsgscua(Chd)gaAfAfAfuccaaccuaaL96 | 1939 | VPuUfagdGu(Tgn)ggauuuUfcdGuagccsgsu | 2048 |
| | 886887.1 | | | | |
| APP | AD- | gsgscua(Chd)gaAfAfAfuccaaccuaaL96 | 1940 | VPuUfagdGu(Tgn)ggaudTuUfcdGuagccsgsu | 2049 |
| | 886888.1 | | | | |
| APP | AD- | gsgscua(Chd)gaAfAfAfuccaaccuaaL96 | 1941 | VPuUfagdGu(Tgn)ggaudTuUfcguagccsgsu | 2050 |
| | 886889.1 | | | | |
| APP | AD- | gsgscua(Chd)GfaAfAfAfuccaaccuaaL96 | 1942 | VPusUfsaggu(Tgn)ggauuuUfcGfuagccsusg | 2051 |
| | 886890.1 | | | | |
| APP | AD- | gsgscua(Chd)gaAfAfAfuccaaccuaaL96 | 1943 | VPusUfsaggu(Tgn)ggauuuUfcdGuagccsusg | 2052 |
| | 886891.1 | | | | |
| APP | AD- | gsgscua(Chd)gaAfAfAfuccaaccuaaL96 | 1944 | VPuUfaggdT(Tgn)ggaudTuUfcdGuagccsusg | 2053 |
| | 886892.1 | | | | |
| APP | AD- | gsgscua(Chd)gaAfAfAfuccaaccuaaL96 | 1945 | VPuUfagdGu(Tgn)ggaudTuUfcdGuagccsusg | 2054 |
| | 886893.1 | | | | |
| APP | AD- | asasaga(Ghd)CfaAfAfAfcuauucagaaL96 | 1946 | VPusUfscugAfaUfAfguuuUfgCfucuuuscsu | 2055 |
| | 886894.1 | | | | |
| APP | AD- | asasag(Ahd)gCfaAfAfAfcuauucagaaL96 | 1947 | VPusUfscugAfaUfAfguuuUfgCfucuuuscsu | 2056 |
| | 886895.1 | | | | |
| APP | AD- | asasagag(Chd)aAfAfAfcuauucagaaL96 | 1948 | VPusUfscugAfaUfAfguuuUfgCfucuuuscsu | 2057 |
| | 886896.1 | | | | |
| APP | AD- | asasagagCfaAfAfAfcua(Uhd)ucagaaL96 | 1949 | VPusUfscugAfaUfAfguuuUfgCfucuuuscsu | 2058 |
| | 886897.1 | | | | |
| APP | AD- | asasagagCfaAfAfAfcuau(Uhd)cagaaL96 | 1950 | VPusUfscugAfaUfAfguuuUfgCfucuuuscsu | 2059 |
| | 886898.1 | | | | |
| APP | AD- | asasagagCfaAfAfAfcuauu(Chd)agaaL96 | 1951 | VPusUfscugAfaUfAfguuuUfgCfucuuuscsu | 2060 |
| | 886899.1 | | | | |
| APP | AD- | asasaga(Ghd)CfaAfAfAfcuauucagaaL96 | 1952 | VPusUfscugAfauaguuuUfgCfucuuuscsu | 2061 |
| | 886900.1 | | | | |
| APP | AD- | asasaga(Ghd)CfaAfAfAfcuauucagaaL96 | 1953 | VPuUfcugAfaUfAfguuuUfgCfucuuuscsu | 2062 |
| | 886901.1 | | | | |
| APP | AD- | asasaga(Ghd)CfaAfAfAfcuauucagaaL96 | 1954 | VPuUfcugAfauaguuuUfgCfucuuuscsu | 2063 |
| | 886902.1 | | | | |
| APP | AD- | asasag(Ahd)gCfaAfAfAfcuauucagaaL96 | 1955 | VPuUfcugAfaUfAfguuuUfgCfucuuuscsu | 2064 |
| | 886903.1 | | | | |
| APP | AD- | asasag(Ahd)gCfaAfAfAfcuauucagaaL96 | 1956 | VPuUfcugAfauaguuuUfgCfucuuuscsu | 2065 |
| | 886904.1 | | | | |
| APP | AD- | asasagag(Chd)aAfAfAfcuauucagaaL96 | 1957 | VPuUfcugAfaUfAfguuuUfgCfucuuuscsu | 2066 |
| | 886905.1 | | | | |
| APP | AD- | asasagag(Chd)aAfAfAfcuauucagaaL96 | 1958 | VPuUfcugAfauaguuuUfgCfucuuuscsu | 2067 |
| | 886906.1 | | | | |
| APP | AD- | asasagag(Chd)aAfaAfcuauucagaaL96 | 1959 | VPuUfcugAfauagudTuUfgCfucuuuscsu | 2068 |
| | 886907.1 | | | | |
| APP | AD- | asasagag(Chd)adAadAcuauucagaaL96 | 1960 | VPuUfcugAfauagudTuUfgCfucuuuscsu | 2069 |
| | 886908.1 | | | | |
| APP | AD- | asasagag(Chd)adAadAcuauucagaaL96 | 1961 | VPuUfcugdAauagudTuUfgdCucuuuscsu | 2070 |
| | 886909.1 | | | | |
| APP | AD- | asasaga(Ghd)CfaAfAfAfcuauucagaaL96 | 1962 | VPusUfscugAfauaguuuUfgCfucuuususg | 2071 |
| | 886910.1 | | | | |
| APP | AD- | asasagagCfaAfAfAfcua(Uhd)ucagaaL96 | 1963 | VPuUfcugAfauaguuuUfgCfucuuususg | 2072 |
| | 886911.1 | | | | |
| APP | AD- | asasagag(Chd)aAfAfAfcuauucagaaL96 | 1964 | VPuUfcugAfauaguuuUfgCfucuuususg | 2073 |
| | 886912.1 | | | | |
| APP | AD- | ususuau(Ghd)AfuUfUfAfcucauuaucaL96 | 1965 | VPusGfsauaAfuGfAfguaaAfuCfauaaasasc | 2074 |
| | 886913.1 | | | | |
| APP | AD- | ususua(Uhd)gAfuUfUfAfcucauuaucaL96 | 1966 | VPusGfsauaAfuGfAfguaaAfuCfauaaasasc | 2075 |
| | 886914.1 | | | | |
| APP | AD- | ususuaug(Ahd)uUfUfAfcucauuaucaL96 | 1967 | VPusGfsauaAfuGfAfguaaAfuCfauaaasasc | 2076 |
| | 886915.1 | | | | |
| APP | AD- | ususuaugAfuUfUfAfcuc(Ahd)uuaucaL96 | 1968 | VPusGfsauaAfuGfAfguaaAfuCfauaaasasc | 2077 |
| | 886916.1 | | | | |
| APP | AD- | ususuaugAfuUfUfAfcuca(Uhd)uaucaL 96 | 1969 | VPusGfsauaAfuGfAfguaaAfuCfauaaasasc | 2078 |
| | 886917.1 | | | | |
| APP | AD- | ususuaugAfuUfUfAfcucau(Uhd)aucaL96 | 1970 | VPusGfsauaAfuGfAfguaaAfuCfauaaasasc | 2079 |
| | 886918.1 | | | | |
| APP | AD- | ususuau(Ghd)AfuUfUfAfcucauuaucaL96 | 1971 | VPusGfsauaAfugaguaaAfuCfauaaasasc | 2080 |
| | 886919.1 | | | | |
| APP | AD- | ususuau(Ghd)AfuUfUfAfcucauuaucaL96 | 1972 | VPusdGsauaAfugaguaaAfuCfauaaasasc | 2081 |
| | 886920.1 | | | | |
| APP | AD- | ususuau(Ghd)AfuUfUfAfcucauuaucaL96 | 1973 | VPudGauaAfugaguaaAfuCfauaaasasc | 2082 |
| | 886921.1 | | | | |
| APP | AD- | ususua(Uhd)gAfuUfUfAfcucauuaucaL96 | 1974 | VPusdGsauaAfugaguaaAfuCfauaaasasc | 2083 |
| | 886922.1 | | | | |
| APP | AD- | ususua(Uhd)gAfuUfUfAfcucauuaucaL96 | 1975 | VPudGauaAfugaguaaAfuCfauaaasasc | 2084 |
| | 886923.1 | | | | |
| APP | AD- | ususuaug(Ahd)uUfUfAfcucauuaucaL96 | 1976 | VPusdGsauaAfugaguaaAfuCfauaaasasc | 2085 |
| | 886924.1 | | | | |
| APP | AD- | ususuaug(Ahd)uUfUfAfcucauuaucaL96 | 1977 | VPudGauaAfugaguaaAfuCfauaaasasc | 2086 |
| | 886925.1 | | | | |
| APP | AD- | ususuaug(Ahd)uUfuAfcucauuaucaL96 | 1978 | VPudGauadAugagudAaAfuCfauaaasasc | 2087 |
| | 886926.1 | | | | |
| APP | AD- | ususuaug(Ahd)uUfudAcucauuaucaL96 | 1979 | VPudGauadAugagudAaAfuCfauaaasasc | 2088 |
| | 886927.1 | | | | |
| APP | AD- | ususuaug(Ahd)uUfudAcucauuaucaL96 | 1980 | VPudGauadAugagudAaAfudCauaaasasc | 2089 |
| | 886928.1 | | | | |
| APP | AD- | ususuau(Ghd)AfuUfUfAfcucauuaucaL96 | 1981 | VPusGfsauaAfugaguaaAfuCfauaaasusg | 2090 |
| | 886929.1 | | | | |
| APP | AD- | ususuaugAfuUfUfAfcuc(Ahd)uuaucaL96 | 1982 | VPusdGsauaAfugaguaaAfuCfauaaasusg | 2091 |
| | 886930.1 | | | | |
| APP | AD- | ususuaug(Ahd)uUfUfAfcucauuaucaL96 | 1983 | VPusdGsauaAfugaguaaAfuCfauaaasusg | 2092 |
| | 886931.1 | | | | |

Table 11 key:
U = uridine-3′-phosphate,
u = 2′-O-methyluridine-3′-phosphate,
us = 2′-O-methyluridine-3′-phosphorothioate,
a = 2′-O-methyladenosine-3′-phosphate,
A = adenosine-3′-phosphate,
as = 2′-O-methyladenosine-3′-phosphorothioate,
(Ahd) = 2′-O-hexadecyl-adenosine-3′-phosphate,
Gf = 2′-fluoroguanosine-3′-phosphate,
Uf = 2′-fluorouridine-3′-phosphate,
Cf = 2′-fluorocytidine-3′-phosphate,
Af = 2′-fluoroadenosine-3′-phosphate,
cs = 2′-O-methylcytidine-3′-phosphate,
VP = Vinylphosphate 5′,
(Agn) = Adenosine-glycol nucleic acid (GNA),
gs = 2′-O-methylguanosine-3′-phosphorothioate,
(Chd) = 2′-O-hexadecyl-cytidine-3′-phosphate,
(Tgn) = Thymidine-glycol nucleic acid (GNA) S-Isomer,
(Ghd) = 2′-O-hexadecyl-guanosine-3′-phosphate,
and
cs = 2′-O-methylcytidine-3′-phosphorothioate.

TABLE 12
Additional APP-Targeting RNAi Agents, Unmodified Sequences.

| | | SEQ | | SEQ |
|---|---|---|---|---|
| Duplex | Sense Sequence | ID | Antisense Sequence | ID |
| Name | (5′ to 3′) | NO | (5′ to 3′) | NO |
| AD- | UAGUGCAUGAAUAGAUUCUCA | 2093 | UGAGAATCUAUUCAUGCACUAGU | 2202 |
| 886823.1 | | | | |
| AD- | UAGUGCAUGAAUAGAUUCUCA | 2094 | UGAGAATCUAUUCAUGCACUAGU | 2203 |
| 886824.1 | | | | |
| AD- | UAGUGCAUGAAUAGAUUCUCA | 2095 | UGAGAATCUAUUCAUGCACUAGU | 2204 |
| 886825.1 | | | | |
| AD- | UAGUGCAUGAAUAGAUUCUCA | 2096 | UGAGAATCUAUUCAUGCACUAGU | 2205 |
| 886826.1 | | | | |
| AD- | UAGUGCAUGAAUAGAUUCUCA | 2097 | UGAGAATCUAUUCAUGCACUAGU | 2206 |
| 886827.1 | | | | |
| AD- | UAGUGCAUGAAUAGAUUCUCA | 2098 | UGAGAATCUAUUCAUGCACUAGU | 2207 |
| 886828.1 | | | | |
| AD- | UAGUGCAUGAAUAGAUUCUCA | 2099 | UGAGAATCUAUUCAUGCACUAGU | 2208 |
| 886829.1 | | | | |
| AD- | UAGUGCAUGAAUAGAUUCUCA | 2100 | UGAGAATCUAUTCAUGCACUAGU | 2209 |
| 886830.1 | | | | |
| AD- | UAGUGCAUGAAUAGAUUCUCA | 2101 | UGAGAATCUAUTCAUGCACUAGU | 2210 |
| 886831.1 | | | | |
| AD- | UAGUGCAUGAAUAGAUUCUCA | 2102 | UGAGAATCUAUUCAUGCACUAGU | 2211 |
| 886832.1 | | | | |
| AD- | UAGUGCAUGAAUAGAUUCUCA | 2103 | UGAGAATCUAUTCAUGCACUAGU | 2212 |
| 886833.1 | | | | |
| AD- | UAGUGCAUGAAUAGAUUCUCA | 2104 | UGAGAATCUAUUCAUGCACUAGU | 2213 |
| 886834.1 | | | | |
| AD- | UAGUGCAUGAAUAGAUUCUCA | 2105 | UGAGAATCUAUUCAUGCACUAGU | 2214 |
| 886836.1 | | | | |
| AD- | UAGUGCAUGAAUAGAUUCUCA | 2106 | UGAGAATCUAUTCAUGCACUAGU | 2215 |
| 886837.1 | | | | |
| AD- | UAGUGCAUGAAUAGAUUCUCA | 2107 | UGAGAATCUAUTCAUGCACUAGU | 2216 |
| 886838.1 | | | | |
| AD- | UAGUGCAUGAAUAGAUUCUCA | 2108 | UGAGAATCUAUUCAUGCACUAGU | 2217 |
| 886839.1 | | | | |
| AD- | UAGUGCAUGAAUAGAUUCUCA | 2109 | UGAGAATCUAUUCAUGCACUAGU | 2218 |
| 886839.2 | | | | |
| AD- | UAGUGCAUGAAUAGAUUCUCA | 2110 | UGAGAATCUAUTCAUGCACUAGU | 2219 |
| 886840.1 | | | | |
| AD- | UAGUGCAUGAAUAGAUUCUCA | 2111 | UGAGAATCUAUTCAUGCACUAGU | 2220 |
| 886841.1 | | | | |
| AD- | UAGUGCAUGAAUAGAUUCUCA | 2112 | UGAGAATCUAUTCAUGCACUAGU | 2221 |
| 886842.1 | | | | |
| AD- | UAGUGCAUGAAUAGAUUCUCA | 2113 | UGAGAATCUAUTCAUGCACUAGU | 2222 |
| 886843.1 | | | | |
| AD- | UAGUGCAUGAAUAGAUUCUCA | 2114 | UGAGAATCUAUTCAUGCACUAGU | 2223 |
| 886844.1 | | | | |
| AD- | UAGUGCAUGAAUAGAUUCUCA | 2115 | UGAGAATCUAUUCAUGCACUAGU | 2224 |
| 886845.1 | | | | |
| AD- | UAGUGCAUGAAUAGAUUCUCA | 2116 | UGAGAAUCUAUUCAUGCACUAGU | 2225 |
| 886846.1 | | | | |
| AD- | UAGUGCAUGAAUAGAUUCUCA | 2117 | UGAGAATCUAUUCAUGCACUAGU | 2226 |
| 886847.1 | | | | |
| AD- | UAGUGCAUGAAUAGAUUCUCA | 2118 | UGAGAATCUAUUCAUGCACUAGU | 2227 |
| 886848.1 | | | | |
| AD- | UAGUGCAUGAAUAGAUUCUCA | 2119 | UGAGAATCUAUUCAUGCACUAGU | 2228 |
| 886849.1 | | | | |
| AD- | UAGUGCAUGAAUAGAUUCUCA | 2120 | UGAGAATCUAUTCAUGCACUAGU | 2229 |
| 886850.1 | | | | |
| AD- | UAGUGCAUGAAUAGAUUCUCA | 2121 | UGAGAATCUAUTCAUGCACUAGU | 2230 |
| 886851.1 | | | | |
| AD- | UAGUGCAUGAAUAGAUUCUCA | 2122 | UGAGAATCUAUTCAUGCACUAGU | 2231 |
| 886852.1 | | | | |
| AD- | UAGUGCAUGAAUAGAUUCUCA | 2123 | UGAGAATCUAUTCAUGCACUAGU | 2232 |
| 886853.1 | | | | |
| AD- | UAGUGCAUGAAUAGAUUCUCA | 2124 | UGAGAATCUAUTCAUGCACUAGU | 2233 |
| 886854.1 | | | | |
| AD- | UAGUGCAUGAAUAGAUUCUCA | 2125 | UGAGAATCUAUUCAUGCACUAGU | 2234 |
| 886855.1 | | | | |
| AD- | UAGUGCAUGAAUAGAUUCUCA | 2126 | UGAGAATCUAUUCAUGCACUAGU | 2235 |
| 886856.1 | | | | |
| AD- | UAGUGCAUGAAUAGAUUCUCA | 2127 | UGAGAATCUAUTCAUGCACUAGU | 2236 |
| 886857.1 | | | | |
| AD- | UAGUGCAUGAAUAGAUUCUCA | 2128 | UGAGAATCUAUTCAUGCACUAGU | 2237 |
| 886858.1 | | | | |
| AD- | UAGUGCAUGAAUAGAUUCUCA | 2129 | UGAGAATCUAUTCAUGCACUAGU | 2238 |
| 886859.1 | | | | |
| AD- | UAGUGCAUGAAUAGAUUCUCA | 2130 | UGAGAATCUAUTCAUGCACUAGU | 2239 |
| 886860.1 | | | | |
| AD- | UAGUGCAUGAAUAGAUUCUCA | 2131 | UGAGAATCUAUUCAUGCACUAUG | 2240 |
| 886861.1 | | | | |
| AD- | UAGUGCAUGAAUAGAUUCUCA | 2132 | UGAGAATCUAUTCAUGCACUAUG | 2241 |
| 886862.1 | | | | |
| AD- | UAGUGCAUGAAUAGAUUCUCA | 2133 | UGAGAATCUAUTCAUGCACUAUG | 2242 |
| 886863.1 | | | | |
| AD- | GGCUACGAAAAUCCAACCUAA | 2134 | UUAGGUTGGAUUUUCGUAGCCGU | 2243 |
| 886864.1 | | | | |
| AD- | GGCUACGAAAAUCCAACCUAA | 2135 | UUAGGUTGGAUUUUCGUAGCCGU | 2244 |
| 886865.1 | | | | |
| AD- | GGCUACGAAAAUCCAACCUAA | 2136 | UUAGGUTGGAUUUUCGUAGCCGU | 2245 |
| 886866.1 | | | | |
| AD- | GGCUACGAAAAUCCAACCUAA | 2137 | UUAGGUTGGAUUUUCGUAGCCGU | 2246 |
| 886867.1 | | | | |
| AD- | GGCUACGAAAAUCCAACCUAA | 2138 | UUAGGUTGGAUUUUCGUAGCCGU | 2247 |
| 886868.1 | | | | |
| AD- | GGCUACGAAAAUCCAACCUAA | 2139 | UUAGGUTGGAUUUUCGUAGCCGU | 2248 |
| 886869.1 | | | | |
| AD- | GGCUACGAAAAUCCAACCUAA | 2140 | UUAGGUTGGAUTUUCGUAGCCGU | 2249 |
| 886870.1 | | | | |
| AD- | GGCUACGAAAAUCCAACCUAA | 2141 | UUAGGUTGGAUTUUCGUAGCCGU | 2250 |
| 886871.1 | | | | |
| AD- | GGCUACGAAAAUCCAACCUAA | 2142 | UUAGGUTGGAUUUUCGUAGCCGU | 2251 |
| 886872.1 | | | | |
| AD- | GGCUACGAAAAUCCAACCUAA | 2143 | UUAGGUTGGAUTUUCGUAGCCGU | 2252 |
| 886873.1 | | | | |
| AD- | GGCUACGAAAAUCCAACCUAA | 2144 | UUAGGUTGGAUTUUCGUAGCCGU | 2253 |
| 886874.1 | | | | |
| AD- | GGCUACGAAAAUCCAACCUAA | 2145 | UUAGGUTGGAUTUUCGUAGCCGU | 2254 |
| 886875.1 | | | | |
| AD- | GGCUACGAAAAUCCAACCUAA | 2146 | UUAGGUTGGAUUUUCGUAGCCGU | 2255 |
| 886876.1 | | | | |
| AD- | GGCUACGAAAAUCCAACCUAA | 2147 | UUAGGUTGGAUTUUCGUAGCCGU | 2256 |
| 886877.1 | | | | |
| AD- | GGCUACGAAAAUCCAACCUAA | 2148 | UUAGGUTGGAUUUUCGUAGCCGU | 2257 |
| 886878.1 | | | | |
| AD- | GGCUACGAAAAUCCAACCUAA | 2149 | UUAGGUTGGAUUUUCGUAGCCGU | 2258 |
| 886879.1 | | | | |
| AD- | GGCUACGAAAAUCCAACCUAA | 2150 | UUAGGUTGGAUTUUCGUAGCCGU | 2259 |
| 886880.1 | | | | |
| AD- | GGCUACGAAAAUCCAACCUAA | 2151 | UUAGGUTGGAUTUUCGUAGCCGU | 2260 |
| 886881.1 | | | | |
| AD- | GGCUACGAAAAUCCAACCUAA | 2152 | UUAGGTTGGAUUUUCGUAGCCGU | 2261 |
| 886882.1 | | | | |
| AD- | GGCUACGAAAAUCCAACCUAA | 2153 | UUAGGTTGGAUUUUCGUAGCCGU | 2262 |
| 886883.1 | | | | |
| AD- | GGCUACGAAAAUCCAACCUAA | 2154 | UUAGGTTGGAUTUUCGUAGCCGU | 2263 |
| 886884.1 | | | | |
| AD- | GGCUACGAAAAUCCAACCUAA | 2155 | UUAGGTTGGAUTUUCGUAGCCGU | 2264 |
| 886885.1 | | | | |
| AD- | GGCUACGAAAAUCCAACCUAA | 2156 | UUAGGUTGGAUUUUCGUAGCCGU | 2265 |
| 886886.1 | | | | |
| AD- | GGCUACGAAAAUCCAACCUAA | 2157 | UUAGGUTGGAUUUUCGUAGCCGU | 2266 |
| 886887.1 | | | | |
| AD- | GGCUACGAAAAUCCAACCUAA | 2158 | UUAGGUTGGAUTUUCGUAGCCGU | 2267 |
| 886888.1 | | | | |
| AD- | GGCUACGAAAAUCCAACCUAA | 2159 | UUAGGUTGGAUTUUCGUAGCCGU | 2268 |
| 886889.1 | | | | |
| AD- | GGCUACGAAAAUCCAACCUAA | 2160 | UUAGGUTGGAUUUUCGUAGCCUG | 2269 |
| 886890.1 | | | | |
| AD- | GGCUACGAAAAUCCAACCUAA | 2161 | UUAGGUTGGAUUUUCGUAGCCUG | 2270 |
| 886891.1 | | | | |
| AD- | GGCUACGAAAAUCCAACCUAA | 2162 | UUAGGTTGGAUTUUCGUAGCCUG | 2271 |
| 886892.1 | | | | |
| AD- | GGCUACGAAAAUCCAACCUAA | 2163 | UUAGGUTGGAUTUUCGUAGCCUG | 2272 |
| 886893.1 | | | | |
| AD- | AAAGAGCAAAACUAUUCAGAA | 2164 | UUCUGAAUAGUUUUGCUCUUUCU | 2273 |
| 886894.1 | | | | |
| AD- | AAAGAGCAAAACUAUUCAGAA | 2165 | UUCUGAAUAGUUUUGCUCUUUCU | 2274 |
| 886895.1 | | | | |
| AD- | AAAGAGCAAAACUAUUCAGAA | 2166 | UUCUGAAUAGUUUUGCUCUUUCU | 2275 |
| 886896.1 | | | | |
| AD- | AAAGAGCAAAACUAUUCAGAA | 2167 | UUCUGAAUAGUUUUGCUCUUUCU | 2276 |
| 886897.1 | | | | |
| AD- | AAAGAGCAAAACUAUUCAGAA | 2168 | UUCUGAAUAGUUUUGCUCUUUCU | 2277 |
| 886898.1 | | | | |
| AD- | AAAGAGCAAAACUAUUCAGAA | 2169 | UUCUGAAUAGUUUUGCUCUUUCU | 2278 |
| 886899.1 | | | | |
| AD- | AAAGAGCAAAACUAUUCAGAA | 2170 | UUCUGAAUAGUUUUGCUCUUUCU | 2279 |
| 886900.1 | | | | |
| AD- | AAAGAGCAAAACUAUUCAGAA | 2171 | UUCUGAAUAGUUUUGCUCUUUCU | 2280 |
| 886901.1 | | | | |
| AD- | AAAGAGCAAAACUAUUCAGAA | 2172 | UUCUGAAUAGUUUUGCUCUUUCU | 2281 |
| 886902.1 | | | | |
| AD- | AAAGAGCAAAACUAUUCAGAA | 2173 | UUCUGAAUAGUUUUGCUCUUUCU | 2282 |
| 886903.1 | | | | |
| AD- | AAAGAGCAAAACUAUUCAGAA | 2174 | UUCUGAAUAGUUUUGCUCUUUCU | 2283 |
| 886904.1 | | | | |
| AD- | AAAGAGCAAAACUAUUCAGAA | 2175 | UUCUGAAUAGUUUUGCUCUUUCU | 2284 |
| 886905.1 | | | | |
| AD- | AAAGAGCAAAACUAUUCAGAA | 2176 | UUCUGAAUAGUUUUGCUCUUUCU | 2285 |
| 886906.1 | | | | |
| AD- | AAAGAGCAAAACUAUUCAGAA | 2177 | UUCUGAAUAGUTUUGCUCUUUCU | 2286 |
| 886907.1 | | | | |
| AD- | AAAGAGCAAAACUAUUCAGAA | 2178 | UUCUGAAUAGUTUUGCUCUUUCU | 2287 |
| 886908.1 | | | | |
| AD- | AAAGAGCAAAACUAUUCAGAA | 2179 | UUCUGAAUAGUTUUGCUCUUUCU | 2288 |
| 886909.1 | | | | |
| AD- | AAAGAGCAAAACUAUUCAGAA | 2180 | UUCUGAAUAGUUUUGCUCUUUUG | 2289 |
| 886910.1 | | | | |
| AD- | AAAGAGCAAAACUAUUCAGAA | 2181 | UUCUGAAUAGUUUUGCUCUUUUG | 2290 |
| 886911.1 | | | | |
| AD- | AAAGAGCAAAACUAUUCAGAA | 2182 | UUCUGAAUAGUUUUGCUCUUUUG | 2291 |
| 886912.1 | | | | |
| AD- | UUUAUGAUUUACUCAUUAUCA | 2183 | UGAUAAUGAGUAAAUCAUAAAAC | 2292 |
| 886913.1 | | | | |
| AD- | UUUAUGAUUUACUCAUUAUCA | 2184 | UGAUAAUGAGUAAAUCAUAAAAC | 2293 |
| 886914.1 | | | | |
| AD- | UUUAUGAUUUACUCAUUAUCA | 2185 | UGAUAAUGAGUAAAUCAUAAAAC | 2294 |
| 886915.1 | | | | |
| AD- | UUUAUGAUUUACUCAUUAUCA | 2186 | UGAUAAUGAGUAAAUCAUAAAAC | 2295 |
| 886916.1 | | | | |
| AD- | UUUAUGAUUUACUCAUUAUCA | 2187 | UGAUAAUGAGUAAAUCAUAAAAC | 2296 |
| 886917.1 | | | | |
| AD- | UUUAUGAUUUACUCAUUAUCA | 2188 | UGAUAAUGAGUAAAUCAUAAAAC | 2297 |
| 886918.1 | | | | |
| AD- | UUUAUGAUUUACUCAUUAUCA | 2189 | UGAUAAUGAGUAAAUCAUAAAAC | 2298 |
| 886919.1 | | | | |
| AD- | UUUAUGAUUUACUCAUUAUCA | 2190 | UGAUAAUGAGUAAAUCAUAAAAC | 2299 |
| 886920.1 | | | | |
| AD- | UUUAUGAUUUACUCAUUAUCA | 2191 | UGAUAAUGAGUAAAUCAUAAAAC | 2300 |
| 886921.1 | | | | |
| AD- | UUUAUGAUUUACUCAUUAUCA | 2192 | UGAUAAUGAGUAAAUCAUAAAAC | 2301 |
| 886922.1 | | | | |
| AD- | UUUAUGAUUUACUCAUUAUCA | 2193 | UGAUAAUGAGUAAAUCAUAAAAC | 2302 |
| 886923.1 | | | | |
| AD- | UUUAUGAUUUACUCAUUAUCA | 2194 | UGAUAAUGAGUAAAUCAUAAAAC | 2303 |
| 886924.1 | | | | |
| AD- | UUUAUGAUUUACUCAUUAUCA | 2195 | UGAUAAUGAGUAAAUCAUAAAAC | 2304 |
| 886925.1 | | | | |
| AD- | UUUAUGAUUUACUCAUUAUCA | 2196 | UGAUAAUGAGUAAAUCAUAAAAC | 2305 |
| 886926.1 | | | | |
| AD- | UUUAUGAUUUACUCAUUAUCA | 2197 | UGAUAAUGAGUAAAUCAUAAAAC | 2306 |
| 886927.1 | | | | |
| AD- | UUUAUGAUUUACUCAUUAUCA | 2198 | UGAUAAUGAGUAAAUCAUAAAAC | 2307 |
| 886928.1 | | | | |
| AD- | UUUAUGAUUUACUCAUUAUCA | 2199 | UGAUAAUGAGUAAAUCAUAAAUG | 2308 |
| 886929.1 | | | | |
| AD- | UUUAUGAUUUACUCAUUAUCA | 2200 | UGAUAAUGAGUAAAUCAUAAAUG | 2309 |
| 886930.1 | | | | |
| AD- | UUUAUGAUUUACUCAUUAUCA | 2201 | UGAUAAUGAGUAAAUCAUAAAUG | 2310 |
| 886931.1 | | | | |

TABLE 13
Additional Human APP-Targeting RNAi Agents,
Modified Sense Sequences and Targets.

| | | Sense | SEQ | mRNA | SEQ |
|---|---|---|---|---|---|
| Duplex | | Sequence | ID | Target | ID |
| Name | target | (5′ to 3′) | NO | Sequence | NO |
| AD- | APP | gsgscua(Chd)ga | 2311 | GGCUACGAAAA | 2315 |
| 961583 | | dAadAuccaaccus | | UCCAACCUAA | |
| | | asa | | | |
| AD- | APP | asasagag(Chd)a | 2312 | AAAGAGCAAAA | 2316 |
| 961584 | | AfaAfcuauucags | | CUAUUCAGAA | |
| | | asa | | | |
| AD- | APP | asasagag(Chd)a | 2313 | AAAGAGCAAAA | 2317 |
| 961585 | | dAadAcuauucags | | CUAUUCAGAA | |
| | | asa | | | |
| AD- | APP | ususuau(Ghd)Af | 2314 | UUUAUGAUUUA | 2318 |
| 961586 | | uUfUfAfcucauua | | CUCAUUAUCA | |
| | | uscsa | | | |

Table 13 key:
U = uridine-3′-phosphate,
u = 2′-O-methyluridine-3′-phosphate,
us = 2′-O-methyluridine-3′-phosphorothioate,
a = 2′-O-methyladenosine-3′-phosphate,
A = adenosine-3′-phosphate,
as = 2′-O-methyladenosine-3′-phosphorothioate,
(Ahd) = 2′-O-hexadecyl-adenosine-3′-phosphate,
Gf = 2′-fluoroguanosine-3′-phosphate,
Uf = 2′-fluorouridine-3′-phosphate,
Cf = 2′-fluorocytidine-3′-phosphate,
Af = 2′-fluoroadenosine-3′-phosphate,
cs = 2′-O-methylcytidine-3′-phosphate,
VP = Vinylphosphate 5′,
(Agn) = Adenosine-glycol nucleic acid (GNA),
gs = 2′-O-methylguanosine-3′-phosphorothioate,
(Chd) = 2′-O-hexadecyl-cytidine-3′-phosphate,
(Tgn) = Thymidine-glycol nucleic acid (GNA) S-Isomer,
(Ghd) = 2′-O-hexadecyl-guanosine-3′-phosphate,
and
cs = 2′-O-methylcytidine-3′-phosphorothioate.

TABLE 14
Additional Human APP-Targeting RNAi Agents,
Modified Antisense Sequences and Targets

| | | Antisense | SEQ | mRNA | SEQ |
|---|---|---|---|---|---|
| Duplex | | Sequence | ID | Target | ID |
| Name | target | (5′ to 3′) | NO | Sequence | NO |
| AD- | APP | VPusUfsaggu(Tgn) | 2319 | UUAGGUTGGAU | 2323 |
| 961583 | | ggaudTuUfcdGuagc | | TUUCGUAGCCG | |
| | | csgsu | | U | |
| AD- | APP | VPuUfcugAfauagud | 2320 | UUCUGAAUAGU | 2324 |
| 961584 | | TuUfgCfucuuuscsu | | TUUGCUCUUUC | |
| | | | | U | |
| AD- | APP | VPuUfcugdAauagud | 2321 | UUCUGAAUAGU | 2325 |
| 961585 | | TuUfgdCucuuuscsu | | TUUGCUCUUUC | |
| | | | | U | |
| AD- | APP | VPusGfsauaAfugag | 2322 | UGAUAAUGAGU | 2326 |
| 961586 | | uaaAfuCfauaaasus | | AAAUCAUAAAU | |
| | | g | | G | |

Table 14 key:
U = uridine-3′-phosphate,
u = 2′-O-methyluridine-3′-phosphate,
us = 2′-O-methyluridine-3′-phosphorothioate,
a = 2′-O-methyladenosine-3′-phosphate,
A = adenosine-3′-phosphate,
as = 2′-O-methyladenosine-3′-phosphorothioate,
(Ahd) = 2′-O-hexadecyl-adenosine-3′-phosphate,
Gf = 2′-fluoroguanosine-3′-phosphate,
Uf = 2′-fluorouridine-3′-phosphate,
Cf = 2′-fluorocytidine-3′-phosphate,
Af = 2′-fluoroadenosine-3′-phosphate,
cs = 2′-O-methylcytidine-3′-phosphate,
VP = Vinylphosphate 5′,
(Agn) = Adenosine-glycol nucleic acid (GNA),
gs = 2′-O-methylguanosine-3′-phosphorothioate,
(Chd) = 2′-O-hexadecyl-cytidine-3′-phosphate,
(Tgn) = Thymidine-glycol nucleic acid (GNA) S-Isomer,
(Ghd) = 2′-O-hexadecyl-guanosine-3′-phosphate,
and
cs = 2′-O-methylcytidine-3′-phosphorothioate.

TABLE 15
hsAPP-Targeting RNAi Agents, Duplex and Target
Sequences for GNA7 C16 control and AU-rich Candidates.

| | | | SEQ | | SEQ | mRNA target | SEQ |
|---|---|---|---|---|---|---|---|
| Chemistry | Duplex | Sense | ID | Antisense | ID | sequence | ID |
| (Target) | Name | Sequence (5′ to 3′) | NO | Sequence (5′ to 3′) | NO | (5′ to 3′) | NO |
| GNA7 | AD- | usasgug(Chd)AfuGfAf | 2327 | asGfsagaa(Tgn)cuauu | 2339 | n/a | n/a |
| C16 | 392927.2 | AfuagauucucuL96 | | cAfuGfcacuasgsu | | | |
| (APP) | | | | | | | |
| AU-rich | AD- | asasagagCfaAfAfAfcu | 2328 | asUfscugAfaUfAfguuu | 2340 | AGAAAGAGCAAA | 2351 |
| seed | 506935.2 | auucagauL96 | | UfgCfucuuuscsu | | ACUAUUCAGAU | |
| (APP) | | | | | | | |
| AU-rich | AD- | ususggccAfaCfAfUfga | 2329 | asUfscacUfaAfUfcaug | 2341 | UCUUGGCCAACA | 2352 |
| seed | 507065.2 | uuagugauL96 | | UfuGfgccaasgsa | | UGAUUAGUGAA | |
| (APP) | | | | | | | |
| AU-rich | AD- | uscsugggUfuGfAfCfaa | 2330 | asUfsugaUfaUfUfuguc | 2342 | GUUCUGGGUUGA | 2353 |
| seed | 507159.2 | auaucaauL96 | | AfaCfccagasasc | | CAAAUAUCAAG | |
| (APP) | | | | | | | |
| AU-rich | AD- | ususuaugAfuUfUfAfcu | 2331 | asGfsauaAfuGfAfguaa | 2343 | GUUUUAUGAUUU | 2354 |
| seed | 507538.2 | cauuaucuL96 | | AfuCfauaaasasc | | ACUCAUUAUCG | |
| (APP) | | | | | | | |
| AU-rich | AD- | asusgccuGfaAfCfUfug | 2332 | asAfsuuaAfuUfCfaagu | 2344 | AGAUGCCUGAAC | 2355 |
| seed | 507624.2 | aauuaauuL96 | | UfcAfggcauscsu | | UUGAAUUAAUC | |
| (APP) | | | | | | | |
| AU-rich | AD- | asgsaugcCfuGfAfAfcu | 2333 | asUfsaauUfcAfAfguuc | 2345 | GUAGAUGCCUGA | 2356 |
| seed | 507724.2 | ugaauuauL96 | | AfgGfcaucusasc | | ACUUGAAUUAA | |
| (APP) | | | | | | | |
| AU-rich | AD- | gscscugaAfcUfUfGfaa | 2334 | asGfsgauUfaAfUfucaa | 2346 | AUGCCUGAACUU | 2357 |
| seed | 507725.2 | uuaauccuL96 | | GfuUfcaggcsasu | | GAAUUAAUCCA | |
| (APP) | | | | | | | |
| AU-rich | AD- | gsusgguuUfgUfGfAfcc | 2335 | asUfsuaaUfuGfGfguca | 2347 | UUGUGGUUUGUG | 2358 |
| seed | 507789.2 | caauuaauL96 | | CfaAfaccacsasa | | ACCCAAUUAAG | |
| (APP) | | | | | | | |
| AU-rich | AD- | csasgaugCfuUfUfAfga | 2336 | asAfsaauCfuCfUfcuaa | 2348 | UUCAGAUGCUUU | 2359 |
| seed | 507874.2 | gagauuuuL96 | | AfgCfaucugsasa | | AGAGAGAUUUU | |
| (APP) | | | | | | | |
| AU-rich | AD- | uscsuugcCfuAfAfGfua | 2337 | asAfsaagGfaAfUfacuu | 2349 | UCUCUUGCCUAA | 2360 |
| seed | 507928.2 | uuccuuuuL96 | | AfgGfcaagasgsa | | GUAUUCCUUUC | |
| (APP) | | | | | | | |
| AU-rich | AD- | ususgcugCfuUfCfUfgc | 2338 | asAfsaauAfuAfGfcaga | 2350 | GAUUGCUGCUUC | 2361 |
| seed | 507949.2 | uauauuuuL96 | | AfgCfagcaasusc | | UGCUAUAUUUG | |
| (APP) | | | | | | | |

Table 15 key:
U = uridine-3′-phosphate,
u = 2′-O-methyluridine-3′-phosphate,
us = 2′-O-methyluridine-3′-phosphorothioate,
a = 2′-O-methyladenosine-3′-phosphate,
A = adenosine-3′-phosphate,
as = 2′-O-methyladenosine-3′-phosphorothioate,
(Ahd) = 2′-O-hexadecyl-adenosine-3′-phosphate,
Gf = 2′-fluoroguanosine-3′-phosphate,
Uf = 2′-fluorouridine-3′-phosphate,
Cf = 2′-fluorocytidine-3′-phosphate,
Af = 2′-fluoroadenosine-3′-phosphate,
cs = 2′-O-methylcytidine-3′-phosphate,
VP = Vinylphosphate 5′,
(Agn) = Adenosine-glycol nucleic acid (GNA),
gs = 2′-O-methylguanosine-3′-phosphorothioate,
(Chd) = 2′-O-hexadecyl-cytidine-3′-phosphate,
(Tgn) = Thymidine-glycol nucleic acid (GNA) S-Isomer,
(Ghd) = 2′-O-hexadecyl-guanosine-3′-phosphate,
and
cs = 2′-O-methylcytidine-3′-phosphorothioate.

TABLE 16
hsAPP-Targeting RNAi Agents, Duplex and Target Sequences for Lead Candidates.

| | | | SEQ | | SEQ |
|---|---|---|---|---|---|
| | | | ID | | ID |
| Duplex | Strand | Oligonucleotide Sequence | NO: | Target Sequence | NO: |
| AD- | Sense | usasgug(Chd)AfuGfAfAfuagauucucaL96 | 2362 | UAGUGCAUGAAUAGAUUCUCA | 2406 |
| 886823.2 | (5′ to 3′) | | | | |
| | Antisense | VPusGfsagaa(Tgn)cuauucAfuGfcacuasgsu | 2363 | UGAGAATCUAUUCAUGCACUAGU | 2407 |
| | (5′ to 3′) | | | | |
| AD- | Sense | usasgug(Chd)AfudGAfAfuagauucucaL96 | 2364 | UAGUGCAUGAAUAGAUUCUCA | 2408 |
| 886839.2 | (5′ to 3′) | | | | |
| | Antisense | VPusdGsagaa(Tgn)cuauucAfudGcacuasgsu | 2365 | UGAGAATCUAUUCAUGCACUAGU | 2409 |
| | (5′ to 3′) | | | | |
| AD- | Sense | usasgug(Chd)audGadAuagauucucaL96 | 2366 | UAGUGCAUGAAUAGAUUCUCA | 2410 |
| 886845.2 | (5′ to 3′) | | | | |
| | Antisense | VPudGagaa(Tgn)cuauUfcAfudGcacuasgsu | 2367 | UGAGAATCUAUUCAUGCACUAGU | 2411 |
| | (5′ to 3′) | | | | |
| AD- | Sense | usasgug(Chd)AfudGAfAfuagauucucaL96 | 2368 | UAGUGCAUGAAUAGAUUCUCA | 2412 |
| 886853.2 | (5′ to 3′) | | | | |
| | Antisense | VPusdGsagdAa(Tgn)cuaudTcAfugcacuasgsu | 2369 | UGAGAATCUAUTCAUGCACUAGU | 2413 |
| | (5′ to 3′) | | | | |
| AD- | Sense | usasgug(Chd)AfudGAfAfuagauucucaL96 | 2370 | UAGUGCAUGAAUAGAUUCUCA | 2414 |
| 886858.2 | (5′ to 3′) | | | | |
| | Antisense | VPudGagdAa(Tgn)cuaudTcAfudGcacuasgsu | 2371 | UGAGAATCUAUTCAUGCACUAGU | 2415 |
| | (5′ to 3′) | | | | |
| AD- | Sense | gsgscua(Chd)GfaAfAfAfuccaaccuaaL96 | 2372 | GGCUACGAAAAUCCAACCUAA | 2416 |
| 886864.2 | (5′ to 3′) | | | | |
| | Antisense | VPusUfsaggu(Tgn)ggauuuUfcGfuagccsgsu | 2373 | UUAGGUTGGAUUUUCGUAGCCGU | 2417 |
| | (5′ to 3′) | | | | |
| AD- | Sense | gsgscua(Chd)gadAadAuccaaccuaaL96 | 2374 | GGCUACGAAAAUCCAACCUAA | 2418 |
| 886873.2 | (5′ to 3′) | | | | |
| | Antisense | VPusUfsaggu(Tgn)ggaudTuUfcdGuagccsgsu | 2375 | UUAGGUTGGAUTUUCGUAGCCGU | 2419 |
| | (5′ to 3′) | | | | |
| AD- | Sense | gsgscua(Chd)gadAadAuccaaccuaaL96 | 2376 | GGCUACGAAAAUCCAACCUAA | 2420 |
| 886877.2 | (5′ to 3′) | | | | |
| | Antisense | VPusUfsaggu(Tgn)ggaudTuUfcguagccsgsu | 2377 | UUAGGUTGGAUTUUCGUAGCCGU | 2421 |
| | (5′ to 3′) | | | | |
| AD- | Sense | gsgscua(Chd)gaAfAfAfuccaaccuaaL96 | 2378 | GGCUACGAAAAUCCAACCUAA | 2422 |
| 886879.2 | (5′ to 3′) | | | | |
| | Antisense | VPusUfsaggu(Tgn)ggauuuUfcdGuagccsgsu | 2379 | UUAGGUTGGAUUUUCGUAGCCGU | 2423 |
| | (5′ to 3′) | | | | |
| AD- | Sense | gsgscua(Chd)gaAfAfAfuccaaccuaaL96 | 2380 | GGCUACGAAAAUCCAACCUAA | 2424 |
| 886883.2 | (5′ to 3′) | | | | |
| | Antisense | VPuUfaggdT(Tgn)ggauuuUfcdGuagccsgsu | 2381 | UUAGGTTGGAUUUUCGUAGCCGU | 2425 |
| | (5′ to 3′) | | | | |
| AD- | Sense | gsgscua(Chd)gaAfAfAfuccaaccuaaL96 | 2382 | GGCUACGAAAAUCCAACCUAA | 2426 |
| 886884.2 | (5′ to 3′) | | | | |
| | Antisense | VPuUfaggdT(Tgn)ggaudTuUfcdGuagccsgsu | 2383 | UUAGGTTGGAUTUUCGUAGCCGU | 2427 |
| | (5′ to 3′) | | | | |
| AD- | Sense | gsgscua(Chd)gaAfAfAfuccaaccuaaL96 | 2384 | GGCUACGAAAAUCCAACCUAA | 2428 |
| 886889.2 | (5′ to 3′) | | | | |
| | Antisense | VPuUfagdGu(Tgn)ggaudTuUfcguagccsgsu | 2385 | UUAGGUTGGAUTUUCGUAGCCGU | 2429 |
| | (5′ to 3′) | | | | |
| AD- | Sense | asasagagCfaAfAfAfcuauu(Chd)agaaL96 | 2386 | AAAGAGCAAAACUAUUCAGAA | 2430 |
| 886899.2 | (5′ to 3′) | | | | |
| | Antisense | VPusUfscugAfaUfAfguuuUfgCfucuuuscsu | 2387 | UUCUGAAUAGUUUUGCUCUUUCU | 2431 |
| | (5′ to 3′) | | | | |
| AD- | Sense | asasaga(Ghd)CfaAfAfAfcuauucagaaL96 | 2388 | AAAGAGCAAAACUAUUCAGAA | 2432 |
| 886900.2 | (5′ to 3′) | | | | |
| | Antisense | VPusUfscugAfauaguuuUfgCfucuuuscsu | 2389 | UUCUGAAUAGUUUUGCUCUUUCU | 2433 |
| | (5′ to 3′) | | | | |
| AD- | Sense | asasagag(Chd)aAfAfAfcuauucagaaL96 | 2390 | AAAGAGCAAAACUAUUCAGAA | 2434 |
| 886906.2 | (5′ to 3′) | | | | |
| | Antisense | VPuUfcugAfauaguuuUfgCfucuuuscsu | 2391 | UUCUGAAUAGUUUUGCUCUUUCU | 2435 |
| | (5′ to 3′) | | | | |
| AD- | Sense | asasagag(Chd)aAfaAfcuauucagaaL96 | 2392 | AAAGAGCAAAACUAUUCAGAA | 2436 |
| 886907.2 | (5′ to 3′) | | | | |
| | Antisense | VPuUfcugAfauagudTuUfgCfucuuuscsu | 2393 | UUCUGAAUAGUTUUGCUCUUUCU | 2437 |
| | (5′ to 3′) | | | | |
| AD- | Sense | asasagag(Chd)adAadAcuauucagaaL96 | 2394 | AAAGAGCAAAACUAUUCAGAA | 2438 |
| 886908.2 | (5′ to 3′) | | | | |
| | Antisense | VPuUfcugAfauagudTuUfgCfucuuuscsu | 2395 | UUCUGAAUAGUTUUGCUCUUUCU | 2439 |
| | (5′ to 3′) | | | | |
| AD- | Sense | asasagag(Chd)adAadAcuauucagaaL96 | 2396 | AAAGAGCAAAACUAUUCAGAA | 2440 |
| 886909.2 | (5′ to 3′) | | | | |
| | Antisense | VPuUfcugdAauagudTuUfgdCucuuuscsu | 2397 | UUCUGAAUAGUTUUGCUCUUUCU | 2441 |
| | (5′ to 3′) | | | | |
| AD- | Sense | ususuau(Ghd)AfuUfUfAfcucauuaucaL96 | 2398 | UUUAUGAUUUACUCAUUAUCA | 2442 |
| 886919.2 | (5′ to 3′) | | | | |
| | Antisense | VPusGfsauaAfugaguaaAfuCfauaaasasc | 2399 | UGAUAAUGAGUAAAUCAUAAAAC | 2443 |
| | (5′ to 3′) | | | | |
| AD- | Sense | ususuaug(Ahd)uUfudAcucauuaucaL96 | 2400 | UUUAUGAUUUACUCAUUAUCA | 2444 |
| 886928.2 | (5′ to 3′) | | | | |
| | Antisense | VPudGauadAugagudAaAfudCauaaasasc | 2401 | UGAUAAUGAGUAAAUCAUAAAAC | 2445 |
| | (5′ to 3′) | | | | |
| AD- | Sense | ususuaugAfuUfUfAfcuc(Ahd)uuaucaL96 | 2402 | UUUAUGAUUUACUCAUUAUCA | 2446 |
| 886930.2 | (5′ to 3′) | | | | |
| | Antisense | VPusdGsauaAfugaguaaAfuCfauaaasusg | 2403 | UGAUAAUGAGUAAAUCAUAAAUG | 2447 |
| | (5′ to 3′) | | | | |
| AD- | Sense | ususuaug(Ahd)uUfUfAfcucauuaucaL96 | 2404 | UUUAUGAUUUACUCAUUAUCA | 2448 |
| 886931.2 | (5′ to 3′) | | | | |
| | Antisense | VPusdGsauaAfugaguaaAfuCfauaaasusg | 2405 | UGAUAAUGAGUAAAUCAUAAAUG | 2449 |
| | (5′ to 3′) | | | | |

Table 16 key:
U = uridine-3′-phosphate,
u = 2′-O-methyluridine-3′-phosphate,
us = 2′-O-methyluridine-3′-phosphorothioate,
a = 2′-O-methyladenosine-3′-phosphate,
A = adenosine-3′-phosphate,
as = 2′-O-methyladenosine-3′-phosphorothioate,
(Ahd) = 2′-O-hexadecyl-adenosine-3′-phosphate,
Gf = 2′-fluoroguanosine-3′-phosphate,
Uf = 2′-fluorouridine-3′-phosphate,
Cf = 2′-fluorocytidine-3′-phosphate,
Af = 2′-fluoroadenosine-3′-phosphate,
cs = 2′-O-methylcytidine-3′-phosphate,
VP = Vinylphosphate 5′,
(Agn) = Adenosine-glycol nucleic acid (GNA),
gs = 2′-O-methylguanosine-3′-phosphorothioate,
(Chd) = 2′-O-hexadecyl-cytidine-3′-phosphate,
(Tgn) = Thymidine-glycol nucleic acid (GNA) S-Isomer,
(Ghd) = 2′-O-hexadecyl-guanosine-3′-phosphate,
and
cs = 2′-O-methylcytidine-3′-phosphorothioate.

TABLE 17
C16 siRNA conjugates identified to knock down APP in in vivo NHP studies

| | | | SEQ | | SEQ |
|---|---|---|---|---|---|
| | | | ID | | ID |
| Duplex | Strand | Oligonucleotide Sequence | NO: | Target Sequence | NO: |
| AD- | Sense | Q363sasaaaucCfaAfCfCfuacaaguuscsa | 2450 | CGAAAAUCCAACCUACAAGUUCU | 2460 |
| 994379 | (5′ to 3′) | | | | |
| | Antisense | VPusGfsaacu(Tgn)guagguUfgGfauuuuscsg | 2451 | AGAACUUGUAGGUUGGAUUUUCG | 2461 |
| | (5′ to 3′) | | | | |
| AD- | Sense | gsgscua(Chd)gadAadAuccaaccusasa | 2452 | ACGGCUACGAAAAUCCAACCUAC | 2462 |
| 961583 | (5′ to 3′) | | | | |
| | Antisense | VPusUfsaggu(Tgn)ggaudTuUfcdGuagccsgsu | 2453 | GUAGGUUGGAUUUUCGUAGCCGU | 2463 |
| | (5′ to 3′) | | | | |
| AD- | Sense | asasagag(Chd)aAfaAfcuauucagsasa | 2454 | AGAAAGAGCAAAACUAUUCAGAU | 2464 |
| 961584 | (5′ to 3′) | | | | |
| | Antisense | VPuUfcugAfauagudTuUfgCfucuuuscsu | 2455 | AUCUGAAUAGUUUUGCUCUUUCU | 2465 |
| | (5′ to 3′) | | | | |
| AD- | Sense | asasagag(Chd)adAadAcuauucagsasa | 2456 | AGAAAGAGCAAAACUAUUCAGAU | 2466 |
| 961585 | (5′ to 3′) | | | | |
| | Antisense | VPuUfcugdAauagudTuUfgdCucuuuscsu | 2457 | AUCUGAAUAGUUUUGCUCUUUCU | 2467 |
| | (5′ to 3′) | | | | |
| AD- | Sense | ususuau(Ghd)AfuUfUfAfcucauuauscsa | 2458 | GUUUUAUGAUUUACUCAUUAUCG | 2468 |
| 961586 | (5′ to 3′) | | | | |
| | Antisense | VPusGfsauaAfugaguaaAfuCfauaaasusg | 2459 | CGAUAAUGAGUAAAUCAUAAAAC | 2469 |
| | (5′ to 3′) | | | | |

TABLE 18
Unmodified base transcripts used in the C16 conjugates of Table 17

| | | | | SEQ |
|---|---|---|---|---|
| | | | | ID |
| Duplex | Strand | Oligo name | Transcript Sequence | NO: |
| AD- | Sense | A-1701871.1 | AAAAUCCAACCUACAAGUUCA | 2470 |
| 994379 | (5′ to 3′) | | | |
| | Antisense | A-882382.1 | UGAACUTGUAGGUUGGAUUUUCG | 2471 |
| | (5′ to 3′) | | | |
| AD- | Sense | A-1770584.1 | GGCUACGAAAAUCCAACCUAA | 2472 |
| 961583 | (5′ to 3′) | | | |
| | Antisense | A-1683088.1 | UUAGGUTGGAUTUUCGUAGCCGU | 2473 |
| | (5′ to 3′) | | | |
| AD- | Sense | A-1770585.1 | AAAGAGCAAAACUAUUCAGAA | 2474 |
| 961584 | (5′ to 3′) | | | |
| | Antisense | A-1683116.1 | UUCUGAAUAGUTUUGCUCUUUCU | 2475 |
| | (5′ to 3′) | | | |
| AD- | Sense | A-1770586.1 | AAAGAGCAAAACUAUUCAGAA | 2476 |
| 961585 | (5′ to 3′) | | | |
| | Antisense | A-1683118.1 | UUCUGAAUAGUTUUGCUCUUUCU | 2477 |
| | (5′ to 3′) | | | |
| AD- | Sense | A-1770587.1 | UUUAUGAUUUACUCAUUAUCA | 2478 |
| 961586 | (5′ to 3′) | | | |
| | Antisense | A-1683134.1 | UGAUAAUGAGUAAAUCAUAAAUG | 2479 |
| | (5′ to 3′) | | | |

TABLE 19
Exemplary Mouse APP-Targeting RNAi Duplexes, Modified Sequences

| | | SEQ | | SEQ | | SEQ |
|---|---|---|---|---|---|---|
| Duplex | | ID | | ID | | ID |
| Name | Sense Sequence (5′ to 3′) | NO | Antisense Sequence (5′ to 3′) | NO | mRNA target sequence | NO |
| AD- | csasugu(Uhd)CfuGfUfGfguaaacucaaL96 | 2480 | VPusUfsgagUfuUfAfccacAfgAfacaugsgsc | 2572 | GCCAUGUUCUGUGGUAAACUCAA | 2664 |
| 397175 | | | | | | |
| AD- | usgsuuc(Uhd)GfuGfGfUfaaacucaacaL96 | 2481 | VPusGfsuugAfgUfUfuaccAfcAfgaacasusg | 2573 | CAUGUUCUGUGGUAAACUCAACA | 2665 |
| 397176 | | | | | | |
| AD- | asusguu(Chd)UfgUfGfGfuaaacucaaaL96 | 2482 | VPusUfsugaGfuUfUfaccaCfaGfaacausgsg | 2574 | CCAUGUUCUGUGGUAAACUCAAC | 2666 |
| 397177 | | | | | | |
| AD- | csusgug(Ghd)UfaAfAfCfucaacaugcaL96 | 2483 | VPusGfscauGfuUfGfaguuUfaCfcacagsasa | 2575 | UUCUGUGGUAAACUCAACAUGCA | 2667 |
| 397178 | | | | | | |
| AD- | gsgsuaa(Ahd)CfuCfAfAfcaugcacauaL96 | 2484 | VPusAfsuguGfcAfUfguugAfgUfuuaccsasc | 2576 | GUGGUAAACUCAACAUGCACAUG | 2668 |
| 397179 | | | | | | |
| AD- | usgsugg(Uhd)AfaAfCfUfcaacaugcaaL96 | 2485 | VPusUfsgcaUfgUfUfgaguUfuAfccacasgsa | 2577 | UCUGUGGUAAACUCAACAUGCAC | 2669 |
| 397180 | | | | | | |
| AD- | gsasaga(Ghd)CfaCfUfAfacuugcacgaL96 | 2486 | VPusCfsgugCfaAfGfuuagUfgCfucuucsusc | 2578 | GAGAAGAGCACUAACUUGCACGA | 2670 |
| 397181 | | | | | | |
| AD- | cscsgcu(Ghd)GfuAfCfUfuugaugucaaL96 | 2487 | VPusUfsgacAfuCfAfaaguAfcCfagcggsgsa | 2579 | UCCCGCUGGUACUUUGAUGUCAC | 2671 |
| 397182 | | | | | | |
| AD- | cscsaug(Uhd)UfcUfGfUfgguaaacucaL96 | 2488 | VPusGfsaguUfuAfCfcacaGfaAfcauggscsg | 2580 | CGCCAUGUUCUGUGGUAAACUCA | 2672 |
| 397183 | | | | | | |
| AD- | gsusggu(Ahd)AfaCfUfCfaacaugcacaL96 | 2489 | VPusGfsugcAfuGfUfugagUfuUfaccacsasg | 2581 | CUGUGGUAAACUCAACAUGCACA | 2673 |
| 397184 | | | | | | |
| AD- | gsasacu(Ghd)CfaGfAfUfcacaaacguaL96 | 2490 | VPusAfscguUfuGfUfgaucUfgCfaguucsasg | 2582 | CUGAACUGCAGAUCACAAACGUG | 2674 |
| 397185 | | | | | | |
| AD- | asasgag(Chd)AfcUfAfAfcuugcacgaaL96 | 2491 | VPusUfscguGfcAfAfguuaGfuGfcucuuscsu | 2583 | AGAAGAGCACUAACUUGCACGAC | 2675 |
| 397186 | | | | | | |
| AD- | asgscac(Uhd)AfaCfUfUfgcacgacuaaL96 | 2492 | VPusUfsaguCfgUfGfcaagUfuAfgugcuscsu | 2584 | AGAGCACUAACUUGCACGACUAU | 2676 |
| 397187 | | | | | | |
| AD- | gscsacu(Ahd)AfcUfUfGfcacgacuauaL96 | 2493 | VPusAfsuagUfcGfUfgcaaGfuUfagugcsusc | 2585 | GAGCACUAACUUGCACGACUAUG | 2677 |
| 397188 | | | | | | |
| AD- | asasagu(Uhd)UfaCfUfCfaagacuaccaL96 | 2494 | VPusGfsguaGfuCfUfugagUfaAfacuuusgsg | 2586 | CCAAAGUUUACUCAAGACUACCA | 2678 |
| 397189 | | | | | | |
| AD- | csgscau(Ghd)AfaCfCfAfgucucugucaL96 | 2495 | VPusGfsacaGfaGfAfcuggUfuCfaugcgscsu | 2587 | AGCGCAUGAACCAGUCUCUGUCC | 2679 |
| 397190 | | | | | | |
| AD- | csascau(Chd)GfuGfAfUfuccuuaccgaL96 | 2496 | VPusCfsgguAfaGfGfaaucAfcGfaugugsgsg | 2588 | CCCACAUCGUGAUUCCUUACCGU | 2680 |
| 397191 | | | | | | |
| AD- | asusgcu(Ghd)AfaGfAfAfguacguccgaL96 | 2497 | VPusCfsggaCfgUfAfcuucUfuCfagcausgsu | 2589 | ACAUGCUGAAGAAGUACGUCCGU | 2681 |
| 397192 | | | | | | |
| AD- | gsasgcg(Chd)AfuGfAfAfccagucucuaL96 | 2498 | VPusAfsgagAfcUfGfguucAfuGfcgcucsgsu | 2590 | ACGAGCGCAUGAACCAGUCUCUG | 2682 |
| 397193 | | | | | | |
| AD- | gsasgca(Ghd)AfaCfUfAfcuccgacgaaL96 | 2499 | VPusUfscguCfgGfAfguagUfuCfugcucscsu | 2591 | AGGAGCAGAACUACUCCGACGAU | 2683 |
| 397194 | | | | | | |
| AD- | csasccc(Ahd)CfaUfCfGfugauuccuuaL96 | 2500 | VPusAfsaggAfaUfCfacgaUfgUfgggugsusg | 2592 | CACACCCACAUCGUGAUUCCUUA | 2684 |
| 397195 | | | | | | |
| AD- | asgsagc(Ahd)CfuAfAfCfuugcacgacaL96 | 2501 | VPusGfsucgUfgCfAfaguuAfgUfgcucususc | 2593 | GAAGAGCACUAACUUGCACGACU | 2685 |
| 397196 | | | | | | |
| AD- | csascua(Ahd)CfuUfGfCfacgacuaugaL96 | 2502 | VPusCfsauaGfuCfGfugcaAfgUfuagugscsu | 2594 | AGCACUAACUUGCACGACUAUGG | 2686 |
| 397197 | | | | | | |
| AD- | csuscaa(Ghd)AfcUfAfCfcagugaaccaL96 | 2503 | VPusGfsguuCfaCfUfgguaGfuCfuugagsusa | 2595 | UACUCAAGACUACCAGUGAACCU | 2687 |
| 397198 | | | | | | |
| AD- | asgscac(Ahd)CfcCfUfAfaagcauuuuaL96 | 2504 | VPusAfsaaaUfgCfUfuuagGfgUfgugcusgsu | 2596 | ACAGCACACCCUAAAGCAUUUUG | 2688 |
| 397199 | | | | | | |
| AD- | asasgga(Ghd)CfaGfAfAfcuacuccgaaL96 | 2505 | VPusUfscggAfgUfAfguucUfgCfuccuuscsu | 2597 | AGAAGGAGCAGAACUACUCCGAC | 2689 |
| 397200 | | | | | | |
| AD- | gsgsagc(Ahd)GfaAfCfUfacuccgacgaL96 | 2506 | VPusCfsgucGfgAfGfuaguUfcUfgcuccsusu | 2598 | AAGGAGCAGAACUACUCCGACGA | 2690 |
| 397201 | | | | | | |
| AD- | gsasaac(Ahd)GfuAfCfAfcauccauccaL96 | 2507 | VPusGfsgauGfgAfUfguguAfcUfguuucsusu | 2599 | AAGAAACAGUACACAUCCAUCCA | 2691 |
| 397202 | | | | | | |
| AD- | csusgaa(Chd)UfgCfAfGfaucacaaacaL96 | 2508 | VPusGfsuuuGfuGfAfucugCfaGfuucagsgsg | 2600 | CCCUGAACUGCAGAUCACAAACG | 2692 |
| 397203 | | | | | | |
| AD- | cscsaca(Uhd)CfgUfGfAfuuccuuaccaL96 | 2509 | VPusGfsguaAfgGfAfaucaCfgAfuguggsgsu | 2601 | ACCCACAUCGUGAUUCCUUACCG | 2693 |
| 397204 | | | | | | |
| AD- | gsusgcc(Chd)GfaCfAfAfgugcaaguuaL96 | 2510 | VPusAfsacuUfgCfAfcuugUfcGfggcacsgsa | 2602 | UCGUGCCCGACAAGUGCAAGUUC | 2694 |
| 397205 | | | | | | |
| AD- | gsascua(Chd)CfaGfUfGfaaccucuucaL96 | 2511 | VPusGfsaagAfgGfUfucacUfgGfuagucsusu | 2603 | AAGACUACCAGUGAACCUCUUCC | 2695 |
| 397206 | | | | | | |
| AD- | gsusccg(Chd)CfaUfCfAfaaaacugguaL96 | 2512 | VPusAfsccaGfuUfUfuugaUfgGfcggacsusu | 2604 | AAGUCCGCCAUCAAAAACUGGUG | 2696 |
| 397207 | | | | | | |
| AD- | gsgsccc(Uhd)CfgAfGfAfauuacaucaaL96 | 2513 | VPusUfsgauGfuAfAfuucuCfgAfgggccsasg | 2605 | CUGGCCCUCGAGAAUUACAUCAC | 2697 |
| 397208 | | | | | | |
| AD- | csasugc(Uhd)GfaAfGfAfaguacguccaL96 | 2514 | VPusGfsgacGfuAfCfuucuUfcAfgcaugsusu | 2606 | AACAUGCUGAAGAAGUACGUCCG | 2698 |
| 397209 | | | | | | |
| AD- | usgscug(Ahd)AfgAfAfGfuacguccguaL96 | 2515 | VPusAfscggAfcGfUfacuuCfuUfcagcasusg | 2607 | CAUGCUGAAGAAGUACGUCCGUG | 2699 |
| 397210 | | | | | | |
| AD- | uscscgc(Chd)AfuCfAfAfaaacuggugaL96 | 2516 | VPusCfsaccAfgUfUfuuugAfuGfgcggascsu | 2608 | AGUCCGCCAUCAAAAACUGGUGU | 2700 |
| 397211 | | | | | | |
| AD- | ususgca(Chd)GfaCfUfAfuggcaugcuaL96 | 2517 | VPusAfsgcaUfgCfCfauagUfcGfugcaasgsu | 2609 | ACUUGCACGACUAUGGCAUGCUG | 2701 |
| 397212 | | | | | | |
| AD- | uscscca(Ghd)GfuCfAfUfgagagaaugaL96 | 2518 | VPusCfsauuCfuCfUfcaugAfcCfugggascsa | 2610 | UGUCCCAGGUCAUGAGAGAAUGG | 2702 |
| 397213 | | | | | | |
| AD- | csusgaa(Ghd)AfaGfUfAfcguccgugcaL96 | 2519 | VPusGfscacGfgAfCfguacUfuCfuucagscsa | 2611 | UGCUGAAGAAGUACGUCCGUGCG | 2703 |
| 397214 | | | | | | |
| AD- | csgsugu(Ghd)AfuCfUfAfcgagcgcauaL96 | 2520 | VPusAfsugcGfcUfCfguagAfuCfacacgsgsa | 2612 | UCCGUGUGAUCUACGAGCGCAUG | 2704 |
| 397215 | | | | | | |
| AD- | usascug(Chd)CfaAfGfAfggucuacccaL96 | 2521 | VPusGfsgguAfgAfCfcucuUfgGfcaguascsu | 2613 | AGUACUGCCAAGAGGUCUACCCU | 2705 |
| 397216 | | | | | | |
| AD- | csasccg(Ahd)GfaGfAfGfaaugucccaaL96 | 2522 | VPusUfsgggAfcAfUfucucUfcUfcggugscsu | 2614 | AGCACCGAGAGAGAAUGUCCCAG | 2706 |
| 397217 | | | | | | |
| AD- | csasagg(Chd)CfuCfAfUfcauguguucaL96 | 2523 | VPusGfsaacAfcAfUfgaugAfgGfccuugsgsg | 2615 | CCCAAGGCCUCAUCAUGUGUUCA | 2707 |
| 397218 | | | | | | |
| AD- | gscsuga(Ahd)GfaAfGfUfacguccgugaL96 | 2524 | VPusCfsacgGfaCfGfuacuUfcUfucagcsasu | 2616 | AUGCUGAAGAAGUACGUCCGUGC | 2708 |
| 397219 | | | | | | |
| AD- | asasgca(Uhd)UfuUfGfAfacaugugcgaL96 | 2525 | VPusCfsgcaCfaUfGfuucaAfaAfugcuususa | 2617 | UAAAGCAUUUUGAACAUGUGCGC | 2709 |
| 397220 | | | | | | |
| AD- | csasccu(Chd)CfgUfGfUfgaucuacgaaL96 | 2526 | VPusUfscguAfgAfUfcacaCfgGfaggugsusg | 2618 | CACACCUCCGUGUGAUCUACGAG | 2710 |
| 397221 | | | | | | |
| AD- | gsasagg(Ahd)GfcAfGfAfacuacuccgaL96 | 2527 | VPusCfsggaGfuAfGfuucuGfcUfccuucsusg | 2619 | CAGAAGGAGCAGAACUACUCCGA | 2711 |
| 397222 | | | | | | |
| AD- | gsasaga(Ahd)AfcAfGfUfacacauccaaL96 | 2528 | VPusUfsggaUfgUfGfuacuGfuUfucuucsusu | 2620 | AAGAAGAAACAGUACACAUCCAU | 2712 |
| 397223 | | | | | | |
| AD- | gsusacu(Ghd)CfcAfAfGfaggucuaccaL96 | 2529 | VPusGfsguaGfaCfCfucuuGfgCfaguacsusg | 2621 | CAGUACUGCCAAGAGGUCUACCC | 2713 |
| 397224 | | | | | | |
| AD- | ascsugc(Chd)AfaGfAfGfgucuacccuaL96 | 2530 | VPusAfsgggUfaGfAfccucUfuGfgcagusasc | 2622 | GUACUGCCAAGAGGUCUACCCUG | 2714 |
| 397225 | | | | | | |
| AD- | ascsuaa(Chd)UfuGfCfAfcgacuauggaL96 | 2531 | VPusCfscauAfgUfCfgugcAfaGfuuagusgsc | 2623 | GCACUAACUUGCACGACUAUGGC | 2715 |
| 397226 | | | | | | |
| AD- | gsusccc(Ahd)UfuCfUfUfuuacggcggaL96 | 2532 | VPusCfscgcCfgUfAfaaagAfaUfgggacsasc | 2624 | GUGUCCCAUUCUUUUACGGCGGA | 2716 |
| 397227 | | | | | | |
| AD- | asasgcu(Ghd)AfcAfAfGfaaggccguuaL96 | 2533 | VPusAfsacgGfcCfUfucuuGfuCfagcuususg | 2625 | CAAAGCUGACAAGAAGGCCGUUA | 2717 |
| 397228 | | | | | | |
| AD- | usgsaca(Ahd)GfaAfGfGfccguuauccaL96 | 2534 | VPusGfsgauAfaCfGfgccuUfcUfugucasgsc | 2626 | GCUGACAAGAAGGCCGUUAUCCA | 2718 |
| 397229 | | | | | | |
| AD- | asgscau(Uhd)UfuGfAfAfcaugugcgcaL96 | 2535 | VPusGfscgcAfcAfUfguucAfaAfaugcususu | 2627 | AAAGCAUUUUGAACAUGUGCGCA | 2719 |
| 397230 | | | | | | |
| AD- | usgsuga(Uhd)CfuAfCfGfagcgcaugaaL96 | 2536 | VPusUfscauGfcGfCfucguAfgAfucacascsg | 2628 | CGUGUGAUCUACGAGCGCAUGAA | 2720 |
| 397231 | | | | | | |
| AD- | csasgcg(Ahd)GfaAfGfAfgcacuaacuaL96 | 2537 | VPusAfsguuAfgUfGfcucuUfcUfcgcugscsa | 2629 | UGCAGCGAGAAGAGCACUAACUU | 2721 |
| 397233 | | | | | | |
| AD- | asgscgu(Ghd)UfcAfAfCfccaaaguuuaL96 | 2538 | VPusAfsaacUfuUfGfgguuGfaCfacgcusgsc | 2630 | GCAGCGUGUCAACCCAAAGUUUA | 2722 |
| 397234 | | | | | | |
| AD- | usgsuca(Ahd)CfcCfAfAfaguuuacucaL96 | 2539 | VPusGfsaguAfaAfCfuuugGfgUfugacascsg | 2631 | CGUGUCAACCCAAAGUUUACUCA | 2723 |
| 397235 | | | | | | |
| AD- | usgsucc(Chd)AfuUfCfUfuuuacggcgaL96 | 2540 | VPusCfsgccGfuAfAfaagaAfuGfggacascsa | 2632 | UGUGUCCCAUUCUUUUACGGCGG | 2724 |
| 397236 | | | | | | |
| AD- | gsusguc(Ahd)AfcCfCfAfaaguuuacuaL96 | 2541 | VPusAfsguaAfaCfUfuuggGfuUfgacacsgsc | 2633 | GCGUGUCAACCCAAAGUUUACUC | 2725 |
| 397237 | | | | | | |
| AD- | asasgau(Chd)CfuGfAfUfaaacuucccaL96 | 2542 | VPusGfsggaAfgUfUfuaucAfgGfaucuusgsg | 2634 | CCAAGAUCCUGAUAAACUUCCCA | 2726 |
| 397238 | | | | | | |
| AD- | asgsauc(Chd)UfgAfUfAfaacuucccaaL96 | 2543 | VPusUfsgggAfaGfUfuuauCfaGfgaucususg | 2635 | CAAGAUCCUGAUAAACUUCCCAC | 2727 |
| 397239 | | | | | | |
| AD- | csusuac(Chd)GfuUfGfCfcuaguugguaL96 | 2544 | VPusAfsccaAfcUfAfggcaAfcGfguaagsgsa | 2636 | UCCUUACCGUUGCCUAGUUGGUG | 2728 |
| 397240 | | | | | | |
| AD- | gsusgug(Uhd)CfcCfAfUfucuuuuacgaL96 | 2545 | VPusCfsguaAfaAfGfaaugGfgAfcacacsusu | 2637 | AAGUGUGUCCCAUUCUUUUACGG | 2729 |
| 397241 | | | | | | |
| AD- | gsusguc(Chd)CfaUfUfCfuuuuacggcaL96 | 2546 | VPusGfsccgUfaAfAfagaaUfgGfgacacsasc | 2638 | GUGUGUCCCAUUCUUUUACGGCG | 2730 |
| 397242 | | | | | | |
| AD- | csasuag(Chd)AfaCfCfGfugauugucaaL96 | 2547 | VPusUfsgacAfaUfCfacggUfuGfcuaugsasc | 2639 | GUCAUAGCAACCGUGAUUGUCAU | 2731 |
| 397243 | | | | | | |
| AD- | gsasacg(Ghd)AfuAfUfGfagaauccaaaL96 | 2548 | VPusUfsuggAfuUfCfucauAfuCfcguucsusg | 2640 | CAGAACGGAUAUGAGAAUCCAAC | 2732 |
| 397244 | | | | | | |
| AD- | usgsugu(Chd)CfcAfUfUfcuuuuacggaL96 | 2549 | VPusCfscguAfaAfAfgaauGfgGfacacascsu | 2641 | AGUGUGUCCCAUUCUUUUACGGC | 2733 |
| 397245 | | | | | | |
| AD- | gscsaac(Chd)GfuGfAfUfugucaucacaL96 | 2550 | VPusGfsugaUfgAfCfaaucAfcGfguugcsusa | 2642 | UAGCAACCGUGAUUGUCAUCACC | 2734 |
| 397246 | | | | | | |
| AD- | gscsagc(Ghd)AfgAfAfGfagcacuaacaL96 | 2551 | VPusGfsuuaGfuGfCfucuuCfuCfgcugcsasu | 2643 | AUGCAGCGAGAAGAGCACUAACU | 2735 |
| 397247 | | | | | | |
| AD- | csasgaa(Uhd)UfcGfGfAfcaugauucaaL96 | 2552 | VPusUfsgaaUfcAfUfguccGfaAfuucugscsa | 2644 | UGCAGAAUUCGGACAUGAUUCAG | 2736 |
| 397248 | | | | | | |
| AD- | uscscug(Ahd)UfaAfAfCfuucccacgaaL96 | 2553 | VPusUfscguGfgGfAfaguuUfaUfcaggasusc | 2645 | GAUCCUGAUAAACUUCCCACGAC | 2737 |
| 397249 | | | | | | |
| AD- | asgsaac(Ghd)GfaUfAfUfgagaauccaaL96 | 2554 | VPusUfsggaUfuCfUfcauaUfcCfguucusgsc | 2646 | GCAGAACGGAUAUGAGAAUCCAA | 2738 |
| 397250 | | | | | | |
| AD- | cscsuua(Chd)CfgUfUfGfccuaguuggaL96 | 2555 | VPusCfscaaCfuAfGfgcaaCfgGfuaaggsasa | 2647 | UUCCUUACCGUUGCCUAGUUGGU | 2739 |
| 397251 | | | | | | |
| AD- | asusccu(Ghd)AfuAfAfAfcuucccacgaL96 | 2556 | VPusCfsgugGfgAfAfguuuAfuCfaggauscsu | 2648 | AGAUCCUGAUAAACUUCCCACGA | 2740 |
| 397252 | | | | | | |
| AD- | cscsuga(Uhd)AfaAfCfUfucccacgacaL96 | 2557 | VPusGfsucgUfgGfGfaaguUfuAfucaggsasu | 2649 | AUCCUGAUAAACUUCCCACGACA | 2741 |
| 397253 | | | | | | |
| AD- | csgsgau(Ghd)GfaUfGfUfuugugagacaL96 | 2558 | VPusGfsucuCfaCfAfaacaUfcCfauccgscsu | 2650 | AGCGGAUGGAUGUUUGUGAGACC | 2742 |
| 397254 | | | | | | |
| AD- | gsascac(Ghd)GfaAfGfAfguacugcauaL96 | 2559 | VPusAfsugcAfgUfAfcucuUfcCfgugucsasa | 2651 | UUGACACGGAAGAGUACUGCAUG | 2743 |
| 397255 | | | | | | |
| AD- | gscsagc(Ahd)GfaAfCfGfgauaugagaaL96 | 2560 | VPusUfscucAfuAfUfccguUfcUfgcugcsasu | 2652 | AUGCAGCAGAACGGAUAUGAGAA | 2744 |
| 397256 | | | | | | |
| AD- | gscsaga(Ahd)CfgGfAfUfaugagaaucaL96 | 2561 | VPusGfsauuCfuCfAfuaucCfgUfucugcsusg | 2653 | CAGCAGAACGGAUAUGAGAAUCC | 2745 |
| 397257 | | | | | | |
| AD- | csasgaa(Chd)GfgAfUfAfugagaauccaL96 | 2562 | VPusGfsgauUfcUfCfauauCfcGfuucugscsu | 2654 | AGCAGAACGGAUAUGAGAAUCCA | 2746 |
| 397258 | | | | | | |
| AD- | ascscgu(Chd)GfcCfAfAfagagacaugaL96 | 2563 | VPusCfsaugUfcUfCfuuugGfcGfacggusgsu | 2655 | ACACCGUCGCCAAAGAGACAUGC | 2747 |
| 397259 | | | | | | |
| AD- | gsusucu(Ghd)UfgGfUfAfaacucaacaaL96 | 2564 | VPusUfsguuGfaGfUfuuacCfaCfagaacsasu | 2656 | AUGUUCUGUGGUAAACUCAACAU | 2748 |
| 397260 | | | | | | |
| AD- | gsgsuac(Uhd)UfuGfAfUfgucacugaaaL96 | 2565 | VPusUfsucaGfuGfAfcaucAfaAfguaccsasg | 2657 | CUGGUACUUUGAUGUCACUGAAG | 2749 |
| 397261 | | | | | | |
| AD- | cscscaa(Ahd)GfuUfUfAfcucaagacuaL96 | 2566 | VPusAfsgucUfuGfAfguaaAfcUfuugggsusu | 2658 | AACCCAAAGUUUACUCAAGACUA | 2750 |
| 397262 | | | | | | |
| AD- | cscsaaa(Ghd)UfuUfAfCfucaagacuaaL96 | 2567 | VPusUfsaguCfuUfGfaguaAfaCfuuuggsgsu | 2659 | ACCCAAAGUUUACUCAAGACUAC | 2751 |
| 397263 | | | | | | |
| AD- | csasuca(Uhd)GfuGfUfUfcaacaugcuaL96 | 2568 | VPusAfsgcaUfgUfUfgaacAfcAfugaugsasg | 2660 | CUCAUCAUGUGUUCAACAUGCUG | 2752 |
| 397264 | | | | | | |
| AD- | asascau(Ghd)CfuGfAfAfgaaguacguaL96 | 2569 | VPusAfscguAfcUfUfcuucAfgCfauguusgsa | 2661 | UCAACAUGCUGAAGAAGUACGUC | 2753 |
| 397265 | | | | | | |
| AD- | ususcug(Uhd)GfgUfAfAfacucaacauaL96 | 2570 | VPusAfsuguUfgAfGfuuuaCfcAfcagaascsa | 2662 | UGUUCUGUGGUAAACUCAACAUG | 2754 |
| 397266 | | | | | | |
| AD- | uscsugu(Ghd)GfuAfAfAfcucaacaugaL96 | 2571 | VPusCfsaugUfuGfAfguuuAfcCfacagasasc | 2663 | GUUCUGUGGUAAACUCAACAUGC | 2755 |
| 397267 | | | | | | |

TABLE 20
Mouse APP Modified Sequences, No “L96” Linker, No Vinyl-Phosphate

| | | SEQ | | SEQ | | SEQ |
|---|---|---|---|---|---|---|
| Duplex | | ID | | ID | | ID |
| Name | Sense Sequence (5′ to 3′) | NO | Antisense Sequence (5′ to 3′) | NO | mRNA target sequence | NO |
| AD- | csasugu(Uhd)CfuGfUfGfguaaacucaa | 2480 | usUfsgagUfuUfAfccacAfgAfacaugsgsc | 2572 | GCCAUGUUCUGUGGUAAACUCAA | 2664 |
| 397175 | | | | | | |
| AD- | usgsuuc(Uhd)GfuGfGfUfaaacucaaca | 2481 | usGfsuugAfgUfUfuaccAfcAfgaacasusg | 2573 | CAUGUUCUGUGGUAAACUCAACA | 2665 |
| 397176 | | | | | | |
| AD- | asusguu(Chd)UfgUfGfGfuaaacucaaa | 2482 | usUfsugaGfuUfUfaccaCfaGfaacausgsg | 2574 | CCAUGUUCUGUGGUAAACUCAAC | 2666 |
| 397177 | | | | | | |
| AD- | csusgug(Ghd)UfaAfAfCfucaacaugca | 2483 | usGfscauGfuUfGfaguuUfaCfcacagsasa | 2575 | UUCUGUGGUAAACUCAACAUGCA | 2667 |
| 397178 | | | | | | |
| AD- | gsgsuaa(Ahd)CfuCfAfAfcaugcacaua | 2484 | usAfsuguGfcAfUfguugAfgUfuuaccsasc | 2576 | GUGGUAAACUCAACAUGCACAUG | 2668 |
| 397179 | | | | | | |
| AD- | usgsugg(Uhd)AfaAfCfUfcaacaugcaa | 2485 | usUfsgcaUfgUfUfgaguUfuAfccacasgsa | 2577 | UCUGUGGUAAACUCAACAUGCAC | 2669 |
| 397180 | | | | | | |
| AD- | gsasaga(Ghd)CfaCfUfAfacuugcacga | 2486 | usCfsgugCfaAfGfuuagUfgCfucuucsusc | 2578 | GAGAAGAGCACUAACUUGCACGA | 2670 |
| 397181 | | | | | | |
| AD- | cscsgcu(Ghd)GfuAfCfUfuugaugucaa | 2487 | usUfsgacAfuCfAfaaguAfcCfagcggsgsa | 2579 | UCCCGCUGGUACUUUGAUGUCAC | 2671 |
| 397182 | | | | | | |
| AD- | cscsaug(Uhd)UfcUfGfUfgguaaacuca | 2488 | usGfsaguUfuAfCfcacaGfaAfcauggscsg | 2580 | CGCCAUGUUCUGUGGUAAACUCA | 2672 |
| 397183 | | | | | | |
| AD- | gsusggu(Ahd)AfaCfUfCfaacaugcaca | 2489 | usGfsugcAfuGfUfugagUfuUfaccacsasg | 2581 | CUGUGGUAAACUCAACAUGCACA | 2673 |
| 397184 | | | | | | |
| AD- | gsasacu(Ghd)CfaGfAfUfcacaaacgua | 2490 | usAfscguUfuGfUfgaucUfgCfaguucsasg | 2582 | CUGAACUGCAGAUCACAAACGUG | 2674 |
| 397185 | | | | | | |
| AD- | asasgag(Chd)AfcUfAfAfcuugcacgaa | 2491 | usUfscguGfcAfAfguuaGfuGfcucuuscsu | 2583 | AGAAGAGCACUAACUUGCACGAC | 2675 |
| 397186 | | | | | | |
| AD- | asgscac(Uhd)AfaCfUfUfgcacgacuaa | 2492 | usUfsaguCfgUfGfcaagUfuAfgugcuscsu | 2584 | AGAGCACUAACUUGCACGACUAU | 2676 |
| 397187 | | | | | | |
| AD- | gscsacu(Ahd)AfcUfUfGfcacgacuaua | 2493 | usAfsuagUfcGfUfgcaaGfuUfagugcsusc | 2585 | GAGCACUAACUUGCACGACUAUG | 2677 |
| 397188 | | | | | | |
| AD- | asasagu(Uhd)UfaCfUfCfaagacuacca | 2494 | usGfsguaGfuCfUfugagUfaAfacuuusgsg | 2586 | CCAAAGUUUACUCAAGACUACCA | 2678 |
| 397189 | | | | | | |
| AD- | csgscau(Ghd)AfaCfCfAfgucucuguca | 2495 | usGfsacaGfaGfAfcuggUfuCfaugcgscsu | 2587 | AGCGCAUGAACCAGUCUCUGUCC | 2679 |
| 397190 | | | | | | |
| AD- | csascau(Chd)GfuGfAfUfuccuuaccga | 2496 | usCfsgguAfaGfGfaaucAfcGfaugugsgsg | 2588 | CCCACAUCGUGAUUCCUUACCGU | 2680 |
| 397191 | | | | | | |
| AD- | asusgcu(Ghd)AfaGfAfAfguacguccga | 2497 | usCfsggaCfgUfAfcuucUfuCfagcausgsu | 2589 | ACAUGCUGAAGAAGUACGUCCGU | 2681 |
| 397192 | | | | | | |
| AD- | gsasgcg(Chd)AfuGfAfAfccagucucua | 2498 | usAfsgagAfcUfGfguucAfuGfcgcucsgsu | 2590 | ACGAGCGCAUGAACCAGUCUCUG | 2682 |
| 397193 | | | | | | |
| AD- | gsasgca(Ghd)AfaCfUfAfcuccgacgaa | 2499 | usUfscguCfgGfAfguagUfuCfugcucscsu | 2591 | AGGAGCAGAACUACUCCGACGAU | 2683 |
| 397194 | | | | | | |
| AD- | csasccc(Ahd)CfaUfCfGfugauuccuua | 2500 | usAfsaggAfaUfCfacgaUfgUfgggugsusg | 2592 | CACACCCACAUCGUGAUUCCUUA | 2684 |
| 397195 | | | | | | |
| AD- | asgsagc(Ahd)CfuAfAfCfuugcacgaca | 2501 | usGfsucgUfgCfAfaguuAfgUfgcucususc | 2593 | GAAGAGCACUAACUUGCACGACU | 2685 |
| 397196 | | | | | | |
| AD- | csascua(Ahd)CfuUfGfCfacgacuauga | 2502 | usCfsauaGfuCfGfugcaAfgUfuagugscsu | 2594 | AGCACUAACUUGCACGACUAUGG | 2686 |
| 397197 | | | | | | |
| AD- | csuscaa(Ghd)AfcUfAfCfcagugaacca | 2503 | usGfsguuCfaCfUfgguaGfuCfuugagsusa | 2595 | UACUCAAGACUACCAGUGAACCU | 2687 |
| 397198 | | | | | | |
| AD- | asgscac(Ahd)CfcCfUfAfaagcauuuua | 2504 | usAfsaaaUfgCfUfuuagGfgUfgugcusgsu | 2596 | ACAGCACACCCUAAAGCAUUUUG | 2688 |
| 397199 | | | | | | |
| AD- | asasgga(Ghd)CfaGfAfAfcuacuccgaa | 2505 | usUfscggAfgUfAfguucUfgCfuccuuscsu | 2597 | AGAAGGAGCAGAACUACUCCGAC | 2689 |
| 397200 | | | | | | |
| AD- | gsgsagc(Ahd)GfaAfCfUfacuccgacga | 2506 | usCfsgucGfgAfGfuaguUfcUfgcuccsusu | 2598 | AAGGAGCAGAACUACUCCGACGA | 2690 |
| 397201 | | | | | | |
| AD- | gsasaac(Ahd)GfuAfCfAfcauccaucca | 2507 | usGfsgauGfgAfUfguguAfcUfguuucsusu | 2599 | AAGAAACAGUACACAUCCAUCCA | 2691 |
| 397202 | | | | | | |
| AD- | csusgaa(Chd)UfgCfAfGfaucacaaaca | 2508 | usGfsuuuGfuGfAfucugCfaGfuucagsgsg | 2600 | CCCUGAACUGCAGAUCACAAACG | 2692 |
| 397203 | | | | | | |
| AD- | cscsaca(Uhd)CfgUfGfAfuuccuuacca | 2509 | usGfsguaAfgGfAfaucaCfgAfuguggsgsu | 2601 | ACCCACAUCGUGAUUCCUUACCG | 2693 |
| 397204 | | | | | | |
| AD- | gsusgcc(Chd)GfaCfAfAfgugcaaguua | 2510 | usAfsacuUfgCfAfcuugUfcGfggcacsgsa | 2602 | UCGUGCCCGACAAGUGCAAGUUC | 2694 |
| 397205 | | | | | | |
| AD- | gsascua(Chd)CfaGfUfGfaaccucuuca | 2511 | usGfsaagAfgGfUfucacUfgGfuagucsusu | 2603 | AAGACUACCAGUGAACCUCUUCC | 2695 |
| 397206 | | | | | | |
| AD- | gsusccg(Chd)CfaUfCfAfaaaacuggua | 2512 | usAfsccaGfuUfUfuugaUfgGfcggacsusu | 2604 | AAGUCCGCCAUCAAAAACUGGUG | 2696 |
| 397207 | | | | | | |
| AD- | gsgsccc(Uhd)CfgAfGfAfauuacaucaa | 2513 | usUfsgauGfuAfAfuucuCfgAfgggccsasg | 2605 | CUGGCCCUCGAGAAUUACAUCAC | 2697 |
| 397208 | | | | | | |
| AD- | csasugc(Uhd)GfaAfGfAfaguacgucca | 2514 | usGfsgacGfuAfCfuucuUfcAfgcaugsusu | 2606 | AACAUGCUGAAGAAGUACGUCCG | 2698 |
| 397209 | | | | | | |
| AD- | usgscug(Ahd)AfgAfAfGfuacguccgua | 2515 | usAfscggAfcGfUfacuuCfuUfcagcasusg | 2607 | CAUGCUGAAGAAGUACGUCCGUG | 2699 |
| 397210 | | | | | | |
| AD- | uscscgc(Chd)AfuCfAfAfaaacugguga | 2516 | usCfsaccAfgUfUfuuugAfuGfgcggascsu | 2608 | AGUCCGCCAUCAAAAACUGGUGU | 2700 |
| 397211 | | | | | | |
| AD- | ususgca(Chd)GfaCfUfAfuggcaugcua | 2517 | usAfsgcaUfgCfCfauagUfcGfugcaasgsu | 2609 | ACUUGCACGACUAUGGCAUGCUG | 2701 |
| 397212 | | | | | | |
| AD- | uscscca(Ghd)GfuCfAfUfgagagaauga | 2518 | usCfsauuCfuCfUfcaugAfcCfugggascsa | 2610 | UGUCCCAGGUCAUGAGAGAAUGG | 2702 |
| 397213 | | | | | | |
| AD- | csusgaa(Ghd)AfaGfUfAfcguccgugca | 2519 | usGfscacGfgAfCfguacUfuCfuucagscsa | 2611 | UGCUGAAGAAGUACGUCCGUGCG | 2703 |
| 397214 | | | | | | |
| AD- | csgsugu(Ghd)AfuCfUfAfcgagcgcaua | 2520 | usAfsugcGfcUfCfguagAfuCfacacgsgsa | 2612 | UCCGUGUGAUCUACGAGCGCAUG | 2704 |
| 397215 | | | | | | |
| AD- | usascug(Chd)CfaAfGfAfggucuaccca | 2521 | usGfsgguAfgAfCfcucuUfgGfcaguascsu | 2613 | AGUACUGCCAAGAGGUCUACCCU | 2705 |
| 397216 | | | | | | |
| AD- | csasccg(Ahd)GfaGfAfGfaaugucccaa | 2522 | usUfsgggAfcAfUfucucUfcUfcggugscsu | 2614 | AGCACCGAGAGAGAAUGUCCCAG | 2706 |
| 397217 | | | | | | |
| AD- | csasagg(Chd)CfuCfAfUfcauguguuca | 2523 | usGfsaacAfcAfUfgaugAfgGfccuugsgsg | 2615 | CCCAAGGCCUCAUCAUGUGUUCA | 2707 |
| 397218 | | | | | | |
| AD- | gscsuga(Ahd)GfaAfGfUfacguccguga | 2524 | usCfsacgGfaCfGfuacuUfcUfucagcsasu | 2616 | AUGCUGAAGAAGUACGUCCGUGC | 2708 |
| 397219 | | | | | | |
| AD- | asasgca(Uhd)UfuUfGfAfacaugugcga | 2525 | usCfsgcaCfaUfGfuucaAfaAfugcuususa | 2617 | UAAAGCAUUUUGAACAUGUGCGC | 2709 |
| 397220 | | | | | | |
| AD- | csasccu(Chd)CfgUfGfUfgaucuacgaa | 2526 | usUfscguAfgAfUfcacaCfgGfaggugsusg | 2618 | CACACCUCCGUGUGAUCUACGAG | 2710 |
| 397221 | | | | | | |
| AD- | gsasagg(Ahd)GfcAfGfAfacuacuccga | 2527 | usCfsggaGfuAfGfuucuGfcUfccuucsusg | 2619 | CAGAAGGAGCAGAACUACUCCGA | 2711 |
| 397222 | | | | | | |
| AD- | gsasaga(Ahd)AfcAfGfUfacacauccaa | 2528 | usUfsggaUfgUfGfuacuGfuUfucuucsusu | 2620 | AAGAAGAAACAGUACACAUCCAU | 2712 |
| 397223 | | | | | | |
| AD- | gsusacu(Ghd)CfcAfAfGfaggucuacca | 2529 | usGfsguaGfaCfCfucuuGfgCfaguacsusg | 2621 | CAGUACUGCCAAGAGGUCUACCC | 2713 |
| 397224 | | | | | | |
| AD- | ascsugc(Chd)AfaGfAfGfgucuacccua | 2530 | usAfsgggUfaGfAfccucUfuGfgcagusasc | 2622 | GUACUGCCAAGAGGUCUACCCUG | 2714 |
| 397225 | | | | | | |
| AD- | ascsuaa(Chd)UfuGfCfAfcgacuaugga | 2531 | usCfscauAfgUfCfgugcAfaGfuuagusgsc | 2623 | GCACUAACUUGCACGACUAUGGC | 2715 |
| 397226 | | | | | | |
| AD- | gsusccc(Ahd)UfuCfUfUfuuacggcgga | 2532 | usCfscgcCfgUfAfaaagAfaUfgggacsasc | 2624 | GUGUCCCAUUCUUUUACGGCGGA | 2716 |
| 397227 | | | | | | |
| AD- | asasgcu(Ghd)AfcAfAfGfaaggccguua | 2533 | usAfsacgGfcCfUfucuuGfuCfagcuususg | 2625 | CAAAGCUGACAAGAAGGCCGUUA | 2717 |
| 397228 | | | | | | |
| AD- | usgsaca(Ahd)GfaAfGfGfccguuaucca | 2534 | usGfsgauAfaCfGfgccuUfcUfugucasgsc | 2626 | GCUGACAAGAAGGCCGUUAUCCA | 2718 |
| 397229 | | | | | | |
| AD- | asgscau(Uhd)UfuGfAfAfcaugugcgca | 2535 | usGfscgcAfcAfUfguucAfaAfaugcususu | 2627 | AAAGCAUUUUGAACAUGUGCGCA | 2719 |
| 397230 | | | | | | |
| AD- | usgsuga(Uhd)CfuAfCfGfagcgcaugaa | 2536 | usUfscauGfcGfCfucguAfgAfucacascsg | 2628 | CGUGUGAUCUACGAGCGCAUGAA | 2720 |
| 397231 | | | | | | |
| AD- | csasgcg(Ahd)GfaAfGfAfgcacuaacua | 2537 | usAfsguuAfgUfGfcucuUfcUfcgcugscsa | 2629 | UGCAGCGAGAAGAGCACUAACUU | 2721 |
| 397233 | | | | | | |
| AD- | asgscgu(Ghd)UfcAfAfCfccaaaguuua | 2538 | usAfsaacUfuUfGfgguuGfaCfacgcusgsc | 2630 | GCAGCGUGUCAACCCAAAGUUUA | 2722 |
| 397234 | | | | | | |
| AD- | usgsuca(Ahd)CfcCfAfAfaguuuacuca | 2539 | usGfsaguAfaAfCfuuugGfgUfugacascsg | 2631 | CGUGUCAACCCAAAGUUUACUCA | 2723 |
| 397235 | | | | | | |
| AD- | usgsucc(Chd)AfuUfCfUfuuuacggcga | 2540 | usCfsgccGfuAfAfaagaAfuGfggacascsa | 2632 | UGUGUCCCAUUCUUUUACGGCGG | 2724 |
| 397236 | | | | | | |
| AD- | gsusguc(Ahd)AfcCfCfAfaaguuuacua | 2541 | usAfsguaAfaCfUfuuggGfuUfgacacsgsc | 2633 | GCGUGUCAACCCAAAGUUUACUC | 2725 |
| 397237 | | | | | | |
| AD- | asasgau(Chd)CfuGfAfUfaaacuuccca | 2542 | usGfsggaAfgUfUfuaucAfgGfaucuusgsg | 2634 | CCAAGAUCCUGAUAAACUUCCCA | 2726 |
| 397238 | | | | | | |
| AD- | asgsauc(Chd)UfgAfUfAfaacuucccaa | 2543 | usUfsgggAfaGfUfuuauCfaGfgaucususg | 2635 | CAAGAUCCUGAUAAACUUCCCAC | 2727 |
| 397239 | | | | | | |
| AD- | csusuac(Chd)GfuUfGfCfcuaguuggua | 2544 | usAfsccaAfcUfAfggcaAfcGfguaagsgsa | 2636 | UCCUUACCGUUGCCUAGUUGGUG | 2728 |
| 397240 | | | | | | |
| AD- | gsusgug(Uhd)CfcCfAfUfucuuuuacga | 2545 | usCfsguaAfaAfGfaaugGfgAfcacacsusu | 2637 | AAGUGUGUCCCAUUCUUUUACGG | 2729 |
| 397241 | | | | | | |
| AD- | gsusguc(Chd)CfaUfUfCfuuuuacggca | 2546 | usGfsccgUfaAfAfagaaUfgGfgacacsasc | 2638 | GUGUGUCCCAUUCUUUUACGGCG | 2730 |
| 397242 | | | | | | |
| AD- | csasuag(Chd)AfaCfCfGfugauugucaa | 2547 | usUfsgacAfaUfCfacggUfuGfcuaugsasc | 2639 | GUCAUAGCAACCGUGAUUGUCAU | 2731 |
| 397243 | | | | | | |
| AD- | gsasacg(Ghd)AfuAfUfGfagaauccaaa | 2548 | usUfsuggAfuUfCfucauAfuCfcguucsusg | 2640 | CAGAACGGAUAUGAGAAUCCAAC | 2732 |
| 397244 | | | | | | |
| AD- | usgsugu(Chd)CfcAfUfUfcuuuuacgga | 2549 | usCfscguAfaAfAfgaauGfgGfacacascsu | 2641 | AGUGUGUCCCAUUCUUUUACGGC | 2733 |
| 397245 | | | | | | |
| AD- | gscsaac(Chd)GfuGfAfUfugucaucaca | 2550 | usGfsugaUfgAfCfaaucAfcGfguugcsusa | 2642 | UAGCAACCGUGAUUGUCAUCACC | 2734 |
| 397246 | | | | | | |
| AD- | gscsagc(Ghd)AfgAfAfGfagcacuaaca | 2551 | usGfsuuaGfuGfCfucuuCfuCfgcugcsasu | 2643 | AUGCAGCGAGAAGAGCACUAACU | 2735 |
| 397247 | | | | | | |
| AD- | csasgaa(Uhd)UfcGfGfAfcaugauucaa | 2552 | usUfsgaaUfcAfUfguccGfaAfuucugscsa | 2644 | UGCAGAAUUCGGACAUGAUUCAG | 2736 |
| 397248 | | | | | | |
| AD- | uscscug(Ahd)UfaAfAfCfuucccacgaa | 2553 | usUfscguGfgGfAfaguuUfaUfcaggasusc | 2645 | GAUCCUGAUAAACUUCCCACGAC | 2737 |
| 397249 | | | | | | |
| AD- | asgsaac(Ghd)GfaUfAfUfgagaauccaa | 2554 | usUfsggaUfuCfUfcauaUfcCfguucusgsc | 2646 | GCAGAACGGAUAUGAGAAUCCAA | 2738 |
| 397250 | | | | | | |
| AD- | cscsuua(Chd)CfgUfUfGfccuaguugga | 2555 | usCfscaaCfuAfGfgcaaCfgGfuaaggsasa | 2647 | UUCCUUACCGUUGCCUAGUUGGU | 2739 |
| 397251 | | | | | | |
| AD- | asusccu(Ghd)AfuAfAfAfcuucccacga | 2556 | usCfsgugGfgAfAfguuuAfuCfaggauscsu | 2648 | AGAUCCUGAUAAACUUCCCACGA | 2740 |
| 397252 | | | | | | |
| AD- | cscsuga(Uhd)AfaAfCfUfucccacgaca | 2557 | usGfsucgUfgGfGfaaguUfuAfucaggsasu | 2649 | AUCCUGAUAAACUUCCCACGACA | 2741 |
| 397253 | | | | | | |
| AD- | csgsgau(Ghd)GfaUfGfUfuugugagaca | 2558 | usGfsucuCfaCfAfaacaUfcCfauccgscsu | 2650 | AGCGGAUGGAUGUUUGUGAGACC | 2742 |
| 397254 | | | | | | |
| AD- | gsascac(Ghd)GfaAfGfAfguacugcaua | 2559 | usAfsugcAfgUfAfcucuUfcCfgugucsasa | 2651 | UUGACACGGAAGAGUACUGCAUG | 2743 |
| 397255 | | | | | | |
| AD- | gscsagc(Ahd)GfaAfCfGfgauaugagaa | 2560 | usUfscucAfuAfUfccguUfcUfgcugcsasu | 2652 | AUGCAGCAGAACGGAUAUGAGAA | 2744 |
| 397256 | | | | | | |
| AD- | gscsaga(Ahd)CfgGfAfUfaugagaauca | 2561 | usGfsauuCfuCfAfuaucCfgUfucugcsusg | 2653 | CAGCAGAACGGAUAUGAGAAUCC | 2745 |
| 397257 | | | | | | |
| AD- | csasgaa(Chd)GfgAfUfAfugagaaucca | 2562 | usGfsgauUfcUfCfauauCfcGfuucugscsu | 2654 | AGCAGAACGGAUAUGAGAAUCCA | 2746 |
| 397258 | | | | | | |
| AD- | ascscgu(Chd)GfcCfAfAfagagacauga | 2563 | usCfsaugUfcUfCfuuugGfcGfacggusgsu | 2655 | ACACCGUCGCCAAAGAGACAUGC | 2747 |
| 397259 | | | | | | |
| AD- | gsusucu(Ghd)UfgGfUfAfaacucaacaa | 2564 | usUfsguuGfaGfUfuuacCfaCfagaacsasu | 2656 | AUGUUCUGUGGUAAACUCAACAU | 2748 |
| 397260 | | | | | | |
| AD- | gsgsuac(Uhd)UfuGfAfUfgucacugaaa | 2565 | usUfsucaGfuGfAfcaucAfaAfguaccsasg | 2657 | CUGGUACUUUGAUGUCACUGAAG | 2749 |
| 397261 | | | | | | |
| AD- | cscscaa(Ahd)GfuUfUfAfcucaagacua | 2566 | usAfsgucUfuGfAfguaaAfcUfuugggsusu | 2658 | AACCCAAAGUUUACUCAAGACUA | 2750 |
| 397262 | | | | | | |
| AD- | cscsaaa(Ghd)UfuUfAfCfucaagacuaa | 2567 | usUfsaguCfuUfGfaguaAfaCfuuuggsgsu | 2659 | ACCCAAAGUUUACUCAAGACUAC | 2751 |
| 397263 | | | | | | |
| AD- | csasuca(Uhd)GfuGfUfUfcaacaugcua | 2568 | usAfsgcaUfgUfUfgaacAfcAfugaugsasg | 2660 | CUCAUCAUGUGUUCAACAUGCUG | 2752 |
| 397264 | | | | | | |
| AD- | asascau(Ghd)CfuGfAfAfgaaguacgua | 2569 | usAfscguAfcUfUfcuucAfgCfauguusgsa | 2661 | UCAACAUGCUGAAGAAGUACGUC | 2753 |
| 397265 | | | | | | |
| AD- | ususcug(Uhd)GfgUfAfAfacucaacaua | 2570 | usAfsuguUfgAfGfuuuaCfcAfcagaascsa | 2662 | UGUUCUGUGGUAAACUCAACAUG | 2754 |
| 397266 | | | | | | |
| AD- | uscsugu(Ghd)GfuAfAfAfcucaacauga | 2571 | usCfsaugUfuGfAfguuuAfcCfacagasasc | 2663 | GUUCUGUGGUAAACUCAACAUGC | 2755 |
| 397267 | | | | | | |

TABLE 21
mmAPP-Targeting RNAi Agents, Unmodified Sequences, Mouse NM_001198823.1 Targeting

| Duplex | | SEQ ID | Position in | | SEQ ID | Position in |
|---|---|---|---|---|---|---|
| Name | Sense Sequence (5′ to 3′) | NO | NM_001198823.1 | Antisense Sequence (5′ to 3′) | NO | NM_001198823.1 |
| AD-397183 | CCAUGUUCUGUGGUAAACUCA | 2756 | 253-273 | UGAGUUUACCACAGAACAUGGCG | 2848 | 251-273 |
| AD-397175 | CAUGUUCUGUGGUAAACUCAA | 2757 | 254-274 | UUGAGUUUACCACAGAACAUGGC | 2849 | 252-274 |
| AD-397177 | AUGUUCUGUGGUAAACUCAAA | 2758 | 255-275 | UUUGAGUUUACCACAGAACAUGG | 2850 | 253-275 |
| AD-397176 | UGUUCUGUGGUAAACUCAACA | 2759 | 256-276 | UGUUGAGUUUACCACAGAACAUG | 2851 | 254-276 |
| AD-397260 | GUUCUGUGGUAAACUCAACAA | 2760 | 257-277 | UUGUUGAGUUUACCACAGAACAU | 2852 | 255-277 |
| AD-397266 | UUCUGUGGUAAACUCAACAUA | 2761 | 258-278 | UAUGUUGAGUUUACCACAGAACA | 2853 | 256-278 |
| AD-397267 | UCUGUGGUAAACUCAACAUGA | 2762 | 259-279 | UCAUGUUGAGUUUACCACAGAAC | 2854 | 257-279 |
| AD-397178 | CUGUGGUAAACUCAACAUGCA | 2763 | 260-280 | UGCAUGUUGAGUUUACCACAGAA | 2855 | 258-280 |
| AD-397180 | UGUGGUAAACUCAACAUGCAA | 2764 | 261-281 | UUGCAUGUUGAGUUUACCACAGA | 2856 | 259-281 |
| AD-39718 | GUGGUAAACUCAACAUGCACA | 2765 | 262-282 | UGUGCAUGUUGAGUUUACCACAG | 2857 | 260-282 |
| AD-397179 | GGUAAACUCAACAUGCACAUA | 2766 | 264-284 | UAUGUGCAUGUUGAGUUUACCAC | 2858 | 262-284 |
| AD-397224 | GUACUGCCAAGAGGUCUACCA | 2767 | 362-382 | UGGUAGACCUCUUGGCAGUACUG | 2859 | 360-382 |
| AD-397216 | UACUGCCAAGAGGUCUACCCA | 2768 | 363-383 | UGGGUAGACCUCUUGGCAGUACU | 2860 | 361-383 |
| AD-397225 | ACUGCCAAGAGGUCUACCCUA | 2769 | 364-384 | UAGGGUAGACCUCUUGGCAGUAC | 2861 | 362-384 |
| AD-397203 | CUGAACUGCAGAUCACAAACA | 2770 | 382-402 | UGUUUGUGAUCUGCAGUUCAGGG | 2862 | 380-402 |
| AD-397185 | GAACUGCAGAUCACAAACGUA | 2771 | 384-404 | UACGUUUGUGAUCUGCAGUUCAG | 2863 | 382-404 |
| AD-397195 | CACCCACAUCGUGAUUCCUUA | 2772 | 473-493 | UAAGGAAUCACGAUGUGGGUGUG | 2864 | 471-493 |
| AD-397204 | CCACAUCGUGAUUCCUUACCA | 2773 | 476-496 | UGGUAAGGAAUCACGAUGUGGGU | 2865 | 474-496 |
| AD-397191 | CACAUCGUGAUUCCUUACCGA | 2774 | 477-497 | UCGGUAAGGAAUCACGAUGUGGG | 2866 | 475-497 |
| AD-397251 | CCUUACCGUUGCCUAGUUGGA | 2775 | 489-509 | UCCAACUAGGCAACGGUAAGGAA | 2867 | 487-509 |
| AD-397240 | CUUACCGUUGCCUAGUUGGUA | 2776 | 490-510 | UACCAACUAGGCAACGGUAAGGA | 2868 | 488-510 |
| AD-397205 | GUGCCCGACAAGUGCAAGUUA | 2777 | 534-554 | UAACUUGCACUUGUCGGGCACGA | 2869 | 532-554 |
| AD-397254 | CGGAUGGAUGUUUGUGAGACA | 2778 | 567-587 | UGUCUCACAAACAUCCAUCCGCU | 2870 | 565-587 |
| AD-397259 | ACCGUCGCCAAAGAGACAUGA | 2779 | 603-623 | UCAUGUCUCUUUGGCGACGGUGU | 2871 | 601-623 |
| AD-397247 | GCAGCGAGAAGAGCACUAACA | 2780 | 622-642 | UGUUAGUGCUCUUCUCGCUGCAU | 2872 | 620-642 |
| AD-397233 | CAGCGAGAAGAGCACUAACUA | 2781 | 623-643 | UAGUUAGUGCUCUUCUCGCUGCA | 2873 | 621-643 |
| AD-397181 | GAAGAGCACUAACUUGCACGA | 2782 | 629-649 | UCGUGCAAGUUAGUGCUCUUCUC | 2874 | 627-649 |
| AD-397186 | AAGAGCACUAACUUGCACGAA | 2783 | 630-650 | UUCGUGCAAGUUAGUGCUCUUCU | 2875 | 628-650 |
| AD-397196 | AGAGCACUAACUUGCACGACA | 2784 | 631-651 | UGUCGUGCAAGUUAGUGCUCUUC | 2876 | 629-651 |
| AD-397187 | AGCACUAACUUGCACGACUAA | 2785 | 633-653 | UUAGUCGUGCAAGUUAGUGCUCU | 2877 | 631-653 |
| AD-397188 | GCACUAACUUGCACGACUAUA | 2786 | 634-654 | UAUAGUCGUGCAAGUUAGUGCUC | 2878 | 632-654 |
| AD-397197 | CACUAACUUGCACGACUAUGA | 2787 | 635-655 | UCAUAGUCGUGCAAGUUAGUGCU | 2879 | 633-655 |
| AD-397226 | ACUAACUUGCACGACUAUGGA | 2788 | 636-656 | UCCAUAGUCGUGCAAGUUAGUGC | 2880 | 634-656 |
| AD-397212 | UUGCACGACUAUGGCAUGCUA | 2789 | 642-662 | UAGCAUGCCAUAGUCGUGCAAGU | 2881 | 640-662 |
| AD-397182 | CCGCUGGUACUUUGAUGUCAA | 2790 | 1064-1084 | UUGACAUCAAAGUACCAGCGGGA | 2882 | 1062-1084 |
| AD-397261 | GGUACUUUGAUGUCACUGAAA | 2791 | 1069-1089 | UUUCAGUGACAUCAAAGUACCAG | 2883 | 1067-1089 |
| AD-397241 | GUGUGUCCCAUUCUUUUACGA | 2792 | 1094-1114 | UCGUAAAAGAAUGGGACACACUU | 2884 | 1092-1114 |
| AD-397245 | UGUGUCCCAUUCUUUUACGGA | 2793 | 1095-1115 | UCCGUAAAAGAAUGGGACACACU | 2885 | 1093-1115 |
| AD-397242 | GUGUCCCAUUCUUUUACGGCA | 2794 | 1096-1116 | UGCCGUAAAAGAAUGGGACACAC | 2886 | 1094-1116 |
| AD-397236 | UGUCCCAUUCUUUUACGGCGA | 2795 | 1097-1117 | UCGCCGUAAAAGAAUGGGACACA | 2887 | 1095-1117 |
| AD-397227 | GUCCCAUUCUUUUACGGCGGA | 2796 | 1098-1118 | UCCGCCGUAAAAGAAUGGGACAC | 2888 | 1096-1118 |
| AD-397255 | GACACGGAAGAGUACUGCAUA | 2797 | 1143-1163 | UAUGCAGUACUCUUCCGUGUCAA | 2889 | 1141-1163 |
| AD-397234 | AGCGUGUCAACCCAAAGUUUA | 2798 | 1176-1196 | UAAACUUUGGGUUGACACGCUGC | 2890 | 1174-1196 |
| AD-397237 | GUGUCAACCCAAAGUUUACUA | 2799 | 1179-1199 | UAGUAAACUUUGGGUUGACACGC | 2891 | 1177-1199 |
| AD-397235 | UGUCAACCCAAAGUUUACUCA | 2800 | 1180-1200 | UGAGUAAACUUUGGGUUGACACG | 2892 | 1178-1200 |
| AD-397262 | CCCAAAGUUUACUCAAGACUA | 2801 | 1186-1206 | UAGUCUUGAGUAAACUUUGGGUU | 2893 | 1184-1206 |
| AD-397263 | CCAAAGUUUACUCAAGACUAA | 2802 | 1187-1207 | UUAGUCUUGAGUAAACUUUGGGU | 2894 | 1185-1207 |
| AD-397189 | AAAGUUUACUCAAGACUACCA | 2803 | 1189-1209 | UGGUAGUCUUGAGUAAACUUUGG | 2895 | 1187-1209 |
| AD-397198 | CUCAAGACUACCAGUGAACCA | 2804 | 1197-1217 | UGGUUCACUGGUAGUCUUGAGUA | 2896 | 1195-1217 |
| AD-397206 | GACUACCAGUGAACCUCUUCA | 2805 | 1202-1222 | UGAAGAGGUUCACUGGUAGUCUU | 2897 | 1200-1222 |
| AD-397238 | AAGAUCCUGAUAAACUUCCCA | 2806 | 1225-1245 | UGGGAAGUUUAUCAGGAUCUUGG | 2898 | 1223-1245 |
| AD-397239 | AGAUCCUGAUAAACUUCCCAA | 2807 | 1226-1246 | UUGGGAAGUUUAUCAGGAUCUUG | 2899 | 1224-1246 |
| AD-397252 | AUCCUGAUAAACUUCCCACGA | 2808 | 1228-1248 | UCGUGGGAAGUUUAUCAGGAUCU | 2900 | 1226-1248 |
| AD-397249 | UCCUGAUAAACUUCCCACGAA | 2809 | 1229-1249 | UUCGUGGGAAGUUUAUCAGGAUC | 2901 | 1227-1249 |
| AD-397253 | CCUGAUAAACUUCCCACGACA | 2810 | 1230-1250 | UGUCGUGGGAAGUUUAUCAGGAU | 2902 | 1228-1250 |
| AD-397217 | CACCGAGAGAGAAUGUCCCAA | 2811 | 1353-1373 | UUGGGACAUUCUCUCUCGGUGCU | 2903 | 1351-1373 |
| AD-397213 | UCCCAGGUCAUGAGAGAAUGA | 2812 | 1368-1388 | UCAUUCUCUCAUGACCUGGGACA | 2904 | 1366-1388 |
| AD-397228 | AAGCUGACAAGAAGGCCGUUA | 2813 | 1423-1443 | UAACGGCCUUCUUGUCAGCUUUG | 2905 | 1421-1443 |
| AD-397229 | UGACAAGAAGGCCGUUAUCCA | 2814 | 1427-1447 | UGGAUAACGGCCUUCUUGUCAGC | 2906 | 1425-1447 |
| AD-397208 | GGCCCUCGAGAAUUACAUCAA | 2815 | 1562-1582 | UUGAUGUAAUUCUCGAGGGCCAG | 2907 | 1560-1582 |
| AD-397218 | CAAGGCCUCAUCAUGUGUUCA | 2816 | 1603-1623 | UGAACACAUGAUGAGGCCUUGGG | 2908 | 1601-1623 |
| AD-397264 | CAUCAUGUGUUCAACAUGCUA | 2817 | 1611-1631 | UAGCAUGUUGAACACAUGAUGAG | 2909 | 1609-1631 |
| AD-397265 | AACAUGCUGAAGAAGUACGUA | 2818 | 1623-1643 | UACGUACUUCUUCAGCAUGUUGA | 2910 | 1621-1643 |
| AD-397209 | CAUGCUGAAGAAGUACGUCCA | 2819 | 1625-1645 | UGGACGUACUUCUUCAGCAUGUU | 2911 | 1623-1645 |
| AD-397192 | AUGCUGAAGAAGUACGUCCGA | 2820 | 1626-1646 | UCGGACGUACUUCUUCAGCAUGU | 2912 | 1624-1646 |
| AD-397210 | UGCUGAAGAAGUACGUCCGUA | 2821 | 1627-1647 | UACGGACGUACUUCUUCAGCAUG | 2913 | 1625-1647 |
| AD-397219 | GCUGAAGAAGUACGUCCGUGA | 2822 | 1628-1648 | UCACGGACGUACUUCUUCAGCAU | 2914 | 1626-1648 |
| AD-397214 | CUGAAGAAGUACGUCCGUGCA | 2823 | 1629-1649 | UGCACGGACGUACUUCUUCAGCA | 2915 | 1627-1649 |
| AD-397199 | AGCACACCCUAAAGCAUUUUA | 2824 | 1666-1686 | UAAAAUGCUUUAGGGUGUGCUGU | 2916 | 1664-1686 |
| AD-397220 | AAGCAUUUUGAACAUGUGCGA | 2825 | 1677-1697 | UCGCACAUGUUCAAAAUGCUUUA | 2917 | 1675-1697 |
| AD-397230 | AGCAUUUUGAACAUGUGCGCA | 2826 | 1678-1698 | UGCGCACAUGUUCAAAAUGCUUU | 2918 | 1676-1698 |
| AD-397221 | CACCUCCGUGUGAUCUACGAA | 2827 | 1746-1766 | UUCGUAGAUCACACGGAGGUGUG | 2919 | 1744-1766 |
| AD-397215 | CGUGUGAUCUACGAGCGCAUA | 2828 | 1752-1772 | UAUGCGCUCGUAGAUCACACGGA | 2920 | 1750-1772 |
| AD-397231 | UGUGAUCUACGAGCGCAUGAA | 2829 | 1754-1774 | UUCAUGCGCUCGUAGAUCACACG | 2921 | 1752-1774 |
| AD-397193 | GAGCGCAUGAACCAGUCUCUA | 2830 | 1764-1784 | UAGAGACUGGUUCAUGCGCUCGU | 2922 | 1762-1784 |
| AD-397190 | CGCAUGAACCAGUCUCUGUCA | 2831 | 1767-1787 | UGACAGAGACUGGUUCAUGCGCU | 2923 | 1765-1787 |
| AD-397222 | GAAGGAGCAGAACUACUCCGA | 2832 | 1850-1870 | UCGGAGUAGUUCUGCUCCUUCUG | 2924 | 1848-1870 |
| AD-397200 | AAGGAGCAGAACUACUCCGAA | 2833 | 1851-1871 | UUCGGAGUAGUUCUGCUCCUUCU | 2925 | 1849-1871 |
| AD-397201 | GGAGCAGAACUACUCCGACGA | 2834 | 1853-1873 | UCGUCGGAGUAGUUCUGCUCCUU | 2926 | 1851-1873 |
| AD-397194 | GAGCAGAACUACUCCGACGAA | 2835 | 1854-1874 | UUCGUCGGAGUAGUUCUGCUCCU | 2927 | 1852-1874 |
| AD-397248 | CAGAAUUCGGACAUGAUUCAA | 2836 | 2167-2187 | UUGAAUCAUGUCCGAAUUCUGCA | 2928 | 2165-2187 |
| AD-397207 | GUCCGCCAUCAAAAACUGGUA | 2837 | 2196-2216 | UACCAGUUUUUGAUGGCGGACUU | 2929 | 2194-2216 |
| AD-397211 | UCCGCCAUCAAAAACUGGUGA | 2838 | 2197-2217 | UCACCAGUUUUUGAUGGCGGACU | 2930 | 2195-2217 |
| AD-397243 | CAUAGCAACCGUGAUUGUCAA | 2839 | 2282-2302 | UUGACAAUCACGGUUGCUAUGAC | 2931 | 2280-2302 |
| AD-397246 | GCAACCGUGAUUGUCAUCACA | 2840 | 2286-2306 | UGUGAUGACAAUCACGGUUGCUA | 2932 | 2284-2306 |
| AD-397223 | GAAGAAACAGUACACAUCCAA | 2841 | 2321-2341 | UUGGAUGUGUACUGUUUCUUCUU | 2933 | 2319-2341 |
| AD-397202 | GAAACAGUACACAUCCAUCCA | 2842 | 2324-2344 | UGGAUGGAUGUGUACUGUUUCUU | 2934 | 2322-2344 |
| AD-397256 | GCAGCAGAACGGAUAUGAGAA | 2843 | 2405-2425 | UUCUCAUAUCCGUUCUGCUGCAU | 2935 | 2403-2425 |
| AD-397257 | GCAGAACGGAUAUGAGAAUCA | 2844 | 2408-2428 | UGAUUCUCAUAUCCGUUCUGCUG | 2936 | 2406-2428 |
| AD-397258 | CAGAACGGAUAUGAGAAUCCA | 2845 | 2409-2429 | UGGAUUCUCAUAUCCGUUCUGCU | 2937 | 2407-2429 |
| AD-397250 | AGAACGGAUAUGAGAAUCCAA | 2846 | 2410-2430 | UUGGAUUCUCAUAUCCGUUCUGC | 2938 | 2408-2430 |
| AD-397244 | GAACGGAUAUGAGAAUCCAAA | 2847 | 2411-2431 | UUUGGAUUCUCAUAUCCGUUCUG | 2939 | 2409-2431 |

TABLE 22
The AD-1397409 luciferase-targeting siRNA control of the
instant disclosure has the following sequences.

| strand | | SEQ ID NO: |
|---|---|---|
| | oligoSeq (5′-3′) | |
| sense | gsgsgag(Uhd)CfaAfAfGfuucuguuusgsa | 2949 |
| antis | VPusCfsaaaCfaGfAfacuuUfgAfcucccsasu | 2950 |
| | transSeq (5′-3′) | |
| sense | GGGAGUCAAAGUUCUGUUUGA | 2951 |
| antis | UCAAACAGAACUUUGACUCCCAU | 2952 |

### EQUIVALENTS

Those skilled in the art will recognize or be able to ascertain using no more than routine experimentation, many equivalents to the specific embodiments and methods described herein. Such equivalents are intended to be encompassed by the scope of the following claims.

We claim:

1. A method for reducing endosome size in a mammalian cell having enlarged endosomes, the method comprising contacting the mammalian cell with an amyloid precursor protein (APP)-targeting double stranded ribonucleic acid inhibitory (dsRNAi) agent in an amount sufficient to reduce endosome size in the mammalian cell, thereby reducing endosome size in the mammalian cell.

2. The method of claim 1, wherein the mammalian cell has a mutation that results in enlarged endosomes.

3. The method of claim 2, wherein the mutation that results in enlarged endosomes is selected from the group consisting of a presenilin 1 (PSEN1) mutation and an APP mutation and combinations thereof.

4. The method of claim 3, wherein the PSEN1 mutation encodes for an amino acid substitution in presenilin 1 polypeptide selected from the group consisting of A136G, A231T, A246E, A260V, A275V, A285V, A396T, A409T, A426P, A431E, A434C, A79V, C263R, C410Y, C92S, D333G, ΔD40, ΔE9, ΔI167, ΔI83/M84, ΔL166, ΔS169, ΔT440, E120D, E120K, E123K, E184D, E184G, E273A, E280A, E280G, E318G, F105I, F176L, F237I, F386S, FI77L, G183V, G206A, G206S, G209R, G209V, G217R, G266S, G378E, G378V, G384A, G394V, H131R, H163R, H163Y, H214D, I143T, I143V, I168T, I202F, I213L, I229F, I238M, I437V, I439V, InsR352, K155_insFI, K239N, L113Q, L134R, L150P, L153V, L166P, L171P, L173W, L174M, L219F, L226F, L235P, L235R, L235V, L248R, L250S, L262F, L271V, L282R, L282V, L286V, L381V, L392V, L418F, L420R, L424V, L435F, L85P, M139V, M146L, M146V, M233L, M233T, N135D, N405S, P117A, P264L, P267S, P284S, P436S, Q222R, Q223R, R108Q, R269G, R278K, R352C, R358Q, R35Q, R377W, S169P, S170F, S178P, S212Y, S230I, S365A, S390I, T116N, T147I, T245P, T274R, T291P, T354I, T99A, V261F, V272A, V391F, V412I, V82L, V89L, V94M, V96F, V97L, W165G, Y115H, Y154N, Y256S, and combinations thereof, with residue numbering as in SEQ ID NO: 3.

5. The method of claim 4, wherein the PSEN1 mutation encodes for an amino acid substitution in presenilin 1 polypeptide selected from the group consisting of M146V, L166P, M233L and A246E, with residue numbering as in SEQ ID NO: 3.

6. The method of claim 3, wherein the APP mutation encodes for an amino acid substitution in amyloid precursor protein selected from the group consisting of KM670/671NL (Swedish), A673V, D678H (Taiwanese), D678N (Tottori), E682K (Leuven), K687N, F690_V695del, A692G (Flemish), E693del, E693G, E693K, E693Q (Dutch), D694N (Iowa), T714A (Iranian), T714I (Austrian), V715A (German), V715M (French), I716F (Iberian), I716M, I716T, I716V (Florida), V717F (Indiana), V717G, V717I (London), V717L, T719N, T719P, M722K, L723P (Australian), and K724N (Belgian), with residue numbering as in SEQ ID NO: 12.

7. The method of claim 6, wherein the APP mutation encodes for an amino acid substitution in amyloid precursor protein selected from the group consisting of KM670/671NL (Swedish), A692G and V717G, with residue numbering as in SEQ ID NO: 12.

8. The method of claim 2, wherein the mammalian cell is homozygous for the mutation that results in enlarged endosomes.

9. The method of claim 1, wherein the mammalian cell is a neuronal cell, optionally a human neuronal cell, optionally a human induced pluripotent stem cell (iPSC) derived neuron.

10. The method of claim 1, wherein the amount of dsRNAi agent sufficient to reduce endosome size in the mammalian cell is less than 10 nM in the environment of the cell, optionally less than 1 nM in the environment of the cell, optionally less than 0.1 nM in the environment of the cell.

11. The method of claim 1, wherein average endosome size in the mammalian cell contacted with the dsRNAi agent is reduced by at least 30%, as compared to a mammalian cell in the absence of the dsRNAi agent, optionally wherein average endosome size in the mammalian cell contacted with the dsRNAi agent is reduced by at least 50%, as compared to a mammalian cell in the absence of the dsRNAi agent.

12. The method of claim 1, wherein the amount of dsRNAi agent is sufficient to reduce the average endosome size in a mammalian cell by at least 30%, as compared to a mammalian cell in the absence of the dsRNAi agent, optionally the amount of dsRNAi agent sufficient to reduce the average endosome size in a mammalian cell by at least 50%, as compared to a mammalian cell in the absence of the dsRNAi agent, optionally wherein endosome size is assayed via immunofluorescent imaging of Rab5.

13. The method of claim 1, wherein endosome size is determined by detecting the size of Rab5-containing intracellular compartments in the mammalian cell, optionally wherein the size of Rab5-containing intracellular compartments is determined via immunofluorescent imaging of Rab5.

14. The method of claim 1, wherein the level of one or more APP C-terminal fragment (CTF) selected from the group consisting of α-CTF and β-CTF is reduced in the contacted mammalian cell, as compared to an appropriate control mammalian cell.

15. The method of claim 1, wherein the amount of dsRNAi agent is sufficient to reduce β-CTF levels in a mammalian cell by at least 30%, as compared to a mammalian cell in the absence of the dsRNAi agent, optionally the amount of dsRNAi agent is sufficient to reduce β-CTF levels in a mammalian cell by at least 50%, as compared to a mammalian cell in the absence of the dsRNAi agent.

16. The method of claim 1, wherein the dsRNAi agent is selected from Tables 2-21.

17. The method of claim 1, wherein the mammalian cell is within a subject.

18. The method of claim 17, wherein the subject is a human.

19. The method of claim 17, wherein the subject is selected from the group consisting of a rhesus monkey, a cynomolgous monkey, a mouse, and a rat.

20. The method of claim 18, wherein the human subject suffers from an APP-associated disorder characterized by enlarged neuronal cell endosomes, optionally wherein the human subject suffers from Alzheimer's disease (AD) or Down syndrome (DS).

21. The method of claim 20, wherein the APP-associated disorder characterized by enlarged neuronal cell endosomes is AD, optionally wherein the APP-associated disorder characterized by enlarged neuronal cell endosomes is early onset familial AD (EOFAD).

22. The method of any one of the preceding claims, wherein APP expression is reduced by at least about 30% in the cell administered the APP-targeting dsRNAi agent, optionally wherein APP expression is reduced by at least about 50% in the cell administered the APP-targeting dsRNAi agent, optionally wherein APP expression is reduced by at least about 80% in the cell administered the APP-targeting dsRNAi agent.

23. A method for identifying a subject as having or at risk of developing a disease or disorder characterized by enlarged endosomes in neuronal cells and selecting a treatment for the subject, the method comprising:
a) obtaining a nucleic acid sample from the subject;
b) identifying the subject as having a mutation in presenilin 1 (PSEN1) or amyloid precursor protein (APP) associated with enlargement of endosomes in neuronal cells having the PSEN1 or APP mutation; and
c) selecting an amyloid precursor protein (APP)-targeting double stranded ribonucleic acid inhibitory (dsRNAi) agent for administration to the subject in an amount sufficient to reduce APP levels in neuronal cells of the subject,
thereby identifying the subject as having or at risk of developing a disease or disorder characterized by enlarged endosomes in neuronal cells and selecting a treatment for the subject.

24. The method of claim 23, wherein the disease or disorder characterized by enlarged endosomes in neuronal cells is Alzheimer's disease (AD), Down syndrome (DS) or frontotemporal dementia (FTD), optionally wherein the AD is early onset familial AD (EOFAD).

25. The method of claim 23, wherein the mutation in PSEN1 or APP associated with enlargement of endosomes in neuronal cells having the PSEN1 or APP mutation is a PSEN1 mutation, optionally wherein the PSEN1 mutation encodes for an amino acid substitution in presenilin 1 polypeptide selected from the group consisting of M146V, L166P and A246E, with residue numbering as in SEQ ID NO: 3.

26. The method of claim 23, wherein the mutation in PSEN1 or APP associated with enlargement of endosomes in neuronal cells having the PSEN1 or APP mutation is an APP mutation, optionally wherein the APP mutation encodes for an amino acid substitution in amyloid precursor protein selected from the group consisting of KM670/671NL (Swedish), A673V, D678H (Taiwanese), D678N (Tottori), E682K (Leuven), K687N, F690_V695del, A692G (Flemish), E693del, E693G, E693K, E693Q (Dutch), D694N (Iowa), T714A (Iranian), T714I (Austrian), V715A (German), V715M (French), I716F (Iberian), I716M, I716T, I716V (Florida), V717F (Indiana), V717G, V717I (London), V717L, T719N, T719P, M722K, L723P (Australian), and K724N (Belgian), with residue numbering as in SEQ ID NO: 12, optionally wherein the APP mutation encodes for an amino acid substitution in amyloid precursor protein selected from the group consisting of KM670/671NL (Swedish), A692G and V717G, with residue numbering as in SEQ ID NO: 12.

27. The method of claim 23, wherein the subject is homozygous for the mutation in PSEN1 or APP.

28. The method of claim 23, wherein the dsRNAi agent is selected from Tables 2-21.

29. The method of claim 23, wherein the subject is a human.

30. The method of claim 23, wherein the subject is selected from the group consisting of a rhesus monkey, a cynomolgous monkey, a mouse, and a rat.

31. The method of claim 23 further comprising administering the selected APP-targeting dsRNAi agent to the subject.

32. The method of claim 31, wherein endosome size in neuronal cells of the subject administered the selected APP-targeting dsRNAi agent is reduced, as compared to an appropriate control and/or an untreated subject, optionally wherein average endosome size in neuronal cells of the subject administered the selected APP-targeting dsRNAi agent is reduced by at least 30%, as compared to an appropriate control and/or an untreated subject, optionally wherein average endosome size in neuronal cells of the subject administered the selected APP-targeting dsRNAi agent is reduced by at least 50%, as compared to an appropriate control and/or an untreated subject.

33. The method of claim 32, wherein endosome size is determined by detecting the size of Rab5-containing intracellular compartments in the neuronal cells of the subject, optionally wherein the size of Rab5-containing intracellular compartments is determined via immunofluorescent imaging of Rab5.

34. The method of claim 31, wherein synaptic transmission of neuronal cells of the subject administered the selected APP-targeting dsRNAi agent is improved, as compared to an appropriate control and/or an untreated subject.

35. The method of claim 31, wherein a symptom of AD or DS selected from the group consisting of short-term memory and cognition is improved in the subject administered the selected APP-targeting dsRNAi agent, as compared to an appropriate control and/or an untreated subject.

36. The method of claim 31, wherein the dose of the selected APP-targeting dsRNAi agent sufficient to reduce APP levels in neuronal cells of the subject is a dose of about 0.01 mg/kg to about 50 mg/kg, optionally a dose of about 2-10 mg/kg.

37. The method of claim 31, wherein the level of one or more APP C-terminal fragment (CTF) selected from the group consisting of α-CTF and β-CTF is reduced in the subject administered the selected APP-targeting dsRNAi agent, as compared to an appropriate control and/or an untreated subject.

38. The method of claim 31, further comprising administering an additional therapeutic agent to the subject.

39. The method of claim 31, wherein the double stranded RNAi agent is administered to the subject intrathecally.

40. The method of any one of claims 31 to 39, wherein APP expression is reduced by at least about 30% in the subject administered the APP-targeting dsRNAi agent, optionally wherein APP expression is reduced by at least about 50% in the subject administered the APP-targeting dsRNAi agent, optionally wherein APP expression is reduced by at least about 80% in the subject administered the APP-targeting dsRNAi agent.

41. A method for identifying a subject as having a disease or disorder characterized by enlarged endosomes in neuronal cells and selecting a treatment for the subject, the method comprising:
a) obtaining a neuronal cell sample or fluid sample from a neuronal cell environment of the subject;
b) identifying the subject as having elevated β-CTF levels in the neuronal cell or in the fluid sample from the neuronal cell environment as an indicator for enlarged endosomes in neuronal cells of the subject; and
c) selecting an amyloid precursor protein (APP)-targeting double stranded ribonucleic acid inhibitory (dsRNAi) agent for administration to the subject in an amount sufficient to reduce β-CTF levels in neuronal cells of the subject,
thereby identifying a subject as having a disease or disorder characterized by enlarged endosomes in neuronal cells and selecting a treatment for the subject.

42. The method of claim 41, wherein the disease or disorder characterized by enlarged endosomes in neuronal cells is Alzheimer's disease (AD), Down syndrome (DS) or frontotemporal dementia (FTD), optionally wherein the AD is early onset familial AD (EOFAD).

43. The method of claim 41, wherein endosome size is determined by detecting the size of Rab5-containing intracellular compartments in the neuronal cells of the subject, optionally wherein the size of Rab5-containing intracellular compartments is determined via immunofluorescent imaging of Rab5.

44. The method of claim 41, wherein the neuronal cell sample or fluid sample from a neuronal cell environment is obtained from the central nervous system or peripheral nervous system of the subject.

45. The method of claim 41, wherein the subject has a mutation in presenilin 1 (PSEN1), optionally wherein the PSEN1 mutation encodes for an amino acid substitution in presenilin 1 polypeptide selected from the group consisting of M146V, L166P and A246E, with residue numbering as in SEQ ID NO: 3, optionally wherein the subject is homozygous for the mutation in PSEN1.

46. The method of claim 41, wherein the subject has a mutation in APP, optionally wherein the APP mutation encodes for an amino acid substitution in amyloid precursor protein selected from the group consisting of KM670/671NL (Swedish), A673V, D678H (Taiwanese), D678N (Tottori), E682K (Leuven), K687N, F690_V695del, A692G (Flemish), E693del, E693G, E693K, E693Q (Dutch), D694N (Iowa), T714A (Iranian), T714I (Austrian), V715A (German), V715M (French), I716F (Iberian), I716M, I716T, I716V (Florida), V717F (Indiana), V717G, V717I (London), V717L, T719N, T719P, M722K, L723P (Australian), and K724N (Belgian), with residue numbering as in SEQ ID NO: 12, optionally wherein the APP mutation encodes for an amino acid substitution in amyloid precursor protein selected from the group consisting of KM670/671NL (Swedish), A692G and V717G, with residue numbering as in SEQ ID NO: 12.

47. The method of claim 41, wherein the dsRNAi agent is selected from Tables 2-21.

48. The method of claim 41, wherein the subject is a human.

49. The method of claim 41, wherein the subject is selected from the group consisting of a rhesus monkey, a cynomolgous monkey, a mouse, and a rat.

50. The method of claim 41 further comprising administering the selected APP-targeting dsRNAi agent to the subject.

51. The method of claim 50, wherein average endosome size in neuronal cells of the subject administered the selected APP-targeting dsRNAi agent is reduced by at least 30%, as compared to an appropriate control and/or an untreated subject, optionally wherein average endosome size in neuronal cells of the subject administered the selected APP-targeting dsRNAi agent is reduced by at least 50%, as compared to an appropriate control and/or an untreated subject.

52. The method of claim 50, wherein synaptic transmission of neuronal cells of the subject administered the selected APP-targeting dsRNAi agent is improved, as compared to an appropriate control and/or an untreated subject.

53. The method of claim 50, wherein a symptom of AD or DS selected from the group consisting of short-term memory and cognition is improved in the subject administered the selected APP-targeting dsRNAi agent, as compared to an appropriate control and/or an untreated subject.

54. The method of claim 50, wherein the dose of the selected APP-targeting dsRNAi agent sufficient to reduce β-CTF levels in neuronal cells of the subject is a dose of about 0.01 mg/kg to about 50 mg/kg, optionally a dose of about 2-10 mg/kg.

55. The method of claim 50, wherein the level of one or more APP C-terminal fragment (CTF) selected from the group consisting of α-CTF and β-CTF is reduced in the subject administered the selected APP-targeting dsRNAi agent, as compared to an appropriate control and/or an untreated subject.

56. The method of claim 50, further comprising administering an additional therapeutic agent to the subject.

57. The method of claim 50, wherein the double stranded RNAi agent is administered to the subject intrathecally.

58. The method of any one of claims 50 to 57, wherein APP expression is reduced by at least about 30% in the subject administered the APP-targeting dsRNAi agent, optionally wherein APP expression is reduced by at least about 50% in the subject administered the APP-targeting dsRNAi agent, optionally wherein APP expression is reduced by at least about 80% in the subject administered the APP-targeting dsRNAi agent.

59. The method of claim 50, wherein the level of β-CTF is reduced in the subject administered the selected APP-targeting dsRNAi agent, as compared to an appropriate control and/or an untreated subject.

60. A method for reducing inflammation and/or expression of Iba1 mRNA in a subject having or at risk of developing Alzheimer's Disease (AD), the method comprising administering to the subject an amyloid precursor protein (APP)-targeting double stranded ribonucleic acid inhibitory (dsRNAi) agent in an amount sufficient to reduce inflammation and/or expression of Iba1 mRNA in the subject.
